(19) United States
(12) Patent Application Publication
(10) Pub. No.: US 2024/0401059 A1
(43) Pub. Date: Dec. 5, 2024

(54) COMPOSITIONS AND METHODS FOR MODULATING PNPLA3 EXPRESSION

(71) Applicant: Dicerna Pharmaceuticals, Inc., Lexington, MA (US)

(72) Inventors: Utsav Saxena, Watertown, MA (US); Henryk T. Dudek, Belmont, MA (US); Marc Abrams, Natick, MA (US); Anton Turanov, Revere, MA (US); Bob Dale Brown, Littleton, MA (US)

(21) Appl. No.: 18/793,462

(22) Filed: Aug. 2, 2024

Related U.S. Application Data
(63) Continuation of application No. 17/719,821, filed on Apr. 13, 2022, now Pat. No. 12,084,662.
(60) Provisional application No. 63/174,932, filed on Apr. 14, 2021.

(51) Int. Cl. C12N 15/113; A61K 31/7125; C07H 21/02; C12P 19/34

(52) U.S. Cl. CPC C12N 15/1137; A61K 31/7125; C07H 21/02; C12N 2310/315; C12N 2310/32; C12N 2310/321; C12N 2310/3515; C12P 19/34

(57) ABSTRACT

Oligonucleotides and compositions including the same are disclosed for inhibiting or reducing patatin-like phospholipase domain-containing protein 3 (PNPLA3) gene expression. Methods of making and using the oligonucleotides also are disclosed, particularly uses relating to treating diseases, disorders and/or conditions associated with PNPLA3 expression.

### CROSS-REFERENCE TO RELATED APPLICATIONS

This application is a continuation application of U.S. application Ser. No. 17/719,821, filed Apr. 13, 2022, which claims priority under 35 U.S.C. § 119(e) from U.S. Provisional Application No. 63/174,932, filed Apr. 14, 2021, the contents of each of which are herein incorporated by reference in their entireties.

### SEQUENCE LISTING

The instant application contains a Sequence Listing which has been submitted electronically in XML file format and is hereby incorporated by reference in its entirety. Said XML copy, created on Jul. 31, 2024, is named “400930_028USC1_211271_SL.xml” and is 1,774,047 bytes in size.

### TECHNICAL FIELD

The disclosure relates generally to biology and medicine, and more particularly it relates to the use of oligonucleotide compositions for inhibiting or reducing patatin-like phospholipase domain-containing protein 3 (PNPLA3) gene expression, as well as to uses thereof, particularly uses relating to treating diseases, disorders and/or conditions associated with PNPLA3.

### BACKGROUND

PNPLA3 is a protein encoded by PNPLA3 and is a protein having triacylglycerol lipase and acylglycerol transacylase activities. Human PNPLA3 is expressed highly in the liver and moderately in the adipose tissue, brain, kidney, and skin.

Of particular interest herein is a mutation of an isoleucine (Ile/I) to a methionine (Met/M) at position 148 in human PNPLA3 (I148M or PNPLA3 148M; i.e., PNPLA3 rs738409). See, Pingitore & Romeo (2019) BIOCHIM. BIOPHYS. ACTA MOL. CELL BIOL. LIPIDS 1864:900-906. Compared to wild-type PNPLA3, PNPLA3 148M lacks lipase activity but appears to have increased transacylase activity. See, Kumari et al. (2012) CELL METAB. 15:691-702.

PNPLA3 148M is strongly associated with a wide spectrum of liver diseases resulting from triglyceride (TG) accumulation, liver injury and fibrosis, including alcoholic hepatitis (AH), alcoholic liver disease (ALD), cirrhosis, hepatocellular carcinoma (HCC), non-alcoholic fatty liver disease (NAFLD), and non-alcoholic steatohepatitis (NASH). It appears that PNPLA3 148M degradation by proteasomes is delayed, which results in an accumulation of the protein on the surface of lipid droplets and which does not allow other proteins to metabolize TGs in hepatocytes. As such, reducing PNPLA3 148M expression may allow other lipases to function normally and reverse the adverse effects of PNPLA3 I148M across a variety of pathologies.

Several RNA-based therapeutics are known for attempting to inhibit or reduce PNPLA3 expression. For example, Intl. Patent Application Publication Nos. WO 2016/130806 and WO 2019/118638 describe double-stranded (ds) RNAi constructs for inhibiting or reducing PNPLA3 expression, as well as methods of using the same for treating or preventing liver diseases such as NAFLD. Also, Intl. Patent Application Publication No. WO 2020/061200 describes antisense oligonucleotides for inhibiting or reducing PNPLA3 expression.

Despite the existence of some therapeutics directed toward PNPLA3, there is a need for additional therapeutics for inhibiting or reducing PNPLA3 expression for treating liver disease.

### BRIEF SUMMARY

To address this need, the disclosure describes compositions and methods for treating a disease, disorder, and/or condition related to PNPLA3 expression. The disclosure is based, in part, on discovering and developing ds oligonucleotides (e.g., RNAi oligonucleotides) for selectively inhibiting and/or reducing PNPLA3 expression in, for example, the liver. Accordingly, target sequences within PNPLA3 have been identified, and RNAi oligonucleotides that bind to these target sequences and inhibit PNPLA3 mRNA expression have been generated. As shown herein, the RNAi oligonucleotides inhibit human and cynomolgus monkey PNPLA3 expression in the liver. Without being bound by theory, the RNAi oligonucleotides herein are useful for treating a disease, disorder or condition associated with PNPLA3 expression (e.g., liver disease such as AH, ALD, cirrhosis, HCC, cholangiocarcinoma (CCA), primary sclerosing cholangitis (PSC), NAFLD, and NASH). In general, the RNAi oligonucleotides herein are useful for treating a disease, disorder, or condition associated with aberrant PNPLA3 expression (e.g., mutant PNPLA3 allele expression). In particular, the RNAi oligonucleotides herein are useful for treating a disease, disorder, or condition associated with mutant PNPLA3 expression.

Accordingly, the disclosure describes RNAi oligonucleotides for reducing or inhibiting PNPLA3 expression that includes a sense strand having a sequence as set forth in Table 1 (e.g., SEQ ID NOs: 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, and 775) or Table 3 (e.g., SEQ ID NOs: 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, and 1163), especially any one of SEQ ID NOs: 787, 843, 867, 871, 937, 1003, 1007, 1017, 1161, or 1163.

Alternatively, the disclosure describes RNAi oligonucleotides for reducing or inhibiting PNPLA3 expression that includes an antisense strand having a sequence as set forth in Table 1 (e.g., SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, and 776) or Table 3 (e.g., SEQ ID NOs: 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, and 1164), especially any one of SEQ ID NOs: 788, 844, 868, 872, 938, 1004, 1008, 1018, 1162, or 1164.

In certain embodiments, the disclosure describes RNAi oligonucleotides for reducing or inhibiting PNPLA3 expression that includes a sense strand having a sequence as set forth in Table A, B, C or D (e.g., SEQ ID NOs: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, or 1300).

In certain embodiments, the disclosure describes RNAi oligonucleotides for reducing or inhibiting PNPLA3 expression that includes an antisense strand having a sequence as set forth in Table A, B, C or D (e.g., SEQ ID NOs: 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, or 1301).

In some embodiments, RNAi oligonucleotides are described for reducing or inhibiting PNPLA3 expression that include an antisense strand and a sense strand, where the antisense strand has a sequence as set forth in Table 1 or Table 3, and where the sense strand has a sequence as set forth in Table 1 or Table 3.

In some embodiments, RNAi oligonucleotides are described for reducing or inhibiting PNPLA3 expression that include an antisense strand and a sense strand, where the antisense strand has a sequence as set forth in Table A, Table B, Table C, or Table D, and where the sense strand has a sequence as set forth in Table A, Table B, Table C, or Table D.

In some embodiments, RNAi oligonucleotides are described for reducing or inhibiting PNPLA3 expression that include an antisense strand and a sense strand, where the antisense and sense strands form a duplex region, and where the antisense strand has a region of complementarity to a PNPLA3 mRNA target sequence of any one of SEQ ID NOS:1167 to 1176.

In any of the embodiments above, the antisense strand is from about 15 nucleotides to about 30 nucleotides in length. In some embodiments, the antisense strand is from about 20 nucleotides to about 25 nucleotides. In some embodiments, the antisense strand is 22 nucleotides in length.

In any of the embodiments above, the sense strand is from about 15 nucleotides to about 50 nucleotides in length. In some instances, the sense strand is from about 20 nucleotides to about 40 nucleotides in length. In some embodiments, the sense strand is 36 nucleotides in length.

In any of the embodiments above, the duplex region is from about 19 nucleotides in length to about 21 nucleotides in length. In certain embodiment, the duplex region is 20 nucleotides in length.

In any of the embodiments above, the region of complementarity is at least 15 contiguous nucleotides in length. In some embodiments, the region of complementarity is from about 19 contiguous nucleotides in length to about 21 contiguous nucleotides in length. In other embodiments, the region of complementarity is 19 contiguous nucleotides in length or 21 contiguous nucleotides in length.

In any of the embodiments above, the RNAi oligonucleotides include on the sense strand a 3′ end a stem-loop set forth as: S1-L-S2, where S1 is complementary to S2, and where L forms a loop between S1 and S2 of about 3 to about 5 nucleotides in length.

In any of the embodiments above, the antisense strand, the sense strand, or both have an overhang sequence. In some embodiments, the antisense strand includes a 3′-overhang of 1 or more nucleotides in length. In other embodiments, the 3′-overhang sequence is 2 nucleotides in length such as, for example, GG.

Oligonucleotides also are described that include an antisense strand and a sense strand, where the antisense strand can be from about 21 nucleotides to about 27 nucleotides in length and has a region of complementarity to PNPLA3, wherein the sense strand includes a stem-loop at its 3′ end set forth as: S1-L-S2, wherein S1 is complementary to S2, wherein L forms a loop between S1 and S2 from about 3 nucleotides to about 5 nucleotides in length, and wherein the antisense strand and the sense strand form a duplex structure of at least about 19 nucleotides in length but are not covalently linked.

In some embodiments, the loop L is a triloop or a tetraloop. In some instances, L is a tetraloop of 4 nucleotides in length. In other embodiments, L includes a sequence 5′-GAAA-3′.

In some embodiments, S1 and S2 are 1-10 nucleotides in length and have the same length. In other embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In other embodiments, S1 and S2 are 6 nucleotides in length. In certain embodiments, the stem-loop comprises the sequence 5′-GCAGCCGAAAGGCUGC-3′ (SEQ ID NO:1177).

In some embodiments, the antisense strand is 27 nucleotides in length and the sense strand is 25 nucleotides in length. In other embodiments, the antisense strand is 22 nucleotides in length and the sense strand is 36 nucleotides in length.

In the embodiments above, the duplex region includes a 3′-overhang sequence on the antisense strand. In some embodiments, the 3′-overhang sequence on the antisense strand is 2 nucleotides in length.

In any of the embodiments above, at least one nucleotide in an oligonucleotide is a modified nucleotide. In some instances, the modified nucleotide includes a 2′-modification such as, for example, 2′-aminoethyl, 2′-fluoro, 2′-O-methyl, 2′-O-methoxyethyl and 2′-deoxy-2′-fluoro-β-arabinonucleic acid. In certain instances, all nucleotides in an oligonucleotide include a 2′-modification such as, for example, 2′-fluoro or 2′-O-methyl.

In any of the embodiments above, at least one nucleotide in an oligonucleotide includes a modified internucleotide linkage. In some embodiments, the modified internucleotide linkage is a phosphorothioate linkage.

In any of the embodiments above, a 4′-carbon of a sugar of a 5′-nucleotide of the antisense strand includes a phosphate analog such as, for example, an oxymethylphosphonate, vinylphosphonate or malonylphosphonate. Alternatively, or optionally, the phosphate analog is a 4′-phosphate analog including 5′-methoxyphosphonate-4′-oxy.

In any of the embodiments above, at least one nucleotide of an oligonucleotide can be conjugated to one or more targeting ligands such as, for example, an amino sugar, carbohydrate, cholesterol, lipid, or polypeptide. In some embodiments, the targeting ligand is a N-acetylgalactosamine (GalNAc) moiety. In other embodiments, the GalNAc moiety is a monovalent GalNAc moiety, a bivalent GalNAc moiety, a trivalent GalNAc moiety, or a tetravalent GalNAc moiety.

In some embodiments, the targeting ligands are conjugated to one or more nucleotides of L of the stem loop. In certain instances, up to 4 nucleotides of L of the stem-loop are each conjugated to a monovalent GalNAc moiety.

In any of the embodiments above, the oligonucleotide is an RNAi oligonucleotide. In some instances, the RNAi oligonucleotide includes a sense strand having a nucleotide sequence as set forth in Table 1 or Table 3, especially any one of SEQ ID NOs: 787, 843, 867, 871, 937, 1003, 1007, 1017, 1161, or 1163. In certain embodiments, the RNAi oligonucleotide includes a sense strand having a nucleotide sequence as set forth in Table A, B, C, or D, especially any one of SEQ ID NOs: 1188, 1190, 1220, 1224, 1230, 1232, 1244, 1246, 1250, or 1254. In some instances, the RNAi oligonucleotide includes an antisense strand having a nucleotide sequence as set for the in Table 1 or Table 3, especially any one of SEQ ID NOs: 788, 844, 868, 872, 938, 1004, 1008, 1018, 1162, or 1164. In some embodiments, the RNAi oligonucleotide includes an antisense strand having a nucleotide sequence as set for the in Table A, B, C or D, especially any one of SEQ ID NOs: 1189, 1191, 1221, 1225, 1231, 1233, 1245, 1247, 1251, or 1255. In certain instances, the RNAi oligonucleotide includes a sense strand having a nucleotide sequence of any one of SEQ ID NOs: 787, 843, 867, 871, 937, 1003, 1007, 1017, 1161, or 1163, or any one of SEQ ID NOs: 1188, 1190, 1220, 1224, 1230, 1232, 1244, 1246, 1250, or 1254 and an antisense strand having a nucleotide sequence of any one of SEQ ID NOs: 788, 844, 868, 872, 938, 1004, 1008, 1018, 1162, or 1164, any one of SEQ ID NOs: 1189, 1191, 1221, 1225, 1231, 1233, 1245, 1247, 1251, or 1255. In certain embodiments, the sense strand and the antisense strand of the RNAi oligonucleotide, respectively, are selected from:
(a) SEQ ID NOs: 787 and 788,
(b) SEQ ID NOs: 843 and 844,
(c) SEQ ID NOs: 867 and 868,
(d) SEQ ID NOs: 871 and 872,
(e) SEQ ID NOs: 937 and 938,
(f) SEQ ID NOs: 1003 and 1004,
(g) SEQ ID NOs: 1007 and 1008,
(h) SEQ ID NOs: 1017 and 1018,
(i) SEQ ID NOs: 1161 and 1162, and
(j) SEQ ID NOs: 1163 and 1164.

In some instances, the RNAi oligonucleotide includes a sense strand having a nucleotide sequence as set forth in Table A, Table B, Table C, or Table D, especially any one of SEQ ID NOs: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, or 1300. In some instances, the RNAi oligonucleotide includes an antisense strand having a nucleotide sequence as set for the in Table A, Table B, Table C, or Table D, especially any one of SEQ ID NOs: 1179, 1181, 1183, 1185, 1187, 1189, 1191, 1193, 1195, 1197, 1199, 1202, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, or 1301. In certain instances, the RNAi oligonucleotide includes a sense strand having a nucleotide sequence of any one of SEQ ID NOs: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, or 1300, and an antisense strand having a nucleotide sequence of any one of SEQ ID NOs: 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, or 1301.

In certain instances, the RNAi oligonucleotide includes a sense strand having a nucleotide sequence of any one of SEQ ID NOs: 1188, 1190, 1200, 1216, 1218, 1220, 1224, 1230, 1232, 1234, 1244, 1246, 1250, 1254, 1262, 1288, 1290, 1292, 1294, 1296, 1298, or 1300, and an antisense strand having a nucleotide sequence of any one of SEQ ID NOs: 1189, 1191, 1201, 1215, 1217, 1219, 1225, 1231, 1233, 1235, 1245, 1247, 1251, 1255, 1263, 1289, 1291, 1295, 1297, 1299, or 1301.

In particular instances, the sense strand and the antisense strand of the RNAi oligonucleotide, respectively, are selected from:
(a) SEQ ID NOs: 1220 and 1221,
(b) SEQ ID NOs: 1224 and 1225,
(c) SEQ ID NOs: 1230 and 1231,
(d) SEQ ID NOs: 1232 and 1233,
(e) SEQ ID NOs: 1188 and 1189,
(f) SEQ ID NOs: 1190 and 1191,
(g) SEQ ID NOs: 1244 and 1245,
(h) SEQ ID NOs: 1250 and 1251-,
(i) SEQ ID NOs: 1254 and 1255, and
(j) SEQ ID NOs: 1246 and 1247.

Oligonucleotides also are described for inhibiting or reducing PNPLA3 expression that include a sense strand and an antisense strand, where the sense strand and the antisense strand form a duplex region, where all nucleotides of the sense strand and the antisense strand include a modification of a base, a sugar and/or an internucleotide linkage, where the antisense strand includes a region of complementarity to a PNPLA3 mRNA target sequence of one of SEQ ID NOs: 1167 to 1176, and where the region of complementarity is at least about 15 contiguous nucleotides in length.

In other aspects, pharmaceutical compositions are described that include at least one oligonucleotide herein and a pharmaceutically acceptable carrier, delivery agent or excipient. In some instances, the pharmaceutical compositions include an additional therapeutic agent such as, for example, an antidiabetic agent or anti-obesity agent.

In other aspects, methods are described for reducing PNPLA3 expression in a cell, a population of cells, a tissue, an organ, or an individual that include at least a step of administering/contacting the cell, the population of cells, the tissue, the organ, or the individual with an oligonucleotide herein or a pharmaceutical composition herein. In some instances, reducing PNPLA3 expression includes reducing an amount or level of PNPLA3 mRNA, an amount or level of PNPLA3 protein, or both in the cell, the population of cells, the tissue, the organ, or the individual. In some instances, the cell, the cell population, the tissue, the organ, or the individual has a disease, disorder, or condition associated with PNPLA3 expression. In certain instances, the disease, disorder, or condition associated with PNPLA3 expression is a cardiometabolic disease, AH, ALD, cirrhosis, HCC, CCA, and other cholangiopathies (such as PSC), NAFLD, and NASH.

In other aspects, methods are described for treating an individual having or suspected of having a disease, disorder or condition associated with PNPLA3 expression. The methods include at least a step of administering to an individual in need thereof an effective amount of an oligonucleotide herein or a pharmaceutical composition herein. In some instances, the disease, disorder, or condition associated with PNPLA3 expression is a cardiometabolic disease, AH, ALD, CCA, cirrhosis, HCC, NAFLD, PSC, and NASH. In some instances, the oligonucleotide or pharmaceutical composition is administered daily, weekly, monthly, quarterly, yearly via SQ administration, especially monthly or quarterly.

In some instances, the individual has cirrhosis, diabetes, hepatic fibrosis, hepatic inflammation, hyperlipidemia, AH, ALD, CCA, cirrhosis, HCC, NAFLD, PSC, NASH, obesity, and/or steatosis.

In any of the embodiments above, the methods comprise additional steps such as measuring or obtaining genotype information, PNPLA3 expression, PNPLA3 protein levels, the individual's weight and/or blood glucose and/or TGs and comparing such obtained values to one or more baseline values or previously obtained values to assess the effectiveness of contacting or administering. In some embodiments, the additional step comprises confirming that the individual has a PNPLA3 I148M variant. In some embodiments, the additional step comprises confirming that the individual does not have a PNPLA3 E434K variant. In some embodiments, the additional step comprises confirming that the individual does not have a protein truncating HSD17B13 variant (rs72613567).

In any of the embodiments above, the methods can include administering the RNAi oligonucleotide or pharmaceutical composition simultaneously, separately, or sequentially with a second composition or a second therapeutic agent. In some embodiments, the second composition or a second therapeutic agent is a PNPLA3 antibody or fragment thereof, an antidiabetic agent or anti-obesity agent. In some embodiments, the second composition or second therapeutic agent is administered with a frequency same as the RNAi oligonucleotide (i.e., every other day, twice a week, or even weekly). In other embodiments, the second composition or second therapeutic agent is administered with a frequency distinct from the RNAi oligonucleotide. Likewise, in other embodiments, the second composition or second therapeutic agent is administered via the same route as the RNAi oligonucleotide (e.g., SQ). In still other embodiments, the second composition or second therapeutic agent is administered via a route that differs from the RNAi oligonucleotide).

In other aspects, uses are described for the RNAi oligonucleotides herein for treating a disease, disorder or condition associated with PNPLA3 expression, which optionally are administered simultaneously, separately, or sequentially (i.e., in combination) with a second composition or second therapeutic agent.

In other aspects, uses are described for the RNAi oligonucleotides herein in manufacturing a medicament for treating a disease, disorder, or condition associated with PNPLA3 expression, where the medicament optionally further includes a second composition or second therapeutic agent.

In other aspects, kits are described that include at least one oligonucleotide herein, an optional pharmaceutically acceptable carrier, and a package insert having instructions for administering the same to an individual having a disease, disorder, or condition associated with PNPLA3 expression.

An advantage of the oligonucleotides and compositions herein is that suppressed PNPLA3 expression, especially PNPLA3 148M, exerts a beneficial effect on the entire spectrum of NAFLD including fibrosis.

### BRIEF DESCRIPTION OF THE DRAWINGS

The advantages, effects, features, and objects other than those set forth above will become more readily apparent when consideration is given to the detailed description below. Such detailed description refers to the following drawing(s), where:

FIG. 1 discloses a schematic depicting the structure and chemical modification pattern of a generic GalNAc-conjugated PNPLA3 oligonucleotide. FIG. 1 discloses SEQ ID NOS 1302-1303, respectively, in order of appearance. The sense strand has a modification pattern M1405, wherein nucleotides at positions 1, 2, 3, 4, 5, 6, 7, 12, 13, 14, 15, 16, 17, 18, 19, 20, 21, 22, 23, 24, 25, 26, 27, 31, 32, 33, 34, 35, and 36 are modified with 2′-O-methyl; nucleotides at positions 8, 9, 10, and 11 are modified with 2′-fluoro; the internucleotide linkage between nucleotides at positions 1 and 2 is a phosphorothioate linkage; and nucleotides at positions 28, 29, and 30 are modified with adem-GalNAc. The antisense strand has a modification pattern M2028, wherein nucleotides at positions 1, 6, 8, 9, 11, 12, 13, 15, 16, 17, 18, 19, 20, 21, and 22 are modified with 2′-O-methyl; nucleotides at positions 2, 3, 4, 5, 7, 10, and 14 are modified with 2′-fluoro; the internucleotide linkages between nucleotides at positions 1 and 2, positions 2 and 3, positions 3 and 4, positions 20 and 21, and positions 21 and 22 are phosphorothioate linkages; and nucleotide at position 1 is a 4′-phosphate analog comprising 5′-methoxyphosphonate-4′-oxy.

FIG. 2 discloses a schematic depicting the structure and chemical modification pattern of an alternative generic GalNAc-conjugated PNPLA3 oligonucleotide. The sense strand has a modification pattern M107, wherein nucleotides at positions 2, 4, 14 and 16 are modified with 2′-O-methyl; nucleotides at positions 1, 3, 5, 6, 7, 8, 9, 10, 11, 12, 13, 15, 17, 18, 19, 20, 21, 22 and 23 are RNA nucleotides; and nucleotides at positions 24 and 25 are DNA nucleotides. The antisense strand has a modification pattern M48, wherein nucleotides at positions 1, 2, 3, 4, 11, 13, 23, 25, 26 and 27 are modified with 2′-O-methyl; and nucleotides at positions 5, 6, 7, 8, 9, 10, 12, 14, 15, 16, 17, 18, 19, 20, 21, 22 and 24 are RNA nucleotides.

### DETAILED DESCRIPTION

### Overview

ALD and NAFLD are serious public health burdens. ALD and NAFLD are chronic liver disorders that begin with hepatic TG accumulation (steatosis) and progress to hepatic inflammation and fibrosis, cirrhosis, and even liver cancer. PNPLA3 148M is a genetic factor that has been shown to be associated with ALD and NAFLD, as well, as cirrhosis, HCC, and liver-related death.

RNA interference (RNAi) is a process of introducing exogeneous RNA into a cell leading to specific degradation of the mRNA encoding the targeted protein with a resultant decrease in target gene expression.

In humans, PNPLA3 is 481 amino acids in length with a predicted molecular weight of 52.865 kD. Exemplary nucleic acid sequences for PNPLA3 can be found in NCBI Ref. Seq. No. NM_025225 (human), NM_054088 or XM_006520346 (mouse), NM_001282324 (rat), XM_015457081 (primate), XM_005567051 (primate) and XM_001109144 (primate). One of skill in the art, however, understands that additional examples of PNPLA3 mRNA sequences are readily available using publicly available databases such as, for example, GenBank and UniProt.

### Definitions

As used herein, “about” means within a statistically meaningful range of a value or values such as, for example, a stated concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the system under study, and can be readily appreciated by one of skill in the art.

As used herein, “administer,” “administering,” “administration” and the like refers to providing a substance (e.g., an oligonucleotide herein or a composition herein) to an individual in a manner that is pharmacologically useful (e.g., to treat a disease, disorder, or condition in the individual).

As used herein, “antisense strand” means an oligonucleotide herein that is complimentary to a region of a target sequence. Likewise, and as used herein, “sense strand” means an oligonucleotide herein that is complimentary to a region of an antisense strand.

As used herein, “asialoglycoprotein receptor” or “ASGPR” means a bipartite C-type lectin formed by a major 48 kDa subunit (ASGPR-1) and minor 40 kDa subunit (ASGPR-2). ASGPR is primarily expressed on the sinusoidal surface of hepatocyte cells and has a major role in binding, internalizing and subsequent clearing of circulating glycoproteins that contain terminal galactose or GalNAc residues (asialoglycoproteins).

As used herein, “attenuate,” “attenuating,” “attenuation” and the like refers to reducing or effectively halting. As a non-limiting example, one or more of the treatments herein may reduce or effectively halt the onset or progression of AH, ALD, CCA, PSC, cirrhosis, HCC, NAFLD, and NASH, as well as related diseases, disorders, and conditions in an individual. This attenuation may be exemplified by, for example, a decrease in one or more aspects (e.g., symptoms, tissue characteristics, and cellular, inflammatory, or immunological activity, etc.) of AH, ALD, cirrhosis, HCC, CCA, PSC, NAFLD, and NASH, as well as related diseases, disorders and conditions, no detectable progression (worsening) of one or more aspects of AH, ALD, cirrhosis, HCC, CCA, PSC, NAFLD, and NASH, as well as related diseases, disorders and conditions, or no detectable aspects of AH, ALD, cirrhosis, HCC, NAFLD, and NASH, as well as related diseases, disorders and conditions in an individual when they might otherwise be expected.

As used herein, “attenuate,” “attenuating,” “attenuation” and the like means reducing or effectively halting. As a non-limiting example, one or more of the treatments herein may reduce or effectively halt the onset or progression of dyslipidemia/hypertriglyceridemia/hyperlipidemia in a subject. This attenuation may be exemplified by, for example, a decrease in one or more aspects (e.g., symptoms, tissue characteristics, and cellular, inflammatory, or immunological activity, etc.) of AH, ALD, cirrhosis, HCC, CCA, PSC, NAFLD, and NASH, as well as related diseases, disorders, and conditions in an individual when they might otherwise be expected.

As used herein, “complementary” means a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “contact,” “contacting” and the like means directly or indirectly introducing or delivering the RNAi into, for example, a cell by facilitating or effecting uptake or absorption into the cell.

As used herein, “deoxyribonucleotide” means a nucleotide having a hydrogen in place of a hydroxyl at the 2′ position of its pentose sugar when compared with a ribonucleotide. A modified deoxyribonucleotide has one or more modifications substitutions of atoms other than at the 2′ position, including modifications or substitutions in or of the nucleobase, sugar, or phosphate group.

As used herein, “double-stranded oligonucleotide” or “ds oligonucleotide” means an oligonucleotide that is substantially in a duplex form. The complementary base-pairing of duplex region(s) of a ds oligonucleotide can be formed between antiparallel sequences of nucleotides of covalently separate nucleic acid strands. Likewise, complementary base-pairing of duplex region(s) of a ds oligonucleotide can be formed between antiparallel sequences of nucleotides of nucleic acid strands that are covalently linked. Moreover, complementary base-pairing of duplex region(s) of a ds oligonucleotide can be formed from single nucleic acid strand that is folded (e.g., via a hairpin) to provide complementary antiparallel sequences of nucleotides that base pair together. A ds oligonucleotide can include two covalently separate nucleic acid strands that are fully duplexed with one another. However, a ds oligonucleotide can include two covalently separate nucleic acid strands that are partially duplexed (e.g., having overhangs at one or both ends). A ds oligonucleotide can include an antiparallel sequence of nucleotides that are partially complementary, and thus, may have one or more mismatches, which may include internal mismatches or end mismatches.

As used herein, “duplex,” in reference to nucleic acids (e.g., oligonucleotides), means a structure formed through complementary base pairing of two antiparallel sequences of nucleotides.

As used herein, “excipient” means a non-therapeutic agent that may be included in a composition herein, for example, to provide or contribute to a desired consistency or stabilizing effect.

As used herein, “hepatocyte” or “hepatocytes” means cells of the parenchymal tissues of the liver. These cells make up about 70%-85% of the liver's mass and manufacture serum albumin, fibronectin (FBN) and the prothrombin group of clotting factors (except for Factors 3 and 4). Markers for hepatocyte lineage cells include, but are not limited to, transthyretin (Ttr), glutamine synthetase (Glul), hepatocyte nuclear factor 1a (Hnf1a) and hepatocyte nuclear factor 4a (Hnf4a). Markers for mature hepatocytes may include, but are not limited to, cytochrome P450 (Cyp3a11), fumarylacetoacetate hydrolase (Fah), glucose 6-phosphate (G6p), albumin (Alb) and OC2-2F8. See, e.g., Huch et al. (2013) NATURE 494:247-50.

As used herein, a “hepatotoxic agent” means a chemical compound, virus or other substance that is itself toxic to the liver or can be processed to form a metabolite that is toxic to the liver. Hepatotoxic agents may include, but are not limited to, carbon tetrachloride (CCl₄), acetaminophen (paracetamol), vinyl chloride, arsenic, chloroform, nonsteroidal anti-inflammatory drugs (such as aspirin and phenylbutazone).

As used herein, “labile linker” means a linker that can be cleaved (e.g., by acidic pH). Likewise, “fairly stable linker” means a linker that cannot be cleaved.

As used herein, “liver inflammation” or “hepatitis” means a physical condition in which the liver becomes swollen, dysfunctional and/or painful, especially as a result of injury or infection, as may be caused by exposure to a hepatotoxic agent. Symptoms may include jaundice, fatigue, weakness, nausea, vomiting, appetite reduction and weight loss. Liver inflammation, if left untreated, may progress to fibrosis, cirrhosis, liver failure or liver cancer.

As used herein, “liver fibrosis,” “hepatic fibrosis” or “fibrosis of the liver” refers to an excessive accumulation in the liver of extracellular matrix proteins, which could include collagens (I, III, and IV), FBN, undulin, elastin, laminin, hyaluronan, and proteoglycans resulting from inflammation and liver cell death. Liver fibrosis, if left untreated, may progress to cirrhosis, liver failure or liver cancer.

As used herein, “loop” means an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “modified internucleotide linkage” means an internucleotide linkage having one or more chemical modifications when compared with a reference internucleotide linkage having a phosphodiester bond. A modified nucleotide can be a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” mean a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “nucleotide” means an organic molecule having a nucleoside (a nucleobase such as, for example, adenine, cytosine, guanine, thymine, or uracil; and a pentose sugar such as, for example, ribose or 2′-deoxyribose; and a phosphate group, which can serve as a monomeric unit of nucleic acid polymers such as deoxyribonucleic acid (DNA) and ribonucleic acid (RNA).

As used herein, “oligonucleotide” means a short nucleic acid molecule (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide (ASO), short siRNA, or ss siRNA. Typically, a ds oligonucleotide is a RNAi oligonucleotide.

As used herein, “overhang” means a terminal non-base pairing nucleotide(s) resulting from one strand or region extending beyond the terminus of a complementary strand with which the one strand or region forms a duplex. An overhang may include one or more unpaired nucleotides extending from a duplex region at the 5′ terminus or 3′ terminus of a ds oligonucleotide. The overhang can be a 3′ or 5′ overhang on the antisense strand or sense strand of a ds oligonucleotides.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) NUCLEIC ACIDS RES. 43:2993-3011).

As used herein, “PNPLA3-associated disease,” “PNPLA3-associated disorder” or “PNPLA3-associated condition” means conditions where increased PNPLA3 expression and/or the presence of, for example, the PNPLA3 I148M variant. Exemplary PNPLA3-associated conditions, diseases or disorders include, but are not limited to, accumulation of fat in the liver, cirrhosis of the liver, fatty liver (steatosis), hepatocellular necrosis, HCC, liver fibrosis, inflammation of the liver, NASH, NAFLD, or obesity.

As used herein, “reduced expression,” and with respect to a gene (e.g., PNPLA3) means a decrease in the amount or level of RNA transcript (e.g., PNPLA3 mRNA) or protein encoded by the gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide herein (e.g., an oligonucleotide having an antisense strand having a nucleotide sequence that is complementary to a nucleotide sequence including PNPLA3 mRNA) may result in a decrease in the amount or level of mRNA, protein, and/or activity (e.g., via degradation of PNPLA3 mRNA by the RNAi pathway) when compared to a cell that is not treated with the ds oligonucleotide. Similarly, and as used herein, “reducing expression” means an act that results in reduced expression of a gene (e.g., PNPLA3). Specifically, and as used herein, “reduction of PNPLA3 expression” means a decrease in the amount or level of PNPLA3 mRNA, PMPLA3 protein, and/or PNPLA3 activity in a cell, a population of cells, a sample, or a subject when compared to an appropriate reference (e.g., a reference cell, population of cells, tissue, or subject).

As used herein, “region of complementarity” means a sequence of nucleotides of a nucleic acid (e.g., a ds oligonucleotide) that is sufficiently complementary to an antiparallel sequence of nucleotides to permit hybridization between the two sequences of nucleotides under appropriate hybridization conditions (e.g., in a phosphate buffer, in a cell, etc.). An oligonucleotide herein includes a targeting sequence having a region of complementary to a mRNA target sequence.

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the nucleobase, sugar, or phosphate group.

As used herein, “RNAi oligonucleotide” refers to either (a) a ds oligonucleotide having a sense strand (passenger) and antisense strand (guide), in which the antisense strand or part of the antisense strand is used by the Argonaute 2 (Ago2) endonuclease in the cleavage of a target mRNA or (b) a ss oligonucleotide having a single antisense strand, where that antisense strand (or part of that antisense strand) is used by the Ago2 endonuclease in the cleavage of a target mRNA.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “subject” means any mammal, including cats, dogs, mice, rats, and primates, especially humans. Moreover, “individual” or “patient” may be used interchangeably with “subject.”

As used herein, “synthetic” refers to a nucleic acid or other molecule that is artificially synthesized (e.g., using a machine such as, for example, a solid-state nucleic acid synthesizer) or that is otherwise not derived from a natural source (e.g., a cell or organism) that normally produces the nucleic acid or other molecule.

As used herein, “targeting ligand” means a molecule (e.g., an amino sugar, carbohydrate, cholesterol, lipid, or polypeptide) that selectively binds to a cognate molecule (e.g., a receptor) of a tissue or cell of interest and that is conjugatable to another substance for targeting another substance to the tissue or cell of interest. For example, a targeting ligand may be conjugated to an oligonucleotide herein for purposes of targeting the oligonucleotide to a specific tissue or cell of interest. A targeting ligand can selectively bind to a cell surface receptor. Accordingly, a targeting ligand, when conjugated to an oligonucleotide, facilitates delivery of the oligonucleotide into a particular cell through selective binding to a receptor expressed on the surface of the cell and endosomal internalization by the cell of the complex comprising the oligonucleotide, targeting ligand, and receptor. Moreover, a targeting ligand can be conjugated to an oligonucleotide via a linker that is cleaved following or during cellular internalization such that the oligonucleotide is released from the targeting ligand in the cell.

As used herein, “tetraloop” means a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C., or at least about 75° C. in 10 mM NaHPO₄ to a hairpin comprising a duplex of at least 2 base pairs (bp) in length. A tetraloop also may stabilize a bp in an adjacent stem duplex by stacking interactions. Additionally, interactions among the nucleotides in a tetraloop include, but are not limited to, non-Watson-Crick base pairing, stacking interactions, hydrogen bonding, and contact interactions (Cheong et al. (1990) NATURE 346:680-682; Heus & Pardi (1991) SCIENCE 253:191-194). Here, a tetraloop can include or can have about 3 to 6 nucleotides, and typically is about 4 to 5 nucleotides. A tetraloop therefore can have 3, 4, 5, or 6 nucleotides, which may or may not be modified (e.g., which may or may not be conjugated to a targeting moiety), especially 4 nucleotides. Any nucleotide may be used in the tetraloop, and standard IUPAC-IUB symbols for such nucleotides may be used as described in Cornish-Bowden (1985) NUCLEIC ACIDS RES. 13:3021-30. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA) and the CUUG tetraloop (Woese et al. (1990) PROC. NATL. ACAD. SCI. USA 87:8467-71; Antao et al. (1991) NUCLEIC ACIDS RES. 19:5901-05). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, e.g., Nakano et al. (2002) BIOCHEM. 41:4281-92; and Shinji et al. (2000) NIPPON KAGAKKAI KOEN YOKOSHU 78:731. Here, the tetraloop can be within a nicked tetraloop structure.

As used herein, “treat” or “treating” means an act of providing care to an individual in need thereof, for example, by administering a therapeutic agent (e.g., an oligonucleotide herein) to the individual for purposes of improving the health and/or well-being of the individual with respect to an existing condition (e.g., a disease, disorder) or to prevent or decrease the likelihood of the occurrence of a condition. Treating also can involve reducing the frequency or severity of at least one sign, symptom or contributing factor of a condition (e.g., disease, disorder) experienced by the individual.

As used herein, “iRNA,” “iRNA agent,” “RNAi,” “RNAi agent” and “RNA interference agent” means an agent that contains RNA and that mediates the targeted cleavage of an RNA transcript via an RNA-induced silencing complex (RISC) pathway. iRNA directs sequence-specific degradation of mRNA via RNA interference. The iRNA modulates, inhibits, or reduces PNPLA3 expression in a cell.

### Compositions

According to some aspects, the disclosure provides oligonucleotides (e.g., double-stranded RNAi oligonucleotides) that reduce, modulate, or inhibit expression of PNPLA3 in the liver. In some embodiments, the oligonucleotides provided herein used to treat of diseases associated with PNPLA3 expression. In some aspects, the disclosure provides methods of treatment a disease associated with PNPLA3 expression by reducing, modulating, or inhibiting PNPLA3 expression in the liver (e.g., in cells comprising the liver).

### Oligonucleotide Inhibitors of PNPLA3 Expression

I. PNPLA3 Target Sequences: The oligonucleotides herein (e.g., RNAi oligonucleotides) are targeted to a target sequence comprising PNPLA3 mRNA (i.e., a PNPLA3 target sequence). In some embodiments, the oligonucleotide or a portion, fragment, or strand thereof (e.g., an antisense strand or a guide strand of a ds RNAi oligonucleotide) binds or anneals to a PNPLA3 target sequence, thereby inhibiting PNPLA3 expression. In some embodiments, the oligonucleotide is targeted to a PNPLA3 target sequence for inhibiting PNPLA3 expression in vivo. In some embodiments, the amount or extent of PNPLA3 expression inhibition by an oligonucleotide targeted to a PNPLA3 target sequence correlates with the potency of the oligonucleotide. In some embodiments, the amount or extent of inhibition of PNPLA3 expression by an oligonucleotide targeted to a PNPLA3 target sequence correlates with the amount or extent of therapeutic benefit in an individual having or suspected of having a disease, disorder, or condition associated with PNPLA3 expression treated with the oligonucleotide.

Through examining and analyzing the nucleotide sequence of PNPLA3 mRNAs, including mRNAs of multiple different species (e.g., human, cynomolgus monkey, and rhesus monkey; see, e.g., Example 1) and as a result of in vitro and in vivo testing (see, e.g., Examples 2-3), it is shown herein that certain nucleotide sequences of PNPLA3 mRNA are more amenable than others to oligonucleotide-based inhibition of PNPLA3 expression and are thus useful as target sequences for the oligonucleotides herein. In some embodiments, a sense strand of an oligonucleotide (e.g., a ds RNAi oligonucleotide) described herein (e.g., in Tables 1 or 3, or tables A, B, C or D) comprises a PNPLA3 target sequence. In some embodiments, a portion or region of the sense strand of an oligonucleotide described herein (e.g., in Tables 1 or 3, or tables A, B, C or D) comprises a PNPLA3 target sequence. In some embodiments, the PNPLA3 target sequence comprises, or consists of, a sequence of any one of SEQ ID NOs:1167 to 1176.

II. PNPLA3 mRNA Targeting Sequences: In some embodiments, the oligonucleotides herein have regions of complementarity to PNPLA3 mRNA (e.g., within a target sequence of PNPLA3 mRNA) for targeting PNPLA3 mRNA in cells and inhibiting PNPLA3 expression. In some embodiments, the oligonucleotides herein comprise a PNPLA3 targeting sequence (e.g., an antisense strand or a guide strand of a ds oligonucleotide) having a region of complementarity that binds or anneals to a PNPLA3 mRNA target sequence by complementary (Watson-Crick) base pairing. The targeting sequence or region of complementarity is of a suitable length and base content to enable binding or annealing of the oligonucleotide (or a strand thereof) to PNPLA3 mRNA for inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 nucleotides in length. Alternatively, the targeting sequence or region of complementarity is about 12 to about 30 (e.g., 12 to 30, 12 to 22, 15 to 25, 17 to 21, 18 to 27, 19 to 27, or 15 to 30) nucleotides in length. Alternatively, the targeting sequence or region of complementarity is about 12, 13, 14, 15, 16, 17, 18, 19, 20, 21, 22, 23, 24, 25, 26, 27, 28, 29, or 30 nucleotides in length. In certain embodiments, the targeting sequence or region of complementarity is 18 nucleotides in length. In certain embodiments, the targeting sequence or region of complementarity is 19 nucleotides in length. In certain embodiments, the targeting sequence or region of complementarity is 20 nucleotides in length. In certain embodiments, the targeting sequence or region of complementarity is 21 nucleotides in length. In certain embodiments, the targeting sequence or region of complementarity is 22 nucleotides in length. In certain embodiments, the targeting sequence or region of complementarity is 23 nucleotides in length. In certain embodiments, the targeting sequence or region of complementarity is 24 nucleotides in length.

In some embodiments, the oligonucleotides herein comprise a targeting sequence or a region of complementarity (e.g., an antisense strand or a guide strand of a double-stranded oligonucleotide) that is fully complementary to PNPLA3 target sequence. In some embodiments, the targeting sequence or region of complementarity is partially complementary to a PNPLA3 target sequence. In some embodiments, the oligonucleotide comprises a targeting sequence or region of complementarity that is fully complementary to a sequence of any one of SEQ ID NOs: 1167 to 1176. In some embodiments, the oligonucleotide comprises a targeting sequence or region of complementarity that is partially complementary to a sequence of any one of SEQ ID NOs: 1167 to 1176.

Alternatively, in some embodiments, the oligonucleotides herein comprise a targeting sequence or region of complementarity that is complementary to a contiguous sequence of nucleotides comprising a PNPLA3 mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20, or 18 to 19 nucleotides in length). In some embodiments, the oligonucleotides comprise a targeting sequence or region of complementarity that is complementary to a contiguous sequence of nucleotides comprising a PNPLA3 mRNA, where the contiguous sequence of nucleotides is 10, 11, 12, 13, 14, 15, 16, 17, 18, 19, or 20 nucleotides in length. In some embodiments, the oligonucleotides comprise a targeting sequence or region of complementarity that is complementary to a contiguous sequence of nucleotides comprising a PNPLA3 mRNA, where the contiguous sequence of nucleotides is 19 nucleotides in length. In some embodiments, the oligonucleotides comprise a targeting sequence or region of complementarity that is complementary to a contiguous sequence of nucleotides comprising a PNPLA3 mRNA, where the contiguous sequence of nucleotides is 20 nucleotides in length. In other embodiments, the oligonucleotides comprise a targeting sequence or a region of complementarity that is complementary to a contiguous sequence of nucleotides of any one of SEQ ID NOs: 1167 to 1176, optionally where the contiguous sequence of nucleotides is 19 nucleotides in length.

With regard to the targeting sequence or region of complementarity of the oligonucleotides herein, it is complementary to contiguous nucleotides of a sequence as set forth in any one of SEQ ID NOs: 1167 to 1176 and spans the entire length of an antisense strand. In some embodiments, the region of complementarity of the oligonucleotides is complementary to contiguous nucleotides of a sequence as set forth in any one of SEQ ID NOs: 1167 to 1176 and spans a portion of the entire length of an antisense strand. In some additional embodiments, the oligonucleotides include a region of complementarity (e.g., on an antisense strand of a ds oligonucleotide) that is at least partially (e.g., fully) complementary to a contiguous stretch of nucleotides spanning nucleotides 1-20 of a sequence as set forth in any one of SEQ ID NOs: 1167 to 1176.

Alternatively, the oligonucleotides herein comprise a targeting sequence or region of complementarity having one or more base pair (bp) mismatches with the corresponding PNPLA3 target sequence. In some embodiments, the targeting sequence or region of complementarity is up to about 1, up to about 2, up to about 3, up to about 4, up to about 5, etc. mismatches with the corresponding PNPLA3 target sequence provided that the ability of the targeting sequence or region of complementarity to bind or anneal to the PNPLA3 mRNA under appropriate hybridization conditions and/or the ability of the oligonucleotide to reduce or inhibit PNPLA3 expression is maintained. Stated differently, the targeting sequence or region of complementarity is no more than 1, no more than 2, no more than 3, no more than 4, or no more than 5 mismatches with the corresponding PNPLA3 target sequence provided that the ability of the targeting sequence or region of complementarity to bind or anneal to the PNPLA3 mRNA under appropriate hybridization conditions and/or the ability of the oligonucleotides to reduce or inhibit PNPLA3 expression is maintained. In some embodiments, the oligonucleotides comprise a targeting sequence or region of complementarity having 1 mismatch with the corresponding target sequence. In some embodiments, the oligonucleotides comprise a targeting sequence or region of complementarity having 2 mismatches with the corresponding target sequence. In some embodiments, the oligonucleotides comprise a targeting sequence or a region of complementarity having 3 mismatches with the corresponding target sequence. In some embodiments, the oligonucleotides comprise a targeting sequence or region of complementarity having 4 mismatches with the corresponding target sequence. In some embodiments, the oligonucleotides comprise a targeting sequence or region of complementarity having 5 mismatches with the corresponding target sequence. In other embodiments, the oligonucleotides comprise a targeting sequence or a region of complementarity more than one mismatch (e.g., 2, 3, 4, 5 or more mismatches) with the corresponding target sequence, where at least 2 (e.g., all) of the mismatches are positioned consecutively (e.g., 2, 3, 4, 5 or more mismatches in a row), or where the mismatches are interspersed in any position throughout the targeting sequence or region of complementarity. In other embodiments, the oligonucleotides comprise a targeting sequence or region of complementarity more than one mismatch (e.g., 2, 3, 4, 5 or more mismatches) with the corresponding target sequence, where at least 2 (e.g., all) of the mismatches are positioned consecutively (e.g., 2, 3, 4, 5 or more mismatches in a row), or where at least one or more non-mismatched base pair is located between the mismatches, or a combination thereof.

III. Types of Oligonucleotides: A variety of oligonucleotide types and/or structures are useful for targeting PNPLA3 mRNA including, but not limited to, RNAi oligonucleotides, antisense oligonucleotides, miRNAs, etc. Any of the oligonucleotide types described herein or elsewhere are contemplated for use as a framework to incorporate a targeting sequence herein for the purposes of inhibiting PNPLA3 expression. In some embodiments, the oligonucleotides herein inhibit PNPLA3 expression by engaging with RNAi pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended ds oligonucleotides where at least one end of at least one strand is extended beyond a duplex targeting region, including structures where one of the strands includes a thermodynamically stabilizing tetraloop structure (see, e.g., U.S. Pat. Nos. 8,513,207 and 8,927,705, as well as Intl. Patent Application Publication No. WO 2010/033225). Such structures include ss extensions (on one or both sides of the molecule) as well as ds extensions.

The oligonucleotides herein engage with the RNAi pathway downstream of the involvement of Dicer (e.g., Dicer cleavage). In some embodiments, the oligonucleotides have an overhang (e.g., of 1, 2, or 3 nucleotides in length) in the 3′ end of the sense strand. In some embodiments, the oligonucleotides (e.g., siRNA) include a 21-nucleotide guide strand that is antisense to a target mRNA (e.g., PNPLA3 mRNA) and a complementary passenger strand, in which both strands anneal to form a 19-bp duplex and 2 nucleotide overhangs at either or both 3′ ends. Longer oligonucleotide designs also are contemplated, including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

The oligonucleotides herein comprise sense and antisense strands that are both in the range of about 17 to about 26 (e.g., 17 to 26, 20 to 25, or 21-23) nucleotides in length. In some embodiments, the oligonucleotides comprise a sense and antisense strand that are both in the range of about 19 to about 22 nucleotides in length. In some embodiments, the sense and antisense strands are of equal length. In some embodiments, the oligonucleotides comprise sense and antisense strands, such that there is a 3′-overhang on either the sense strand or the antisense strand, or both the sense and antisense strand. In some instances, for oligonucleotides having sense and antisense strands that are both in the range of about 21 to about 23 nucleotides in length, a 3′-overhang on the sense, antisense or both sense and antisense strands is 1 or 2 nucleotides in length. In some embodiments, the oligonucleotides comprise a guide strand of 22 nucleotides and a passenger strand of 20 nucleotides, where there is a blunt end on the right side of the molecule (3′ end of passenger strand/5′ end of guide strand) and a 2 nucleotide 3′ guide strand overhang on the left side of the molecule (5′ end of the passenger strand/3′ end of the guide strand). In such molecules, there is a 20-bp duplex region.

Other oligonucleotide designs for use herein include: 16-mer siRNAs (see, e.g., “NUCLEIC ACIDS IN CHEMISTRY & BIOLOGY,” Blackburn (ed.), Royal Society of Chemistry, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-32), fork siRNAs (see, e.g., Hohjoh (2004) FEBS LETT. 557:193-198), ss siRNAs (see, e.g., Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (sisiRNA; see, e.g., Bramsen et al. (2007) NUCLEIC ACIDS RES. 35:5886-97). Further non-limiting examples of oligonucleotide structures that may be used herein to reduce or inhibit PNPLA3 expression are miRNA, shRNA, and short siRNA (see, e.g., Hamilton et al. (2002) EMBO J. 21:4671-79; see also, US Patent Application Publication No. 2009/0099115).

Alternatively, the oligonucleotides herein are single-stranded (ss). Such structures include, but are not limited to, ss RNAi molecules. Recent efforts have demonstrated the activity of ss RNAi molecules (see, e.g., Matsui et al. (2016) MOL. THER. 24:946-55). In some embodiments, the oligonucleotides are ASOs. An ASO is a ss oligonucleotide that has a nucleobase sequence which, when written or depicted in the 5′ to 3′ direction, includes a reverse complement of a targeted segment of a particular nucleic acid and is suitably modified (e.g., as a gapmer) to induce RNaseH-mediated cleavage of its target RNA in cells or (e.g., as a mixmer) so as to inhibit translation of the target mRNA in cells. ASOs for use herein are modified in any suitable manner known in the art including, for example, as shown in U.S. Pat. No. 9,567,587 (including, for example, length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) ANNU. REV. PHARMACOL. 57:81-105).

IV. Double-Stranded RNAi Oligonucleotides: ds oligonucleotides for targeting PNPLA3 mRNA and inhibiting PNPLA3 expression (e.g., via the RNAi pathway) comprising a sense strand (i.e., a passenger strand) and an antisense strand (i.e., a guide strand). In some embodiments, the sense strand and antisense strand are separate strands and are not covalently linked. In some embodiments, the sense strand and antisense strand are covalently linked.

In some embodiments, the sense strand comprises a first region (R1) and a second region (R2), where R2 comprises a first subregion (S1), a tetraloop (L) or triloop (triL), and a second subregion (S2), where L or triL is located between S1 and S2, and where S1 and S2 form a second duplex (D2). D2 has various lengths. In some embodiments, D2 is about 1 to about 6 bp in length. In some embodiments, D2 is 2-6, 3-6, 4-6, 5-6, 1-5, 2-5, 3-5 or 4-5 bp in length. In other embodiments, D2 is 1, 2, 3, 4, 5 or 6 bp in length. In certain embodiments, D2 is 6 bp in length.

In some embodiments, R1 of the sense strand and the antisense strand forms a first duplex (D1). In some embodiments, D1 is at least about 15 (e.g., at least 15, at least 16, at least 17, at least 18, at least 19, at least 20 or at least 21) nucleotides in length. In some embodiments, D1 is about 12 to about 30 nucleotides in length (e.g., 12 to 30, 12 to 27, 15 to 22, 18 to 22, 18 to 25, 18 to 27, 18 to 30 or 21 to 30 nucleotides in length). In other embodiments, D1 is at least 12 nucleotides in length (e.g., at least 12, at least 15, at least 20, at least 25 or at least 30 nucleotides in length). In other embodiments, D1 is 12, 13, 14, 15, 16, 17, 18, 19, 20, 21, 22, 23, 24, 25, 26, 27, 28, 29 or 30 nucleotides in length. In certain embodiments, D1 is 20 nucleotides in length. In some instances, D1 does not span the entire length of the sense strand and/or antisense strand. In some instances, D1 spans the entire length of either the sense strand or antisense strand or both. In certain instances, D1 spans the entire length of both the sense strand and the antisense strand.

In certain instances, a ds oligonucleotide herein includes a sense strand having a sequence of any one of SEQ ID NOs: 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, and 775, and an antisense strand having a complementary sequence selected from SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, and 776, as is arranged in Table 1. Alternatively, a ds oligonucleotide herein includes a sense strand having a sequence of any one of SEQ ID NOs: 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, and 1163, and an antisense strand having a complementary sequence selected from SEQ ID NOs: 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, and 1164, as is arranged in Table 3. In certain instances, the sense strand is any one of SEQ ID NOs: 787, 843, 867, 871, 937, 1003, 1007, 1017, 1161, or 1163, and the antisense strand is any one of SEQ ID NOs: 788, 844, 868, 872, 938, 1004, 1008, 1018, 1162, or 1164.

In certain embodiments, the disclosure describes RNAi oligonucleotides for reducing or inhibiting PNPLA3 expression that includes a sense strand having a sequence as set forth in Table A, B, C, or D (e.g., SEQ ID NOs: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, or 1300).

In certain embodiments, the disclosure describes RNAi oligonucleotides for reducing or inhibiting PNPLA3 expression that includes an antisense strand having a sequence as set forth in Table A, B, C, or D (e.g., SEQ ID NOs: 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, or 1301). In certain embodiments, the disclosure describes RNAi oligonucleotides for reducing or inhibiting PNPLA3 expression that includes a sense strand having a sequence as set forth in Table A, B, C, or D (e.g., SEQ ID Nos: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298 or 1300).

In certain embodiments, the disclosure describes RNAi oligonucleotides for reducing or inhibiting PNPLA3 expression that includes an antisense strand having a sequence as set forth in Table A, B, C, or D (e.g., SEQ ID NOs: 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, or 1301).

In some embodiments, RNAi oligonucleotides are described for reducing or inhibiting PNPLA3 expression that include an antisense strand and a sense strand, where the antisense strand has a sequence as set forth in Table A, Table B, Table C, or Table D, and where the sense strand has a sequence as set forth in Table A, Table B, Table C, or Table D.

In some instances, the RNAi oligonucleotide includes a sense strand having a nucleotide sequence as set forth in Table A, Table B, Table C, or Table D, especially any one of SEQ ID NOs: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, or 1300. In some instances, the RNAi oligonucleotide includes an antisense strand having a nucleotide sequence as set for the in Table A, Table B, Table C, or Table D, especially any one of SEQ ID NOs: 1179, 1181, 1183, 1185, 1187, 1189, 1191, 1193, 1195, 1197, 1199, 1202, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, or 1301. In certain instances, the RNAi oligonucleotide includes a sense strand having a nucleotide sequence of any one of SEQ ID NOs: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, or 1300, and an antisense strand having a nucleotide sequence of any one of SEQ ID NOs: 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, or 1301.

In certain instances, the RNAi oligonucleotide includes a sense strand having a nucleotide sequence of any one of SEQ ID NOs: 1188, 1190, 1200, 1216, 1218, 1220, 1224, 1230, 1232, 1234, 1244, 1246, 1250, 1254, 1262, 1288, 1290, 1292, 1294, 1296, 1298, or 1300, and an antisense strand having a nucleotide sequence of any one of SEQ ID NOs: 1189, 1191, 1201, 1215, 1217, 1221, 1225, 1231, 1233, 1235, 1245, 1247, 1251, 1255, 1263, 1289, 1291, 1295, 1297, 1299, or 1301.

In certain instances, the RNAi oligonucleotide includes a sense strand having a nucleotide sequence of any one of SEQ ID NOs: 1188, 1190, 1220, 1224, 1230, 1232, 1244, 1246, 1250, or 1254, and an antisense strand having a nucleotide sequence of any one of SEQ ID NOs: 1189, 1191, 1221, 1225, 1231, 1233, 1245, 1247, 1251, or 1255.

In certain instances, the sense strand and the antisense strand of the RNAi oligonucleotide, respectively, are selected from:
(a) SEQ ID NOs: 1220 and 1221,
(b) SEQ ID NOs: 1224 and 1225,
(c) SEQ ID NOs: 1230 and 1231,
(d) SEQ ID NOs: 1232 and 1233,
(e) SEQ ID NOs: 1188 and 1189,
(f) SEQ ID NOs: 1190 and 1191,
(g) SEQ ID NOs: 1244 and 1245,
(h) SEQ ID NOs: 1250 and 1251-,
(i) SEQ ID NOs: 1254 and 1255, and
(j) SEQ ID NOs: 1246 and 1247.

One of skill in the art appreciates that in some instances, the sequences presented in the Sequence Listing is referred to in describing the structure of an oligonucleotide (e.g., a ds oligonucleotide) or other nucleic acid. In such instances, the actual oligonucleotide or other nucleic acid has one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a ds oligonucleotide herein includes a 25-nucleotide sense strand and a 27-nucleotide antisense strand that when acted upon by a Dicer enzyme results in an antisense strand that is incorporated into the mature RISC. In certain instances, the sense strand of the ds oligonucleotide is longer than 27 nucleotides (e.g., 28, 29, 30, 31, 32, 33, 34, 35, 36, 37, 38, 39, or 40 nucleotides). In certain instances, the sense strand of the ds oligonucleotide is longer than 25 nucleotides (e.g., 26, 27, 28, 29, or 30 nucleotides).

In some instances, the ds oligonucleotides herein have one 5′ end that is thermodynamically less stable when compared to the other 5′ end. In some embodiments, the ds oligonucleotide is asymmetric and includes a blunt end at the 3′ end of a sense strand and a 3′-overhang at the 3′ end of an antisense strand. In some embodiments, the 3′-overhang on the antisense strand is about 1 to about 8 nucleotides in length (e.g., 1, 2, 3, 4, 5, 6, 7 or 8 nucleotides in length). Typically, a ds oligonucleotide for RNAi has a two-nucleotide overhang on the 3′ end of the antisense (guide) strand. However, other overhangs are possible. In some embodiments, an overhang is a 3′-overhang having a length of between about 1 to about 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in other instances, the overhang is a 5′-overhang comprising a length of between about 1 to about 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

In some instances, two terminal nucleotides on the 3′ end of an antisense strand are modified. In some instances, the two terminal nucleotides on the 3′ end of the antisense strand are complementary with the target mRNA (e.g., PNPLA3 mRNA). In other instances, the two terminal nucleotides on the 3′ end of the antisense strand are not complementary with the target mRNA. In some instances, two terminal nucleotides on each 3′ end of an oligonucleotide in the nicked tetraloop structure are GG. Typically, one or both of the two terminal GG nucleotides on each 3′ end of a ds oligonucleotide is not complementary with the target mRNA.

In some instances, there is one or more (e.g., 1, 2, 3, 4, or 5) mismatch(s) between the sense and antisense strand. If there is more than one mismatch between the sense and antisense strand, they may be positioned consecutively (e.g., 2, 3, or more in a row), or interspersed throughout the region of complementarity. In some instances, the 3′ end of the sense strand contains one or more mismatches. In certain instances, two mismatches are incorporated at the 3′ end of the sense strand. In some instances, base mismatches, or destabilization of segments at the 3′ end of the sense strand of the oligonucleotide improves or increases the potency of the ds oligonucleotide.

A. Antisense Strands: The oligonucleotides (e.g., a ds oligonucleotide) herein for targeting PNPLA3 mRNA and inhibiting PNPLA3 expression include an antisense strand including a sequence as set forth in the antisense strands of Table 1 or Table 3, or Table A, B, C or D. In some instances, the oligonucleotides include an antisense strand having at least about 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 788, 844, 868, 872, 938, 1004, 1008, 1018, 1162, or 1164, or an antisense strand having a nucleotide sequence of any one of SEQ ID NOs: 1189, 1191, 1221, 1225, 1231, 1233, 1245, 1247, 1251, or 1255

Further, the oligonucleotides (e.g., a ds oligonucleotide) herein can include an antisense strand of up to about 40 nucleotides in length (e.g., up to 40, up to 35, up to 30, up to 27, up to 25, up to 21, up to 19, up to 17, or up to 12 nucleotides in length). In some instances, the oligonucleotides can have an antisense strand of at least about 12 nucleotides in length (e.g., at least 12, at least 15, at least 19, at least 21, at least 22, at least 25, at least 27, at least 30, at least 35, or at least 38 nucleotides in length). Alternatively, the oligonucleotides can have an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40, or 32 to 40) nucleotides in length. In certain instances, the oligonucleotide can have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

As noted above, the antisense strand of the oligonucleotides herein may be referred to as the “guide strand.” For example, the antisense strand that engages with RISC and that binds to an Argonaute protein such as Ago2, or that engages with or that binds to one or more similar factors, and directs silencing of a target gene, the antisense strand is referred to as a guide strand (or “passenger strand”).

B. Sense Strands: The oligonucleotides (e.g., a ds oligonucleotide) herein for targeting PNPLA3 mRNA and inhibiting PNPLA3 expression include a sense strand sequence including a sequence as set forth in the sense strands of Table 1 or Table 3, or Table A, B, C, or D. In some instances, the oligonucleotides include a sense strand that having at least about 12 (e.g., at least 13, at least 14, at least 15, at least 16, at least 17, at least 18, at least 19, at least 20, at least 21, at least 22, or at least 23) contiguous nucleotides of a sequence as set forth in in any one of SEQ ID NOs: 787, 843, 867, 871, 937, 1003, 1007, 1017, 1161, or 1163.

Further, the oligonucleotides (e.g., ads oligonucleotide) herein include a sense strand (or passenger strand) of up to about 40 nucleotides in length (e.g., up to 40, up to 36, up to 30, up to 27, up to 25, up to 21, up to 19, up to 17, or up to 12 nucleotides in length). In some instances, the oligonucleotides can have a sense strand of at least about 12 nucleotides in length (e.g., at least 12, at least 15, at least 19, at least 21, at least 25, at least 27, at least 30, at least 36, or at least 38 nucleotides in length). Alternatively, the oligonucleotides can have a sense strand in a range of about 12 to about 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In certain instances, the oligonucleotides can have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

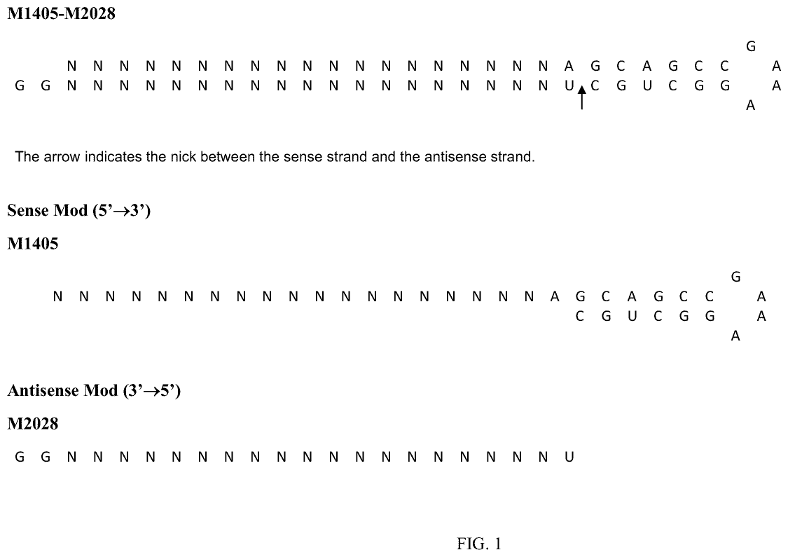

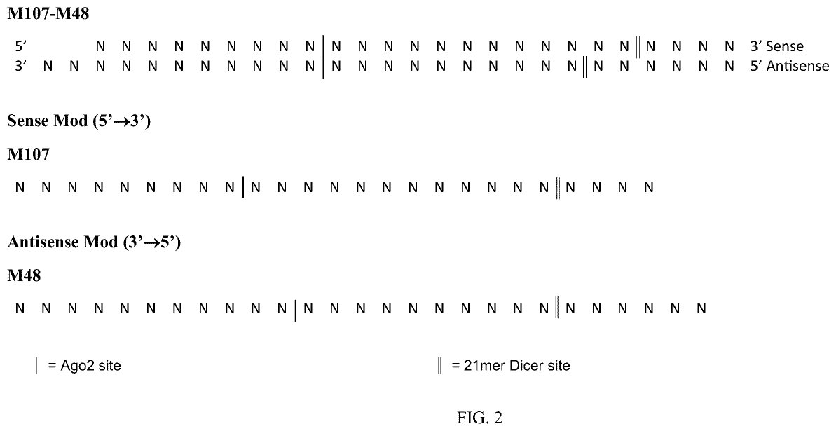

In some embodiments, the sense strand comprises a stem-loop structure at its 3′ end. In other embodiments, the sense strand comprises a stem-loop structure at its 5′ end. In additional embodiments, the stem is a duplex of about 2, 3, 4, 5, 6, 7, 8, 9, 10, 11, 12, 13, or 14 bp in length. In some embodiments, the stem-loop provides the oligonucleotides protection against degradation (e.g., enzymatic degradation) and facilitates or improves targeting and/or delivery to a target cell, tissue, or organ (e.g., the liver), or both. For example, the loop of the stem-loop provides nucleotides having one or more modifications that facilitate, improve, or increase targeting to a target mRNA (e.g., a PNPLA3 mRNA), inhibiting of target gene expression (e.g., PNPLA3 expression), and/or delivering to a target cell, tissue, or organ (e.g., the liver), or both. In some embodiments, the stem-loop itself or modification(s) to the stem-loop do not substantially affect the inherent gene expression inhibition activity of the oligonucleotide, but facilitates, improves, or increases stability (e.g., provides protection against degradation) and/or delivery of the oligonucleotide to a target cell, tissue, or organ (e.g., the liver). In certain embodiments, the oligonucleotides comprise a sense strand including (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9, or 10 nucleotides in length). In certain embodiments, the loop (L) is 4 nucleotides in length. FIGS. 1 and 2 depict non-limiting examples of such an oligonucleotide. In some embodiments the loop (L) of the stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands and combinations thereof.

### V. Oligonucleotide Modifications

A. Sugar Modifications: A modified sugar (also referred to herein as a sugar analog) includes a modified deoxyribose or ribose moiety in which, for example, one or more modifications occur at the 2′, 3′, 4′ and/or 5′ carbon position of the sugar. A modified sugar also includes non-natural, alternative, carbon structures such as those present in locked nucleic acids (“LNA”; see, e.g., Koshkin et al. (1998) TETRAHEDRON 54:3607-3630), unlocked nucleic acids (“UNA”; see, e.g., Snead et al. (2013) MOL. THER-NUC. ACIDS 2:e103) and bridged nucleic acids (“BNA”; see, e.g., Imanishi & Obika (2002) CHEM. COMMUN. 16:1653-1659).

In some embodiments, the nucleotide modification in the sugar is a 2′-modification such as, for example, 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA), or 2′-deoxy-2′-fluoro-β-d-arabinonucleic acid (2′-FANA). In certain embodiments, the modification is 2′-F, 2′-OMe, or 2′-MOE. In other embodiments, the modification in the sugar is a modification of the sugar ring, which includes modification of one or more carbons of the sugar ring. For example, the modification in the sugar is a 2′-oxygen of the sugar linked to a 1′-carbon or 4′-carbon of the sugar, or a 2′-oxygen linked to the 1′-carbon or 4′-carbon via an ethylene or methylene bridge. In other embodiments, the modification is an acyclic sugar that lacks a 2′-carbon to 3′-carbon bond. In other embodiments, the modification is a thiol group such as, for example, in the 4′ position of the sugar.

The oligonucleotides herein include at least 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand comprises at least 1 modified nucleotide (e.g., at least 1, at least 5, at least 10, at least 15, at least 20, at least 25, at least 30, at least 35, or more). In some embodiments, the antisense strand comprises at least 1 modified nucleotide (e.g., at least 1, at least 5, at least 10, at least 15, at least 20, or more).

In certain embodiments, all nucleotides of the sense strand are modified. Likewise, all nucleotides of the antisense strand are modified. In some embodiments, all the nucleotides of the oligonucleotides herein (i.e., both the sense strand and the antisense strand) are modified. As above, and in some embodiments, the modified nucleotide is a 2′-modification (e.g., a 2′-F, 2′-OMe, 2′-MOE, and/or 2′-deoxy-2′-fluoro-β-d-arabinonucleic acid). In certain embodiments, the modified nucleotide is a 2′-modification such as, for example, a 2′-F or a 2′-OMe.

Moreover, the oligonucleotides herein have different modification patterns. In some embodiments, the modified oligonucleotides comprise an antisense strand having a modification pattern as set forth in any one of Tables A, B, C or D and comprise a sense strand sequence having a modification pattern as set forth in any one of Tables A, B, C or D (as well as FIG. 1). In some embodiments, one or more of positions 8, 9, 10, or 11 of the sense strand are modified with a 2′-F. In other embodiments, the sugar moiety at each nucleotide at positions 1 to 7 and 12 to 20 in the sense strand is modified with a 2′-OMe.

In some embodiments, the antisense strand includes 3 nucleotides that are modified at the 2′-position of the sugar moiety with a 2′-F. In some embodiments, the sugar moiety at positions 2, 5, and 14 and optionally up to 3 of the nucleotides at positions 1, 3, 7, and 10 of the antisense strand are modified with a 2′-F. In other embodiments, the sugar moiety at positions 2, 5, and 14 of the antisense strand is modified with a 2′-F. In other embodiments, the sugar moiety at positions 1, 2, 5, and 14 of the antisense strand is modified with a 2′-F. In still other instances, the sugar moiety at positions 1, 2, 3, 5, 7, and 14 of the antisense strand is modified with a 2′-F. In yet other embodiments, the sugar moiety at positions 1, 2, 3, 5, 10, and 14 of the antisense strand is modified with a 2′-F. In yet other embodiments, the sugar moiety at positions 2, 3, 5, 7, 10, and 14 of the antisense strand is modified with a 2′-F.

B. 5′-Terminal Phosphates: 5′-terminal phosphate groups can be used to enhance the interaction of the oligonucleotides herein with Ago2. However, oligonucleotides having a 5′-phosphate group may be susceptible to degradation via phosphatases or other enzymes, which can limit their bioavailability in vivo. In some embodiments, the oligonucleotides (e.g., a ds oligonucleotide) comprise analogs of 5′ phosphates that are resistant to such degradation. Examples of such phosphate analogs include, but are not limited to, oxymethylphosphonate, vinylphosphonate, malonyl phosphonate, or a combination thereof. In certain embodiments the 3′ end of a strand of the oligonucleotides is attached to a chemical moiety that mimics the electrostatic and steric properties of a natural 5′-phosphate group (“phosphate mimic”).

Alternatively, or additionally, the oligonucleotides herein have a phosphate analog at a 4′-carbon position of the sugar (referred to as a “4′-phosphate analog”). See, e.g., Intl. Patent Application Publication No. WO 2018/045317. In some embodiments, the oligonucleotides herein include a 4′-phosphate analog at a 5′-terminal nucleotide. In some embodiments, the phosphate analog is an oxymethylphosphonate, in which the oxygen atom of the oxymethyl group is bound to the sugar moiety (e.g., at its 4′-carbon) or analog thereof. In other embodiments, the 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, the 4′-phosphate analog is an oxymethylphosphonate, which is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si (CH₃)₃, or a protecting group. In certain embodiments, the alkyl group is CH₂CH₃. In certain other embodiments, R is independently selected from H, CH₃, or CH₂CH₃.

C. Modified Internucleoside Linkages: In addition to the above modifications, the oligonucleotides herein comprise a modified internucleoside linkage. In some instances, phosphate modifications or substitutions result in oligonucleotides that comprise at least about 1 (e.g., at least 1, at least 2, at least 3, or at least 5) modified internucleotide linkages. In some embodiments, the oligonucleotides comprise about 1 to about 10 (e.g., 1 to 10, 2 to 8, 4 to 6, 3 to 10, 5 to 10, 1 to 5, 1 to 3, or 1 to 2) modified internucleotide linkages. In certain additional embodiments, the oligonucleotides comprise 1, 2, 3, 4, 5, 6, 7, 8, 9, or 10 modified internucleotide linkages.

Examples of modified internucleotide linkages include, but are not limited, to, a phosphorodithioate linkage, a phosphorothioate linkage, a phosphotriester linkage, a thionoalkylphosphonate linkage, a thionalkylphosphotriester linkage, a phosphoramidite linkage, a phosphonate linkage, or a boranophosphate linkage. In some embodiments, at least one modified internucleotide linkage of any one of the oligonucleotides as disclosed herein is a phosphorothioate linkage.

In some embodiments, the oligonucleotides herein comprise a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In other embodiments, the oligonucleotides comprise a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

D. Base Modifications: In addition to the above modifications, the oligonucleotides herein also comprise one or more modified nucleobases. In some embodiments, modified nucleobases (also referred to herein as base analogs) are linked at the 1′ position of a nucleotide sugar moiety. In certain embodiments, the modified nucleobase is a nitrogenous base. In certain other embodiments, the modified nucleobase does not contain nitrogen atom. See, e.g., US Patent Application Publication No. 2008/0274462. In some embodiments, the modified nucleotide is a universal base. However, in certain embodiments, the modified nucleotide does not contain a nucleobase (abasic).

With regard to universal bases, they comprise a heterocyclic moiety located at the 1′ position of a nucleotide sugar moiety in a modified nucleotide, or the equivalent position in a nucleotide sugar moiety substitution, that, when present in a duplex, is positioned opposite more than one type of base without substantially altering structure of the duplex. Moreover, and compared to a reference ss nucleic acid (e.g., oligonucleotide) that is fully complementary to a target nucleic acid, a ss nucleic acid having a universal base forms a duplex with the target nucleic acid that has a lower Tₘ than a duplex formed with the complementary nucleic acid. However, when compared to a reference ss nucleic acid in which the universal base has been replaced with a base to generate a single mismatch, the ss nucleic acid having the universal base forms a duplex with the target nucleic acid that has a higher Tₘ than a duplex formed with the nucleic acid having the mismatched base.

Exemplary universal-binding nucleotides include, but are not limited to, inosine, 1-β-D-ribofuranosyl-5-nitroindole and/or 1-β-D-ribofuranosyl-3-nitropyrrole (see, e.g., US Patent Application Publication No. 2007/0254362; Van Aerschot et al. (1995) NUCLEIC ACIDS RES. 23:4363-70; Loakes et al. (1995) NUCLEIC ACIDS RES. 23:2361-66; and Loakes & Brown (1994) NUCLEIC ACIDS RES. 22:4039-43).

E. Reversible Modifications: While certain modifications to protect the oligonucleotides herein from the in vivo environment before reaching target cells can be made, they also can reduce the potency or activity of the oligonucleotides once they reach the cytosol of the target cell. Reversible modifications therefore can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules are chemically modified with cyclic disulfide moieties to mask the negative charge created by the internucleotide diphosphate linkages and to improve cellular uptake and nuclease resistance. See, US Patent Application Publication No. 2011/0294869, Intl. Patent Application Publication Nos. WO 2014/088920 and WO 2015/188197, and Meade et al. (2014) NAT. BIOTECHNOL. 32:1256-63. This reversible modification of the internucleotide diphosphate linkages is designed to be cleaved intracellularly by the reducing environment of the cytosol (e.g., glutathione). Earlier examples include neutralizing phosphotriester modifications that are reported to be cleavable inside cells (see, e.g., Dellinger et al. (2003) J. AM. CHEM. SOC. 125:940-50).

Some reversible modifications protect the oligonucleotides during in vivo administration (e.g., transit through the blood and/or lysosomal/endosomal compartments of a cell) where the oligonucleotides will be exposed to nucleases and other harsh environmental conditions (e.g., pH). When released into the cytosol of a cell where the levels of glutathione are higher compared to extracellular space, the modification is reversed, and the result is cleaved oligonucleotides. Using reversible, glutathione-sensitive moieties, it is possible to introduce sterically larger chemical groups into the oligonucleotides when compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the oligonucleotides, such as nuclease resistance, lipophilicity, charge, thermal stability, specificity, and reduced immunogenicity. In some instances, the structure of the glutathione-sensitive moiety can be engineered to modify the kinetics of its release.

In some embodiments, the glutathione-sensitive moiety is attached to the sugar of the nucleotide. In certain embodiments, the glutathione-sensitive moiety is attached to the 2′-carbon of the sugar of a modified nucleotide. Additionally, or alternatively, the glutathione-sensitive moiety is attached to the 5′-carbon of a sugar, particularly when the modified nucleotide is the 5′-terminal nucleotide of the oligonucleotides. Additionally, or alternatively, the glutathione-sensitive moiety is attached to the 3′-carbon of sugar, particularly when the modified nucleotide is the 3′-terminal nucleotide of the oligonucleotides. In some embodiments, the glutathione-sensitive moiety includes a sulfonyl group (see, e.g., Intl. Patent Application Publication No. WO 2018/039364).

### VI. Targeting Ligands

It is desirable to target the oligonucleotides herein to one or more cells or one or more organs. Such a strategy can help to avoid undesirable effects in other organs or to avoid undue loss of the oligonucleotides to cells, tissue or organs that would not benefit therefrom. Accordingly, the oligonucleotides can be modified to facilitate targeting and/or delivering to a tissue, cell, or organ (e.g., to facilitate delivering the oligonucleotides to the liver). In some embodiments, the oligonucleotides are modified to facilitate delivery of the oligonucleotide to the hepatocytes of the liver. In certain embodiments, the oligonucleotides comprise at least one nucleotide (e.g., 1, 2, 3, 4, 5, 6, or more nucleotides) conjugated to one or more targeting ligand(s).

Exemplary targeting ligands include, but are not limited to, a carbohydrate, amino sugar, cholesterol, peptide, polypeptide, protein, or part of a protein (e.g., an antibody or antibody fragment), or lipid. In some embodiments, the targeting ligand is an aptamer. For example, the targeting ligand is an RGD peptide for targeting tumor vasculature or glioma cells, CREKA peptide for targeting tumor vasculature or stoma, transferrin, lactoferrin or an aptamer for targeting transferrin receptors expressed on CNS vasculature, or an anti-EGFR antibody for targeting EGFR on glioma cells. In certain embodiments, the targeting ligand is one or more GalNAc moieties.

In some embodiments, 1 or more (e.g., 1, 2, 3, 4, 5, or 6) nucleotides of the oligonucleotides each can be conjugated to a separate targeting ligand. In some embodiments, 2 to 4 nucleotides of the oligonucleotides each are conjugated to a separate targeting ligand. In some embodiments, targeting ligands can be conjugated to 2 to 4 nucleotides at either ends of the sense strand or antisense strand (e.g., targeting ligands are conjugated to a 2 to 4 nucleotide overhang or extension on the 5′ or 3′ end of the sense strand or antisense strand) such that the targeting ligands resemble bristles of a toothbrush and the oligonucleotides resemble a toothbrush. For example, the oligonucleotides comprise a stem-loop at either the 5′ or 3′ end of the sense strand and 1, 2, 3, or 4 nucleotides of the loop of the stem may be individually conjugated to a targeting ligand. In some embodiments, the oligonucleotides (e.g., a ds oligonucleotide) comprise a stem-loop at the 3′ end of the sense strand, where the loop of the stem-loop includes a triloop or a tetraloop, and where the 3 or 4 nucleotides of the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to the oligonucleotides herein are used to target them to the ASGPR expressed on cells. In some embodiments, the oligonucleotides are conjugated to at least one or more GalNAc moieties, where the GalNAc moieties target the oligonucleotides to an ASGPR expressed on human liver cells (e.g., human hepatocytes).

The oligonucleotides herein are conjugated directly or indirectly to a monovalent GalNAc. In some embodiments, the oligonucleotides are conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, the oligonucleotides are conjugated to one or more bivalent GalNAc, trivalent GalNAc or tetravalent GalNAc moieties.

In some embodiments, 1 or more (e.g., 1, 2, 3, 4, 5, or 6) nucleotides of the oligonucleotides herein each can be conjugated to a GalNAc moiety. In some embodiments, 2 to 4 nucleotides of a tetraloop each are conjugated to a separate GalNAc. In other embodiments, 1 to 3 nucleotides of a triloop each are conjugated to a separate GalNAc. In some embodiments, the targeting ligands are conjugated to 2 to 4 nucleotides at either end of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotides resemble a toothbrush. In some embodiments, the GalNAc moieties are conjugated to a nucleotide of the sense strand. For example, 4 GalNAc moieties are conjugated to nucleotides in the tetraloop of the sense strand, where each GalNAc moiety is conjugated to 1 nucleotide. In certain embodiments, 3 GalNAc moieties are conjugated to nucleotides in the tetraloop of the sense strand, where each GalNAc moiety is conjugated to 1 nucleotide.

In certain embodiments, the oligonucleotides comprise a monovalent GalNAc attached to a guanine nucleotide referred to as [ademG-GalNAc] or 2′-aminodiethoxymethanol-Guanine-GalNAc, as depicted below:

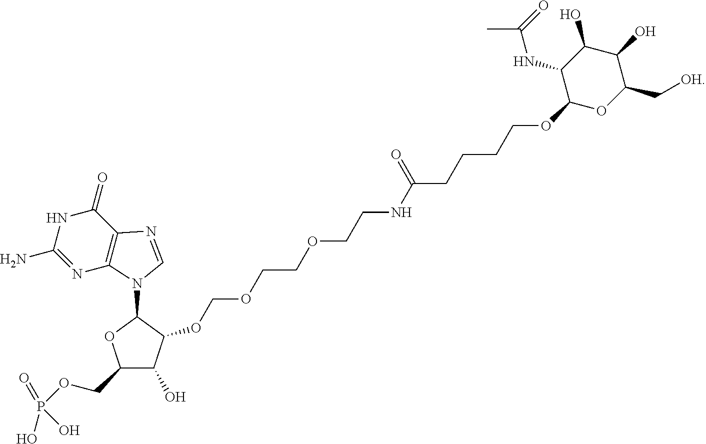 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In certain embodiments, the oligonucleotides herein comprise a monovalent GalNAc attached to an adenine nucleotide, referred to as [ademA-GalNAc] or 2′-aminodiethoxymethanol-Adenine-GalNAc, as depicted below:

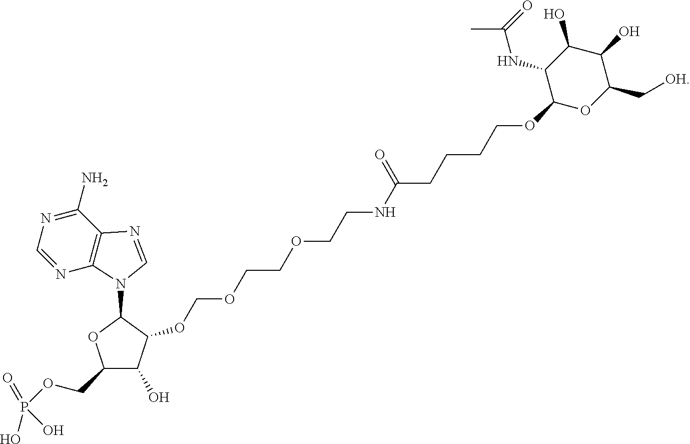

An example of such conjugation is shown below for a loop having from 5′ to 3′, the nucleotide sequence GAAA (L=linker, X=heteroatom), where stem attachment points are shown. Such a loop is present, for example, at positions 27-30 of the sense strand listed in Table 3, A, B, C or D and as shown in FIG. 1. In the chemical formula, [symbol] is used to describe an attachment point to the oligonucleotide strand:

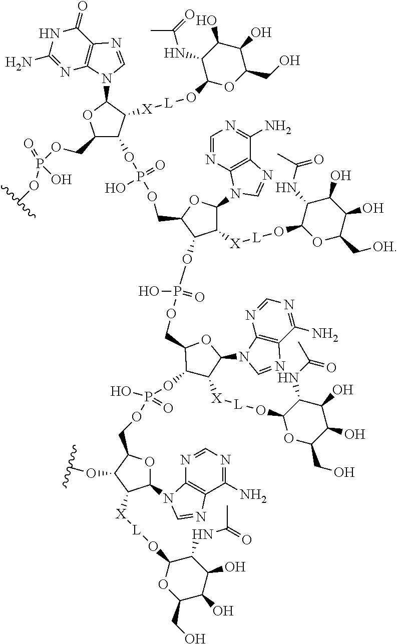

Appropriate methods or chemistry (e.g., click chemistry) are used to link a targeting ligand to a nucleotide. One way of conjugating the targeting ligand to a nucleotide is by using a click linker. In some embodiments, an acetal-based linker is used to conjugate the targeting ligand to a nucleotide of any one of the oligonucleotides herein. Acetal-based linkers are disclosed, for example, in Intl. Patent Application Publication No. WO 2016/100401. In some instances, the linker is a labile linker. However, in other instances, the linker is stable. An example is shown below for a loop having from 5′ to 3′, the nucleotides GAAA, in which GalNAc moieties are attached to nucleotides of the loop using an acetal linker. Such a loop is present, for example, at positions 27-30 of the any one of the sense strands listed in Table 3, A, B, C or D. In the chemical formula, [symbol] is an attachment point to the oligonucleotide strand:

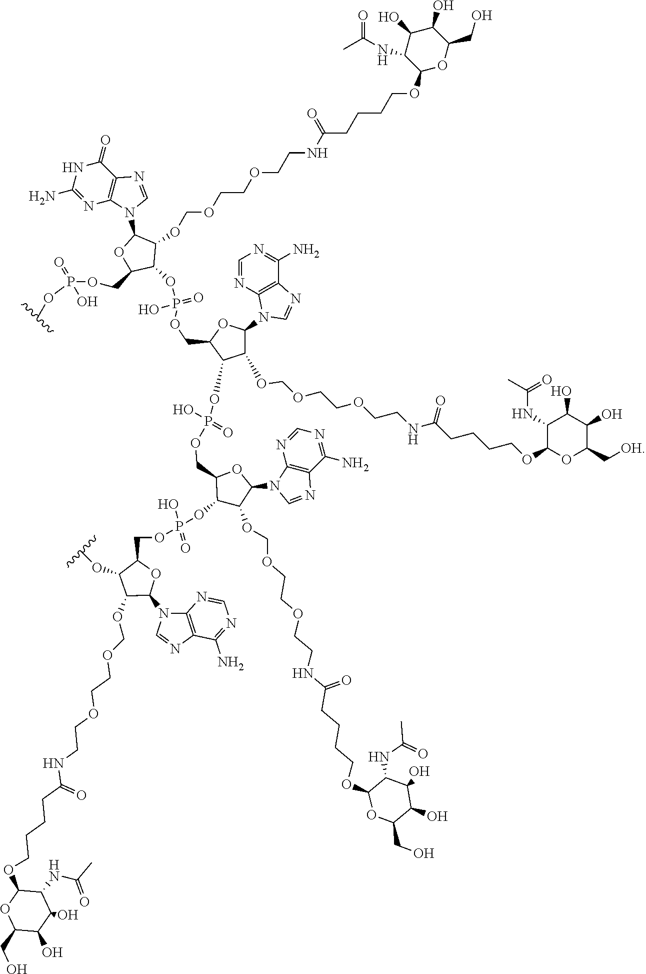

In some embodiments, a duplex extension (e.g., of up to 3, 4, 5, or 6 bp in length) is provided between the targeting ligand (e.g., a GalNAc moiety) and the oligonucleotides herein (e.g., a ds oligonucleotide). In other embodiments, the oligonucleotides do not have a GalNAc conjugated thereto.

### Formulations and Pharmaceutical Compositions

The oligonucleotides herein are incorporated into a formulation or pharmaceutical composition. Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, the oligonucleotides herein are formulated in buffer solutions such as phosphate buffered saline solutions, liposomes, micellar structures, and capsids.

Formulations of oligonucleotides with cationic lipids are used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc.), or FuGene 6 (Roche), all of which are used according to the manufacturer's instructions.

Accordingly, in some embodiments, the formulations herein comprise a liposome, a lipid, a lipid complex, a microsphere, a microparticle, a nanosphere, or a nanoparticle (such as a lipid nanoparticle) or may be otherwise formulated for administration to the cells, tissues, organs, or body of an individual in need thereof (see, e.g., Remington, “The Science and Practice of Pharmacy” (L. V. Allen Jr., ed., 22^{nd} Edition, Pharmaceutical Press, 2013).

In some embodiments, the formulations herein further comprise an excipient, which can confer to a composition improved stability, improved absorption, improved solubility and/or therapeutic enhancement of the active ingredient. In some embodiments, the excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, the oligonucleotides herein are lyophilized for extending shelf-life and then made into a solution before use (e.g., administration to an individual). Accordingly, the excipient in a pharmaceutical composition including one or more of the oligonucleotides is a lyoprotectant (e.g., mannitol, lactose, polyethylene glycol, or polyvinylpyrrolidone) or a collapse temperature modifier (e.g., dextran, Ficoll™, or gelatin).

Pharmaceutical compositions are formulated to be compatible with its intended route of administration. Routes of administration include, but are not limited to, parenteral (e.g., intravenous, intramuscular, intraperitoneal, intradermal, and subcutaneous), oral (e.g., inhalation), transdermal (e.g., topical), transmucosal, and rectal administration.

Pharmaceutical compositions suitable for injectable use comprise sterile aqueous solutions (where water soluble) or dispersions and sterile powders for the extemporaneous preparation of sterile injectable solutions or dispersion. For intravenous administration, suitable carriers include, but are not limited to, physiological saline, bacteriostatic water, Cremophor EL™ (BASF) or phosphate buffered saline (PBS). The carrier is a solvent or dispersion medium containing, for example, water, ethanol, polyol (e.g., glycerol, propylene glycol, liquid polyethylene glycol, and the like), as well as suitable mixtures thereof. In many embodiments, it will be preferable to comprise in the compositions with isotonic agents such as, for example, sugars, polyalcohols such as mannitol, sorbitol and/or sodium chloride. Sterile injectable solutions are prepared by incorporating the oligonucleotides herein in a required amount in a selected solvent with one or a combination of ingredients enumerated above, as required, followed by filtered sterilization.

Moreover, the pharmaceutical compositions comprise at least about 0.1% of a therapeutic agent (e.g., one or more of the oligonucleotides herein) or more, although the percentage of the therapeutic agent may be between about 1% to about 80% or more of the weight or volume of the total composition. Factors such as solubility, bioavailability, biological half-life, route of administration, product shelf life, as well as other pharmacological considerations will be contemplated by one of skill in the art of preparing such pharmaceutical formulations, and as such, a variety of dosages and treatment regimens may be desirable.

Even though several examples are directed toward liver-targeted delivery of at least one of the oligonucleotides herein, targeting of other tissues also is contemplated.

### Kits

The oligonucleotides herein can be incorporated into a kit comprising one or more of the oligonucleotides herein, and instructions for use. In some embodiments, the kit comprises one or more of the oligonucleotides, and a package insert containing instructions for use of the kit and/or any component thereof. In other embodiments, the kit comprises a suitable container, one or more of the oligonucleotides, one or more controls, and various buffers, reagents, enzymes, and other standard ingredients as are known in the art.

In some embodiments, the container can be at least one vial, well, test tube, flask, bottle, syringe, or other container means, into which the one or more oligonucleotides are placed, and in some instances, suitably aliquoted. In other embodiments, where an additional component is provided, the kit contains additional containers into which this component is placed. The kits also comprise a means for containing the one or more oligonucleotides and any other reagent in close confinement for commercial sale. Such containers include injection or blow-molded plastic containers into which the desired vials are retained. Containers and/or kits comprise labeling with instructions for use and/or warnings.

In some embodiments, the kit comprises one or more oligonucleotides herein, and a pharmaceutically acceptable carrier, or a pharmaceutical composition comprising one or more of the oligonucleotides and instructions for treating or delaying progression of a disease, disorder, or condition associated with PNPLA3 expression in an individual in need thereof.

### METHODS

### Methods of Making

The oligonucleotides herein are made using methods and/or techniques known to one of skill in the art such as, for example, by using conventional nucleic acid solid phase synthesis. The polynucleotides of the oligonucleotides are assembled on a suitable nucleic acid synthesizer utilizing standard nucleotide or nucleoside precursors (e.g., phosphoramidites). Automated nucleic acid synthesizers, including DNA/RNA synthesizers, are commercially available from, for example, Applied Biosystems (Foster City, CA), BioAutomation (Irving, TX), and GE Healthcare Life Sciences (Pittsburgh, PA).

As one of skill in the art understands, other methods and/or techniques of synthesizing the oligonucleotides herein are used. Additionally, the various synthetic steps are performed in an alternate sequence or order to give the desired compounds. Other synthetic chemistry transformations, protecting groups (e.g., for hydroxyl, amino, etc. present on the bases), and protecting group methodologies (protection and deprotection) useful in synthesizing the oligonucleotides are known in the art and are described in, for example, Larock, “Comprehensive Organic Transformations,” VCH Publishers (1989); Greene & Wuts, PROTECTIVE GROUPS IN ORGANIC SYNTHESIS, 2^{nd} Ed., John Wiley & Sons (1991); Fieser & Fieser, Fieser and Fieser's Reagents for Organic Synthesis, John Wiley & Sons (1994); and Paquette, ed., ENCYCLOPEDIA OF REAGENTS FOR ORGANIC SYNTHESIS, John Wiley & Sons (1995).

### Methods of Using

### I. Methods of Reducing PNPLA3 Expression in Cells, Tissue, Organs, and Organisms

The oligonucleotides herein are used to reduce PNPLA3 mRNA in cells, tissues, organs, or individuals. The methods comprise the steps described herein, and these may be, but not necessarily, carried out in the sequence as described. Other sequences, however, also are conceivable. Moreover, individual, or multiple steps are carried out either in parallel and/or overlapping in time and/or individually or in multiply repeated steps. Furthermore, the methods comprise additional, unspecified steps.

The methods comprise contacting or delivering to a cell, population of cells, tissues, organs, or individuals an effective amount any of the oligonucleotides herein (e.g., a ds oligonucleotide) for reducing PNPLA3 expression. In some embodiments, reduced PNPLA3 expression is determined by measuring a reduction in the amount or level of PNPLA3 mRNA, PNPLA3 protein, or PNPLA3 activity in a cell.

With regard to an appropriate cell type, the cell type is any cell that expresses mRNA (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue, and skin). In some embodiments, the cell is a primary cell obtained from an individual. In some embodiments, the primary cell has undergone a limited number of passages such that the cell substantially maintains is natural phenotypic properties. In some embodiments, the cell is an ex vivo, in vivo, or in vitro cell (i.e., such that one or more of the oligonucleotides herein can be delivered to the cell in culture or to an organism in which the cell resides).

In some embodiments, the oligonucleotides herein are delivered to a cell or population of cells using a nucleic acid delivery method known in the art including, but not limited to, injecting a solution containing the oligonucleotides, bombarding by particles covered by the oligonucleotides, exposing the cell or population of cells to a solution containing the oligonucleotides, or electroporating cell membranes in the presence of the oligonucleotides. Other methods known in the art for delivering oligonucleotides to cells are used such as, for example, lipid-mediated carrier transport, chemical-mediated transport, and cationic liposome transfection such as calcium phosphate, and others.

Reduced PNPLA3 expression is determined by an assay or technique that evaluates one or more molecules, properties or characteristics of a cell or population of cells associated with PNPLA3 expression (e.g., using a PNPLA3 expression biomarker) or by an assay or technique that evaluates molecules that are directly indicative of PNPLA3 expression in a cell or population of cells (e.g., PNPLA3 mRNA or PNPLA3 protein). In some embodiments, the extent to which the oligonucleotides reduce PNPLA3 expression are evaluated by comparing PNPLA3 expression in a cell or population of cells contacted with the oligonucleotides to a control cell or population of cells (e.g., a cell or population of cells not contacted with the oligonucleotides or contacted with a control oligonucleotide). In some embodiments, a control amount or level of PNPLA3 expression in a control cell or population of cells is predetermined, such that the control amount or level need not be measured in every instance the assay or technique is performed. The predetermined level or value takes a variety of forms including, but not limited to, a single cut-off value, such as a median or mean.

Contacting or delivering the oligonucleotides herein (e.g., a ds oligonucleotide) to a cell or a population of cells result in reduced PNPLA3 expression. In some embodiments, reduced PNPLA3 expression is relative to a control amount or level of PNPLA3 expression in the cell or the population of cells not contacted with the oligonucleotides or contacted with a control oligonucleotide. In some embodiments, reduced PNPLA3 expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of PNPLA3 expression. In some embodiments, the control amount or level of PNPLA3 expression is an amount or level of PNPLA3 mRNA and/or PNPLA3 protein in the cell or the population of cells that has not been contacted with oligonucleotides herein. In some embodiments, the effect of delivery of the oligonucleotides to the cell or the population of cells according to a method herein is assessed after any finite period or amount of time (e.g., minutes, hours, days, weeks, and/or months). For example, PNPLA3 expression is determined in the cell or the population of cells at least about 4 hours, about 8 hours, about 12 hours, about 18 hours, or about 24 hours. Alternatively, PNPLA3 expression is determined in the cell or the population of cells at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotides to the cell or population of cells. In other embodiments, PNPLA3 expression is determined in the cell or the population of cells at least about 1 month, about 2 months, about 3 months, about 4 months, about 5 months, or about 6 months or more after contacting or delivering the oligonucleotides to the cell or the population of cells.

In some embodiments, the oligonucleotides herein are delivered in the form of a transgene that is engineered to express in a cell one or more of the oligonucleotides or strands (e.g., sense and antisense strands). For example, the oligonucleotides are delivered using a transgene engineered to express any oligonucleotide herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, the transgenes are injected directly to an individual.

### II. Methods of Treatment:

Methods for treating an individual having, suspected of having or at risk of developing, a disease, disorder, or condition associated with PNPLA3 expression comprise administering at least one or more of the oligonucleotides herein. Additionally, methods for treating or attenuating in an individual, an onset or progression of a disease, disorder, or condition associated with PNPLA3 expression comprise using one or more of the oligonucleotides herein. Furthermore, methods for achieving one or more therapeutic benefits in an individual having a disease, disorder, or condition associated with PNPLA3 expression comprise providing one or more of the oligonucleotides herein. In some embodiments, the individual can be treated by administering a therapeutically effective amount of any one or more of the oligonucleotides of any of the above disclosed embodiments. In some embodiments, the treatment comprises reducing PNPLA3 expression. In some embodiments, the individual is treated therapeutically. In some embodiments, the individual is treated prophylactically. In all of these embodiments, the oligonucleotide is selected from Table A, B, C or D.

In some embodiments, the one or more oligonucleotides, or a pharmaceutical composition including the same, is administered to the individual having a disease, disorder, or condition associated with PNPLA3 expression such that PNPLA3 expression is reduced in the individual, thereby treating the individual. In some embodiments, an amount or level of PNPLA3 mRNA is reduced in the individual. In other embodiments, an amount or level of PNPLA3 protein is reduced in the individual. In still other embodiments, an amount or level of PNPLA3 activity is reduced in the individual. In yet other embodiments, an amount or level of liver TG (e.g., one or more TG(s) or total TGs in liver) is reduced in the individual, especially in the liver. In still other instances, an amount or level of liver inflammation can be reduced. In still other instances, an amount of level of liver fibrosis is reduced. In still other embodiments, an amount or level of plasma AST, plasma ALT, or even Pro-C3 is reduced. In any of the above disclosed embodiments, the oligonucleotides comprise a sense strand having a nucleotide sequence of any one of SEQ ID NOs: 1188, 1190, 1220, 1224, 1230, 1232, 1244, 1246, 1250 or 1254, and an antisense strand having a nucleotide sequence of any one of SEQ ID NOs: 1189, 1191, 1221, 1225, 1231, 1233, 1245, 1247, 1251 or 1255.

In some embodiments, PNPLA3 expression is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to PNPLA3 expression prior to administering the one or more oligonucleotides or pharmaceutical composition thereof. In other embodiments, PNPLA3 expression is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to PNPLA3 expression in an individual (e.g., a reference or control subject) not receiving the one or more oligonucleotides or pharmaceutical composition or receiving a control oligonucleotide, pharmaceutical composition or treatment.

In certain embodiments, an amount or level of PNPLA3 mRNA is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of PNPLA3 mRNA prior to administering the one or more oligonucleotides or pharmaceutical composition thereof. In some embodiments, the amount or level of PNPLA3 mRNA is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of PNPLA3 mRNA in an individual (e.g., a reference or control subject) not administered the one or more oligonucleotides or pharmaceutical composition or administered a control oligonucleotide, pharmaceutical composition, or treatment.

In certain embodiments, an amount or level of PNPLA3 protein is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of PNPLA3 protein prior to administering the one or more oligonucleotides or pharmaceutical composition thereof. In other embodiments, an amount or level of PNPLA3 protein is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of PNPLA3 protein in an individual (e.g., a reference or control subject) not administered the one or more oligonucleotides or pharmaceutical composition or administered a control oligonucleotide, pharmaceutical composition or treatment.

In certain embodiments, an amount or level of PNPLA3 activity is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of PNPLA3 activity prior to administering the one or more oligonucleotides or pharmaceutical composition thereof. In some embodiments, the amount or level of PNPLA3 activity is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of PNPLA3 activity in an individual (e.g., a reference or control subject) not administered the one or more oligonucleotides or pharmaceutical composition or administered a control oligonucleotide, pharmaceutical composition or treatment.

In certain embodiments, an amount or level of TG, especially liver TG, can be reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of TG prior to administering the one or more oligonucleotides or pharmaceutical composition thereof. In some embodiments, the amount or level of TG is reduced in the individual by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of TG in an individual (e.g., a reference or control subject) not administered the one or more oligonucleotides or pharmaceutical composition or administered a control oligonucleotide, pharmaceutical composition or treatment.

Here, PNPLA3 expression, the amount or level of PNPLA3 mRNA, PNPLA3 protein, PNPLA3 activity, liver TG, or any combination thereof, is reduced in a cell (e.g., a hepatocyte), a population or a group of cells (e.g., an organoid), a tissue (e.g., liver tissue), a sample (e.g., a liver biopsy sample), an organ (e.g., liver), blood or a fraction thereof (e.g., plasma), or any other biological material obtained or isolated from the individual. In some embodiments, PNPLA3 expression, the amount or level of PNPLA3 mRNA, PNPLA3 protein, PNPLA3 activity, TG, or any combination thereof, is reduced in more than one type of cell (e.g., a hepatocyte and one or more other type(s) of cell), more than one groups of cells, more than one type of tissue (e.g., liver tissue and one or more other type(s) of tissue), more than one type of sample (e.g., a liver biopsy sample and one or more other type(s) of biopsy sample), more than one organ (e.g., liver and one or more other organ(s)), more than one fraction of blood (e.g., plasma and one or more other blood fraction(s)) obtained or isolated from the subject.

Examples of a disease, disorder, or condition associated with PNPLA3 expression include, but are not limited to, AH, ALD, HCC, CCA, PSC, NAFLD, NASH, fatty liver (steatosis), inflammation of the liver, liver fibrosis, cirrhosis of the liver, or a combination thereof.

Because of their high specificity, the oligonucleotides herein specifically target mRNAs of target genes of cells, tissues, or organs (e.g., liver). In preventing disease, the target gene is the one that is required for initiation or maintenance of the disease or that has been identified as being associated with a higher risk of contracting the disease. In treating disease, one or more of the oligonucleotides are brought into contact with the cells, tissue or organ exhibiting or responsible for mediating the disease. For example, an oligonucleotide substantially identical to all or part of a wild-type (i.e., native) or mutated gene associated with a disorder or condition associated with PNPLA3 expression is brought into contact with or introduced into a cell or tissue type of interest such as a hepatocyte or other liver cell.

In some embodiments, the target gene is from any mammal, such as a human. Any gene may be silenced according to the methods herein. Moreover, the methods herein typically involve administering to an individual a therapeutically effective amount of one or more oligonucleotides herein, that is, an amount capable of producing a desirable therapeutic result. The therapeutically acceptable amount is an amount that therapeutically treats a disease or disorder or condition. The appropriate dosage for any one individual will depend on certain factors, including the individual's size, body surface area, age, the composition to be administered, the active ingredient(s) in the composition, time and route of administration, general health, and other therapeutic agents being administered concurrently.

In the methods, the individual is administered any one of the oligonucleotides or compositions herein either enterally (e.g., orally, by gastric feeding tube, by duodenal feeding tube, via gastrostomy, or rectally), parenterally (e.g., subcutaneous injection, intravenous injection or infusion, intra-arterial injection or infusion, intraosseous infusion, intramuscular injection, intracerebral injection, intracerebroventricular injection, or intrathecal), topically (e.g., epicutaneous, inhalational, via eye drops, or through a mucous membrane), or by direct injection into a target organ (e.g., the liver of a subject). Typically, the oligonucleotides or compositions are administered intravenously or subcutaneously.

As a non-limiting set of examples, the oligonucleotides or compositions herein typically are administered quarterly (once every three months), bi-monthly (once every two months), monthly or weekly. For example, the oligonucleotides or compositions are administered every week or at intervals of two, or three weeks. In certain embodiments, the oligonucleotides, or compositions are administered daily. In some embodiments, an individual is administered one or more loading doses of the oligonucleotides or compositions followed by one or more maintenance doses of the oligonucleotides or compositions.

In some embodiments, the individual is a human, a non-human primate, or other mammalian subject. In other embodiments, the individual is a domesticated animal such as a dog or a cats; livestock such as a horse, cattle, pig, sheep, goat, or chicken; and animals such as a mouse, rat, guinea pig or hamster.

### III. Medical Uses

The oligonucleotides herein can be used, or adapted for use, to treat an individual (e.g., a human having a disease, disorder, or condition associated with PNPLA3 expression) that would benefit from reducing PNPLA3 expression. In some embodiments, the oligonucleotides are provided for use, or adapted for use, to treat an individual having a disease, disorder, or condition associated with PNPLA3 expression. Also, the oligonucleotides are provided for use, or adaptable for use, in the manufacture of a medicament or pharmaceutical composition for treating a disease, disorder, or condition associated with PNPLA3 expression. In other embodiments, the oligonucleotides are provided for use, or adaptable for use, in targeting PNPLA3 mRNA and reducing PNPLA3 expression (e.g., via the RNAi pathway). In other embodiments, the oligonucleotides are provided for use, or adaptable for use, in targeting PNPLA3 mRNA and reducing an amount or level of PNPLA3 mRNA, PNPLA3 protein, and/or PNPLA3 activity.

In some embodiments, the methods comprise selecting an individual for treatment based upon the individual having a marker (e.g., a biomarker) for a disease, disorder, or condition associated with PNPLA3 expression, or someone predisposed to the same, such as, but not limited to, PNPLA3 mRNA, PNPLA3 protein or a combination thereof. Likewise, and as detailed below, the methods also comprise additional steps such as, for example, measuring or obtaining a baseline value for a marker of PNPLA3 expression (e.g., PNPLA3 protein) and then comparing such obtained value to one or more other baseline values or values obtained after the individual is administered one or more of the oligonucleotides to assess the effectiveness of treatment.

### EXAMPLES

The following non-limiting examples are offered for purposes of illustration, not limitation.

### Synthesis of Oligonucleotides

### Example 1: Preparing ds RNAi Oligonucleotides

Oligonucleotide synthesizing and purifying: The ds RNAi oligonucleotides in the Examples are chemically synthesized using methods described herein. Generally, ds RNAi oligonucleotides are synthesized using solid phase oligonucleotide synthesis methods as described for 19-23mer siRNAs (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158).

Individual RNA strands are synthesized and HPLC purified according to standard methods (Integrated DNA Technologies). For example, RNA oligonucleotides are synthesized using solid phase phosphoramidite chemistry, deprotected, and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84). The oligomers are purified using ion-exchange high performance liquid chromatography (IE-HPLC) on an Amersham Source 15Q column (1.0 cm×25 cm; Amersham Pharmacia Biotech) using a 15 min step-linear gradient. The gradient varies from 90:10 Buffers A:B to 52:48 Buffers A:B, where Buffer A is 100 mM Tris pH 8.5 and Buffer B is 100 mM Tris pH 8.5, 1 M NaCl. Samples are monitored at 260 nm and peaks corresponding to the full-length oligonucleotide species are collected, pooled, desalted on NAP-5 columns, and lyophilized.

The purity of each oligomer is determined by capillary electrophoresis (CE) on a Beckman PACE 5000 (Beckman Coulter, Inc.). The CE capillaries have a 100 μm inner diameter and contain ssDNA 100R Gel (Beckman-Coulter). Typically, about 0.6 nmole of oligonucleotide is injected into a capillary, is run in an electric field of 444 V/cm and is detected by UV absorbance at 260 nm. Denaturing Tris-Borate-7 M-urea running buffer is purchased from Beckman-Coulter. Oligoribonucleotides are obtained that are at least 90% pure as assessed by CE for use in experiments described below. Compound identity is verified by matrix-assisted laser desorption ionization time-of-flight (MALDI-TOF) mass spectroscopy on a Voyager DE™ Biospectometry Work Station (Applied Biosystems) following the manufacturer's recommended protocol. Relative molecular masses of all oligomers are obtained, often within 0.2% of expected molecular mass.

Preparing duplexes: ss RNA oligomers are resuspended (e.g., at 100 μM concentration) in duplex buffer having 100 mM potassium acetate, 30 mM HEPES, pH 7.5. Complementary sense and antisense strands are mixed in equal molar amounts to yield a final solution of, for example, 50 μM duplex. Samples are heated to 100° C. for 5 min in RNA buffer (IDT) and are allowed to cool to room temperature before use. The ds RNA oligonucleotides are stored at −20° C. ss RNA oligomers are stored lyophilized or in nuclease-free water at −80° C.

### In Vitro Function

### Example 2: RNAi Oligonucleotide Inhibition of PNPLA3 Expression In Vitro

PNPLA3 target sequence identifying: To identify RNAi oligonucleotide inhibitors of PNPLA3 expression, a computer-based algorithm is used to computationally generate PNPLA3 target sequences suitable for assaying PNPLA3 expression inhibition by the RNAi pathway. The algorithm provides RNAi oligonucleotide guide (antisense) strand sequences that are complementary to suitable PNPLA3 target sequences of human PNPLA3 mRNA (e.g., SEQ ID NO: 1). Some of the guide strand sequences identified by the algorithm also are complementary to the corresponding PNPLA3 target sequence of monkey PNPLA mRNA. Some of the antisense (guide) strand sequences identified by the algorithm also are complementary to the corresponding PNPLA3 target sequence of monkey PNPLA3 mRNA (e.g., SEQ ID NO:5). From this, 384 ds RNAi oligonucleotides (formatted as DsiRNA oligonucleotides) are generated, each with a unique antisense strand having a region of complementarity to a PNPLA3 target sequence identified by the algorithm.

TABLE 1
DsiRNAs (unmodified) Targeting Human PNPLA3 mRNA and Controls Evaluated in Cells.

| | Passenger | SEQ ID | Guide | SEQ ID |
|---|---|---|---|---|
| DsiRNA | (sense) | NO: | (antisense) | NO: |
| 1 | UCUGCAGGUCCUCUCAGAUCUUGUG | 9 | CACAAGAUCUGAGAGGACCUGCAGAGU | 10 |
| 2 | AGGUCCUCUCAGAUCUUGUGCGGAA | 11 | UUCCGCACAAGAUCUGAGAGGACCUGC | 12 |
| 3 | AUUGGCAUCUUCCAUCCAUCCUUCA | 13 | UGAAGGAUGGAUGGAAGAUGCCAAUGU | 14 |
| 4 | AUCUUCCAUCCAUCCUUCAACUUAA | 15 | UUAAGUUGAAGGAUGGAUGGAAGAUGC | 16 |
| 5 | UCUUCCAUCCAUCCUUCAACUUAAG | 17 | CUUAAGUUGAAGGAUGGAUGGAAGAUG | 18 |
| 6 | CCAGAGUGUCUGAUGGGGAAAACGU | 19 | ACGUUUUCCCCAUCAGACACUCUGGUA | 20 |
| 7 | GAGUGUCUGAUGGGGAAAACGUUCU | 21 | AGAACGUUUUCCCCAUCAGACACUCUG | 22 |
| 8 | AUGGGGAAAACGUUCUGGUGUCUGA | 23 | UCAGACACCAGAACGUUUUCCCCAUCA | 24 |
| 9 | GGGGAAAACGUUCUGGUGUCUGACU | 25 | AGUCAGACACCAGAACGUUUUCCCCAU | 26 |
| 10 | GGGAAAACGUUCUGGUGUCUGACUU | 27 | AAGUCAGACACCAGAACGUUUUCCCCA | 28 |
| 11 | GGAAAACGUUCUGGUGUCUGACUUU | 29 | AAAGUCAGACACCAGAACGUUUUCCCC | 30 |
| 12 | GAAAACGUUCUGGUGUCUGACUUUC | 31 | GAAAGUCAGACACCAGAACGUUUUCCC | 32 |
| 13 | AAAACGUUCUGGUGUCUGACUUUCG | 33 | CGAAAGUCAGACACCAGAACGUUUUCC | 34 |
| 14 | AAACGUUCUGGUGUCUGACUUUCGG | 35 | CCGAAAGUCAGACACCAGAACGUUUUC | 36 |
| 15 | AACGUUCUGGUGUCUGACUUUCGGU | 37 | ACCGAAAGUCAGACACCAGAACGUUUU | 38 |
| 16 | ACGUUCUGGUGUCUGACUUUCGGUC | 39 | GACCGAAAGUCAGACACCAGAACGUUU | 40 |
| 17 | GUUCUGGUGUCUGACUUUCGGUCCA | 41 | UGGACCGAAAGUCAGACACCAGAACGU | 42 |
| 18 | GACGAAGUCGUGGAUGCCUUGGUAU | 43 | AUACCAAGGCAUCCACGACUUCGUCUU | 44 |
| 19 | ACGAAGUCGUGGAUGCCUUGGUAUG | 45 | CAUACCAAGGCAUCCACGACUUCGUCU | 46 |
| 20 | CGAAGUCGUGGAUGCCUUGGUAUGU | 47 | ACAUACCAAGGCAUCCACGACUUCGUC | 48 |
| 21 | CAGAGGCGUGCGAUAUGUGGAUGGA | 49 | UCCAUCCACAUAUCGCACGCCUCUGAA | 50 |
| 22 | GCGAUAUGUGGAUGGAGGAGUGAGU | 51 | ACUCACUCCUCCAUCCACAUAUCGCAC | 52 |
| 23 | GAUGGAGGAGUGAGUGACAACGUAC | 53 | GUACGUUGUCACUCACUCCUCCAUCCA | 54 |
| 24 | GAGGAGUGAGUGACAACGUACCCUU | 55 | AAGGGUACGUUGUCACUCACUCCUCCA | 56 |
| 25 | GAGUGAGUGACAACGUACCCUUCAU | 57 | AUGAAGGGUACGUUGUCACUCACUCCU | 58 |
| 26 | AGUGAGUGACAACGUACCCUUCAUU | 59 | AAUGAAGGGUACGUUGUCACUCACUCC | 60 |
| 27 | GUGAGUGACAACGUACCCUUCAUUG | 61 | CAAUGAAGGGUACGUUGUCACUCACUC | 62 |
| 28 | UGAGUGACAACGUACCCUUCAUUGA | 63 | UCAAUGAAGGGUACGUUGUCACUCACU | 64 |
| 29 | GAGUGACAACGUACCCUUCAUUGAU | 65 | AUCAAUGAAGGGUACGUUGUCACUCAC | 66 |
| 30 | AGUGACAACGUACCCUUCAUUGAUG | 67 | CAUCAAUGAAGGGUACGUUGUCACUCA | 68 |
| 31 | GUGACAACGUACCCUUCAUUGAUGC | 69 | GCAUCAAUGAAGGGUACGUUGUCACUC | 70 |
| 32 | UGACAACGUACCCUUCAUUGAUGCC | 71 | GGCAUCAAUGAAGGGUACGUUGUCACU | 72 |
| 33 | GACAACGUACCCUUCAUUGAUGCCA | 73 | UGGCAUCAAUGAAGGGUACGUUGUCAC | 74 |
| 34 | CAACGUACCCUUCAUUGAUGCCAAA | 75 | UUUGGCAUCAAUGAAGGGUACGUUGUC | 76 |
| 35 | AACGUACCCUUCAUUGAUGCCAAAA | 77 | UUUUGGCAUCAAUGAAGGGUACGUUGU | 78 |
| 36 | ACGUACCCUUCAUUGAUGCCAAAAC | 79 | GUUUUGGCAUCAAUGAAGGGUACGUUG | 80 |
| 37 | CGUACCCUUCAUUGAUGCCAAAACA | 81 | UGUUUUGGCAUCAAUGAAGGGUACGUU | 82 |
| 38 | UACCCUUCAUUGAUGCCAAAACAAC | 83 | GUUGUUUUGGCAUCAAUGAAGGGUACG | 84 |
| 39 | UUGAUGCCAAAACAACCAUCACCGU | 85 | ACGGUGAUGGUUGUUUUGGCAUCAAUG | 86 |
| 40 | GAUGCCAAAACAACCAUCACCGUGU | 87 | ACACGGUGAUGGUUGUUUUGGCAUCAA | 88 |
| 41 | AUGGGGAGUACGACAUCUGCCCUAA | 89 | UUAGGGCAGAUGUCGUACUCCCCAUAG | 90 |
| 42 | GUACGACAUCUGCCCUAAAGUCAAG | 91 | CUUGACUUUAGGGCAGAUGUCGUACUC | 92 |
| 43 | GACAUCUGCCCUAAAGUCAAGUCCA | 93 | UGGACUUGACUUUAGGGCAGAUGUCGU | 94 |
| 44 | ACAUCUGCCCUAAAGUCAAGUCCAC | 95 | GUGGACUUGACUUUAGGGCAGAUGUCG | 96 |
| 45 | GUCAAGUCCACGAACUUUCUUCAUG | 97 | CAUGAAGAAAGUUCGUGGACUUGACUU | 98 |
| 46 | UCAAGUCCACGAACUUUCUUCAUGU | 99 | ACAUGAAGAAAGUUCGUGGACUUGACU | 100 |
| 47 | CAAGUCCACGAACUUUCUUCAUGUG | 101 | CACAUGAAGAAAGUUCGUGGACUUGAC | 102 |
| 48 | GUCCACGAACUUUCUUCAUGUGGAC | 103 | GUCCACAUGAAGAAAGUUCGUGGACUU | 104 |
| 49 | CCACGAACUUUCUUCAUGUGGACAU | 105 | AUGUCCACAUGAAGAAAGUUCGUGGAC | 106 |
| 50 | CACGAACUUUCUUCAUGUGGACAUC | 107 | GAUGUCCACAUGAAGAAAGUUCGUGGA | 108 |
| 51 | ACGAACUUUCUUCAUGUGGACAUCA | 109 | UGAUGUCCACAUGAAGAAAGUUCGUGG | 110 |
| 52 | CGAACUUUCUUCAUGUGGACAUCAC | 111 | GUGAUGUCCACAUGAAGAAAGUUCGUG | 112 |
| 53 | GAACUUUCUUCAUGUGGACAUCACC | 113 | GGUGAUGUCCACAUGAAGAAAGUUCGU | 114 |
| 54 | AACUUUCUUCAUGUGGACAUCACCA | 115 | UGGUGAUGUCCACAUGAAGAAAGUUCG | 116 |
| 55 | ACUUUCUUCAUGUGGACAUCACCAA | 117 | UUGGUGAUGUCCACAUGAAGAAAGUUC | 118 |
| 56 | CUUUCUUCAUGUGGACAUCACCAAG | 119 | CUUGGUGAUGUCCACAUGAAGAAAGUU | 120 |
| 57 | UUUCUUCAUGUGGACAUCACCAAGC | 121 | GCUUGGUGAUGUCCACAUGAAGAAAGU | 122 |
| 58 | UUCUUCAUGUGGACAUCACCAAGCU | 123 | AGCUUGGUGAUGUCCACAUGAAGAAAG | 124 |
| 59 | CUUCAUGUGGACAUCACCAAGCUCA | 125 | UGAGCUUGGUGAUGUCCACAUGAAGAA | 126 |
| 60 | UUCAUGUGGACAUCACCAAGCUCAG | 127 | CUGAGCUUGGUGAUGUCCACAUGAAGA | 128 |
| 61 | UCAUGUGGACAUCACCAAGCUCAGU | 129 | ACUGAGCUUGGUGAUGUCCACAUGAAG | 130 |
| 62 | CAUGUGGACAUCACCAAGCUCAGUC | 131 | GACUGAGCUUGGUGAUGUCCACAUGAA | 132 |
| 63 | AUGUGGACAUCACCAAGCUCAGUCU | 133 | AGACUGAGCUUGGUGAUGUCCACAUGA | 134 |
| 64 | UGUGGACAUCACCAAGCUCAGUCUA | 135 | UAGACUGAGCUUGGUGAUGUCCACAUG | 136 |
| 65 | GUGGACAUCACCAAGCUCAGUCUAC | 137 | GUAGACUGAGCUUGGUGAUGUCCACAU | 138 |
| 66 | UGGACAUCACCAAGCUCAGUCUACG | 139 | CGUAGACUGAGCUUGGUGAUGUCCACA | 140 |
| 67 | AGCUUUUGUCCCCCCGGAUCUCAAG | 141 | CUUGAGAUCCGGGGGGACAAAAGCUCU | 142 |
| 68 | AAGGUGCUGGGAGAGAUAUGCCUUC | 143 | GAAGGCAUAUCUCUCCCAGCACCUUGA | 144 |
| 69 | GGGAGAGAUAUGCCUUCGAGGAUAU | 145 | AUAUCCUCGAAGGCAUAUCUCUCCCAG | 146 |
| 70 | GGAGAGAUAUGCCUUCGAGGAUAUU | 147 | AAUAUCCUCGAAGGCAUAUCUCUCCCA | 148 |
| 71 | AGAGAUAUGCCUUCGAGGAUAUUUG | 149 | CAAAUAUCCUCGAAGGCAUAUCUCUCC | 150 |
| 72 | GAGAUAUGCCUUCGAGGAUAUUUGG | 151 | CCAAAUAUCCUCGAAGGCAUAUCUCUC | 152 |
| 73 | AGAUAUGCCUUCGAGGAUAUUUGGA | 153 | UCCAAAUAUCCUCGAAGGCAUAUCUCU | 154 |
| 74 | GAUAUGCCUUCGAGGAUAUUUGGAU | 155 | AUCCAAAUAUCCUCGAAGGCAUAUCUC | 156 |
| 72 | AUAUGCCUUCGAGGAUAUUUGGAUG | 157 | CAUCCAAAUAUCCUCGAAGGCAUAUCU | 158 |
| 76 | AUGCCUUCGAGGAUAUUUGGAUGCA | 159 | UGCAUCCAAAUAUCCUCGAAGGCAUAU | 160 |
| 77 | UGCCUUCGAGGAUAUUUGGAUGCAU | 161 | AUGCAUCCAAAUAUCCUCGAAGGCAUA | 162 |
| 78 | GCCUUCGAGGAUAUUUGGAUGCAUU | 163 | AAUGCAUCCAAAUAUCCUCGAAGGCAU | 164 |
| 79 | UCAGGUUCUUGGAAGAGAAGGGCAU | 165 | AUGCCCUUCUCUUCCAAGAACCUGAAU | 166 |
| 80 | CAGGUUCUUGGAAGAGAAGGGCAUC | 167 | GAUGCCCUUCUCUUCCAAGAACCUGAA | 168 |
| 81 | AGGUUCUUGGAAGAGAAGGGCAUCU | 169 | AGAUGCCCUUCUCUUCCAAGAACCUGA | 170 |
| 82 | GGUUCUUGGAAGAGAAGGGCAUCUG | 171 | CAGAUGCCCUUCUCUUCCAAGAACCUG | 172 |
| 83 | UGGAAGAGAAGGGCAUCUGCAACAG | 173 | CUGUUGCAGAUGCCCUUCUCUUCCAAG | 174 |
| 84 | UGAAGUCAUCCUCAGAAGGGAUGGA | 175 | UCCAUCCCUUCUGAGGAUGACUUCAGG | 176 |
| 85 | GAAGUCAUCCUCAGAAGGGAUGGAU | 177 | AUCCAUCCCUUCUGAGGAUGACUUCAG | 178 |
| 86 | AAGUCAUCCUCAGAAGGGAUGGAUC | 179 | GAUCCAUCCCUUCUGAGGAUGACUUCA | 180 |
| 87 | GUCAUCCUCAGAAGGGAUGGAUCCU | 181 | AGGAUCCAUCCCUUCUGAGGAUGACUU | 182 |
| 88 | CAUCCUCAGAAGGGAUGGAUCCUGA | 183 | UCAGGAUCCAUCCCUUCUGAGGAUGAC | 184 |
| 89 | AUCCUCAGAAGGGAUGGAUCCUGAG | 185 | CUCAGGAUCCAUCCCUUCUGAGGAUGA | 186 |
| 90 | CUAGACCACCUGCGUCUCAGCAUCC | 187 | GGAUGCUGAGACGCAGGUGGUCUAGCA | 188 |
| 91 | AGUGAAGAAAUGAAAGACAAAGGUG | 189 | CACCUUUGUCUUUCAUUUCUUCACUCA | 190 |
| 92 | GUGAAGAAAUGAAAGACAAAGGUGG | 191 | CCACCUUUGUCUUUCAUUUCUUCACUC | 192 |
| 93 | UGAAGAAAUGAAAGACAAAGGUGGA | 193 | UCCACCUUUGUCUUUCAUUUCUUCACU | 194 |
| 94 | GAAGAAAUGAAAGACAAAGGUGGAU | 195 | AUCCACCUUUGUCUUUCAUUUCUUCAC | 196 |
| 95 | AAGAAAUGAAAGACAAAGGUGGAUA | 197 | UAUCCACCUUUGUCUUUCAUUUCUUCA | 198 |
| 96 | AGAAAUGAAAGACAAAGGUGGAUAC | 199 | GUAUCCACCUUUGUCUUUCAUUUCUUC | 200 |
| 97 | GAAAUGAAAGACAAAGGUGGAUACA | 201 | UGUAUCCACCUUUGUCUUUCAUUUCUU | 202 |
| 98 | AAAUGAAAGACAAAGGUGGAUACAU | 203 | AUGUAUCCACCUUUGUCUUUCAUUUCU | 204 |
| 99 | AAUGAAAGACAAAGGUGGAUACAUG | 205 | CAUGUAUCCACCUUUGUCUUUCAUUUC | 206 |
| 100 | AUGAAAGACAAAGGUGGAUACAUGA | 207 | UCAUGUAUCCACCUUUGUCUUUCAUUU | 208 |
| 101 | UGAAAGACAAAGGUGGAUACAUGAG | 209 | CUCAUGUAUCCACCUUUGUCUUUCAUU | 210 |
| 102 | GAAAGACAAAGGUGGAUACAUGAGC | 211 | GCUCAUGUAUCCACCUUUGUCUUUCAU | 212 |
| 103 | AAGACAAAGGUGGAUACAUGAGCAA | 213 | UUGCUCAUGUAUCCACCUUUGUCUUUC | 214 |
| 104 | AGACAAAGGUGGAUACAUGAGCAAG | 215 | CUUGCUCAUGUAUCCACCUUUGUCUUU | 216 |
| 105 | GACAAAGGUGGAUACAUGAGCAAGA | 217 | UCUUGCUCAUGUAUCCACCUUUGUCUU | 218 |
| 106 | ACAAAGGUGGAUACAUGAGCAAGAU | 219 | AUCUUGCUCAUGUAUCCACCUUUGUCU | 220 |
| 107 | AAGGUGGAUACAUGAGCAAGAUUUG | 221 | CAAAUCUUGCUCAUGUAUCCACCUUUG | 222 |
| 108 | AGGUGGAUACAUGAGCAAGAUUUGC | 223 | GCAAAUCUUGCUCAUGUAUCCACCUUU | 224 |
| 109 | UGGAUACAUGAGCAAGAUUUGCAAC | 225 | GUUGCAAAUCUUGCUCAUGUAUCCACC | 226 |
| 110 | GGAUACAUGAGCAAGAUUUGCAACU | 227 | AGUUGCAAAUCUUGCUCAUGUAUCCAC | 228 |
| 111 | GAUACAUGAGCAAGAUUUGCAACUU | 229 | AAGUUGCAAAUCUUGCUCAUGUAUCCA | 230 |
| 112 | AUACAUGAGCAAGAUUUGCAACUUG | 231 | CAAGUUGCAAAUCUUGCUCAUGUAUCC | 232 |
| 113 | UACAUGAGCAAGAUUUGCAACUUGC | 233 | GCAAGUUGCAAAUCUUGCUCAUGUAUC | 234 |
| 114 | ACAUGAGCAAGAUUUGCAACUUGCU | 235 | AGCAAGUUGCAAAUCUUGCUCAUGUAU | 236 |
| 115 | CAUGAGCAAGAUUUGCAACUUGCUA | 237 | UAGCAAGUUGCAAAUCUUGCUCAUGUA | 238 |
| 116 | AUGAGCAAGAUUUGCAACUUGCUAC | 239 | GUAGCAAGUUGCAAAUCUUGCUCAUGU | 240 |
| 117 | UGAGCAAGAUUUGCAACUUGCUACC | 241 | GGUAGCAAGUUGCAAAUCUUGCUCAUG | 242 |
| 118 | GAGCAAGAUUUGCAACUUGCUACCC | 243 | GGGUAGCAAGUUGCAAAUCUUGCUCAU | 244 |
| 119 | AGCAAGAUUUGCAACUUGCUACCCA | 245 | UGGGUAGCAAGUUGCAAAUCUUGCUCA | 246 |
| 120 | GCAAGAUUUGCAACUUGCUACCCAU | 247 | AUGGGUAGCAAGUUGCAAAUCUUGCUC | 248 |
| 121 | CAAGAUUUGCAACUUGCUACCCAUU | 249 | AAUGGGUAGCAAGUUGCAAAUCUUGCU | 250 |
| 122 | AAGAUUUGCAACUUGCUACCCAUUA | 251 | UAAUGGGUAGCAAGUUGCAAAUCUUGC | 252 |
| 123 | AGAUUUGCAACUUGCUACCCAUUAG | 253 | CUAAUGGGUAGCAAGUUGCAAAUCUUG | 254 |
| 124 | GAUUUGCAACUUGCUACCCAUUAGG | 255 | CCUAAUGGGUAGCAAGUUGCAAAUCUU | 256 |
| 125 | AUUUGCAACUUGCUACCCAUUAGGA | 257 | UCCUAAUGGGUAGCAAGUUGCAAAUCU | 258 |
| 126 | UUUGCAACUUGCUACCCAUUAGGAU | 259 | AUCCUAAUGGGUAGCAAGUUGCAAAUC | 260 |
| 127 | UUGCAACUUGCUACCCAUUAGGAUA | 261 | UAUCCUAAUGGGUAGCAAGUUGCAAAU | 262 |
| 128 | UGCAACUUGCUACCCAUUAGGAUAA | 263 | UUAUCCUAAUGGGUAGCAAGUUGCAAA | 264 |
| 129 | GCAACUUGCUACCCAUUAGGAUAAU | 265 | AUUAUCCUAAUGGGUAGCAAGUUGCAA | 266 |
| 130 | CAACUUGCUACCCAUUAGGAUAAUG | 267 | CAUUAUCCUAAUGGGUAGCAAGUUGCA | 268 |
| 131 | CUUGCUACCCAUUAGGAUAAUGUCU | 269 | AGACAUUAUCCUAAUGGGUAGCAAGUU | 270 |
| 132 | UUGCUACCCAUUAGGAUAAUGUCUU | 271 | AAGACAUUAUCCUAAUGGGUAGCAAGU | 272 |
| 133 | UGCUACCCAUUAGGAUAAUGUCUUA | 273 | UAAGACAUUAUCCUAAUGGGUAGCAAG | 274 |
| 134 | AUUAGGAUAAUGUCUUAUGUAAUGC | 275 | GCAUUACAUAAGACAUUAUCCUAAUGG | 276 |
| 135 | UUAGGAUAAUGUCUUAUGUAAUGCU | 277 | AGCAUUACAUAAGACAUUAUCCUAAUG | 278 |
| 136 | CUGUGGAAUCUGCCAUUGCGAUUGU | 279 | ACAAUCGCAAUGGCAGAUUCCACAGGC | 280 |
| 137 | GGAAUCUGCCAUUGCGAUUGUCCAG | 281 | CUGGACAAUCGCAAUGGCAGAUUCCAC | 282 |
| 138 | GAAUCUGCCAUUGCGAUUGUCCAGA | 283 | UCUGGACAAUCGCAAUGGCAGAUUCCA | 284 |
| 139 | AAUCUGCCAUUGCGAUUGUCCAGAG | 285 | CUCUGGACAAUCGCAAUGGCAGAUUCC | 286 |
| 140 | AUCUGCCAUUGCGAUUGUCCAGAGA | 287 | UCUCUGGACAAUCGCAAUGGCAGAUUC | 288 |
| 141 | CCAUUGCGAUUGUCCAGAGACUGGU | 289 | ACCAGUCUCUGGACAAUCGCAAUGGCA | 290 |
| 142 | CAUUGCGAUUGUCCAGAGACUGGUG | 291 | CACCAGUCUCUGGACAAUCGCAAUGGC | 292 |
| 143 | AUUGCGAUUGUCCAGAGACUGGUGA | 293 | UCACCAGUCUCUGGACAAUCGCAAUGG | 294 |
| 144 | UUGCGAUUGUCCAGAGACUGGUGAC | 295 | GUCACCAGUCUCUGGACAAUCGCAAUG | 296 |
| 145 | UGCGAUUGUCCAGAGACUGGUGACA | 297 | UGUCACCAGUCUCUGGACAAUCGCAAU | 298 |
| 146 | GCGAUUGUCCAGAGACUGGUGACAU | 299 | AUGUCACCAGUCUCUGGACAAUCGCAA | 300 |
| 147 | CGAUUGUCCAGAGACUGGUGACAUG | 301 | CAUGUCACCAGUCUCUGGACAAUCGCA | 302 |
| 148 | AUUGUCCAGAGACUGGUGACAUGGC | 303 | GCCAUGUCACCAGUCUCUGGACAAUCG | 304 |
| 149 | GUCCAGAGACUGGUGACAUGGCUUC | 305 | GAAGCCAUGUCACCAGUCUCUGGACAA | 306 |
| 150 | CCAGAGACUGGUGACAUGGCUUCCA | 307 | UGGAAGCCAUGUCACCAGUCUCUGGAC | 308 |
| 151 | CAGAGACUGGUGACAUGGCUUCCAG | 309 | CUGGAAGCCAUGUCACCAGUCUCUGGA | 310 |
| 152 | AGAGACUGGUGACAUGGCUUCCAGA | 311 | UCUGGAAGCCAUGUCACCAGUCUCUGG | 312 |
| 153 | GAGACUGGUGACAUGGCUUCCAGAU | 313 | AUCUGGAAGCCAUGUCACCAGUCUCUG | 314 |
| 154 | AGACUGGUGACAUGGCUUCCAGAUA | 315 | UAUCUGGAAGCCAUGUCACCAGUCUCU | 316 |
| 155 | GACUGGUGACAUGGCUUCCAGAUAU | 317 | AUAUCUGGAAGCCAUGUCACCAGUCUC | 318 |
| 156 | ACUGGUGACAUGGCUUCCAGAUAUG | 319 | CAUAUCUGGAAGCCAUGUCACCAGUCU | 320 |
| 157 | CUGGUGACAUGGCUUCCAGAUAUGC | 321 | GCAUAUCUGGAAGCCAUGUCACCAGUC | 322 |
| 158 | GACAUGGCUUCCAGAUAUGCCCGAC | 323 | GUCGGGCAUAUCUGGAAGCCAUGUCAC | 324 |
| 159 | CAUGGCUUCCAGAUAUGCCCGACGA | 325 | UCGUCGGGCAUAUCUGGAAGCCAUGUC | 326 |
| 160 | CAGAUAUGCCCGACGAUGUCCUGUG | 327 | CACAGGACAUCGUCGGGCAUAUCUGGA | 328 |
| 161 | CUCACAGGUGUUCACUCGAGUGCUG | 329 | CAGCACUCGAGUGAACACCUGUGAGGU | 330 |
| 162 | CACUCGAGUGCUGAUGUGUCUGCUC | 331 | GAGCAGACACAUCAGCACUCGAGUGAA | 332 |
| 163 | CUCGAGUGCUGAUGUGUCUGCUCCC | 333 | GGGAGCAGACACAUCAGCACUCGAGUG | 334 |
| 164 | CCCAAAUGCCAGUGAGCAGCCAACA | 335 | UGUUGGCUGCUCACUGGCAUUUGGGAC | 336 |
| 165 | UCAGGUCCAGCCUGAACUUCUUCUU | 337 | AAGAAGAAGUUCAGGCUGGACCUGAGG | 338 |
| 166 | CAGGUCCAGCCUGAACUUCUUCUUG | 339 | CAAGAAGAAGUUCAGGCUGGACCUGAG | 340 |
| 167 | AGGUCCAGCCUGAACUUCUUCUUGG | 341 | CCAAGAAGAAGUUCAGGCUGGACCUGA | 342 |
| 168 | CAAUAAAGUACCUGCUGGUGCUGAG | 343 | CUCAGCACCAGCAGGUACUUUAUUGCC | 344 |
| 169 | AAUAAAGUACCUGCUGGUGCUGAGG | 345 | CCUCAGCACCAGCAGGUACUUUAUUGC | 346 |
| 170 | AUAAAGUACCUGCUGGUGCUGAGGG | 347 | CCCUCAGCACCAGCAGGUACUUUAUUG | 348 |
| 171 | CUCUCCACCUUUCCCAGUUUUUCAC | 349 | GUGAAAAACUGGGAAAGGUGGAGAGCC | 350 |
| 172 | CUCCACCUUUCCCAGUUUUUCACUA | 351 | UAGUGAAAAACUGGGAAAGGUGGAGAG | 352 |
| 173 | UCCACCUUUCCCAGUUUUUCACUAG | 353 | CUAGUGAAAAACUGGGAAAGGUGGAGA | 354 |
| 174 | CCACCUUUCCCAGUUUUUCACUAGA | 355 | UCUAGUGAAAAACUGGGAAAGGUGGAG | 356 |
| 175 | CACCUUUCCCAGUUUUUCACUAGAG | 357 | CUCUAGUGAAAAACUGGGAAAGGUGGA | 358 |
| 176 | ACCUUUCCCAGUUUUUCACUAGAGA | 359 | UCUCUAGUGAAAAACUGGGAAAGGUGG | 360 |
| 177 | CCUUUCCCAGUUUUUCACUAGAGAA | 361 | UUCUCUAGUGAAAAACUGGGAAAGGUG | 362 |
| 178 | AGUUUUUCACUAGAGAAGAGUCUGU | 363 | ACAGACUCUUCUCUAGUGAAAAACUGG | 364 |
| 179 | UCUAGCAGAUUCUUUCAGAGGUGCU | 365 | AGCACCUCUGAAAGAAUCUGCUAGACU | 366 |
| 180 | AGCAGAUUCUUUCAGAGGUGCUAAA | 367 | UUUAGCACCUCUGAAAGAAUCUGCUAG | 368 |
| 181 | GCAGAUUCUUUCAGAGGUGCUAAAG | 369 | CUUUAGCACCUCUGAAAGAAUCUGCUA | 370 |
| 182 | CAGAUUCUUUCAGAGGUGCUAAAGU | 371 | ACUUUAGCACCUCUGAAAGAAUCUGCU | 372 |
| 183 | AGAUUCUUUCAGAGGUGCUAAAGUU | 373 | AACUUUAGCACCUCUGAAAGAAUCUGC | 374 |
| 184 | GAUUCUUUCAGAGGUGCUAAAGUUU | 375 | AAACUUUAGCACCUCUGAAAGAAUCUG | 376 |
| 185 | AUUCUUUCAGAGGUGCUAAAGUUUC | 377 | GAAACUUUAGCACCUCUGAAAGAAUCU | 378 |
| 186 | AGGUGCUAAAGUUUCCCAUCUUUGU | 379 | ACAAAGAUGGGAAACUUUAGCACCUCU | 380 |
| 187 | GGUGCUAAAGUUUCCCAUCUUUGUG | 381 | CACAAAGAUGGGAAACUUUAGCACCUC | 382 |
| 188 | GUGCUAAAGUUUCCCAUCUUUGUGC | 383 | GCACAAAGAUGGGAAACUUUAGCACCU | 384 |
| 189 | GCUAAAGUUUCCCAUCUUUGUGCAG | 385 | CUGCACAAAGAUGGGAAACUUUAGCAC | 386 |
| 190 | CUAAAGUUUCCCAUCUUUGUGCAGC | 387 | GCUGCACAAAGAUGGGAAACUUUAGCA | 388 |
| 191 | UAAAGUUUCCCAUCUUUGUGCAGCU | 389 | AGCUGCACAAAGAUGGGAAACUUUAGC | 390 |
| 192 | AAAGUUUCCCAUCUUUGUGCAGCUA | 391 | UAGCUGCACAAAGAUGGGAAACUUUAG | 392 |
| 193 | AAGUUUCCCAUCUUUGUGCAGCUAC | 393 | GUAGCUGCACAAAGAUGGGAAACUUUA | 394 |
| 194 | AGUUUCCCAUCUUUGUGCAGCUACC | 395 | GGUAGCUGCACAAAGAUGGGAAACUUU | 396 |
| 195 | GUUUCCCAUCUUUGUGCAGCUACCU | 397 | AGGUAGCUGCACAAAGAUGGGAAACUU | 398 |
| 196 | AUCUUUGUGCAGCUACCUCCGCAUU | 399 | AAUGCGGAGGUAGCUGCACAAAGAUGG | 400 |
| 197 | GUGCAGCUACCUCCGCAUUGCUGUG | 401 | CACAGCAAUGCGGAGGUAGCUGCACAA | 402 |
| 198 | CCAGCCUCUGAGCUGAGUUGGUUUU | 403 | AAAACCAACUCAGCUCAGAGGCUGGGA | 404 |
| 199 | CAGCCUCUGAGCUGAGUUGGUUUUA | 405 | UAAAACCAACUCAGCUCAGAGGCUGGG | 406 |
| 200 | AGCCUCUGAGCUGAGUUGGUUUUAU | 407 | AUAAAACCAACUCAGCUCAGAGGCUGG | 408 |
| 201 | GCCUCUGAGCUGAGUUGGUUUUAUG | 409 | CAUAAAACCAACUCAGCUCAGAGGCUG | 410 |
| 202 | CCUCUGAGCUGAGUUGGUUUUAUGA | 411 | UCAUAAAACCAACUCAGCUCAGAGGCU | 412 |
| 203 | CUCUGAGCUGAGUUGGUUUUAUGAA | 413 | UUCAUAAAACCAACUCAGCUCAGAGGC | 414 |
| 204 | UCUGAGCUGAGUUGGUUUUAUGAAA | 415 | UUUCAUAAAACCAACUCAGCUCAGAGG | 416 |
| 205 | UGAGCUGAGUUGGUUUUAUGAAAAG | 417 | CUUUUCAUAAAACCAACUCAGCUCAGA | 418 |
| 206 | UGAGUUGGUUUUAUGAAAAGCUAGG | 419 | CCUAGCUUUUCAUAAAACCAACUCAGC | 420 |
| 207 | GAGUUGGUUUUAUGAAAAGCUAGGA | 421 | UCCUAGCUUUUCAUAAAACCAACUCAG | 422 |
| 208 | UUGGUUUUAUGAAAAGCUAGGAAGC | 423 | GCUUCCUAGCUUUUCAUAAAACCAACU | 424 |
| 209 | UGGUUUUAUGAAAAGCUAGGAAGCA | 425 | UGCUUCCUAGCUUUUCAUAAAACCAAC | 426 |
| 210 | GGUUUUAUGAAAAGCUAGGAAGCAA | 427 | UUGCUUCCUAGCUUUUCAUAAAACCAA | 428 |
| 211 | UUUUAUGAAAAGCUAGGAAGCAACC | 429 | GGUUGCUUCCUAGCUUUUCAUAAAACC | 430 |
| 212 | UUAUGAAAAGCUAGGAAGCAACCUU | 431 | AAGGUUGCUUCCUAGCUUUUCAUAAAA | 432 |
| 213 | UAUGAAAAGCUAGGAAGCAACCUUU | 433 | AAAGGUUGCUUCCUAGCUUUUCAUAAA | 434 |
| 214 | AUGAAAAGCUAGGAAGCAACCUUUC | 435 | GAAAGGUUGCUUCCUAGCUUUUCAUAA | 436 |
| 215 | CCAGCACUUAACUCUAAUACAUCAG | 437 | CUGAUGUAUUAGAGUUAAGUGCUGGAC | 438 |
| 216 | CAGCACUUAACUCUAAUACAUCAGC | 439 | GCUGAUGUAUUAGAGUUAAGUGCUGGA | 440 |
| 217 | UAAUACAUCAGCAUGCGUUAAUUCA | 441 | UGAAUUAACGCAUGCUGAUGUAUUAGA | 442 |
| 218 | AAUACAUCAGCAUGCGUUAAUUCAG | 443 | CUGAAUUAACGCAUGCUGAUGUAUUAG | 444 |
| 219 | AGCAUGCGUUAAUUCAGCUGGUUGG | 445 | CCAACCAGCUGAAUUAACGCAUGCUGA | 446 |
| 220 | GCAUGCGUUAAUUCAGCUGGUUGGG | 447 | CCCAACCAGCUGAAUUAACGCAUGCUG | 448 |
| 221 | UGCGUUAAUUCAGCUGGUUGGGAAA | 449 | UUUCCCAACCAGCUGAAUUAACGCAUG | 450 |
| 222 | GCGUUAAUUCAGCUGGUUGGGAAAU | 451 | AUUUCCCAACCAGCUGAAUUAACGCAU | 452 |
| 223 | CGUUAAUUCAGCUGGUUGGGAAAUG | 453 | CAUUUCCCAACCAGCUGAAUUAACGCA | 454 |
| 224 | GUUAAUUCAGCUGGUUGGGAAAUGA | 455 | UCAUUUCCCAACCAGCUGAAUUAACGC | 456 |
| 225 | UUAAUUCAGCUGGUUGGGAAAUGAC | 457 | GUCAUUUCCCAACCAGCUGAAUUAACG | 458 |
| 226 | UAAUUCAGCUGGUUGGGAAAUGACA | 459 | UGUCAUUUCCCAACCAGCUGAAUUAAC | 460 |
| 227 | AUUCAGCUGGUUGGGAAAUGACACC | 461 | GGUGUCAUUUCCCAACCAGCUGAAUUA | 462 |
| 228 | UUCAGCUGGUUGGGAAAUGACACCA | 463 | UGGUGUCAUUUCCCAACCAGCUGAAUU | 464 |
| 229 | UCAGCUGGUUGGGAAAUGACACCAG | 465 | CUGGUGUCAUUUCCCAACCAGCUGAAU | 466 |
| 230 | CAGCUGGUUGGGAAAUGACACCAGG | 467 | CCUGGUGUCAUUUCCCAACCAGCUGAA | 468 |
| 231 | AGCUGGUUGGGAAAUGACACCAGGA | 469 | UCCUGGUGUCAUUUCCCAACCAGCUGA | 470 |
| 232 | GCUGGUUGGGAAAUGACACCAGGAA | 471 | UUCCUGGUGUCAUUUCCCAACCAGCUG | 472 |
| 233 | GCAGAGGGUCCCUUACUGACUGUUU | 473 | AAACAGUCAGUAAGGGACCCUCUGCAC | 474 |
| 234 | CAGAGGGUCCCUUACUGACUGUUUC | 475 | GAAACAGUCAGUAAGGGACCCUCUGCA | 476 |
| 235 | AGAGGGUCCCUUACUGACUGUUUCG | 477 | CGAAACAGUCAGUAAGGGACCCUCUGC | 478 |
| 236 | CCUAUUAAUGGUCAGACUGUUCCAG | 479 | CUGGAACAGUCUGACCAUUAAUAGGGC | 480 |
| 237 | CUAUUAAUGGUCAGACUGUUCCAGC | 481 | GCUGGAACAGUCUGACCAUUAAUAGGG | 482 |
| 238 | UAUUAAUGGUCAGACUGUUCCAGCA | 483 | UGCUGGAACAGUCUGACCAUUAAUAGG | 484 |
| 239 | AUUAAUGGUCAGACUGUUCCAGCAU | 485 | AUGCUGGAACAGUCUGACCAUUAAUAG | 486 |
| 240 | UUAAUGGUCAGACUGUUCCAGCAUG | 487 | CAUGCUGGAACAGUCUGACCAUUAAUA | 488 |
| 241 | UAAUGGUCAGACUGUUCCAGCAUGA | 489 | UCAUGCUGGAACAGUCUGACCAUUAAU | 490 |
| 242 | AAUGGUCAGACUGUUCCAGCAUGAG | 491 | CUCAUGCUGGAACAGUCUGACCAUUAA | 492 |
| 243 | AGAACGACACUGCCUGUCAGGUGGU | 493 | ACCACCUGACAGGCAGUGUCGUUCUUG | 494 |
| 244 | CGACACUGCCUGUCAGGUGGUCUGC | 495 | GCAGACCACCUGACAGGCAGUGUCGUU | 496 |
| 245 | AACCUUGACUACUAAAAACGUCUCC | 497 | GGAGACGUUUUUAGUAGUCAAGGUUAU | 498 |
| 246 | UUUAGAACACCUUUUUCACCUAACU | 499 | AGUUAGGUGAAAAAGGUGUUCUAAAAU | 500 |
| 247 | UUAGAACACCUUUUUCACCUAACUA | 501 | UAGUUAGGUGAAAAAGGUGUUCUAAAA | 502 |
| 248 | UAGAACACCUUUUUCACCUAACUAA | 503 | UUAGUUAGGUGAAAAAGGUGUUCUAAA | 504 |
| 249 | AGAACACCUUUUUCACCUAACUAAA | 505 | UUUAGUUAGGUGAAAAAGGUGUUCUAA | 506 |
| 250 | GAACACCUUUUUCACCUAACUAAAA | 507 | UUUUAGUUAGGUGAAAAAGGUGUUCUA | 508 |
| 251 | AACACCUUUUUCACCUAACUAAAAU | 509 | AUUUUAGUUAGGUGAAAAAGGUGUUCU | 510 |
| 252 | ACACCUUUUUCACCUAACUAAAAUA | 511 | UAUUUUAGUUAGGUGAAAAAGGUGUUC | 512 |
| 253 | CACCUUUUUCACCUAACUAAAAUAA | 513 | UUAUUUUAGUUAGGUGAAAAAGGUGUU | 514 |
| 254 | ACCUUUUUCACCUAACUAAAAUAAU | 515 | AUUAUUUUAGUUAGGUGAAAAAGGUGU | 516 |
| 255 | CUUUUUCACCUAACUAAAAUAAUGU | 517 | ACAUUAUUUUAGUUAGGUGAAAAAGGU | 518 |
| 256 | UUUUCACCUAACUAAAAUAAUGUUU | 519 | AAACAUUAUUUUAGUUAGGUGAAAAAG | 520 |
| 257 | UUCACCUAACUAAAAUAAUGUUUAA | 521 | UUAAACAUUAUUUUAGUUAGGUGAAAA | 522 |
| 258 | UCACCUAACUAAAAUAAUGUUUAAA | 523 | UUUAAACAUUAUUUUAGUUAGGUGAAA | 524 |
| 259 | CACCUAACUAAAAUAAUGUUUAAAG | 525 | CUUUAAACAUUAUUUUAGUUAGGUGAA | 526 |
| 260 | ACCUAACUAAAAUAAUGUUUAAAGA | 527 | UCUUUAAACAUUAUUUUAGUUAGGUGA | 528 |
| 261 | CCUAACUAAAAUAAUGUUUAAAGAG | 529 | CUCUUUAAACAUUAUUUUAGUUAGGUG | 530 |
| 262 | CUAACUAAAAUAAUGUUUAAAGAGU | 531 | ACUCUUUAAACAUUAUUUUAGUUAGGU | 532 |
| 263 | UAACUAAAAUAAUGUUUAAAGAGUU | 533 | AACUCUUUAAACAUUAUUUUAGUUAGG | 534 |
| 264 | AACUAAAAUAAUGUUUAAAGAGUUU | 535 | AAACUCUUUAAACAUUAUUUUAGUUAG | 536 |
| 265 | ACUAAAAUAAUGUUUAAAGAGUUUU | 537 | AAAACUCUUUAAACAUUAUUUUAGUUA | 538 |
| 266 | CUAAAAUAAUGUUUAAAGAGUUUUG | 539 | CAAAACUCUUUAAACAUUAUUUUAGUU | 540 |
| 267 | UAAAAUAAUGUUUAAAGAGUUUUGU | 541 | ACAAAACUCUUUAAACAUUAUUUUAGU | 542 |
| 268 | AAGAGUUUUGUAUAAAAAUGUAAGG | 543 | CCUUACAUUUUUAUACAAAACUCUUUA | 544 |
| 269 | GUUUUGUAUAAAAAUGUAAGGAAGC | 545 | GCUUCCUUACAUUUUUAUACAAAACUC | 546 |
| 270 | UUUUGUAUAAAAAUGUAAGGAAGCG | 547 | CGCUUCCUUACAUUUUUAUACAAAACU | 548 |
| 271 | UUUGUAUAAAAAUGUAAGGAAGCGU | 549 | ACGCUUCCUUACAUUUUUAUACAAAAC | 550 |
| 272 | UUGUAUAAAAAUGUAAGGAAGCGUU | 551 | AACGCUUCCUUACAUUUUUAUACAAAA | 552 |
| 273 | UGUAUAAAAAUGUAAGGAAGCGUUG | 553 | CAACGCUUCCUUACAUUUUUAUACAAA | 554 |
| 274 | GUAUAAAAAUGUAAGGAAGCGUUGU | 555 | ACAACGCUUCCUUACAUUUUUAUACAA | 556 |
| 275 | AUGUAAGGAAGCGUUGUUACCUGUU | 557 | AACAGGUAACAACGCUUCCUUACAUUU | 558 |
| 276 | UUUUGUAUUAUGUGAAUCAGUGAGA | 559 | UCUCACUGAUUCACAUAAUACAAAAUU | 560 |
| 277 | UUUGUAUUAUGUGAAUCAGUGAGAU | 561 | AUCUCACUGAUUCACAUAAUACAAAAU | 562 |
| 278 | UUGUAUUAUGUGAAUCAGUGAGAUG | 563 | CAUCUCACUGAUUCACAUAAUACAAAA | 564 |
| 279 | UGUAUUAUGUGAAUCAGUGAGAUGU | 565 | ACAUCUCACUGAUUCACAUAAUACAAA | 566 |
| 280 | GUAUUAUGUGAAUCAGUGAGAUGUU | 567 | AACAUCUCACUGAUUCACAUAAUACAA | 568 |
| 281 | UAUUAUGUGAAUCAGUGAGAUGUUA | 569 | UAACAUCUCACUGAUUCACAUAAUACA | 570 |
| 282 | AUUAUGUGAAUCAGUGAGAUGUUAG | 571 | CUAACAUCUCACUGAUUCACAUAAUAC | 572 |
| 283 | UUAUGUGAAUCAGUGAGAUGUUAGU | 573 | ACUAACAUCUCACUGAUUCACAUAAUA | 574 |
| 284 | UAUGUGAAUCAGUGAGAUGUUAGUA | 575 | UACUAACAUCUCACUGAUUCACAUAAU | 576 |
| 285 | AUGUGAAUCAGUGAGAUGUUAGUAG | 577 | CUACUAACAUCUCACUGAUUCACAUAA | 578 |
| 286 | UGUGAAUCAGUGAGAUGUUAGUAGA | 579 | UCUACUAACAUCUCACUGAUUCACAUA | 580 |
| 287 | GUGAAUCAGUGAGAUGUUAGUAGAA | 581 | UUCUACUAACAUCUCACUGAUUCACAU | 582 |
| 288 | GUGAGAUGUUAGUAGAAUAAGCCUU | 583 | AAGGCUUAUUCUACUAACAUCUCACUG | 584 |
| 289 | UUUUCUAUUUAUGCAUUUGAGUACA | 585 | UGUACUCAAAUGCAUAAAUAGAAAAAA | 586 |
| 290 | UUUCUAUUUAUGCAUUUGAGUACAG | 587 | CUGUACUCAAAUGCAUAAAUAGAAAAA | 588 |
| 291 | UUCUAUUUAUGCAUUUGAGUACAGT | 589 | ACUGUACUCAAAUGCAUAAAUAGAAAA | 590 |
| 292 | CUAUUUAUGCAUUUGAGUACAGUAC | 591 | GUACUGUACUCAAAUGCAUAAAUAGAA | 592 |
| 293 | UGCUCAAACUGUUAAAUGUUGGAAA | 593 | UUUCCAACAUUUAACAGUUUGAGCACA | 594 |
| 294 | GCUCAAACUGUUAAAUGUUGGAAAA | 595 | UUUUCCAACAUUUAACAGUUUGAGCAC | 596 |
| 295 | CUCAAACUGUUAAAUGUUGGAAAAG | 597 | CUUUUCCAACAUUUAACAGUUUGAGCA | 598 |
| 296 | UCAAACUGUUAAAUGUUGGAAAAGA | 599 | UCUUUUCCAACAUUUAACAGUUUGAGC | 600 |
| 297 | CAAACUGUUAAAUGUUGGAAAAGAA | 601 | UUCUUUUCCAACAUUUAACAGUUUGAG | 602 |
| 298 | AAACUGUUAAAUGUUGGAAAAGAAA | 603 | UUUCUUUUCCAACAUUUAACAGUUUGA | 604 |
| 299 | AACUGUUAAAUGUUGGAAAAGAAAG | 605 | CUUUCUUUUCCAACAUUUAACAGUUUG | 606 |
| 300 | ACUGUUAAAUGUUGGAAAAGAAAGA | 607 | UCUUUCUUUUCCAACAUUUAACAGUUU | 608 |
| 301 | CUGUUAAAUGUUGGAAAAGAAAGAT | 609 | AUCUUUCUUUUCCAACAUUUAACAGUU | 610 |
| 302 | UGUUAAAUGUUGGAAAAGAAAGATA | 611 | UAUCUUUCUUUUCCAACAUUUAACAGU | 612 |
| 303 | GUUAAAUGUUGGAAAAGAAAGAUAC | 613 | GUAUCUUUCUUUUCCAACAUUUAACAG | 614 |
| 304 | UUAAAUGUUGGAAAAGAAAGAUACA | 615 | UGUAUCUUUCUUUUCCAACAUUUAACA | 616 |
| 305 | UAAAUGUUGGAAAAGAAAGAUACAA | 617 | UUGUAUCUUUCUUUUCCAACAUUUAAC | 618 |
| 306 | GCACUUGACUGAGAAGACAGACCCT | 619 | AGGGUCUGUCUUCUCAGUCAAGUGCUU | 620 |
| 307 | GAGAAAAGAGGCUACUUGUGAAAAT | 621 | AUUUUCACAAGUAGCCUCUUUUCUCAA | 622 |
| 308 | AGAAAAGAGGCUACUUGUGAAAATA | 623 | UAUUUUCACAAGUAGCCUCUUUUCUCA | 624 |
| 309 | GAAAAGAGGCUACUUGUGAAAAUAA | 625 | UUAUUUUCACAAGUAGCCUCUUUUCUC | 626 |
| 310 | AAAAGAGGCUACUUGUGAAAAUAAT | 627 | AUUAUUUUCACAAGUAGCCUCUUUUCU | 628 |
| 311 | AAAGAGGCUACUUGUGAAAAUAATG | 629 | CAUUAUUUUCACAAGUAGCCUCUUUUC | 630 |
| 312 | AAGAGGCUACUUGUGAAAAUAAUGA | 631 | UCAUUAUUUUCACAAGUAGCCUCUUUU | 632 |
| 313 | AGAGGCUACUUGUGAAAAUAAUGAG | 633 | CUCAUUAUUUUCACAAGUAGCCUCUUU | 634 |
| 314 | GAGGCUACUUGUGAAAAUAAUGAGC | 635 | GCUCAUUAUUUUCACAAGUAGCCUCUU | 636 |
| 315 | GGCUACUUGUGAAAAUAAUGAGCCC | 637 | GGGCUCAUUAUUUUCACAAGUAGCCUC | 638 |
| 316 | CUACUUGUGAAAAUAAUGAGCCCCC | 639 | GGGGGCUCAUUAUUUUCACAAGUAGCC | 640 |
| 317 | UGAACCUGCCUUCUUACAUCUUGAG | 641 | CUCAAGAUGUAAGAAGGCAGGUUCAAA | 642 |
| 318 | AAAGUUACAAGUUUCUUUUCCCAAG | 643 | CUUGGGAAAAGAAACUUGUAACUUUCC | 644 |
| 319 | AAGUUACAAGUUUCUUUUCCCAAGT | 645 | ACUUGGGAAAAGAAACUUGUAACUUUC | 646 |
| 320 | AGUUACAAGUUUCUUUUCCCAAGTT | 647 | AACUUGGGAAAAGAAACUUGUAACUUU | 648 |
| 321 | AGUUUCUUUUCCCAAGUUUCCCAGT | 649 | ACUGGGAAACUUGGGAAAAGAAACUUG | 650 |
| 322 | CAACAGUAUUUUCUAAUAACCAGTA | 651 | UACUGGUUAUUAGAAAAUACUGUUGGC | 652 |
| 323 | AACAGUAUUUUCUAAUAACCAGUAT | 653 | AUACUGGUUAUUAGAAAAUACUGUUGG | 654 |
| 324 | ACAGUAUUUUCUAAUAACCAGUATA | 655 | UAUACUGGUUAUUAGAAAAUACUGUUG | 656 |
| 325 | CAGUAUUUUCUAAUAACCAGUAUAT | 657 | AUAUACUGGUUAUUAGAAAAUACUGUU | 658 |
| 326 | UUGUGAUUGUUAUCAGGAAAAAATA | 659 | UAUUUUUUCCUGAUAACAAUCACAAUA | 660 |
| 327 | UGUGAUUGUUAUCAGGAAAAAAUAT | 661 | AUAUUUUUUCCUGAUAACAAUCACAAU | 662 |
| 328 | GUGAUUGUUAUCAGGAAAAAAUATA | 663 | UAUAUUUUUUCCUGAUAACAAUCACAA | 664 |
| 329 | UGAUUGUUAUCAGGAAAAAAUAUAT | 665 | AUAUAUUUUUUCCUGAUAACAAUCACA | 666 |
| 330 | GAUUGUUAUCAGGAAAAAAUAUATT | 667 | AAUAUAUUUUUUCCUGAUAACAAUCAC | 668 |
| 331 | AUUGUUAUCAGGAAAAAAUAUAUTA | 669 | UAAUAUAUUUUUUCCUGAUAACAAUCA | 670 |
| 332 | UUGUUAUCAGGAAAAAAUAUAUUAA | 671 | UUAAUAUAUUUUUUCCUGAUAACAAUC | 672 |
| 333 | UGUUAUCAGGAAAAAAUAUAUUAAA | 673 | UUUAAUAUAUUUUUUCCUGAUAACAAU | 674 |
| 334 | GUUAUCAGGAAAAAAUAUAUUAAAT | 675 | AUUUAAUAUAUUUUUUCCUGAUAACAA | 676 |
| 335 | UUAUCAGGAAAAAAUAUAUUAAATG | 677 | CAUUUAAUAUAUUUUUUCCUGAUAACA | 678 |
| 336 | UAUCAGGAAAAAAUAUAUUAAAUGG | 679 | CCAUUUAAUAUAUUUUUUCCUGAUAAC | 680 |
| 337 | AUCAGGAAAAAAUAUAUUAAAUGGC | 681 | GCCAUUUAAUAUAUUUUUUCCUGAUAA | 682 |
| 338 | AGGAAAAAAUAUAUUAAAUGGCUGA | 683 | UCAGCCAUUUAAUAUAUUUUUUCCUGA | 684 |
| 339 | GGAAAAAAUAUAUUAAAUGGCUGAT | 685 | AUCAGCCAUUUAAUAUAUUUUUUCCUG | 686 |
| 340 | GAAAAAAUAUAUUAAAUGGCUGATA | 687 | UAUCAGCCAUUUAAUAUAUUUUUUCCU | 688 |
| 341 | AAAAAAUAUAUUAAAUGGCUGAUAG | 689 | CUAUCAGCCAUUUAAUAUAUUUUUUCC | 690 |
| 342 | UAUUUUCUUUCUGCUUUUAAAAATT | 691 | AAUUUUUAAAAGCAGAAAGAAAAUACG | 692 |
| 343 | AUUUUCUUUCUGCUUUUAAAAAUTA | 693 | UAAUUUUUAAAAGCAGAAAGAAAAUAC | 694 |
| 344 | UCUUUCUGCUUUUAAAAAUUAUUCA | 695 | UGAAUAAUUUUUAAAAGCAGAAAGAAA | 696 |
| 345 | CUUUCUGCUUUUAAAAAUUAUUCAG | 697 | CUGAAUAAUUUUUAAAAGCAGAAAGAA | 698 |
| 346 | UUUCUGCUUUUAAAAAUUAUUCAGG | 699 | CCUGAAUAAUUUUUAAAAGCAGAAAGA | 700 |
| 347 | CUACUAAAAACACAAAAAUUAGCCA | 701 | UGGCUAAUUUUUGUGUUUUUAGUAGAG | 702 |
| 348 | CAAGAUAAGGAAAUCAGGAAGUGTA | 703 | UACACUUCCUGAUUUCCUUAUCUUGAU | 704 |
| 349 | AAGAUAAGGAAAUCAGGAAGUGUAA | 705 | UUACACUUCCUGAUUUCCUUAUCUUGA | 706 |
| 350 | AGAUAAGGAAAUCAGGAAGUGUAAT | 707 | AUUACACUUCCUGAUUUCCUUAUCUUG | 708 |
| 351 | GAUAAGGAAAUCAGGAAGUGUAATA | 709 | UAUUACACUUCCUGAUUUCCUUAUCUU | 710 |
| 352 | AUAAGGAAAUCAGGAAGUGUAAUAT | 711 | AUAUUACACUUCCUGAUUUCCUUAUCU | 712 |
| 353 | UAAGGAAAUCAGGAAGUGUAAUATT | 713 | AAUAUUACACUUCCUGAUUUCCUUAUC | 714 |
| 354 | AAGGAAAUCAGGAAGUGUAAUAUTC | 715 | GAAUAUUACACUUCCUGAUUUCCUUAU | 716 |
| 355 | AGGAAAUCAGGAAGUGUAAUAUUCT | 717 | AGAAUAUUACACUUCCUGAUUUCCUUA | 718 |
| 356 | GGAAAUCAGGAAGUGUAAUAUUCTT | 719 | AAGAAUAUUACACUUCCUGAUUUCCUU | 720 |
| 357 | GAAAUCAGGAAGUGUAAUAUUCUTA | 721 | UAAGAAUAUUACACUUCCUGAUUUCCU | 722 |
| 358 | CUAUGAAUGCAUUCUUAUUUCUUCT | 723 | AGAAGAAAUAAGAAUGCAUUCAUAGGC | 724 |
| 359 | UAUGAAUGCAUUCUUAUUUCUUCTT | 725 | AAGAAGAAAUAAGAAUGCAUUCAUAGG | 726 |
| 360 | CUACCACACCCAGCUAGUUUUUUTT | 727 | AAAAAAAACUAGCUGGGUGUGGUAGUG | 728 |
| 361 | CCACACCCAGCUAGUUUUUUUUUGT | 729 | ACAAAAAAAAACUAGCUGGGUGUGGUA | 730 |
| 362 | CACACCCAGCUAGUUUUUUUUUGTA | 731 | UACAAAAAAAAACUAGCUGGGUGUGGU | 732 |
| 363 | GCUAGGAUUACAGGUGUGAGCUACC | 733 | GGUAGCUCACACCUGUAAUCCUAGCAC | 734 |
| 364 | CUAGGAUUACAGGUGUGAGCUACCA | 735 | UGGUAGCUCACACCUGUAAUCCUAGCA | 736 |
| 365 | CCAUGCCUGGUCCAACAUUCUUCAT | 737 | AUGAAGAAUGUUGGACCAGGCAUGGUA | 738 |
| 366 | UGCAGAGUAUGAGCCUGAUUUUGTT | 739 | AACAAAAUCAGGCUCAUACUCUGCACU | 740 |
| 367 | GCAGAGUAUGAGCCUGAUUUUGUTT | 741 | AAACAAAAUCAGGCUCAUACUCUGCAC | 742 |
| 368 | CAGAGUAUGAGCCUGAUUUUGUUTA | 743 | UAAACAAAAUCAGGCUCAUACUCUGCA | 744 |
| 369 | AGAGUAUGAGCCUGAUUUUGUUUAA | 745 | UUAAACAAAAUCAGGCUCAUACUCUGC | 746 |
| 370 | GAGUAUGAGCCUGAUUUUGUUUAAA | 747 | UUUAAACAAAAUCAGGCUCAUACUCUG | 748 |
| 371 | GGGUGAAACCCCAUCUCUACUAAAA | 749 | UUUUAGUAGAGAUGGGGUUUCACCCAG | 750 |
| 372 | GUGAAACCCCAUCUCUACUAAAAAA | 751 | UUUUUUAGUAGAGAUGGGGUUUCACCC | 752 |
| 373 | UGAAACCCCAUCUCUACUAAAAAAT | 753 | AUUUUUUAGUAGAGAUGGGGUUUCACC | 754 |
| 374 | GAAACCCCAUCUCUACUAAAAAATG | 755 | CAUUUUUUAGUAGAGAUGGGGUUUCAC | 756 |
| 375 | AAACCCCAUCUCUACUAAAAAAUGC | 757 | GCAUUUUUUAGUAGAGAUGGGGUUUCA | 758 |
| 376 | AACCCCAUCUCUACUAAAAAAUGCA | 759 | UGCAUUUUUUAGUAGAGAUGGGGUUUC | 760 |
| 377 | CCCAUCUCUACUAAAAAAUGCAAAA | 761 | UUUUGCAUUUUUUAGUAGAGAUGGGGU | 762 |
| 378 | AUCAAAACCCUUAUGGCAGACUGTT | 763 | AACAGUCUGCCAUAAGGGUUUUGAUAU | 764 |
| 379 | UAUUUUAUUUGUCGUGCUUAUAUGT | 765 | ACAUAUAAGCACGACAAAUAAAAUACA | 766 |
| 380 | GUGUUGCCCAAGUUUCUAUGGUGAA | 767 | UUCACCAUAGAAACUUGGGCAACACAU | 768 |
| 381 | GCCCAAGUUUCUAUGGUGAACGGTA | 769 | UACCGUUCACCAUAGAAACUUGGGCAA | 770 |
| 382 | CCCAAGUUUCUAUGGUGAACGGUAT | 771 | AUACCGUUCACCAUAGAAACUUGGGCA | 772 |
| 383 | ACUUUCAGCAUGAGAAAAUAACUCC | 773 | GGAGUUAUUUUCUCAUGCUGAAAGUGA | 774 |
| 384 | CUUUCAGCAUGAGAAAAUAACUCCT | 775 | AGGAGUUAUUUUCUCAUGCUGAAAGUG | 776 |

In vitro cell-based assays: The ability of each of the 384 DsiRNAs listed in Table 1 to inhibit PNPLA3 expression is determined using in vitro cell-based assays. Further, the nucleotide sequences for the passenger strand and guide strand of the DsiRNAs have a distinct pattern of modified nucleotides and phosphorothioate linkages (see, e.g., FIG. 2). Briefly, HuH-7 human liver cells stably expressing PNPLA3 are transfected with each of the DsiRNAs (0.5 nM) in separate wells of a multi-well cell culture plate. Cells are maintained for 24 hr. following transfection, and then levels of remaining PNPLA3 mRNA from the transfected cells are determined using TAQMAN®-based qPCR assays. Two qPCR assays, a 3′ assay and a 5′ assay, are used to determine mRNA levels as measured by HEX and FAM probes, respectively.

The results of the HuH-7 cell-based assay with the DsiRNAs are shown in Table 2. Table 2 shows the results of the HuH-7 cell-based assay with 384 DsiRNAs that have guide strands that are complementary to human and monkey PNPLA3 mRNA (“double-common”). Transfection of a double-common DsiRNA that results in less than or equal to 30% PNPLA3 mRNA remaining in the cells when compared to negative controls is considered a candidate PNPLA3 expression inhibitor (referred to herein as a “hit”).

TABLE 2
Double-Common DsiRNA PNPLA3 Knockdown in HuH-7 Cells, 0.5 nM 24 hr-5′
and -3′ Assays % mRNA Remaining (normalized to HPRT1 and SFRS9 vs Mock Control).

| | PNPLA3-F495 | | PNPLA3-F595 | | Averages | |
|---|---|---|---|---|---|---|
| | % | % | % | % | Average % | Average |
| DsiRNA | Remaining | SEM | Remaining | SEM | Remaining | % SEM |
| 1 | — | — | 41.276 | 5.253 | 41.2760 | 5.2530 |
| 2 | 24.048 | 5.091 | 33.293 | 8.521 | 28.6705 | 6.8060 |
| 3 | 23.686 | 3.496 | 36.027 | 2.441 | 29.8565 | 2.9685 |
| 4 | 20.851 | 2.728 | 31.34 | 4.013 | 26.0955 | 3.3705 |
| 5 | 19.609 | 4.442 | 30.014 | 4.678 | 24.8115 | 4.5600 |
| 6 | 17.737 | 2.25 | 38.518 | 5.495 | 28.1275 | 3.8725 |
| 7 | 42.296 | 6.654 | — | — | 42.2960 | 6.6540 |
| 8 | 30.019 | 2.843 | 35.275 | 5.401 | 32.6470 | 4.1220 |
| 9 | 107.259 | 15.824 | 77.986 | 22.381 | 92.6225 | 19.1025 |
| 10 | 69.476 | 36.895 | 83.554 | 28.116 | 76.5150 | 32.5055 |
| 11 | 39.447 | 10.162 | 31.949 | 3.604 | 35.6980 | 6.8830 |
| 12 | — | — | 31.418 | 19.31 | 31.4180 | 19.3100 |
| 13 | 82.71 | 31.376 | 81.104 | 20.936 | 81.9070 | 26.1560 |
| 14 | 35.657 | 4.964 | 54.326 | 6.388 | 44.9915 | 5.6760 |
| 15 | 32.849 | 4.61 | 28.693 | 2.08 | 30.7710 | 3.3450 |
| 16 | — | — | — | — | — | — |
| 17 | — | — | 38.503 | 11.845 | 38.5030 | 11.8450 |
| 18 | 35.771 | 6.677 | 53.554 | 4.637 | 44.6625 | 5.6570 |
| 19 | 13.754 | 5.708 | 23.172 | 6.037 | 18.4630 | 5.8725 |
| 20 | 30.11 | 15.649 | 40.907 | 11.249 | 35.5085 | 13.4490 |
| 21 | 29.368 | 4.354 | 51.409 | 11.578 | 40.3885 | 7.9660 |
| 22 | 55.824 | 19.324 | 56.734 | 15.375 | 56.2790 | 17.3495 |
| 23 | 27.853 | 4.585 | 30.09 | 5.153 | 28.9715 | 4.8690 |
| 24 | 38.262 | 20.656 | 42.106 | 12.679 | 40.1840 | 16.6675 |
| 25 | 27.359 | 2.988 | 33.97 | 4.654 | 30.6645 | 3.8210 |
| 26 | 34.444 | 22.868 | 75.891 | 39.63 | 55.1675 | 31.2490 |
| 27 | 52.207 | 13.773 | 37.602 | 4.715 | 44.9045 | 9.2440 |
| 28 | 27.654 | 4.589 | 26.822 | 3.115 | 27.2380 | 3.8520 |
| 29 | 40.199 | 5.272 | 35.055 | 4.257 | 37.6270 | 4.7645 |
| 30 | 27.071 | 6.617 | 27.672 | 4.172 | 27.3715 | 5.3945 |
| 31 | 26.453 | 4.251 | 31.149 | 2.751 | 28.8010 | 3.5010 |
| 32 | 44.932 | 8.656 | 32.93 | 3.5 | 38.9310 | 6.0780 |
| 33 | 97.873 | 67.085 | 132.571 | 104.973 | 115.2220 | 86.0290 |
| 34 | 27.054 | 5.653 | 44.814 | 11.339 | 35.9340 | 8.4960 |
| 35 | 178.389 | 145.305 | 220.361 | 218.004 | 199.3750 | 181.6545 |
| 36 | 22.973 | 7.112 | 39.55 | 6.188 | 31.2615 | 6.6500 |
| 37 | 32.844 | 4.1 | 40.917 | 5.782 | 36.8805 | 4.9410 |
| 38 | 36.642 | 13.921 | 34.387 | 13.321 | 35.5145 | 13.6210 |
| 39 | 43.801 | 18.504 | 54.98 | 32.264 | 49.3905 | 25.3840 |
| 40 | 38.178 | 18.519 | 103.356 | 40.968 | 70.7670 | 29.7435 |
| 41 | 33.309 | 13.99 | 36.814 | 10.58 | 35.0615 | 12.2850 |
| 42 | 25.27 | 4.156 | 26.624 | 4.591 | 25.9470 | 4.3735 |
| 43 | 22.02 | 2.478 | 39.928 | 6.08 | 30.9740 | 4.2790 |
| 44 | 19.086 | 1.83 | 28.238 | 2.134 | 23.6620 | 1.9820 |
| 45 | 60.907 | 30.543 | 77.064 | 28.815 | 68.9855 | 29.6790 |
| 46 | 32.715 | 4.298 | 45.502 | 4.177 | 39.1085 | 4.2375 |
| 47 | 32.36 | 7.13 | 50.54 | 6.722 | 41.4500 | 6.9260 |
| 48 | 28.239 | 8.334 | 52.43 | 10.568 | 40.3345 | 9.4510 |
| 49 | 49.22 | 7.374 | 44.676 | 7.588 | 46.9480 | 7.4810 |
| 50 | 64.843 | 20.454 | 53.307 | 13.88 | 59.0750 | 17.1670 |
| 51 | 31.516 | 10.842 | 28.143 | 3.648 | 29.8295 | 7.2450 |
| 52 | 38.404 | 7.542 | 29.373 | 4.595 | 33.8885 | 6.0685 |
| 53 | 55.094 | 5.175 | 65.67 | 8.678 | 60.3820 | 6.9265 |
| 54 | 56.104 | 4.672 | 48.547 | 3.664 | 52.3255 | 4.1680 |
| 55 | 78.803 | 6.549 | 50.57 | 4.167 | 64.6865 | 5.3580 |
| 56 | 56.816 | 6.257 | 42.303 | 4.046 | 49.5595 | 5.1515 |
| 57 | 22.321 | 1.477 | 24.024 | 1.797 | 23.1725 | 1.6370 |
| 58 | 42.104 | 8.901 | 86.02 | 10.205 | 64.0620 | 9.5530 |
| 59 | 37.617 | 4.933 | 41.69 | 3.548 | 39.6535 | 4.2405 |
| 60 | 49.219 | 7.084 | 45.388 | 4.609 | 47.3035 | 5.8465 |
| 61 | 28.515 | 4.427 | 34.695 | 4.696 | 31.6050 | 4.5615 |
| 62 | 24.048 | 2.942 | 27.275 | 2.209 | 25.6615 | 2.5755 |
| 63 | 33.399 | 2.377 | 34.226 | 3.995 | 33.8125 | 3.1860 |
| 64 | 17.981 | 2.846 | 34.171 | 6.769 | 26.0760 | 4.8075 |
| 65 | 27.426 | 1.916 | 31.928 | 4.955 | 29.6770 | 3.4355 |
| 66 | 26.094 | 3.248 | 23.97 | 3.488 | 25.0320 | 3.3680 |
| 67 | 21.158 | 2.466 | 24.938 | 2.599 | 23.0480 | 2.5325 |
| 68 | 35.62 | 4.572 | 34.261 | 2.97 | 34.9405 | 3.7710 |
| 69 | 21.882 | 3.332 | 33.373 | 3.622 | 27.6275 | 3.4770 |
| 70 | 40.405 | 4.812 | 42.552 | 4.601 | 41.4785 | 4.7065 |
| 71 | 22.1 | 2.422 | 34.351 | 2.553 | 28.2255 | 2.4875 |
| 72 | 33.555 | 4.796 | 36.455 | 3.012 | 35.0050 | 3.9040 |
| 73 | 25.822 | 3.189 | 34.328 | 2.337 | 30.0750 | 2.7630 |
| 74 | 35.156 | 8.593 | 42.12 | 6.588 | 38.6380 | 7.5905 |
| 75 | 30.278 | 2.948 | 39.251 | 2.5 | 34.7645 | 2.7240 |
| 76 | 54.252 | 11.633 | 55.948 | 7.739 | 55.1000 | 9.6860 |
| 77 | 32.7 | 2.948 | 32.575 | 2.223 | 32.6375 | 2.5855 |
| 78 | 47.513 | 9.443 | 41.285 | 5.205 | 44.3990 | 7.3240 |
| 79 | 27.208 | 6.712 | 30.186 | 2.302 | 28.6970 | 4.5070 |
| 80 | 41.53 | 8.596 | 40.294 | 7.195 | 40.9120 | 7.8955 |
| 81 | 24.351 | 5.957 | 30.338 | 1.908 | 27.3445 | 3.9325 |
| 82 | 32.801 | 2.291 | 36.67 | 2.213 | 34.7355 | 2.2520 |
| 83 | 24.892 | 2.701 | 31.062 | 3.006 | 27.9770 | 2.8535 |
| 84 | 37.631 | 5.376 | 33.714 | 3.496 | 35.6725 | 4.4360 |
| 85 | 36.987 | 1.676 | 39.783 | 1.568 | 38.3850 | 1.6220 |
| 86 | 54.723 | 3.207 | 55.297 | 3.214 | 55.0100 | 3.2105 |
| 87 | 77.564 | 4.28 | 69.537 | 3.448 | 73.5505 | 3.8640 |
| 88 | 52.849 | 5.817 | 56.297 | 6.566 | 54.5730 | 6.1915 |
| 89 | 26.699 | 2.653 | 41.509 | 3.837 | 34.1040 | 3.2450 |
| 90 | 30.182 | 4.019 | 42.498 | 2.662 | 36.3400 | 3.3405 |
| 91 | 25.266 | 4.57 | 27.045 | 2.857 | 26.1555 | 3.7135 |
| 92 | 26.577 | 3.612 | 30.034 | 2.501 | 28.3055 | 3.0565 |
| 93 | 26.538 | 2.286 | 34 | 2.564 | 30.2690 | 2.4250 |
| 94 | 48.767 | 5.695 | 30.921 | 2.911 | 39.8440 | 4.3030 |
| 95 | 30.369 | 4.104 | 32.567 | 4.019 | 31.4680 | 4.0615 |
| 96 | 33.205 | 8.907 | 38.475 | 5.665 | 35.8400 | 7.2860 |
| 97 | 14.726 | 1.561 | 11.303 | 0.555 | 13.0145 | 1.0580 |
| 98 | 22.478 | 4.813 | 16.728 | 2.957 | 19.6030 | 3.8850 |
| 99 | 18.627 | 2.295 | 16.015 | 1.822 | 17.3210 | 2.0585 |
| 100 | 13.055 | 2.262 | 11.896 | 1.013 | 12.4755 | 1.6375 |
| 101 | 15.753 | 2.573 | 11.243 | 0.971 | 13.4980 | 1.7720 |
| 102 | 24.287 | 1.728 | 15.82 | 0.749 | 20.0535 | 1.2385 |
| 103 | 17.645 | 3.371 | 10.338 | 1.306 | 13.9915 | 2.3385 |
| 104 | 22.114 | 6.098 | 12.625 | 2.184 | 17.3695 | 4.1410 |
| 105 | 33.791 | 5.469 | 18.178 | 2.526 | 25.9845 | 3.9975 |
| 106 | 34.826 | 4.934 | 16.945 | 1.304 | 25.8855 | 3.1190 |
| 107 | 25.682 | 1.01 | 13.707 | 0.505 | 19.6945 | 0.7575 |
| 108 | 23.086 | 1.758 | 12.884 | 1.012 | 17.9850 | 1.3850 |
| 109 | 20.041 | 3.132 | 10.126 | 1.13 | 15.0835 | 2.1310 |
| 110 | 28.606 | 2.003 | 13.448 | 0.804 | 21.0270 | 1.4035 |
| 111 | 43.662 | 4.981 | 20.448 | 1.281 | 32.0550 | 3.1310 |
| 112 | 20.181 | 1.863 | 12.302 | 1.629 | 16.2415 | 1.7460 |
| 113 | 20.934 | 6.186 | 15.977 | 2.091 | 18.4555 | 4.1385 |
| 114 | 38.491 | 7.949 | 23.525 | 4.479 | 31.0080 | 6.2140 |
| 115 | 21.566 | 1.511 | 14.963 | 0.928 | 18.2645 | 1.2195 |
| 116 | 18.051 | 1.808 | 11.863 | 1.114 | 14.9570 | 1.4610 |
| 117 | 15.962 | 2.707 | 11.739 | 0.976 | 13.8505 | 1.8415 |
| 118 | 37.771 | 4.761 | 25.737 | 2.365 | 31.7540 | 3.5630 |
| 119 | 47.032 | 11.179 | 22.511 | 4.506 | 34.7715 | 7.8425 |
| 120 | 66.607 | 3.571 | 32.035 | 2.004 | 49.3210 | 2.7875 |
| 121 | 35.025 | 9.645 | 15.594 | 4.295 | 25.3095 | 6.9700 |
| 122 | 33.49 | 9.414 | 14.094 | 2.996 | 23.7920 | 6.2050 |
| 123 | 27.725 | 6.288 | 15.172 | 2.581 | 21.4485 | 4.4345 |
| 124 | 29.415 | 3.434 | 10.892 | 1.452 | 20.1535 | 2.4430 |
| 125 | 21.307 | 2.786 | 12.129 | 1.254 | 16.7180 | 2.0200 |
| 126 | 26.243 | 4.681 | 8.668 | 1.375 | 17.4555 | 3.0280 |
| 127 | 27.085 | 4.693 | 10.576 | 1.527 | 18.8305 | 3.1100 |
| 128 | 20.768 | 4.12 | 12.92 | 2.463 | 16.8440 | 3.2915 |
| 129 | 18.319 | 1.349 | 13.094 | 1.028 | 15.7065 | 1.1885 |
| 130 | 28.795 | 2.276 | 15.029 | 1.164 | 21.9120 | 1.7200 |
| 131 | 17.799 | 2.265 | 12.145 | 0.808 | 14.9720 | 1.5365 |
| 132 | 27.693 | 7.219 | 12.343 | 1.574 | 20.0180 | 4.3965 |
| 133 | 24.005 | 2.76 | 13.061 | 0.765 | 18.5330 | 1.7625 |
| 134 | 21.903 | 3.009 | 11.031 | 1.763 | 16.4670 | 2.3860 |
| 135 | 14.058 | 1.645 | 10.457 | 1.234 | 12.2575 | 1.4395 |
| 136 | 15.21 | 4.068 | 12.22 | 0.846 | 13.7150 | 2.4570 |
| 137 | 21.482 | 3.191 | 15.113 | 1.482 | 18.2975 | 2.3365 |
| 138 | 24.386 | 4.677 | 13.694 | 1.354 | 19.0400 | 3.0155 |
| 139 | 26.608 | 4.511 | 17.112 | 1.574 | 21.8600 | 3.0425 |
| 140 | 32.503 | 3.692 | 20.895 | 3.738 | 26.6990 | 3.7150 |
| 141 | 23.794 | 1.508 | 16.334 | 1.555 | 20.0640 | 1.5315 |
| 142 | 31.213 | 7.279 | 15.566 | 3.053 | 23.3895 | 5.1660 |
| 143 | 27.265 | 4.596 | 18.117 | 2.525 | 22.6910 | 3.5605 |
| 144 | 36.408 | 5.816 | 22.612 | 3.627 | 29.5100 | 4.7215 |
| 145 | 57.79 | 8.838 | 24.119 | 3.876 | 40.9545 | 6.3570 |
| 146 | 49.794 | 6.232 | 59.38 | 7.58 | 54.5870 | 6.9060 |
| 147 | 47.063 | 10.454 | 33.805 | 4.167 | 40.4340 | 7.3105 |
| 148 | 37.932 | 3.753 | 30.31 | 3.198 | 34.1210 | 3.4755 |
| 149 | 61.573 | 12.448 | 30.541 | 3.873 | 46.0570 | 8.1605 |
| 150 | 58.524 | 6.231 | 39.629 | 3.33 | 49.0765 | 4.7805 |
| 151 | 43.03 | 4.916 | 21.046 | 2.885 | 32.0380 | 3.9005 |
| 152 | 44.872 | 8.77 | 21.007 | 2.636 | 32.9395 | 5.7030 |
| 153 | 40.396 | 5.688 | 22.825 | 2.132 | 31.6105 | 3.9100 |
| 154 | 65.539 | 13.255 | 45.417 | 6.461 | 55.4780 | 9.8580 |
| 155 | 48.703 | 9.937 | 31.374 | 2.867 | 40.0385 | 6.4020 |
| 156 | 37.181 | 4.937 | 29.617 | 2.937 | 33.3990 | 3.9370 |
| 157 | 35.186 | 4.535 | 17.878 | 1.581 | 26.5320 | 3.0580 |
| 158 | 51.661 | 2.695 | 33.002 | 1.991 | 42.3315 | 2.3430 |
| 159 | 30.77 | 4.109 | 19.233 | 1.542 | 25.0015 | 2.8255 |
| 160 | 36.094 | 3.542 | 17.974 | 1.657 | 27.0340 | 2.5995 |
| 161 | 35.409 | 2.366 | 33.483 | 2.737 | 34.4460 | 2.5515 |
| 162 | 33.433 | 2.074 | 26.398 | 3.543 | 29.9155 | 2.8085 |
| 163 | 42.491 | 4.39 | 22.276 | 1.604 | 32.3835 | 2.9970 |
| 164 | 33.078 | 3.228 | 32.84 | 3.386 | 32.9590 | 3.3070 |
| 165 | 45.275 | 3.204 | 39.092 | 3.755 | 42.1835 | 3.4795 |
| 166 | 29.602 | 1.738 | 24.219 | 1.898 | 26.9105 | 1.8180 |
| 167 | 27.784 | 3.443 | 22.843 | 1.792 | 25.3135 | 2.6175 |
| 168 | 74.683 | 10.617 | 49.732 | 3.286 | 62.2075 | 6.9515 |
| 169 | 48.336 | 4.662 | 44.463 | 3.058 | 46.3995 | 3.8600 |
| 170 | 50.128 | 9.574 | 41.486 | 4.115 | 45.8070 | 6.8445 |
| 171 | 47.25 | 9.041 | 37.456 | 4.455 | 42.3530 | 6.7480 |
| 172 | 53.153 | 7.175 | 30.582 | 6.503 | 41.8675 | 6.8390 |
| 173 | 39.084 | 6.244 | 25.727 | 2.191 | 32.4055 | 4.2175 |
| 174 | 45.005 | 1.875 | 33.581 | 2.379 | 39.2930 | 2.1270 |
| 175 | 47.646 | 6.625 | 26.629 | 4.836 | 37.1375 | 5.7305 |
| 176 | 55.395 | 7.326 | 34.511 | 6.658 | 44.9530 | 6.9920 |
| 177 | 33.771 | 3.981 | 34.434 | 3.484 | 34.1025 | 3.7325 |
| 178 | 67.077 | 10.657 | 56.357 | 7.847 | 61.7170 | 9.2520 |
| 179 | 35.008 | 4.54 | 28.283 | 2.932 | 31.6455 | 3.7360 |
| 180 | 42.068 | 3.42 | 28.527 | 1.676 | 35.2975 | 2.5480 |
| 181 | 40.143 | 2.884 | 23.643 | 3.134 | 31.8930 | 3.0090 |
| 182 | 55.98 | 12.403 | 27.675 | 2.591 | 41.8275 | 7.4970 |
| 183 | 45.856 | 4.074 | 26.554 | 3.411 | 36.2050 | 3.7425 |
| 184 | 47.638 | 13.905 | 23.569 | 4.462 | 35.6035 | 9.1835 |
| 185 | 29.627 | 2.072 | 28.727 | 2.788 | 29.1770 | 2.4300 |
| 186 | 25.87 | 1.617 | 24.308 | 1.641 | 25.0890 | 1.6290 |
| 187 | 28.469 | 3.789 | 28.409 | 2.228 | 28.4390 | 3.0085 |
| 188 | 43.256 | 3.532 | 29.637 | 1.678 | 36.4465 | 2.6050 |
| 189 | 35.294 | 2.99 | 26.036 | 1.809 | 30.6650 | 2.3995 |
| 190 | 41.63 | 3.818 | 31.807 | 1.941 | 36.7185 | 2.8795 |
| 191 | 33.432 | 2.727 | 26.418 | 1.23 | 29.9250 | 1.9785 |
| 192 | 38.533 | 3.055 | 26.167 | 2.676 | 32.3500 | 2.8655 |
| 193 | 52.145 | 7.633 | 66.647 | 7.649 | 59.3960 | 7.6410 |
| 194 | 78.674 | 19.973 | 77.211 | 18.407 | 77.9425 | 19.1900 |
| 195 | 61.3 | 14.568 | 47.323 | 8.818 | 54.3115 | 11.6930 |
| 196 | 82.319 | 28.729 | 49.804 | 10.153 | 66.0615 | 19.4410 |
| 197 | 47.078 | 7.783 | 46.383 | 6.365 | 46.7305 | 7.0740 |
| 198 | 43.554 | 13.721 | 39.919 | 8.064 | 41.7365 | 10.8925 |
| 199 | 40.89 | 12.488 | 44.564 | 5.499 | 42.7270 | 8.9935 |
| 200 | 46.423 | 6.179 | 34.111 | 2.888 | 40.2670 | 4.5335 |
| 201 | 44.093 | 8.793 | 52.982 | 7.827 | 48.5375 | 8.3100 |
| 202 | 51.569 | 12.11 | 47.774 | 10.111 | 49.6715 | 11.1105 |
| 203 | 39.021 | 4.874 | 51.153 | 5.821 | 45.0870 | 5.3475 |
| 204 | 52.055 | 9.223 | 31.552 | 3.951 | 41.8035 | 6.5870 |
| 205 | 41.044 | 5.775 | 34.923 | 3.705 | 37.9835 | 4.7400 |
| 206 | 31.351 | 3.687 | 32.129 | 2.472 | 31.7400 | 3.0795 |
| 207 | 43.82 | 4.633 | 28.098 | 1.853 | 35.9590 | 3.2430 |
| 208 | 36.337 | 6.743 | 29.077 | 3.006 | 32.7070 | 4.8745 |
| 209 | 36.945 | 7.059 | 29.934 | 3.642 | 33.4395 | 5.3505 |
| 210 | 37.592 | 3.376 | 37.793 | 2.909 | 37.6925 | 3.1425 |
| 211 | 36.686 | 3.72 | 35.615 | 3.93 | 36.1505 | 3.8250 |
| 212 | 36.654 | 4.758 | 32.036 | 2.656 | 34.3450 | 3.7070 |
| 213 | 42.177 | 6.122 | 31.067 | 4.128 | 36.6220 | 5.1250 |
| 214 | 37.527 | 8.471 | 32.699 | 4.645 | 35.1130 | 6.5580 |
| 215 | 37.302 | 6.865 | 29.908 | 3.386 | 33.6050 | 5.1255 |
| 216 | 39.019 | 8.467 | 28.36 | 2.817 | 33.6895 | 5.6420 |
| 217 | 53.521 | 13.053 | 25.512 | 3.686 | 39.5165 | 8.3695 |
| 218 | 41.043 | 11.011 | 27.355 | 5.791 | 34.1990 | 8.4010 |
| 219 | 51.988 | 10.693 | 31.329 | 3.319 | 41.6585 | 7.0060 |
| 220 | 54.023 | 6.301 | 41.112 | 4.76 | 47.5675 | 5.5305 |
| 221 | 58.76 | 6.462 | 39.758 | 2.97 | 49.2590 | 4.7160 |
| 222 | 236.787 | 43.566 | 243.617 | 53.774 | 240.2020 | 48.6700 |
| 223 | 61.825 | 6.104 | 39.095 | 4.733 | 50.4600 | 5.4185 |
| 224 | 60.335 | 8.345 | 42.762 | 5.15 | 51.5485 | 6.7475 |
| 225 | 42.422 | 4.628 | 33.442 | 3.115 | 37.9320 | 3.8715 |
| 226 | 48.764 | 5.455 | 26.922 | 2.335 | 37.8430 | 3.8950 |
| 227 | 57.581 | 7.579 | 42.222 | 4.06 | 49.9015 | 5.8195 |
| 228 | 46.242 | 5.713 | 32.37 | 2.362 | 39.3060 | 4.0375 |
| 229 | 59.85 | 6.227 | 37.452 | 3.019 | 48.6510 | 4.6230 |
| 230 | 42.275 | 3.602 | 32.725 | 2.969 | 37.5000 | 3.2855 |
| 231 | 54.799 | 3.621 | 33.443 | 2.238 | 44.1210 | 2.9295 |
| 232 | 52.778 | 5.345 | 35.823 | 3.952 | 44.3005 | 4.6485 |
| 233 | 54.107 | 6.101 | 33.629 | 3.781 | 43.8680 | 4.9410 |
| 234 | 57.658 | 5.363 | 42.349 | 3.414 | 50.0035 | 4.3885 |
| 235 | 39.81 | 6.669 | 29.299 | 2.818 | 34.5545 | 4.7435 |
| 236 | 49.078 | 4.885 | 31.99 | 2.007 | 40.5340 | 3.4460 |
| 237 | 57.23 | 6.733 | 36.45 | 3.148 | 46.8400 | 4.9405 |
| 238 | 51.689 | 4.587 | 33.588 | 2.816 | 42.6385 | 3.7015 |
| 239 | 44.895 | 5.883 | 33.621 | 3.034 | 39.2580 | 4.4585 |
| 240 | 59.327 | 4.174 | 43.637 | 4.972 | 51.4820 | 4.5730 |
| 241 | 63.766 | 13.264 | 50.677 | 5.209 | 57.2215 | 9.2365 |
| 242 | 66.835 | 12.5 | 52.004 | 7.592 | 59.4195 | 10.0460 |
| 243 | 54.29 | 6.239 | 38.992 | 4.219 | 46.6410 | 5.2290 |
| 244 | 72.508 | 13.528 | 36.851 | 5.599 | 54.6795 | 9.5635 |
| 245 | 65.646 | 9.098 | 55.163 | 6.389 | 60.4045 | 7.7435 |
| 246 | 51.051 | 6.557 | 46.828 | 6.46 | 48.9395 | 6.5085 |
| 247 | 60.801 | 6.585 | 41.571 | 5.728 | 51.1860 | 6.1565 |
| 248 | 50.195 | 6.174 | 39.365 | 4.661 | 44.7800 | 5.4175 |
| 249 | 37.378 | 5.543 | 42.906 | 6.632 | 40.1420 | 6.0875 |
| 250 | 43.321 | 5.067 | 30.966 | 3.133 | 37.1435 | 4.1000 |
| 251 | 45.472 | 4.875 | 32.346 | 3.885 | 38.9090 | 4.3800 |
| 252 | 43.062 | 3.621 | 28.848 | 2.106 | 35.9550 | 2.8635 |
| 253 | 37.701 | 3.518 | 25.509 | 2.268 | 31.6050 | 2.8930 |
| 254 | 58.2 | 18.808 | 36.005 | 9.635 | 47.1025 | 14.2215 |
| 255 | 31.77 | 3.926 | 27.901 | 4.411 | 29.8355 | 4.1685 |
| 256 | 38.708 | 3.847 | 36.377 | 3.262 | 37.5425 | 3.5545 |
| 257 | 30.508 | 3.645 | 31.26 | 2.622 | 30.8840 | 3.1335 |
| 258 | 44.359 | 5 | 34.497 | 2.636 | 39.4280 | 3.8180 |
| 259 | 67.139 | 7.485 | 40.465 | 2.943 | 53.8020 | 5.2140 |
| 260 | 66.589 | 6.837 | 39.106 | 3.29 | 52.8475 | 5.0635 |
| 261 | 48.322 | 10.679 | 26.008 | 2.39 | 37.1650 | 6.5345 |
| 262 | 50.546 | 4.737 | 32.219 | 2.427 | 41.3825 | 3.5820 |
| 263 | 59.307 | 7.82 | 29.231 | 2.967 | 44.2690 | 5.3935 |
| 264 | 97.302 | 11.857 | 53.08 | 6.094 | 75.1910 | 8.9755 |
| 265 | 76.539 | 14.949 | 85.905 | 13.021 | 81.2220 | 13.9850 |
| 266 | 57.905 | 6.615 | 64.596 | 7.651 | 61.2505 | 7.1330 |
| 267 | 69.77 | 6.607 | 53.074 | 4.276 | 61.4220 | 5.4415 |
| 268 | 48.668 | 10.684 | 40.916 | 5.466 | 44.7920 | 8.0750 |
| 269 | 47.46 | 7.531 | 42.844 | 4.1 | 45.1520 | 5.8155 |
| 270 | 70.579 | 11.73 | 61.976 | 6.502 | 66.2775 | 9.1160 |
| 271 | 115.007 | 12.121 | 67.306 | 4.174 | 91.1565 | 8.1475 |
| 272 | 82.348 | 12.571 | 104.237 | 16.188 | 93.2925 | 14.3795 |
| 273 | 72.215 | 14.678 | 53.943 | 7.425 | 63.0790 | 11.0515 |
| 274 | 49.167 | 8.023 | 49.69 | 6.118 | 49.4285 | 7.0705 |
| 275 | 41.439 | 3.584 | 29.44 | 2.996 | 35.4395 | 3.2900 |
| 276 | 53.402 | 8.54 | 50.701 | 3.872 | 52.0515 | 6.2060 |
| 277 | 56.148 | 3.835 | 32.342 | 3.153 | 44.2450 | 3.4940 |
| 278 | 67.762 | 15.057 | 45.771 | 7.974 | 56.7665 | 11.5155 |
| 279 | 53.393 | 3.781 | 31.896 | 2.753 | 42.6445 | 3.2670 |
| 280 | 62.738 | 14.71 | 39.414 | 4.46 | 51.0760 | 9.5850 |
| 281 | 40.384 | 8.034 | 38.263 | 3.196 | 39.3235 | 5.6150 |
| 282 | 40.763 | 5.446 | 40.712 | 4.898 | 40.7375 | 5.1720 |
| 283 | 46.465 | 10.212 | 46.647 | 6.183 | 46.5560 | 8.1975 |
| 284 | 38.536 | 4.547 | 41.452 | 2.836 | 39.9940 | 3.6915 |
| 285 | 42.301 | 4.416 | 43.768 | 4.259 | 43.0345 | 4.3375 |
| 286 | 46.313 | 8.357 | 38.798 | 5.187 | 42.5555 | 6.7720 |
| 287 | 50.769 | 6.505 | 43.597 | 4.509 | 47.1830 | 5.5070 |
| 288 | 38.562 | 8.297 | 37.896 | 4.33 | 38.2290 | 6.3135 |
| 289 | 97.407 | 9.944 | 84.032 | 7.979 | 90.7195 | 8.9615 |
| 290 | 96.276 | 16.596 | 80.344 | 14.757 | 88.3100 | 15.6765 |
| 291 | 97.306 | 19.495 | 65.589 | 6.382 | 81.4475 | 12.9385 |
| 292 | 55.475 | 9.264 | 45.599 | 8.096 | 50.5370 | 8.6800 |
| 293 | 62.494 | 8.981 | 44.842 | 6.356 | 53.6680 | 7.6685 |
| 294 | 64.097 | 10.104 | 45.876 | 4.721 | 54.9865 | 7.4125 |
| 295 | 54.142 | 14.352 | 44.557 | 6.065 | 49.3495 | 10.2085 |
| 296 | 45.915 | 5.238 | 42.595 | 4.868 | 44.2550 | 5.0530 |
| 297 | 52.489 | 10.839 | 40.88 | 3.707 | 46.6845 | 7.2730 |
| 298 | 63.515 | 15.058 | 48.368 | 7.137 | 55.9415 | 11.0975 |
| 299 | 68.855 | 5.952 | 39.142 | 4.218 | 53.9985 | 5.0850 |
| 300 | 41.279 | 2.923 | 42.539 | 2.744 | 41.9090 | 2.8335 |
| 301 | 61.222 | 5.21 | 35.294 | 2.489 | 48.2580 | 3.8495 |
| 302 | 61.31 | 7.946 | 54.541 | 6.669 | 57.9255 | 7.3075 |
| 303 | 72.478 | 6.439 | 34.292 | 3.108 | 53.3850 | 4.7735 |
| 304 | 67.572 | 16.336 | 42.125 | 6.87 | 54.8485 | 11.6030 |
| 305 | 50.921 | 8.983 | 33.358 | 4.008 | 42.1395 | 6.4955 |
| 306 | 57.53 | 5.6 | 69.86 | 6.547 | 63.6950 | 6.0735 |
| 307 | 64.026 | 11.056 | 39.867 | 4.008 | 51.9465 | 7.5320 |
| 308 | 49.409 | 3.059 | 42.979 | 2.509 | 46.1940 | 2.7840 |
| 309 | 54.897 | 4.814 | 31.307 | 1.962 | 43.1020 | 3.3880 |
| 310 | 41.554 | 2.628 | 37.128 | 1.841 | 39.3410 | 2.2345 |
| 311 | 51.666 | 8.47 | 40.257 | 4.367 | 45.9615 | 6.4185 |
| 312 | 49.509 | 6.575 | 28.02 | 4.538 | 38.7645 | 5.5565 |
| 313 | — | — | 194.826 | 68.134 | 194.8260 | 68.1340 |
| 314 | 121.989 | 16.822 | 138.651 | 21 | 130.3200 | 18.9110 |
| 315 | 80.309 | 31.932 | 61.6 | 21.185 | 70.9545 | 26.5585 |
| 316 | 92.927 | 25.688 | 67.475 | 19.158 | 80.2010 | 22.4230 |
| 317 | 90.794 | 26.314 | 68.783 | 17.49 | 79.7885 | 21.9020 |
| 318 | 78.946 | 5.722 | 89.051 | 5.366 | 83.9985 | 5.5440 |
| 319 | 78.528 | 22.516 | 68.248 | 14.662 | 73.3880 | 18.5890 |
| 320 | 88.504 | 27.66 | 74.364 | 17.313 | 81.4340 | 22.4865 |
| 321 | 80.819 | 11.586 | 72.082 | 9.362 | 76.4505 | 10.4740 |
| 322 | 66.905 | 11.106 | 59.407 | 7.519 | 63.1560 | 9.3125 |
| 323 | 56.665 | 7.201 | 65.996 | 7.423 | 61.3305 | 7.3120 |
| 324 | 58.472 | 14.789 | 52.989 | 6.283 | 55.7305 | 10.5360 |
| 325 | 73.835 | 10.27 | 45.629 | 3.865 | 59.7320 | 7.0675 |
| 326 | 83.653 | 12.949 | 55.705 | 5.376 | 69.6790 | 9.1625 |
| 327 | 48.135 | 5.765 | 47.686 | 2.973 | 47.9105 | 4.3690 |
| 328 | 58.846 | 13.768 | 64.173 | 8.7 | 61.5095 | 11.2340 |
| 329 | 46.418 | 3.234 | 43.86 | 4.767 | 45.1390 | 4.0005 |
| 330 | 48.925 | 5.8 | 47.32 | 5.483 | 48.1225 | 5.6415 |
| 331 | 60.81 | 13.53 | 36.129 | 3.04 | 48.4695 | 8.2850 |
| 332 | 56.142 | 10.276 | 41.861 | 5.196 | 49.0015 | 7.7360 |
| 333 | 57.072 | 5.485 | 46.44 | 1.828 | 51.7560 | 3.6565 |
| 334 | 57.031 | 14.153 | 47.644 | 5.986 | 52.3375 | 10.0695 |
| 335 | 63.616 | 8.355 | 62.766 | 7.336 | 63.1910 | 7.8455 |
| 336 | 61.994 | 10.795 | 49.157 | 4.36 | 55.5755 | 7.5775 |
| 337 | 109.966 | 11.513 | 73.307 | 11.438 | 91.6365 | 11.4755 |
| 338 | 110.715 | 15.908 | 92.237 | 13.006 | 101.4760 | 14.4570 |
| 339 | 75.364 | 14.03 | 67.654 | 10.63 | 71.5090 | 12.3300 |
| 340 | 109.658 | 7.631 | 67.114 | 6.374 | 88.3860 | 7.0025 |
| 341 | 81.366 | 8.105 | 61.479 | 4 | 71.4225 | 6.0525 |
| 342 | 64.415 | 15.53 | 79.511 | 24.66 | 71.9630 | 20.0950 |
| 343 | 74.24 | 10.057 | 46.431 | 5.731 | 60.3355 | 7.8940 |
| 344 | 86.963 | 13.807 | 54.298 | 8.525 | 70.6305 | 11.1660 |
| 345 | 71.074 | 6.207 | 57.396 | 6.551 | 64.2350 | 6.3790 |
| 346 | 91.626 | 10.114 | 74.902 | 8.206 | 83.2640 | 9.1600 |
| 347 | 77.149 | 8.202 | 53.758 | 5.371 | 65.4535 | 6.7865 |
| 348 | 80.631 | 9.944 | 56.546 | 8.401 | 68.5885 | 9.1725 |
| 349 | 60.505 | 5.38 | 37.207 | 4.379 | 48.8560 | 4.8795 |
| 350 | 64.783 | 11.132 | 46.827 | 8.602 | 55.8050 | 9.8670 |
| 351 | 70.732 | 7.768 | 42.967 | 3.442 | 56.8495 | 5.6050 |
| 352 | 85.716 | 13.405 | 56.756 | 5.896 | 71.2360 | 9.6505 |
| 353 | 49.942 | 6.657 | 49.317 | 3.253 | 49.6295 | 4.9550 |
| 354 | 38.757 | 3.56 | 37.798 | 2.633 | 38.2775 | 3.0965 |
| 355 | 45.029 | 2.934 | 38.812 | 2.665 | 41.9205 | 2.7995 |
| 356 | 38.688 | 3.896 | 34.3 | 2.617 | 36.4940 | 3.2565 |
| 357 | 52.918 | 9.736 | 35.223 | 3.54 | 44.0705 | 6.6380 |
| 358 | 52.294 | 3.336 | 44.793 | 5.552 | 48.5435 | 4.4440 |
| 359 | 53.158 | 3.096 | 41.327 | 3.233 | 47.2425 | 3.1645 |
| 360 | 103.51 | 27.753 | 62.923 | 11.824 | 83.2165 | 19.7885 |
| 361 | 122.759 | 25.999 | 106.083 | 13.134 | 114.4210 | 19.5665 |
| 362 | 106.559 | 19.744 | 101.161 | 14.303 | 103.8600 | 17.0235 |
| 363 | 109.834 | 19.943 | 79.727 | 14.271 | 94.7805 | 17.1070 |
| 364 | 102.297 | 10.688 | 78.932 | 9.566 | 90.6145 | 10.1270 |
| 365 | 82.014 | 8.363 | 73.591 | 5.609 | 77.8025 | 6.9860 |
| 366 | 67.391 | 23.514 | 69.264 | 11.51 | 68.3275 | 17.5120 |
| 367 | 95.165 | 15.862 | 42.233 | 5.102 | 68.6990 | 10.4820 |
| 368 | 95.977 | 20.567 | 51.602 | 9.654 | 73.7895 | 15.1105 |
| 369 | 65.153 | 14.652 | 46.363 | 7.328 | 55.7580 | 10.9900 |
| 370 | 58.571 | 7.578 | 50.496 | 6.145 | 54.5335 | 6.8615 |
| 371 | 84.037 | 13.739 | 52.431 | 5.879 | 68.2340 | 9.8090 |
| 372 | 67.564 | 8.585 | 48.839 | 4.307 | 58.2015 | 6.4460 |
| 373 | 57.82 | 10.735 | 38.625 | 4.956 | 48.2225 | 7.8455 |
| 374 | 84.402 | 27.865 | 37.659 | 6.895 | 61.0305 | 17.3800 |
| 375 | 72.533 | 12.7 | 45.661 | 5.549 | 59.0970 | 9.1245 |
| 376 | 68.34 | 7.43 | 44.068 | 4.428 | 56.2040 | 5.9290 |
| 377 | 70.417 | 6.309 | 53.79 | 6.279 | 62.1035 | 6.2940 |
| 378 | 61.427 | 3.64 | 47.649 | 2.676 | 54.5380 | 3.1580 |
| 379 | 95.519 | 18.074 | 57.492 | 7.143 | 76.5055 | 12.6085 |
| 380 | 65.481 | 5.625 | 42.825 | 2.2 | 54.1530 | 3.9125 |
| 381 | 59.761 | 9.824 | 33.808 | 2.686 | 46.7845 | 6.2550 |
| 382 | 58.179 | 4.499 | 36.306 | 3.105 | 47.2425 | 3.8020 |
| 383 | 73.232 | 3.793 | 41.961 | 2.581 | 57.5965 | 3.1870 |
| 384 | 69.482 | 14.129 | 34.575 | 5.662 | 52.0285 | 9.8955 |

These results show that DsiRNAs designed to target human PNPLA3 mRNA inhibit PNPLA3 expression in cells (as determined by a reduced amount of PNPLA3 mRNA in DsiRNA-transfected cells) and that the nucleotide sequences including the DsiRNA hits are useful for generating RNAi oligonucleotides to inhibit PNPLA3 expression. Further, these results demonstrate that multiple PNPLA3 target sequences are suitable for the RNAi-mediated inhibition of PNPLA3 expression.

### In Vivo Function

### Example 3: RNAi Oligonucleotide Inhibition of PNPLA3 Expression In Vivo

Of the 384 DsiRNAs screened in the HuH-7 cell-based assays described in Example 2, the nucleotide sequences of 192 DsiRNAs hits (Table 3) are selected for further evaluation in vivo. Briefly, the nucleotide sequences the selected DsiRNAs are used to generate 194 corresponding ds RNAi oligonucleotides including a nicked tetraloop GalNAc-conjugated structure (referred to herein as “GalNAc-conjugated PNPLA3 oligonucleotides”) having a 36-mer sense (passenger) strand and a 22-mer antisense (guide) strand. Further, the nucleotide sequences for the passenger strand and guide strand of the GalNAc-conjugated PNPLA3 oligonucleotides have a distinct pattern of modified nucleotides and phosphorothioate linkages (see, e.g., FIG. 1 for a schematic of the generic structure and chemical modification pattern of the GalNAc-conjugated PNPLA3 oligonucleotides). The three adenosine nucleotides of the tetraloop each are conjugated to a GalNAc moiety (CAS #: 14131-60-3).

TABLE 3
GalXC-PNPLA3: 192 GalXC Compound In Vitro Screen.

| PNPLA3 | Passenger | Guide | PNPLA3-F495 | | PNPLA3-F595 | |
|---|---|---|---|---|---|---|
| Oligo- | (sense) | (antisense) | % | % | % | % |
| nucleotide | (SEQ ID NO:) | (SEQ ID NO:) | Remaining | SEM | Remaining | SEM |
| 1 | 777 | 778 | 47.485 | 10.196 | 42.315 | 9.325 |
| 2 | 779 | 780 | 88.393 | 13.374 | 74.853 | 8.613 |
| 3 | 781 | 782 | 71.522 | 13.389 | 62.163 | 8.489 |
| 4 | 783 | 784 | 48.2 | 12.808 | 59.051 | 15.527 |
| 5 | 785 | 786 | 87.111 | 20.654 | 74.136 | 14.422 |
| 6 | 787 | 788 | 52.772 | 4.946 | 32.37 | 3.842 |
| 7 | 789 | 790 | 60 | 4.856 | 56.847 | 3.105 |
| 8 | 791 | 792 | 89.452 | 17.034 | 90.454 | 16.078 |
| 9 | 793 | 794 | 76.773 | 16.13 | 58.479 | 11.665 |
| 10 | 795 | 796 | 85.463 | 9.685 | 66.419 | 7.575 |
| 11 | 797 | 798 | 108.216 | 4.243 | 70.258 | 3.457 |
| 12 | 799 | 800 | 57.964 | 8.916 | 47.12 | 9.746 |
| 13 | 801 | 802 | 91.802 | 15.466 | 80.834 | 14.834 |
| 14 | 803 | 804 | 108.246 | 8.747 | 102.424 | 9.238 |
| 15 | 805 | 806 | 102.556 | 8.612 | 91.909 | 8.507 |
| 16 | 807 | 808 | 123.63 | 8.863 | 110.429 | 6.112 |
| 17 | 809 | 810 | 70.82 | 7.935 | 48.198 | 3.957 |
| 18 | 811 | 812 | 105.252 | 5.434 | 76.73 | 5.427 |
| 19 | 813 | 814 | 73.737 | 7.411 | 63.414 | 3.163 |
| 20 | 815 | 816 | 53.275 | 5.084 | 40.621 | 3.768 |
| 21 | 817 | 818 | 73.13 | 8.636 | 54.227 | 4.991 |
| 22 | 819 | 820 | 69.662 | 5.077 | 65.603 | 4.833 |
| 23 | 821 | 822 | 115.648 | 7.396 | 93.812 | 6.834 |
| 24 | 823 | 824 | 113.539 | 8.877 | 95.22 | 8.13 |
| 25 | 825 | 826 | 72.315 | 22.04 | 65.007 | 19.295 |
| 26 | 827 | 828 | 52.756 | 8.42 | 52.681 | 4.521 |
| 27 | 829 | 830 | 50.002 | 5.36 | 45.103 | 6.461 |
| 28 | 831 | 832 | 65.906 | 5.919 | 61.42 | 3.683 |
| 29 | 833 | 834 | 85.13 | 15.786 | 81.4 | 13.245 |
| 30 | 835 | 836 | 94.2 | 7.683 | 92.984 | 6.53 |
| 31 | 837 | 838 | 79.41 | 9.652 | 78.856 | 9.311 |
| 32 | 839 | 840 | 110.611 | 10.798 | 121.904 | 11.977 |
| 33 | 841 | 842 | 61.132 | 15.106 | 54.942 | 10.832 |
| 34 | 843 | 844 | 22.997 | 2.613 | 26.894 | 3.012 |
| 35 | 845 | 846 | 69.893 | 7.912 | 71.053 | 6.942 |
| 36 | 847 | 848 | 42.209 | 6.514 | 37.063 | 6.765 |
| 37 | 849 | 850 | 32.369 | 6.443 | 30.326 | 5.18 |
| 38 | 851 | 852 | 98.013 | 5.195 | 99.493 | 5.551 |
| 39 | 853 | 854 | 108.745 | 12.02 | 109.98 | 14.348 |
| 40 | 855 | 856 | 108.242 | 5.703 | 106.183 | 7.088 |
| 41 | 857 | 858 | 62.227 | 18.716 | 47.683 | 7.008 |
| 42 | 859 | 860 | 77.369 | 5.452 | 72.082 | 4.805 |
| 43 | 861 | 862 | 97.72 | 6.492 | 104.918 | 2.468 |
| 44 | 863 | 864 | 95.316 | 16.953 | 81.092 | 8.169 |
| 45 | 865 | 866 | 65.563 | 3.105 | 63.593 | 5.933 |
| 46 | 867 | 868 | 45.891 | 2.704 | 41.178 | 2.034 |
| 47 | 869 | 870 | 70.44 | 8.4 | 69.907 | 11.675 |
| 48 | 871 | 872 | 42.793 | 4.981 | 41.802 | 4.578 |
| 49 | 873 | 874 | 69.377 | 12.858 | 75.563 | 14.64 |
| 50 | 875 | 876 | 94.54 | 12.65 | 113.746 | 13.701 |
| 51 | 877 | 878 | 96.644 | 9.928 | 88.742 | 7.236 |
| 52 | 879 | 880 | 57.675 | 7.9 | 54.023 | 9.11 |
| 53 | 881 | 882 | 45.257 | 4.671 | 41.066 | 8.781 |
| 54 | 883 | 884 | 64.216 | 13.821 | 57.326 | 9.308 |
| 55 | 885 | 886 | 80.64 | 10.056 | 83.19 | 12.71 |
| 56 | 887 | 888 | 53.489 | 6.288 | 60.359 | 10.933 |
| 57 | 889 | 890 | 43.058 | 8.468 | 67.338 | 7.905 |
| 58 | 891 | 892 | 92.209 | 4.675 | 88.981 | 4.725 |
| 59 | 893 | 894 | 125.612 | 12.186 | 131.574 | 15.729 |
| 60 | 895 | 896 | 56.646 | 7.227 | 58.75 | 7.18 |
| 61 | 897 | 898 | 63.963 | 4.129 | 64.01 | 3.511 |
| 62 | 899 | 900 | 35.42 | 4.93 | 43.575 | 3.839 |
| 63 | 901 | 902 | 80.942 | 5.673 | 99.785 | 7.605 |
| 64 | 903 | 904 | 28.438 | 9.671 | 41.409 | 10.949 |
| 65 | 905 | 906 | 78.01 | 12.28 | 70.329 | 13.215 |
| 66 | 907 | 908 | 63.519 | 7.735 | 56.465 | 6.226 |
| 67 | 909 | 910 | 102.482 | 9.653 | 86.816 | 7.212 |
| 68 | 911 | 912 | 73.153 | 6.21 | 76.354 | 7.251 |
| 69 | 913 | 914 | 115.319 | 14.233 | 109.375 | 11.395 |
| 70 | 915 | 916 | 50.507 | 16.055 | 61.744 | 17.75 |
| 71 | 917 | 918 | 64.122 | 18.86 | 67.106 | 19.792 |
| 72 | 919 | 920 | 45.025 | 11.057 | 36.962 | 8.699 |
| 73 | 921 | 922 | 64.691 | 7.735 | 70.342 | 7.008 |
| 74 | 923 | 924 | 91.612 | 17.728 | 73.763 | 9.075 |
| 75 | 925 | 926 | 30.626 | 5.128 | 34.089 | 2.646 |
| 76 | 927 | 928 | 85.724 | 5.965 | 98.425 | 5.178 |
| 77 | 929 | 930 | 77.837 | 24.988 | 65.493 | 20.924 |
| 78 | 931 | 932 | 66.549 | 13.96 | 70.219 | 13.374 |
| 79 | 933 | 934 | 88.812 | 9.773 | 93.579 | 14.174 |
| 80 | 935 | 936 | 126.281 | 13.138 | 125.405 | 9.847 |
| 81 | 937 | 938 | 33.474 | 12.471 | 36.472 | 7.102 |
| 82 | 939 | 940 | 36.521 | 6.105 | 36.344 | 12.791 |
| 83 | 941 | 942 | 79.756 | 28.563 | 79.725 | 24.95 |
| 84 | 943 | 944 | 91.352 | 9.565 | 103.633 | 12.963 |
| 85 | 945 | 946 | 79.374 | 11.693 | 88.146 | 11.711 |
| 86 | 947 | 948 | 104.203 | 12.127 | 111.907 | 13.828 |
| 87 | 949 | 950 | 78.157 | 5.154 | 100.371 | 7.36 |
| 88 | 951 | 952 | 117.96 | 7.199 | 130.066 | 9.106 |
| 89 | 953 | 954 | 84.233 | 7.043 | 78.918 | 4.772 |
| 90 | 955 | 956 | 70.825 | 5.146 | 76.977 | 8.968 |
| 91 | 957 | 958 | 59.621 | 7.853 | 65.538 | 7.22 |
| 92 | 959 | 960 | 91.443 | 6.954 | 99.109 | 6.096 |
| 93 | 961 | 962 | 46.844 | 11.633 | 51.957 | 9.244 |
| 94 | 963 | 964 | 81.508 | 6.389 | 87.195 | 8.633 |
| 95 | 965 | 966 | 91.805 | 8.83 | 108.782 | 9.098 |
| 96 | 967 | 968 | 96.733 | 4.922 | 125.597 | 5.479 |
| 97 | 969 | 970 | 92.658 | 10.451 | 110.074 | 12.159 |
| 98 | 971 | 972 | 100.916 | 7.648 | 107.715 | 9.441 |
| 99 | 973 | 974 | 108.731 | 9.204 | 130.037 | 11.933 |
| 100 | 975 | 976 | 80.886 | 6.51 | 82.486 | 5.492 |
| 101 | 977 | 978 | 114.601 | 6.354 | 125.063 | 7.668 |
| 102 | 979 | 980 | 64.486 | 7.636 | 81.933 | 8.887 |
| 103 | 981 | 982 | 48.127 | 7.673 | 70.594 | 9.464 |
| 104 | 983 | 984 | 98.266 | 10.023 | 125.396 | 13.292 |
| 105 | 985 | 986 | 78.835 | 8.422 | 85.896 | 6.258 |
| 106 | 987 | 988 | 65.38 | 6.01 | 75.699 | 5.561 |
| 107 | 989 | 990 | 82.332 | 16.715 | 110.985 | 21.204 |
| 108 | 991 | 992 | 50.805 | 3.714 | 49.059 | 3.238 |
| 109 | 993 | 994 | 82.297 | 10.387 | 74.612 | 6.957 |
| 110 | 995 | 996 | 56.254 | 4.26 | 51.783 | 3.569 |
| 111 | 997 | 998 | 37.191 | 3.673 | 43.781 | 4.781 |
| 112 | 999 | 1000 | 54.832 | 3.334 | 45.421 | 4.277 |
| 113 | 1001 | 1002 | 24.729 | 2.434 | 22.466 | 2.637 |
| 114 | 1003 | 1004 | 34.923 | 6.177 | 40.356 | 5.286 |
| 115 | 1005 | 1006 | 42.682 | 3.081 | 57.453 | 2.391 |
| 116 | 1007 | 1008 | 30.325 | 3.011 | 34.404 | 1.82 |
| 117 | 1009 | 1010 | 70.118 | 7.78 | 89.223 | 8.905 |
| 118 | 1011 | 1012 | 43.339 | 6.479 | 44.854 | 6.904 |
| 119 | 1013 | 1014 | 104.192 | 5.672 | 110.895 | 6.371 |
| 120 | 1015 | 1016 | 95.131 | 8.872 | 108.847 | 8.363 |
| 121 | 1017 | 1018 | 22.097 | 2.259 | 25.862 | 3.07 |
| 122 | 1019 | 1020 | 50.635 | 11.129 | 59.072 | 10.096 |
| 123 | 1021 | 1022 | 68.368 | 9.482 | 90.191 | 15.479 |
| 124 | 1023 | 1024 | 50.658 | 2.598 | 62.686 | 2.149 |
| 125 | 1025 | 1026 | 67.692 | 4.608 | 104.474 | 6.486 |
| 126 | 1027 | 1028 | 60.169 | 6.81 | 103.156 | 11.309 |
| 127 | 1029 | 1030 | 37.137 | 3.679 | 49.778 | 7.312 |
| 128 | 1031 | 1032 | 87.29 | 4.881 | 127.439 | 10.802 |
| 129 | 1033 | 1034 | 41.409 | 6.549 | 47.012 | 11.236 |
| 130 | 1035 | 1036 | 32.213 | 7.175 | 43.875 | 10.706 |
| 131 | 1037 | 1038 | 77.807 | 7.013 | 109.299 | 9.799 |
| 132 | 1039 | 1040 | 37.026 | 6.758 | 49.133 | 8.307 |
| 133 | 1041 | 1042 | 57.723 | 5.921 | 70.531 | 6.977 |
| 134 | 1043 | 1044 | 94.403 | 10.058 | 130.999 | 12.973 |
| 135 | 1045 | 1046 | 42.258 | 3.137 | 56.368 | 4.699 |
| 136 | 1047 | 1048 | 77.667 | 6.622 | 106.147 | 4.946 |
| 137 | 1049 | 1050 | 51.002 | 3.36 | 62.009 | 6.073 |
| 138 | 1051 | 1052 | 38.522 | 4.21 | 43.297 | 7.219 |
| 139 | 1053 | 1054 | 75.103 | 5.866 | 115.191 | 7.77 |
| 140 | 1055 | 1056 | 46.405 | 3.737 | 57.89 | 3.035 |
| 141 | 1057 | 1058 | 34.308 | 4.135 | 44.278 | 3.532 |
| 142 | 1059 | 1060 | 36.66 | 3.327 | 48.931 | 4.389 |
| 143 | 1061 | 1062 | 36.323 | 6.447 | 50.414 | 7.284 |
| 144 | 1063 | 1064 | 48.346 | 4.964 | 69.682 | 5.951 |
| 145 | 1065 | 1066 | 71.349 | 8.498 | 63.576 | 12.402 |
| 146 | 1067 | 1068 | 66.855 | 7.354 | 54.028 | 10.308 |
| 147 | 1069 | 1070 | 58.02 | 6.725 | 81.61 | 6.225 |
| 148 | 1071 | 1072 | 46.107 | 4.599 | 47.578 | 5.721 |
| 149 | 1073 | 1074 | 79.89 | 11.647 | 94.885 | 12.091 |
| 150 | 1075 | 1076 | 51.93 | 6.887 | 60.564 | 5.631 |
| 151 | 1077 | 1078 | 67.023 | 11.379 | 78.549 | 9.92 |
| 152 | 1079 | 1080 | 69.029 | 12.304 | 64.36 | 9.232 |
| 153 | 1081 | 1082 | 56.372 | 11.395 | 50.897 | 6.456 |
| 154 | 1083 | 1084 | 54.923 | 8.02 | 56.301 | 7.96 |
| 155 | 1085 | 1086 | 47.848 | 4.993 | 52.756 | 4.92 |
| 156 | 1087 | 1088 | 72.997 | 5.423 | 89.098 | 8.279 |
| 157 | 1089 | 1090 | 49.577 | 9.222 | 61.752 | 9.967 |
| 158 | 1091 | 1092 | 57.345 | 5.967 | 55.082 | 5.124 |
| 159 | 1093 | 1094 | 49.719 | 8.465 | 50.474 | 6.318 |
| 160 | 1095 | 1096 | 66.221 | 8.527 | 60.526 | 6.042 |
| 161 | 1097 | 1098 | 59.431 | 5.38 | 51.367 | 4.575 |
| 162 | 1099 | 1100 | 44.241 | 2.416 | 42.17 | 2.266 |
| 163 | 1101 | 1102 | 67.893 | 7.786 | 85.144 | 7.578 |
| 164 | 1103 | 1104 | 54.101 | 3.156 | 64.663 | 5.874 |
| 165 | 1105 | 1106 | 74.353 | 11.203 | 73.215 | 11.059 |
| 166 | 1107 | 1108 | 91.603 | 5.407 | 78.535 | 4.566 |
| 167 | 1109 | 1110 | 86.259 | 4.861 | 84.54 | 5.247 |
| 168 | 1111 | 1112 | 100.862 | 9.39 | 83.472 | 3.848 |
| 169 | 1113 | 1114 | 64.233 | 6.416 | 55.446 | 5.259 |
| 170 | 1115 | 1116 | 82.913 | 8.265 | 67.499 | 6.098 |
| 171 | 1117 | 1118 | 63.834 | 4.157 | 66.965 | 7.54 |
| 172 | 1119 | 1120 | 87.059 | 6.502 | 80.608 | 3.299 |
| 173 | 1121 | 1122 | 52.896 | 8.619 | 50.91 | 8.301 |
| 174 | 1123 | 1124 | 89.834 | 8.687 | 60.792 | 7.648 |
| 175 | 1125 | 1126 | 56.928 | 16.722 | 55.37 | 13.438 |
| 176 | 1127 | 1128 | 91.989 | 35.495 | 98.901 | 26.361 |
| 177 | 1129 | 1130 | 78.111 | 6.12 | 85.965 | 5.555 |
| 178 | 1131 | 1132 | 81.351 | 5.528 | 68.852 | 4.702 |
| 179 | 1133 | 1134 | 82.851 | 10.171 | 87.313 | 8.301 |
| 180 | 1135 | 1136 | 94.367 | 3.317 | 87.13 | 5.434 |
| 181 | 1137 | 1138 | 108.095 | 11.973 | 99.858 | 9.769 |
| 182 | 1139 | 1140 | 95.753 | 5.346 | 87.366 | 6.613 |
| 183 | 1141 | 1142 | 88.199 | 15.408 | 67.131 | 13.665 |
| 184 | 1143 | 1144 | 102.928 | 9.669 | 84.541 | 9.709 |
| 185 | 1145 | 1146 | 48.692 | 11.647 | 48.288 | 10.808 |
| 186 | 1147 | 1148 | 84.114 | 6.361 | 82.348 | 3.469 |
| 187 | 1149 | 1150 | 89.182 | 14.563 | 92.44 | 12.983 |
| 188 | 1151 | 1152 | 85.958 | 10.066 | 85.508 | 8.055 |
| 189 | 1153 | 1154 | 56.68 | 12.506 | 63.775 | 15.034 |
| 190 | 1155 | 1156 | 77.223 | 11.068 | 75.609 | 8.161 |
| 191 | 1157 | 1158 | 85.696 | 6.162 | 92.128 | 6.673 |
| 192 | 1159 | 1160 | 50.761 | 3.422 | 51.217 | 2.474 |

Mouse studies: Various GalNAc-conjugated PNPLA3 oligonucleotides, some of which are listed in Table 3, are evaluated in hydrodynamic injection (HDI) mouse model. Additional HDI studies are listed in tables 4, 5 and 6. For these HDI studies, the mice are engineered to transiently express human PNPLA3 mRNA in hepatocytes. A GalNAc-conjugated PNPLA3 oligonucleotide control (SEQ ID NOs: 1165 & 1166) is used as a benchmark control. Briefly, 6-8-week-old female CD-1 mice are treated SQ with a GalNAc-conjugated PNPLA3 oligonucleotide at a dose level of 1 mg/kg. Three days later (72 hr.), the mice are hydrodynamically injected with a DNA plasmid encoding the full human PNPLA3 gene under control of a ubiquitous cytomegalovirus (CMV) promoter sequence. One day after introduction of the plasmid, liver samples are collected. Total RNA derived from these mice are subjected to qRT-PCR analysis for PNPLA3 mRNA, relative to mice treated only with an identical volume of PBS. The values are normalized for transfection efficiency using the NeoR gene included on the plasmid.

As shown in Tables 4, 5 and 6 a number of the GalNAc-conjugated PNPLA3 oligonucleotides tested inhibited PNPLA3 expression, as determined by a reduced amount of PNPLA3 mRNA in liver samples from oligonucleotide-treated mice relative to mice treated with PBS. The mean % of remaining PNPLA3 mRNA in liver samples of mice treated with the benchmark GalNAc-conjugated PNPLA3 oligonucleotide control relative to mice treated with PBS. Table 4 shows that 18 of the 21 GalNAc-conjugated PNPLA3 oligonucleotides inhibit PNPLA3 expression to a greater extent than the reference GalNAc-conjugated PNPLA3 oligonucleotide used as control. Sequences of these oligonucleotides along with the modification patterns and SEQ ID NOs. is disclosed in Table A

TABLE 4
discloses single 0.5 mg/kg GalXC-PNPLA3 S.c. Dose, Day 3 HDI, Day 4 Takedown, Liver qPCR

| | Group | | | | | | | | | | | |
|---|---|---|---|---|---|---|---|---|---|---|---|---|
| | | −1170 | | | | | | | | | | |
| Animal | PBS | (Ref.) | −643 | −1110 | −1162 | −1170 | −1699 | −1703 | −1708 | −2104 | −2143 | −2155 |
| 1 | 105.7 | 96.7 | 78.2 | | 69.2 | 76.5 | 65.0 | 41.6 | 68.1 | 56.7 | 82.5 | 60.7 |
| 2 | 104.5 | 66.6 | 101.7 | 88.0 | 72.0 | 89.3 | 82.9 | 81.8 | 98.1 | 69.3 | 61.6 | 81.5 |
| 3 | 89.9 | 115.5 | 96.8 | 99.7 | 54.2 | 23.3 | 53.0 | 46.5 | 85.6 | 41.6 | 77.7 | 70.0 |
| 4 | 99.9 | 57.1 | 64.1 | 92.2 | 84.4 | 26.7 | 69.6 | 48.6 | 61.8 | 29.3 | 92.5 | 81.0 |
| 5 | | 107.2 | 64.2 | 70.4 | 118.2 | 75.0 | 39.4 | 63.4 | 39.7 | 35.5 | 89.1 | 61.2 |
| Average: | 100.0 | 88.6 | 81.0 | 87.6 | 79.6 | 58.1 | 62.0 | 56.4 | 70.6 | 46.5 | 80.7 | 70.9 |
| SEM: | 3.6 | 11.4 | 7.9 | 6.2 | 10.8 | 13.8 | 7.4 | 7.3 | 10.0 | 7.3 | 5.4 | 4.5 |

| | Group | | | | | | | | | |
|---|---|---|---|---|---|---|---|---|---|---|
| Animal | −2156 | −2157 | −2163 | −2165 | −2170 | −2195 | −2200 | −2204 | −2205 | −2210 |
| 1 | 63.1 | 104.4 | 41.1 | 93.9 | 66.8 | 91.3 | 144.7 | 76.6 | 57.0 | 64.5 |
| 2 | 76.4 | 81.6 | 31.0 | 50.7 | 48.1 | 57.0 | 109.8 | 77.0 | 33.9 | |
| 3 | 80.5 | 123.1 | 30.8 | 36.6 | 47.8 | 28.7 | 87.5 | 104.5 | 26.8 | 58.7 |
| 4 | 83.0 | 107.6 | 36.3 | 52.3 | 46.8 | 56.0 | 102.6 | 64.1 | 41.6 | 60.3 |
| 5 | 42.4 | 153.9 | 50.1 | 23.5 | 89.2 | 48.6 | 84.1 | 173.5 | 36.6 | 83.0 |
| Average: | 69.1 | 114.1 | 37.9 | 51.4 | 59.7 | 56.3 | 105.7 | 99.1 | 39.2 | 66.6 |
| SEM: | 7.5 | 11.9 | 3.6 | 11.8 | 8.3 | 10.1 | 10.8 | 19.7 | 5.1 | 5.6 |

Table 5 and 6 show 2 additional sets of HDI Studies with 18 GalNAc-conjugated PNPLA3 oligonucleotides in each set using the same reference oligonucleotide. Sequences of these oligonucleotides along with the modification patterns and SEQ ID NOs. are disclosed in Table B and Table C respectively.

Based on these results, 10 GalNAc-conjugated PNPLA3 oligonucleotides are selected for evaluation of their ability to inhibit PNPLA3 expression in non-human primates (NHPs). The GalNAc-conjugated PNPLA3 oligonucleotides have chemically modified nucleotides of the pattern as shown in FIG. 1.

TABLE 5
discloses a Single 1 mg/kg s.c. dose, HDI 3 days post-dose, harvest 4 days post-dose

| | Group | | | | | | | | | | | | | | | | | | |
|---|---|---|---|---|---|---|---|---|---|---|---|---|---|---|---|---|---|---|---|
| | | | | | | | | | | | | | | | | | | | −2205 |
| Animal | PBS | −644 | −745 | −1126 | −1129 | −1130 | −1143 | −1147 | −1152 | −1229 | −1698 | −2137 | −2138 | −2140 | −2142 | −2144 | −2146 | −2149 | (ref) |
| 1 | | 57.2 | 116.3 | 35.0 | 66.3 | 108.6 | 41.3 | 60.4 | | 111.7 | 60.0 | 80.2 | 101.1 | 39.0 | 96.0 | 48.4 | 53.5 | | 18.1 |
| 2 | 120.1 | 55.2 | 121.8 | 41.0 | 82.9 | 68.5 | | 59.3 | 55.1 | 80.1 | 112.9 | 68.0 | 69.9 | 24.1 | 47.9 | | 78.1 | | 38.9 |
| 3 | 129.1 | 143.6 | 82.2 | 94.9 | 53.4 | 91.1 | 30.0 | | | 86.2 | 71.9 | 120.8 | 45.0 | 41.4 | 78.2 | 34.9 | | 23.5 | 65.7 |
| 4 | 50.8 | | 102.2 | 57.9 | 77.6 | 145.3 | 34.4 | 43.4 | 39.8 | 101.5 | 88.7 | | 47.0 | 41.0 | 95.5 | 37.6 | 47.1 | 26.2 | 36.7 |
| 5 | | 27.0 | 114.3 | 42.3 | 117.2 | 125.9 | 13.9 | 41.8 | 64.5 | 89.1 | 116.6 | 44.5 | 29.1 | 47.1 | 54.0 | 25.5 | | 16.5 | 38.2 |
| Average: | 100.0 | 70.7 | 107.4 | 54.2 | 79.5 | 107.9 | 29.9 | 51.3 | 53.1 | 93.7 | 90.0 | 78.4 | 58.4 | 38.5 | 74.3 | 36.6 | 59.6 | 22.1 | 39.5 |
| SEM: | 24.8 | 25.2 | 7.1 | 10.9 | 10.7 | 13.3 | 5.8 | 5.0 | 7.2 | 5.7 | 11.1 | 16.0 | 12.5 | 3.9 | 10.1 | 4.7 | 9.5 | 2.9 | 7.6 |

TABLE 6
discloses a Single 1 mg/kg s.c. dose, HDI 3 days post-dose, harvest 4 days post-dose

| | Group | | | | | | | | | | | | | | | | | | |
|---|---|---|---|---|---|---|---|---|---|---|---|---|---|---|---|---|---|---|---|
| | | | | | | | | | | | | | | | | | | | −2205 |
| Animal | PBS | −637 | −641 | −923 | −1116 | −1125 | −1136 | −1151 | −1157 | −1158 | −1173 | −1541 | −1163 | −1614 | −1697 | −2136 | −2176 | −2222 | (ref) |
| 1 | | 80.3 | 114.9 | 88.9 | 48.9 | 203.5 | 156.1 | 49.0 | 113.6 | 113.6 | 42.2 | 91.1 | 62.5 | 56.4 | 24.4 | 145.3 | 64.1 | 97.3 | 26.9 |
| 2 | 76.6 | 140.7 | 116.7 | 122.7 | 78.6 | 115.6 | | 101.3 | 72.8 | 95.2 | 99.6 | 50.1 | | 79.5 | 57.3 | 85.7 | 28.2 | 137.8 | 58.6 |
| 3 | 78.7 | 115.4 | 57.8 | 142.4 | 122.0 | 119.8 | 130.1 | 91.9 | 186.4 | 134.1 | 20.5 | 135.3 | 110.5 | 60.2 | 104.6 | 65.7 | 71.4 | 107.5 | 34.6 |
| 4 | 133.6 | 90.3 | 84.3 | 85.6 | 150.0 | 80.0 | | | 98.2 | 113.6 | 25.0 | | 203.1 | 44.8 | | 132.5 | 44.8 | 50.1 | 52.7 |
| 5 | 111.1 | 119.9 | 198.0 | 75.0 | 41.6 | 166.4 | 84.6 | 121.0 | 114.0 | | 44.2 | 45.9 | 96.0 | 44.9 | 52.0 | 179.6 | 35.4 | 90.3 | 55.6 |
| Average: | 100.0 | 109.4 | 114.3 | 102.9 | 88.2 | 137.1 | 123.6 | 90.8 | 117.0 | 114.1 | 46.3 | 80.6 | 118.0 | 57.2 | 59.6 | 121.8 | 48.8 | 96.6 | 45.7 |
| SEM: | 13.7 | 10.8 | 23.6 | 12.7 | 20.9 | 21.6 | 20.9 | 15.2 | 18.9 | 7.9 | 14.1 | 20.9 | 30.1 | 6.4 | 16.7 | 20.6 | 8.3 | 14.2 | 6.3 |

Table 7 discloses an additional HDI Study using the best GalNAc-conjugated PNPLA3 oligonucleotides (hits) based on the results obtained from the previous studies and previous 3 HDI screens, as disclosed in tables 4, 5 and 6. Sequences of the oligonucleotides along with the modification patterns and SEQ ID NOs. used in this HDI screen are disclosed in Table D.

TABLE 7
comparison of hits from earlier studies and 3 HDI screens with Single 1 mg/kg s.c.
dose, HDI 3 days post-dose, harvest 4 days post-dose.

| | Group | | | | | | | | | | | | |
|---|---|---|---|---|---|---|---|---|---|---|---|---|---|
| Animal | PBS | −639 | −644 | −729 | −735 | −743 | −744 | −838 | −1126 | −1143 | −1147 | −1152 | −1173 |
| 1 | 98.7 | 58.8 | 22.9 | 114.6 | 119.1 | 106.5 | 94.7 | 45.3 | 64.6 | 24.8 | 70.5 | 55.2 | 26.0 |
| 2 | 99.9 | 62.3 | 30.7 | 108.6 | 85.0 | 105.4 | 55.4 | 48.1 | 30.4 | 16.8 | 24.7 | 71.6 | 10.7 |
| 3 | 143.0 | 87.9 | 10.1 | 106.3 | 97.4 | 76.2 | 61.1 | 91.8 | 39.1 | 18.4 | 33.4 | 50.5 | 18.2 |
| 4 | 115.1 | 89.6 | 19.2 | 99.5 | 84.0 | 99.8 | 56.5 | 68.5 | 61.2 | 26.7 | 59.3 | 58.2 | 10.8 |
| 5 | 89.2 | 91.4 | 41.1 | 94.5 | 105.8 | 115.0 | 113.8 | 48.6 | 54.6 | 30.3 | 47.2 | 59.8 | 35.5 |
| Average: | 109.2 | 78.0 | 24.8 | 104.7 | 98.3 | 100.6 | 76.3 | 60.5 | 50.0 | 23.4 | 47.0 | 59.1 | 20.2 |
| SEM: | 9.4 | 7.2 | 5.3 | 3.5 | 6.6 | 6.6 | 11.9 | 8.9 | 6.6 | 2.5 | 8.3 | 3.5 | 4.7 |

| | Group | | | | | | | | | |
|---|---|---|---|---|---|---|---|---|---|---|
| Animal | −1697 | −1699 | −1703 | −2140 | −2144 | −2149 | −2156 | −2195 | −2205 | PBS |
| 1 | 88.2 | 18.7 | 44.9 | 29.4 | 13.4 | 32.7 | 76.2 | 62.3 | 36.2 | 85.1 |
| 2 | 53.5 | 27.3 | 34.6 | 22.1 | 20.4 | 20.1 | 56.4 | 48.8 | 29.2 | 127.9 |
| 3 | 72.2 | 43.1 | 34.3 | 51.1 | 31.8 | 21.1 | 55.1 | 31.2 | 40.9 | 97.7 |
| 4 | 27.8 | 33.0 | 22.2 | 34.2 | 73.1 | 17.1 | 40.8 | 43.5 | 30.7 | 74.6 |
| 5 | 25.0 | 26.1 | 23.9 | 19.1 | 59.8 | 16.7 | 62.5 | 88.6 | 30.5 | 68.9 |
| Average: | 53.3 | 29.6 | 32.0 | 31.2 | 39.7 | 21.5 | 58.2 | 54.9 | 33.5 | 90.8 |
| SEM: | 12.3 | 4.1 | 4.1 | 5.6 | 11.5 | 2.9 | 5.7 | 9.8 | 2.2 | 10.5 |

NHP studies: 10 GalNAc-conjugated PNPLA3 oligonucleotides selected from table 7 are evaluated in cynomolgus monkeys (Macaca fascicularis). Here, the NHPs are grouped so that their mean body weights (about 5.4 kg) are comparable between the control and experimental groups. Each cohort contains two male and three female subjects. The GalNAc-conjugated PNPLA3 oligonucleotides are administered SQ on Study Day 0. Blood samples are collected on Study Days −8, −5 and 0, and weekly after dosing. Ultrasound-guided core needle liver biopsies are collected on Study Days −7, 28 and 56. At each time point, total RNA derived from the liver biopsy samples is subjected to qRT-PCR analysis to measure PNPLA3 mRNA in oligonucleotide-treated monkeys relative to monkeys treated with a comparable volume of PBS. To normalize the data, the measurements are made relative to the geometric mean of two reference genes, PPIB and 18S rRNA. As shown in Table 8 (Day −7), Table 9 (Day 28), and Table 10 (Day 56), treating NHPs with the GalNAc-conjugated PNPLA3 oligonucleotides inhibits PNPLA3 expression in the liver, as determined by a reduced amount of PNPLA3 mRNA in liver samples from oligonucleotide-treated NHPs relative to NHPs treated with PBS. For all time points evaluated, PNPLA3 oligonucleotides 34, 81 and 121 inhibit PNPLA3 expression to a greater extent than the benchmark PBS and time-matched controls. From the same NHP study, inhibition of PNPLA3 expression is also determined by measuring PNPLA3 protein in serum prepared from the pre-dose and weekly blood samples by ELISA. Taken together, these results demonstrate that treating NHPs with GalNAc-conjugated PNPLA3 oligonucleotides reduces the amount of PNPLA3 mRNA in the liver and concomitantly reduces the amount of PNPLA3 protein in the serum.

TABLE 8
PNPLA3 mRNA Knockdown of Select GalNAc-Conjugated
PNPLA3 Oligonucleotides in NHP at Day −7.

| | Passenger | Guide | |
|---|---|---|---|
| | (sense) | (antisense) | Mean % KD, Rel To |
| Sequence | (SEQ ID NO:) | (SEQ ID NO:) | Time-Matched PBS |
| PBS | — | — | 0 |
| 644 | 1220 | 1221 | 29 |
| 1126 | 1224 | 1225 | −4.9 |
| 1143 | 1230 | 1231 | 43 |
| 1147 | 1232 | 1233 | −2.5 |
| 1699 | 1188 | 1189 | 44 |
| 1703 | 1190 | 1191 | 28 |
| 2140 | 1244 | 1245 | 27 |
| 2149 | 1250 | 1251 | 20 |
| 1697 | 1254 | 1255 | 5.9 |
| 2144 | 1246 | 1247 | −15 |

TABLE 9
PNPLA3 mRNA Knockdown of Select GalNAc-Conjugated PNPLA3
Oligonucleotides in NHP at Day 28.

| | | | Mean | Mean | Mean % |
|---|---|---|---|---|---|
| | Passenger | Guide | % KD, Rel | % KD, | KD, Rel To |
| | (sense) | (antisense) | To Time- | Rel to | Individual |
| | (SEQ | (SEQ | Matched | mean of | Animal |
| Sequence | ID NO:) | ID NO:) | PBS | pre-dose | Predose |
| PBS | — | — | 0 | −9.8 | −30 |
| 644 | 1220 | 1221 | 34 | −3 | −17 |
| 1126 | 1224 | 1225 | 50 | 48 | 36 |
| 1143 | 1230 | 1231 | 54 | 11 | −0.1 |
| 1147 | 1232 | 1233 | −2.4 | −9.8 | 11 |
| 1699 | 1188 | 1189 | 65 | 32 | 30 |
| 1703 | 1190 | 1191 | 60 | 39 | 19 |
| 2140 | 1244 | 1245 | 65 | 47 | 40 |
| 2149 | 1250 | 1251 | 64 | 51 | 48 |
| 1697 | 1254 | 1255 | 49 | 40 | 42 |
| 2144 | 1246 | 1247 | 50 | 53 | 51 |

TABLE 10
PNPLA3 mRNA Knockdown of Select GalNAc-Conjugated
PNPLA3 Oligonucleotides in NHP at Day 56.

| | | | | | Mean % |
|---|---|---|---|---|---|
| | | | Mean | Mean | KD, Rel |
| | Passenger | Guide | % KD, Rel | % KD, | to |
| | (sense) | (antisense) | to Time- | Rel to | Individual |
| | (SEQ | (SEQ | Matched | Mean of | Animal |
| Sequence | ID NO:) | ID NO:) | PBS | Pre-Dose | Predose |
| PBS | — | — | 0 | 5 | −12 |
| 644 | 1220 | 1221 | 46 | 28 | 28 |
| 1126 | 1224 | 1225 | 39 | 45 | 27 |
| 1143 | 1230 | 1231 | 35 | −9.2 | −32 |
| 1147 | 1232 | 1233 | 28 | 34 | 29 |
| 1699 | 1188 | 1189 | 51 | 18 | 14 |
| 1703 | 1190 | 1191 | 5.5 | −24 | −45 |
| 2140 | 1244 | 1245 | −34 | −73 | −100 |
| 2149 | 1250 | 1251 | 48 | 39 | 36 |
| 1697 | 1254 | 1255 | 32 | 31 | 31 |
| 2144 | 1246 | 1247 | −9.8 | 9.7 | 4.4 |

Taken together, these results show that GalNAc-conjugated PNPLA3 oligonucleotides designed to target human PNPLA3 mRNA inhibit PNPLA3 expression in vivo (as determined by the reduction of the amount of PNPLA3 mRNA and PNPLA3 protein in treated animals).

### SEQUENCES

The following nucleic acid sequences and/or amino acid sequences are referred to in the disclosure and are provided below for reference.

SEQ ID NO: 1 - wild-type human PNPLA3 (2753 bp; NCBI Ref. Seq. No. NM_025225.3)
agagagcgcttgcgggcgccgggcggagctgctgcggatcaggacccgagccgattcccgatcccgacccagatcctaacccgc
gcccccgccccgccgccgccgccatgtacgacgcagagcgcggctggagcttgtccttcgcgggctgcggcttcctgggcttctac
cacgtcggggcgacccgctgcctgagcgagcacgccccgcacctcctccgcgacgcgcgcatgttgttcggcgcttcggccgggg
cgttgcactgcgtcggcgtcctctccggtatcccgctggagcagactctgcaggtcctctcagatcttgtgcggaaggccaggagtcg
gaacattggcatcttccatccatccttcaacttaagcaagttcctccgacagggtctctgcaaatgcctcccggccaatgtccaccagctc
atctccggcaaaataggcatctctcttaccagagtgtctgatggggaaaacgttctggtgtctgactttcggtccaaagacgaagtcgtg
gatgccttggtatgttcctgcttcatccccttctacagtggccttatccctccttccttcagaggcgtgcgatatgtggatggaggagtgag
tgacaacgtacccttcattgatgccaaaacaaccatcaccgtgtcccccttctatggggagtacgacatctgccctaaagtcaagtccac
gaactttcttcatgtggacatcaccaagctcagtctacgcctctgcacagggaacctctaccttctctcgagagcttttgtccccccggat
ctcaaggtgctgggagagatatgccttcgaggatatttggatgcattcaggttcttggaagagaagggcatctgcaacaggccccagc
caggcctgaagtcatcctcagaagggatggatcctgaggtcgccatgcccagctgggcaaacatgagtctggattcttccccggagtc
ggctgccttggctgtgaggctggagggagatgagctgctagaccacctgcgtctcagcatcctgccctgggatgagagcatcctgga
caccctctcgcccaggctcgctacagcactgagtgaagaaatgaaagacaaaggtggatacatgagcaagatttgcaacttgctaccc
attaggataatgtcttatgtaatgctgccctgtaccctgcctgtggaatctgccattgcgattgtccagagactggtgacatggcttccaga
tatgcccgacgatgtcctgtggttgcagtgggtgacctcacaggtgttcactcgagtgctgatgtgtctgctccccgcctccaggtccca
aatgccagtgagcagccaacaggcctccccatgcacacctgagcaggactggccctgctggactccctgctcccccaagggctgtc
cagcagagaccaaagcagaggccaccccgcggtccatcctcaggtccagcctgaacttcttcttgggcaataaagtacctgctggtg
ctgaggggctctccacctttcccagtttttcactagagaagagtctgtgagtcacttgaggaggcgagtctagcagattctttcagaggtg
ctaaagtttcccatctttgtgcagctacctccgcattgctgtgtagtgacccctgcctgtgacgtggaggatcccagcctctgagctgagt
tggttttatgaaaagctaggaagcaacctttcgcctgtgcagcggtccagcacttaactctaatacatcagcatgcgttaattcagctggtt
gggaaatgacaccaggaagcccagtgcagagggtcccttactgactgtttcgtggccctattaatggtcagactgttccagcatgaggt
tcttagaatgacaggtgtttggatgggtgggggccttgtgatggggggtaggctggcccatgtgtgatcttgtggggtggagggaaga
gaatagcatgatcccacttccccatgctgtgggaaggggtgcagttcgtccccaagaacgacactgcctgtcaggtggtctgcaaaga
tgataaccttgactactaaaaacgtctccatggcgggggtaacaagatgataatctacttaattttagaacacctttttcacctaactaaaat
aatgtttaaagagttttgtataaaaatgtaaggaagcgttgttacctgttgaattttgtattatgtgaatcagtgagatgttagtagaataagcc
ttaaaaaaaaaaaaatcggttgggtgcagtggcacacggctgtaatcccagcactttgggaggccaaggttggcagatcacctgaggt
caggagttcaagaccagtctggccaacatagcaaaaccctgtctctactaaaaatacaaaaattatctgggcatggtggtgcatgcctgt
aatcccagctattcggaaggctgaggcaggagaatcacttgaacccaggaggcggaggttgcggtgagctgagattgcaccatttca
ttccagcctgggcaacatgagtgaaagtctgactcaaaaaaaaaaaatttaaaaaacaaaataatctagtgtgcagggcattcacctca
gccccccaggcaggagccaagcacagcaggagcttccgcctcctctccactggagcacacaacttgaacctggcttattttctgcagg
gaccagccccacatggtcagtgagtttctccccatgtgtggcgatgagagagtgtagaaataaagacacaagacaaagaga
SEQ ID NO: 2 - wild-type human PNPLA3 (481 aa; NCBI Ref. Seq. No. NP_079501.2)
MYDAERGWSLSFAGCGFLGFYHVGATRCLSEHAPHLLRDARMLFGASAGALHCVG
VLSGIPLEQTLQVLSDLVRKARSRNIGIFHPSFNLSKFLRQGLCKCLPANVHQLISGKI
GISLTRVSDGENVLVSDFRSKDEVVDALVCSCFIPFYSGLIPPSFRGVRYVDGGVSDN
VPFIDAKTTITVSPFYGEYDICPKVKSTNFLHVDITKLSLRLCTGNLYLLSRAFVPPDL
KVLGEICLRGYLDAFRFLEEKGICNRPQPGLKSSSEGMDPEVAMPSWANMSLDSSPE
SAALAVRLEGDELLDHLRLSILPWDESILDTLSPRLATALSEEMKDKGGYMSKICNLL
PIRIMSYVMLPCTLPVESAIAIVQRLVTWLPDMPDDVLWLQWVTSQVFTRVLMCLLP
ASRSQMPVSSQQASPCTPEQDWPCWTPCSPKGCPAETKAEATPRSILRSSLNFFLGNK
VPAGAEGLSTFPSFSLEKSL
SEQ ID NO: 3 - human PNPLA3 I148M (variant) mRNA
agagagcgcttgcgggcgccgggcggagctgctgcggatcaggacccgagccgattcccgatcccgacccagatcctaacccgc
gcccccgccccgccgccgccgccatgtacgacgcagagcgcggctggagcttgtccttcgcgggctgcggcttcctgggcttctac
cacgtcggggcgacccgctgcctgagcgagcacgccccgcacctcctccgcgacgcgcgcatgttgttcggcgcttcggccgggg
cgttgcactgcgtcggcgtcctctccggtatcccgctggagcagactctgcaggtcctctcagatcttgtgcggaaggccaggagtcg
gaacattggcatcttccatccatccttcaacttaagcaagttcctccgacagggtctctgcaaatgcctcccggccaatgtccaccagctc
atctccggcaaaataggcatctctcttaccagagtgtctgatggggaaaacgttctggtgtctgactttcggtccaaagacgaagtcgtg
gatgccttggtatgttcctgcttcatgcccttctacagtggccttatccctccttccttcagaggcgtgcgatatgtggatggaggagtgag
tgacaacgtacccttcattgatgccaaaacaaccatcaccgtgtcccccttctatggggagtacgacatctgccctaaagtcaagtccac
gaactttcttcatgtggacatcaccaagctcagtctacgcctctgcacagggaacctctaccttctctcgagagcttttgtccccccggat
ctcaaggtgctgggagagatatgccttcgaggatatttggatgcattcaggttcttggaagagaagggcatctgcaacaggccccagc
caggcctgaagtcatcctcagaagggatggatcctgaggtcgccatgcccagctgggcaaacatgagtctggattcttccccggagtc
ggctgccttggctgtgaggctggagggagatgagctgctagaccacctgcgtctcagcatcctgccctgggatgagagcatcctgga
caccctctcgcccaggctcgctacagcactgagtgaagaaatgaaagacaaaggtggatacatgagcaagatttgcaacttgctaccc
attaggataatgtcttatgtaatgctgccctgtaccctgcctgtggaatctgccattgcgattgtccagagactggtgacatggcttccaga
tatgcccgacgatgtcctgtggttgcagtgggtgacctcacaggtgttcactcgagtgctgatgtgtctgctccccgcctccaggtccca
aatgccagtgagcagccaacaggcctccccatgcacacctgagcaggactggccctgctggactccctgctcccccaagggctgtc
cagcagagaccaaagcagaggccaccccgcggtccatcctcaggtccagcctgaacttcttcttgggcaataaagtacctgctggtg
ctgaggggctctccacctttcccagtttttcactagagaagagtctgtgagtcacttgaggaggcgagtctagcagattctttcagaggtg
ctaaagtttcccatctttgtgcagctacctccgcattgctgtgtagtgacccctgcctgtgacgtggaggatcccagcctctgagctgagt
tggttttatgaaaagctaggaagcaacctttcgcctgtgcagcggtccagcacttaactctaatacatcagcatgcgttaattcagctggtt
gggaaatgacaccaggaagcccagtgcagagggtcccttactgactgtttcgtggccctattaatggtcagactgttccagcatgaggt
tcttagaatgacaggtgtttggatgggtgggggccttgtgatggggggtaggctggcccatgtgtgatcttgtggggtggagggaaga
gaatagcatgatcccacttccccatgctgtgggaaggggtgcagttcgtccccaagaacgacactgcctgtcaggtggtctgcaaaga
tgataaccttgactactaaaaacgtctccatggcgggggtaacaagatgataatctacttaattttagaacacctttttcacctaactaaaat
aatgtttaaagagttttgtataaaaatgtaaggaagcgttgttacctgttgaattttgtattatgtgaatcagtgagatgttagtagaataagcc
ttaaaaaaaaaaaaatcggttgggtgcagtggcacacggctgtaatcccagcactttgggaggccaaggttggcagatcacctgaggt
caggagttcaagaccagtctggccaacatagcaaaaccctgtctctactaaaaatacaaaaattatctgggcatggtggtgcatgcctgt
aatcccagctattcggaaggctgaggcaggagaatcacttgaacccaggaggcggaggttgcggtgagctgagattgcaccatttca
ttccagcctgggcaacatgagtgaaagtctgactcaaaaaaaaaaaatttaaaaaacaaaataatctagtgtgcagggcattcacctca
gccccccaggcaggagccaagcacagcaggagcttccgcctcctctccactggagcacacaacttgaacctggcttattttctgcagg
gaccagccccacatggtcagtgagtttctccccatgtgtggcgatgagagagtgtagaaataaagacacaagacaaagaga
SEQ ID NO: 4 - human PNPLA3 I148M (variant)
MYDAERGWSLSFAGCGFLGFYHVGATRCLSEHAPHLLRDARMLFGASAGALHCVG
VLSGIPLEQTLQVLSDLVRKARSRNIGIFHPSFNLSKFLRQGLCKCLPANVHQLISGKI
GISLTRVSDGENVLVSDFRSKDEVVDALVCSCFMPFYSGLIPPSFRGVRYVDGGVSD
NVPFIDAKTTITVSPFYGEYDICPKVKSTNFLHVDITKLSLRLCTGNLYLLSRAFVPPD
LKVLGEICLRGYLDAFRFLEEKGICNRPQPGLKSSSEGMDPEVAMPSWANMSLDSSP
ESAALAVRLEGDELLDHLRLSILPWDESILDTLSPRLATALSEEMKDKGGYMSKICNL
LPIRIMSYVMLPCTLPVESAIAIVQRLVTWLPDMPDDVLWLQWVTSQVFTRVLMCLL
PASRSQMPVSSQQASPCTPEQDWPCWTPCSPKGCPAETKAEATPRSILRSSLNFFLGN
KVPAGAEGLSTFPSFSLEKSL
SEQ ID NO: 5 - primate PNPLA3 (2291 bp; NCBI Ref. Seq. No. XM_015457081.1)
cgagggaggcggggcggacgtcgcgcgtggaaagcccgggcggagacgcggcggctgggtcacgagcgcttgcgggcgccc
ggcggagctgctgcggatcaggacccgagccgatccccgatcccgactccgatccggatccgcgcccccgcccccgccccgccat
gtacgacgccgagcgcggctggagcttgtccttcgcgggctgcggcttcctgggcttctaccacgtcggggcgacccggtgcctga
gcgagcacgccccgcacctcctccgcgacgcgcgcatgttgttcggcgcctcggccggggcgttgcactgcgtcggcgtcctctcc
gggatcccgctggagcagactctgcaggtcctctcagatcttgtccggaaggccaggagtcggaacattggtatcttccatccatcctt
caacataggcaagttcctccgacaggatctctacaaatacctcccggccaatgtccaccagctcatctctggcaaaatatgcgtctcact
caccagagtgtctgatggggaaaacgttctggtgtctgactttcagtccaaagacgaagtcgtggatgccttgatttgttcctgcttcatcc
ctttctacagtggccttatccctccttccttcagaggcgtgcgatatgtggatggaggagcgagtgacaacgtacccttcattgatgccaa
gacaaccatcaccgtgtcgcccttctatggggagtacgacatctgccctaaagtcaagtccaccaactttcttcatgtggacatcaccaa
gctcagcctacgcctctgcacagggaacctctaccttctctcaagagcgtttgtccccccggatctcaaggtgctgggagagatatgcc
ttcgaggatatttggacgcgttcaggttcttggaagagaagggcatctgcaacaagccccagcggggtctgaagtcatcctcagaagg
gatggattctgaggtcactgcgcccggctgggaaaacacaagtctggattcttccccggagccggctgccttggctatgaggctggat
ggagatgagctgctagaccacctgcgtctcagcatcctgccctgggatgagagcatcctggacaccctgtcgcccgagctcgctaca
gtgagtgaagcaatgaaagacaaaggtggatacatgagcaagatttgcaacttgctacccattaggataatatcttatgtgatgctgccc
tgtaccctgcctgtggagtctgccattgcgattgtccagagactggtgacatggcttccagatatgcccgacgatgtgcagtggctgca
gtgggtgacctcacaggtcttcactcgagcgctgatgtgtctgcttcccgcctccaggtcccaaatgccagtgagcagcgaacaggcc
tccccatgcaaaccggagcaggactggcactgctggactccctgctcccccgaggactgtcctgcagaggccaaagcagaggctac
cccacggtccatcctcaggtccagcctgaacttcttctggggcaataaagtacctgctggtgctgaggggctctccacctttcccagtttt
tcactggagaagaatttgtgagtcatttgaggaggcgagtctaggagattctttcagaggtgctaaagcttcccatctttgtgcagctacct
ccgcattgccgtgtagtgacccctgcctgtgacgtggaggatcccagcctctgagctgagttggttttatgaaaagctaggaagcaatg
tttggtctgtgcagcagtccagcacttaagtctaatacgtcagcatgcgttagttcagctggttgggaaatgacaccgggaagcctagcg
cagagggtcccttactgactatttcatggtcctattaatggtcagactgttccagtgtgaggttcttagaatgactagtgtttggatgggtgg
gggccttgtggtggggggtgggctggcctatgtgtgatcttgtggggtggaaggaagagagtagcacaatcccacctccccatgccg
tgggaaggggtgcacttggttcccaagaaggacactgcctgtcaggtggcctgcaaatataataaccttgacaactaaaaacctctcca
tgggggtgggaggtaccaagataataaccgatttacattttagagcacctttttcacctaactaaaataatgtttaaagagttttatataaaa
atgtaaggaagagttgttatctgttgaattttgtattatatgaatcagtgagatgttaatagaataagcctt
SEQ ID NO: 6 - primate PNPLA3 (480 aa; NCBI Ref. Seq. No. XP_015312567.1)
MYDAERGWSLSFAGCGFLGFYHVGATRCLSEHAPHLLRDARMLFGASAGALHCVG
VLSGIPLEQTLQVLSDLVRKARSRNIGIFHPSFNIGKFLRQDLYKYLPANVHQLISGKI
CVSLTRVSDGENVLVSDFQSKDEVVDALICSCFIPFYSGLIPPSFRGVRYVDGGASDN
VPFIDAKTTITVSPFYGEYDICPKVKSTNFLHVDITKLSLRLCTGNLYLLSRAFVPPDL
KVLGEICLRGYLDAFRFLEEKGICNKPQRGLKSSSEGMDSEVTAPGWENTSLDSSPEP
AALAMRLDGDELLDHLRLSILPWDESILDTLSPELATVSEAMKDKGGYMSKICNLLPI
RIISYVMLPCTLPVESAIAIVQRLVTWLPDMPDDVQWLQWVTSQVFTRALMCLLPAS
RSQMPVSSEQASPCKPEQDWHCWTPCSPEDCPAEAKAEATPRSILRSSLNFFWGNKV
PAGAEGLSTFPSFSLEKNL
SEQ ID NO: 7 - mouse PNPLA3 (4675 bp; NCBI Ref. Seq. No. XM_006520346.5)
cacccgaagacagcttaggcggctgcggctctttaagctcagagcagcaacaccgggagcagagctgaactgcagcgccgcccgg
agcttcaagcaccatgtatgacccagagcgccgctggagcctgtcgtttgcaggctgcggcttcctgggcttctaccacgtcggggct
acgctatgtctgagcgagcgcgccccgcacctcctccgcgatgcgcgcactttctttggctgctcggccggtgcactgcacgcggtca
ccttcgtgtgcagtctccctctcggtgcgtccacggccactacccaggccccgcgtgcggggaggggttgctacacctggggaatcg
gtaacactttccggggtgcccgaagaaacctgtccggagagctctcatccttcccggtgccgttgagcactcagctggagaccctggg
cctgtcacctggcgtgggatttcccggggccggctggagcagacgcgctccgagcatcttcttcctgacccgctctggcgccctggtc
ctgtcagctgggtcatccctgagcagcagggaacgacgcaggtttgccgggtcctctggtccctgagccgcagaaggccgtataatg
gagatcctcatggacctcgtgcggaaagccaggagccgcaacatcggcaccctccacccgttcttcaacattaacaagtgcatcaga
gacgggctccaggagagcctcccagacaatgtccaccaggtcatttctggcaaggttcacatctcactcaccagggtgtcggatggg
gagaacgtgctggtgtctgagttccattccaaagacgaagtcgtggatgccctggtgtgttcctgcttcattcccctcttctctggcctaat
ccctccttccttccgaggcgagcggtacgtggacggaggagtgagcgacaacgtccctgtgctggatgccaaaaccaccatcacggt
gtcacctttctacggtgagcatgacatctgccccaaagtcaagtccaccaacttcttccacgtgaatatcaccaacctcagcctccgcct
ctgcactgggaacctccaacttctgaccagagcgctcttcccgtctgatgtgaaggtgatgggagagctgtgctatcaagggtacctgg
acgccttccggttcctggaggagaatggcatctgtaacgggccacagcgcagcctgagtctgtccttggtggcgccagaagcctgctt
ggaaaatggcaaacttgtgggagacaaggtgccagtcagcctatgctttacagatgagaacatctgggagacactgtcccccgagct
cagcacagctctgagtgaagcgattaaggacagggagggctacctgagcaaagtctgcaacctcctgcccgtcaggatcctgtccta
catcatgctgccctgcagtctgcccgtggagtcggctatcgctgcagtccacaggctggtgacatggctccctgatatccaggatgata
tccagtggctacaatgggcgacatcccaggtttgtgcccgaatgacgatgtgcctgctcccctctaccagcatgagattcctgggacaa
tcccaattccttggcctccattgtatcaaagggctgaaaaccaaagggaaggcacagctgtctcttcagcatgcctcttctgccagaacc
actgcaaggtttggtgctcaggctgtgcaaacattctagcaatgtttgactcagtgtcaagcaggtgacaaggaacatggtgctgtgtgg
ggggaacccatggcccaggtgagggcttattggtgggtgaagctgtgggtgttcaggtggtggagaaggccttaagggatgggact
gacacctcagcactgaaggcaggaggaagctgtggctctgggttgcacccctgcctggctccaccctctctggcatctgtagaagtta
cagctggttcttcctctcagccccatgctcccagaaataagactcagacccaaattatagttacaaataccttggccatatagctaggctc
ttctcagactagctcataacttaactcattaattttaacctccatcctgccacatggctggtggcctgtgctcaggtaccatgagtccagctc
ttcacatctttccggatgaatcttccataattctttctgcctcctggatgttccaccttctattccaccttttcctataggccatggttttgtttttgt
tttttttttccaaatttaatttaattaattaatttatttatttttggtttttcgagacagggtttctctgtatcgccctggctgtcctggaactcactatg
taagccaggctggcctcaaactcagaaatccgcctgcctctgcctcctgagtgctgggattaaaggcgtgcgcaaccatgcccggtgt
ggtttttttttttttttaattgacaggtggatgcatctatataatccataacatattctctctacaggtatctattaggttttgggtgaggtgtggag
ttctagggaactctgagagaaattcctggggagtaagtggtttatcaagttgattggaggagtttttaatgctatggacagacagacagaa
ggacaacagcatagtcggggctaccagggagttcaggccccggcatcggagatagaagcaggatggggtctttgaagagattctga
gcccacacagcagaggagggactctctctttagagcttttgaggatgagggaggttgactgcaagagcctacagccaggctcgaggc
aggcagggggtggggagcaggatgtaaaccccttcgatgctgacagactcacttctggggtaaaatattatgagatgcctgtcagtgt
ctgtgaagagacctgagcagagtctggattctgacatcaatcatgttcttacaatactgaagacctgagagcctgcaatcttggtttgtaa
attgctggtctccgtgcttccagtgaacttggacattcttctcatggttggtccaggagaggccaaagctgagggcaccctgccttccac
ccccagtccagcttgaccttttatctggagcaacagtgtctagatgatgggtgggtgaggggtgctatactgtctgtccctctgggaagg
gttctgttacttttggaggcagctaggaagtttctctgtgcagctgccccctggtgctgtgtggtgacctcattgcctgtgaccccaggatc
acaggatctgggctaaagtggtagtccatagaaaccaaagacaatgatttggtgtttagaaagctactcttggtctgggtgaagtctggt
gcttaagggctatcacaaagagcgtgtcaaaccatctctcagcctgtgagtcagtggggagcccaagggcatcagtgtttggaaactg
gaatccaaaccgggcaatctcggaaggaaactgtttaggaattgtgatgggacgggccgtggctgtctctgaaaagggcctgccaga
taacttattacttttaaggacacctttggctcttactaatttataaagcattttatataaacacaccagggagtgcatggtgaactacacgtat
gatcagttaagtggggctagaattaggtagggagagcatcggacctctgcctcctcaacctcaacttgcttgctttctccactggctcca
aatctttgtatagtcatcagccatgaccacctctctccctccccatctactaccagcagcgttaatgggaataagtacccacttctctcagg
tgtactatacagctgtgggtgtggtgtgtgtttcctgtaattcacactttagaaaggaaacaagcaaacaaaagaaaccaggtgctgccc
atactcctaagtgtagacagtgaaggtgtgtgtctcccatgcctgagtctcctggaggcctagtgagctccaggttcatgcaagcacatc
aggaggaatcatataatctcagcacggttgatccagatgggataagaaaggactctgggagagagaatgtggttctagagacaaagt
gtctaggctacacagaagataagactgtcccaaggaaagaaaagaaaccaggaactagggtgcagctcagttgtcagaggacttctc
taggcttgaagcccagagtccaatctcagcaccttataaactgtggagtgacaggcagtgacatcggcctgtaatcccaacactcaag
cagtagaggcaagaggatcataagttcaaggtcttccttggctatttagggagttggaggttagctctggctacatgagaccctgtctca
aaaaaaaaaaaaaaaaaaagtagaaacttctgccttgctttgagctgcccctttctggacgtttctcatcagtagagaatattcctgccac
cctatcagacaaaactcccactggtttggagtctctccattctcaggaacacctcaggagtcagacagtgagcagcagggagcaatgt
cttgacttgtaagccccttagcaaggctggttcatttgtttattaaaagcaggtgtgggtgaatttatgcaaatgagtatgcaaactagtgg
aacagcagaaggattgaatggatacaccaaaaataaccacaactgtttaagggaaaagggtccataataaatgtggggaacaaaaaa
caaataaatgtgatttttttta
SEQ ID NO: 8 - mouse PNPLA3 (367 aa; NCBI Ref. Seq. No. XP_006520409.1)
MEILMDLVRKARSRNIGTLHPFFNINKCIRDGLQESLPDNVHQVISGKVHISLTRVSD
GENVLVSEFHSKDEVVDALVCSCFIPLFSGLIPPSFRGERYVDGGVSDNVPVLDAKTT
ITVSPFYGEHDICPKVKSTNFFHVNITNLSLRLCTGNLQLLTRALFPSDVKVMGELCY
QGYLDAFRFLEENGICNGPQRSLSLSLVAPEACLENGKLVGDKVPVSLCFTDENIWET
LSPELSTALSEAIKDREGYLSKVCNLLPVRILSYIMLPCSLPVESAIAAVHRLVTWLPDI
QDDIQWLQWATSQVCARMTMCLLPSTSMRFLGQSQFLGLHCIKGLKTKGKAQLSLQ
HASSARTTARFGAQAVQTF
SEQ ID NO: 9 - DsiRNA 1 passenger (sense) strand
UCUGCAGGUCCUCUCAGAUCUUGUG
SEQ ID NO: 10 - DsiRNA 1 guide (antisense) strand
CACAAGAUCUGAGAGGACCUGCAGAGU
SEQ ID NO: 11 - DsiRNA 2 passenger (sense) strand
AGGUCCUCUCAGAUCUUGUGCGGAA
SEQ ID NO: 12 - DsiRNA 2 guide (antisense) strand
UUCCGCACAAGAUCUGAGAGGACCUGC
SEQ ID NO: 13 - DsiRNA 3 passenger (sense) strand
AUUGGCAUCUUCCAUCCAUCCUUCA
SEQ ID NO: 14 - DsiRNA 3 guide (antisense) strand
UGAAGGAUGGAUGGAAGAUGCCAAUGU
SEQ ID NO: 15 - DsiRNA 4 passenger (sense) strand
AUCUUCCAUCCAUCCUUCAACUUAA
SEQ ID NO: 16 - DsiRNA 4 guide (antisense) strand
UUAAGUUGAAGGAUGGAUGGAAGAUGC
SEQ ID NO: 17 - DsiRNA 5 passenger (sense) strand
UCUUCCAUCCAUCCUUCAACUUAAG
SEQ ID NO: 18 - DsiRNA 5 guide (antisense) strand
CUUAAGUUGAAGGAUGGAUGGAAGAUG
SEQ ID NO: 19 - DsiRNA 6 passenger (sense) strand
CCAGAGUGUCUGAUGGGGAAAACGU
SEQ ID NO: 20 - DsiRNA 6 guide (antisense) strand
ACGUUUUCCCCAUCAGACACUCUGGUA
SEQ ID NO: 21 - DsiRNA 7 passenger (sense) strand
GAGUGUCUGAUGGGGAAAACGUUCU
SEQ ID NO: 22 - DsiRNA 7 guide (antisense) strand
AGAACGUUUUCCCCAUCAGACACUCUG
SEQ ID NO: 23 - DsiRNA 8 passenger (sense) strand
AUGGGGAAAACGUUCUGGUGUCUGA
SEQ ID NO: 24 - DsiRNA 8 guide (antisense) strand
UCAGACACCAGAACGUUUUCCCCAUCA
SEQ ID NO: 25 - DsiRNA 9 passenger (sense) strand
GGGGAAAACGUUCUGGUGUCUGACU
SEQ ID NO: 26 - DsiRNA 9 guide (antisense) strand
AGUCAGACACCAGAACGUUUUCCCCAU
SEQ ID NO: 27 - DsiRNA 10 passenger (sense) strand
GGGAAAACGUUCUGGUGUCUGACUU
SEQ ID NO: 28 - DsiRNA 10 guide (antisense) strand
AAGUCAGACACCAGAACGUUUUCCCCA
SEQ ID NO: 29 - DsiRNA 11 passenger (sense) strand
GGAAAACGUUCUGGUGUCUGACUUU
SEQ ID NO: 30 - DsiRNA 11 guide (antisense) strand
AAAGUCAGACACCAGAACGUUUUCCCC
SEQ ID NO: 31 - DsiRNA 12 passenger (sense) strand
GAAAACGUUCUGGUGUCUGACUUUC
SEQ ID NO: 32 - DsiRNA 12 guide (antisense) strand
GAAAGUCAGACACCAGAACGUUUUCCC
SEQ ID NO: 33 - DsiRNA 13 passenger (sense) strand
AAAACGUUCUGGUGUCUGACUUUCG
SEQ ID NO: 34 - DsiRNA 13 guide (antisense) strand
CGAAAGUCAGACACCAGAACGUUUUCC
SEQ ID NO: 35 - DsiRNA 14 passenger (sense) strand
AAACGUUCUGGUGUCUGACUUUCGG
SEQ ID NO: 36 - DsiRNA 14 guide (antisense) strand
CCGAAAGUCAGACACCAGAACGUUUUC
SEQ ID NO: 37 - DsiRNA 15 passenger (sense) strand
AACGUUCUGGUGUCUGACUUUCGGU
SEQ ID NO: 38 - DsiRNA 15 guide (antisense) strand
ACCGAAAGUCAGACACCAGAACGUUUU
SEQ ID NO: 39 - DsiRNA 16 passenger (sense) strand
ACGUUCUGGUGUCUGACUUUCGGUC
SEQ ID NO: 40 - DsiRNA 16 guide (antisense) strand
GACCGAAAGUCAGACACCAGAACGUUU
SEQ ID NO: 41 - DsiRNA 17 passenger (sense) strand
GUUCUGGUGUCUGACUUUCGGUCCA
SEQ ID NO: 42 - DsiRNA 17 guide (antisense) strand
UGGACCGAAAGUCAGACACCAGAACGU
SEQ ID NO: 43 - DsiRNA 18 passenger (sense) strand
GACGAAGUCGUGGAUGCCUUGGUAU
SEQ ID NO: 44 - DsiRNA 18 guide (antisense) strand
AUACCAAGGCAUCCACGACUUCGUCUU
SEQ ID NO: 45 - DsiRNA 19 passenger (sense) strand
ACGAAGUCGUGGAUGCCUUGGUAUG
SEQ ID NO: 46 - DsiRNA 19 guide (antisense) strand
CAUACCAAGGCAUCCACGACUUCGUCU
SEQ ID NO: 47 - DsiRNA 20 passenger (sense) strand
CGAAGUCGUGGAUGCCUUGGUAUGU
SEQ ID NO: 48 - DsiRNA 20 guide (antisense) strand
ACAUACCAAGGCAUCCACGACUUCGUC
SEQ ID NO: 49 - DsiRNA 21 passenger (sense) strand
CAGAGGCGUGCGAUAUGUGGAUGGA
SEQ ID NO: 50 - DsiRNA 21 guide (antisense) strand
UCCAUCCACAUAUCGCACGCCUCUGAA
SEQ ID NO: 51 - DsiRNA 22 passenger (sense) strand
GCGAUAUGUGGAUGGAGGAGUGAGU
SEQ ID NO: 52 - DsiRNA 22 guide (antisense) strand
ACUCACUCCUCCAUCCACAUAUCGCAC
SEQ ID NO: 53 - DsiRNA 23 passenger (sense) strand
GAUGGAGGAGUGAGUGACAACGUAC
SEQ ID NO: 54 - DsiRNA 23 guide (antisense) strand
GUACGUUGUCACUCACUCCUCCAUCCA
SEQ ID NO: 55 - DsiRNA 24 passenger (sense) strand
GAGGAGUGAGUGACAACGUACCCUU
SEQ ID NO: 56 - DsiRNA 24 guide (antisense) strand
AAGGGUACGUUGUCACUCACUCCUCCA
SEQ ID NO: 57 - DsiRNA 25 passenger (sense) strand
GAGUGAGUGACAACGUACCCUUCAU
SEQ ID NO: 58 - DsiRNA 25 guide (antisense) strand
AUGAAGGGUACGUUGUCACUCACUCCU
SEQ ID NO: 59 - DsiRNA 26 passenger (sense) strand
AGUGAGUGACAACGUACCCUUCAUU
SEQ ID NO: 60 - DsiRNA 26 guide (antisense) strand
AAUGAAGGGUACGUUGUCACUCACUCC
SEQ ID NO: 61 - DsiRNA 27 passenger (sense) strand
GUGAGUGACAACGUACCCUUCAUUG
SEQ ID NO: 62 - DsiRNA 27 guide (antisense) strand
CAAUGAAGGGUACGUUGUCACUCACUC
SEQ ID NO: 63 - DsiRNA 28 passenger (sense) strand
UGAGUGACAACGUACCCUUCAUUGA
SEQ ID NO: 64 - DsiRNA 28 guide (antisense) strand
UCAAUGAAGGGUACGUUGUCACUCACU
SEQ ID NO: 65 - DsiRNA 29 passenger (sense) strand
GAGUGACAACGUACCCUUCAUUGAU
SEQ ID NO: 66 - DsiRNA 29 guide (antisense) strand
AUCAAUGAAGGGUACGUUGUCACUCAC
SEQ ID NO: 67 - DsiRNA 30 passenger (sense) strand
AGUGACAACGUACCCUUCAUUGAUG
SEQ ID NO: 68 - DsiRNA 30 guide (antisense) strand
CAUCAAUGAAGGGUACGUUGUCACUCA
SEQ ID NO: 69 - DsiRNA 31 passenger (sense) strand
GUGACAACGUACCCUUCAUUGAUGC
SEQ ID NO: 70 - DsiRNA 31 guide (antisense) strand
GCAUCAAUGAAGGGUACGUUGUCACUC
SEQ ID NO: 71 - DsiRNA 32 passenger (sense) strand
UGACAACGUACCCUUCAUUGAUGCC
SEQ ID NO: 72 - DsiRNA 32 guide (antisense) strand
GGCAUCAAUGAAGGGUACGUUGUCACU
SEQ ID NO: 73 - DsiRNA 33 passenger (sense) strand
GACAACGUACCCUUCAUUGAUGCCA
SEQ ID NO: 74 - DsiRNA 33 guide (antisense) strand
UGGCAUCAAUGAAGGGUACGUUGUCAC
SEQ ID NO: 75 - DsiRNA 34 passenger (sense) strand
CAACGUACCCUUCAUUGAUGCCAAA
SEQ ID NO: 76 - DsiRNA 34 guide (antisense) strand
UUUGGCAUCAAUGAAGGGUACGUUGUC
SEQ ID NO: 77 - DsiRNA 35 passenger (sense) strand
AACGUACCCUUCAUUGAUGCCAAAA
SEQ ID NO: 78 - DsiRNA 35 guide (antisense) strand
UUUUGGCAUCAAUGAAGGGUACGUUGU
SEQ ID NO: 79 - DsiRNA 36 passenger (sense) strand
ACGUACCCUUCAUUGAUGCCAAAAC
SEQ ID NO: 80 - DsiRNA 36 guide (antisense) strand
GUUUUGGCAUCAAUGAAGGGUACGUUG
SEQ ID NO: 81 - DsiRNA 37 passenger (sense) strand
CGUACCCUUCAUUGAUGCCAAAACA
SEQ ID NO: 82 - DsiRNA 37 guide (antisense) strand
UGUUUUGGCAUCAAUGAAGGGUACGUU
SEQ ID NO: 83 - DsiRNA 38 passenger (sense) strand
UACCCUUCAUUGAUGCCAAAACAAC
SEQ ID NO: 84 - DsiRNA 38 guide (antisense) strand
GUUGUUUUGGCAUCAAUGAAGGGUACG
SEQ ID NO: 85 - DsiRNA 39 passenger (sense) strand
UUGAUGCCAAAACAACCAUCACCGU
SEQ ID NO: 86 - DsiRNA 39 guide (antisense) strand
ACGGUGAUGGUUGUUUUGGCAUCAAUG
SEQ ID NO: 87 - DsiRNA 40 passenger (sense) strand
GAUGCCAAAACAACCAUCACCGUGU
SEQ ID NO: 88 - DsiRNA 40 guide (antisense) strand
ACACGGUGAUGGUUGUUUUGGCAUCAA
SEQ ID NO: 89 - DsiRNA 41 passenger (sense) strand
AUGGGGAGUACGACAUCUGCCCUAA
SEQ ID NO: 90 - DsiRNA 41 guide (antisense) strand
UUAGGGCAGAUGUCGUACUCCCCAUAG
SEQ ID NO: 91 - DsiRNA 42 passenger (sense) strand
GUACGACAUCUGCCCUAAAGUCAAG
SEQ ID NO: 92 - DsiRNA 42 guide (antisense) strand
CUUGACUUUAGGGCAGAUGUCGUACUC
SEQ ID NO: 93 - DsiRNA 43 passenger (sense) strand
GACAUCUGCCCUAAAGUCAAGUCCA
SEQ ID NO: 94 - DsiRNA 43 guide (antisense) strand
UGGACUUGACUUUAGGGCAGAUGUCGU
SEQ ID NO: 95 - DsiRNA 44 passenger (sense) strand
ACAUCUGCCCUAAAGUCAAGUCCAC
SEQ ID NO: 96 - DsiRNA 44 guide (sense) strand
GUGGACUUGACUUUAGGGCAGAUGUCG
SEQ ID NO: 97 - DsiRNA 45 passenger (sense) strand
GUCAAGUCCACGAACUUUCUUCAUG
SEQ ID NO: 98 - DsiRNA 45 guide (antisense) strand
CAUGAAGAAAGUUCGUGGACUUGACUU
SEQ ID NO: 99 - DsiRNA 46 passenger (sense) strand
UCAAGUCCACGAACUUUCUUCAUGU
SEQ ID NO: 100 - DsiRNA 46 guide (antisense) strand
ACAUGAAGAAAGUUCGUGGACUUGACU
SEQ ID NO: 101 - DsiRNA 47 passenger (sense) strand
CAAGUCCACGAACUUUCUUCAUGUG
SEQ ID NO: 102 - DsiRNA 47 guide (antisense) strand
CACAUGAAGAAAGUUCGUGGACUUGAC
SEQ ID NO: 103 - DsiRNA 48 passenger (sense) strand
GUCCACGAACUUUCUUCAUGUGGAC
SEQ ID NO: 104 - DsiRNA 48 guide (antisense) strand
GUCCACAUGAAGAAAGUUCGUGGACUU
SEQ ID NO: 105 - DsiRNA 49 passenger (sense) strand
CCACGAACUUUCUUCAUGUGGACAU
SEQ ID NO: 106 - DsiRNA 49 guide (antisense) strand
AUGUCCACAUGAAGAAAGUUCGUGGAC
SEQ ID NO: 107 - DsiRNA 50 passenger (sense) strand
CACGAACUUUCUUCAUGUGGACAUC
SEQ ID NO: 108 - DsiRNA 50 guide (antisense) strand
GAUGUCCACAUGAAGAAAGUUCGUGGA
SEQ ID NO: 109 - DsiRNA 51 passenger (sense) strand
ACGAACUUUCUUCAUGUGGACAUCA
SEQ ID NO: 110 - DsiRNA 51 guide (antisense) strand
UGAUGUCCACAUGAAGAAAGUUCGUGG
SEQ ID NO: 111 - DsiRNA 52 passenger (sense) strand
CGAACUUUCUUCAUGUGGACAUCAC
SEQ ID NO: 112 - DsiRNA 52 guide (antisense) strand
GUGAUGUCCACAUGAAGAAAGUUCGUG
SEQ ID NO: 113 - DsiRNA 53 passenger (sense) strand
GAACUUUCUUCAUGUGGACAUCACC
SEQ ID NO: 114 - DsiRNA 53 guide (antisense) strand
GGUGAUGUCCACAUGAAGAAAGUUCGU
SEQ ID NO: 115 - DsiRNA 54 passenger (sense) strand
AACUUUCUUCAUGUGGACAUCACCA
SEQ ID NO: 116 - DsiRNA 54 guide (antisense) strand
UGGUGAUGUCCACAUGAAGAAAGUUCG
SEQ ID NO: 117 - DsiRNA 55 passenger (sense) strand
ACUUUCUUCAUGUGGACAUCACCAA
SEQ ID NO: 118 - DsiRNA 55 guide (antisense) strand
UUGGUGAUGUCCACAUGAAGAAAGUUC
SEQ ID NO: 119 - DsiRNA 56 passenger (sense) strand
CUUUCUUCAUGUGGACAUCACCAAG
SEQ ID NO: 120 - DsiRNA 56 guide (antisense) strand
CUUGGUGAUGUCCACAUGAAGAAAGUU
SEQ ID NO: 121 - DsiRNA 57 passenger (sense) strand
UUUCUUCAUGUGGACAUCACCAAGC
SEQ ID NO: 122 - DsiRNA 57 guide (antisense) strand
GCUUGGUGAUGUCCACAUGAAGAAAGU
SEQ ID NO: 123 - DsiRNA 58 passenger (sense) strand
UUCUUCAUGUGGACAUCACCAAGCU
SEQ ID NO: 124 - DsiRNA 58 guide (antisense) strand
AGCUUGGUGAUGUCCACAUGAAGAAAG
SEQ ID NO: 125 - DsiRNA 59 passenger (sense) strand
CUUCAUGUGGACAUCACCAAGCUCA
SEQ ID NO: 126 - DsiRNA 59 guide (antisense) strand
UGAGCUUGGUGAUGUCCACAUGAAGAA
SEQ ID NO: 127 - DsiRNA 60 passenger (sense) strand
UUCAUGUGGACAUCACCAAGCUCAG
SEQ ID NO: 128 - DsiRNA 60 guide (antisense) strand
CUGAGCUUGGUGAUGUCCACAUGAAGA
SEQ ID NO: 129 - DsiRNA 61 passenger (sense) strand
UCAUGUGGACAUCACCAAGCUCAGU
SEQ ID NO: 130 - DsiRNA 61 guide (antisense) strand
ACUGAGCUUGGUGAUGUCCACAUGAAG
SEQ ID NO: 131 - DsiRNA 62 passenger (sense) strand
CAUGUGGACAUCACCAAGCUCAGUC
SEQ ID NO: 132 - DsiRNA 62 guide (antisense) strand
GACUGAGCUUGGUGAUGUCCACAUGAA
SEQ ID NO: 133 - DsiRNA 63 passenger (sense) strand
AUGUGGACAUCACCAAGCUCAGUCU
SEQ ID NO: 134 - DsiRNA 63 guide (antisense) strand
AGACUGAGCUUGGUGAUGUCCACAUGA
SEQ ID NO: 135 - DsiRNA 64 passenger (sense) strand
UGUGGACAUCACCAAGCUCAGUCUA
SEQ ID NO: 136 - DsiRNA 64 guide (antisense) strand
UAGACUGAGCUUGGUGAUGUCCACAUG
SEQ ID NO: 137 - DsiRNA 65 passenger (sense) strand
GUGGACAUCACCAAGCUCAGUCUAC
SEQ ID NO: 138 - DsiRNA 65 guide (antisense) strand
GUAGACUGAGCUUGGUGAUGUCCACAU
SEQ ID NO: 139 - DsiRNA 66 passenger (sense) strand
UGGACAUCACCAAGCUCAGUCUACG
SEQ ID NO: 140 - DsiRNA 66 guide (antisense) strand
CGUAGACUGAGCUUGGUGAUGUCCACA
SEQ ID NO: 141 - DsiRNA 67 passenger (sense) strand
AGCUUUUGUCCCCCCGGAUCUCAAG
SEQ ID NO: 142 - DsiRNA 67 guide (antisense) strand
CUUGAGAUCCGGGGGGACAAAAGCUCU
SEQ ID NO: 143 - DsiRNA 68 passenger (sense) strand
AAGGUGCUGGGAGAGAUAUGCCUUC
SEQ ID NO: 144 - DsiRNA 68 guide (antisense) strand
GAAGGCAUAUCUCUCCCAGCACCUUGA
SEQ ID NO: 145 - DsiRNA 69 passenger (sense) strand
GGGAGAGAUAUGCCUUCGAGGAUAU
SEQ ID NO: 146 - DsiRNA 69 guide (antisense) strand
AUAUCCUCGAAGGCAUAUCUCUCCCAG
SEQ ID NO: 147 - DsiRNA 70 passenger (sense) strand
GGAGAGAUAUGCCUUCGAGGAUAUU
SEQ ID NO: 148 - DsiRNA 70 guide (antisense) strand
AAUAUCCUCGAAGGCAUAUCUCUCCCA
SEQ ID NO: 149 - DsiRNA 71 passenger (sense) strand
AGAGAUAUGCCUUCGAGGAUAUUUG
SEQ ID NO: 150 - DsiRNA 71 guide (antisense) strand
CAAAUAUCCUCGAAGGCAUAUCUCUCC
SEQ ID NO: 151 - DsiRNA 72 passenger (sense) strand
GAGAUAUGCCUUCGAGGAUAUUUGG
SEQ ID NO: 152 - DsiRNA 72 guide (antisense) strand
CCAAAUAUCCUCGAAGGCAUAUCUCUC
SEQ ID NO: 153 - DsiRNA 73 passenger (sense) strand
AGAUAUGCCUUCGAGGAUAUUUGGA
SEQ ID NO: 154 - DsiRNA 73 guide (antisense) strand
UCCAAAUAUCCUCGAAGGCAUAUCUCU
SEQ ID NO: 155 - DsiRNA 74 passenger (sense) strand
GAUAUGCCUUCGAGGAUAUUUGGAU
SEQ ID NO: 156 - DsiRNA 74 guide (antisense) strand
AUCCAAAUAUCCUCGAAGGCAUAUCUC
SEQ ID NO: 157 - DsiRNA 75 passenger (sense) strand
AUAUGCCUUCGAGGAUAUUUGGAUG
SEQ ID NO: 158 - DsiRNA 75 guide (antisense) strand
CAUCCAAAUAUCCUCGAAGGCAUAUCU
SEQ ID NO: 159 - DsiRNA 76 passenger (sense) strand
AUGCCUUCGAGGAUAUUUGGAUGCA
SEQ ID NO: 160 - DsiRNA 76 guide (antisense) strand
UGCAUCCAAAUAUCCUCGAAGGCAUAU
SEQ ID NO: 161 - DsiRNA 77 passenger (sense) strand
UGCCUUCGAGGAUAUUUGGAUGCAU
SEQ ID NO: 162 - DsiRNA 77 guide (antisense) strand
AUGCAUCCAAAUAUCCUCGAAGGCAUA
SEQ ID NO: 163 - DsiRNA 78 passenger (sense) strand
GCCUUCGAGGAUAUUUGGAUGCAUU
SEQ ID NO: 164 - DsiRNA 78 guide (antisense) strand
AAUGCAUCCAAAUAUCCUCGAAGGCAU
SEQ ID NO: 165 - DsiRNA 79 passenger (sense) strand
UCAGGUUCUUGGAAGAGAAGGGCAU
SEQ ID NO: 166 - DsiRNA 79 guide (antisense) strand
AUGCCCUUCUCUUCCAAGAACCUGAAU
SEQ ID NO: 167 - DsiRNA 80 passenger (sense) strand
CAGGUUCUUGGAAGAGAAGGGCAUC
SEQ ID NO: 168 - DsiRNA 80 guide (antisense) strand
GAUGCCCUUCUCUUCCAAGAACCUGAA
SEQ ID NO: 169 - DsiRNA 81 passenger (sense) strand
AGGUUCUUGGAAGAGAAGGGCAUCU
SEQ ID NO: 170 - DsiRNA 81 guide (antisense) strand
AGAUGCCCUUCUCUUCCAAGAACCUGA
SEQ ID NO: 171 - DsiRNA 82 passenger (sense) strand
GGUUCUUGGAAGAGAAGGGCAUCUG
SEQ ID NO: 172 - DsiRNA 82 guide (antisense) strand
CAGAUGCCCUUCUCUUCCAAGAACCUG
SEQ ID NO: 173 - DsiRNA 83 passenger (sense) strand
UGGAAGAGAAGGGCAUCUGCAACAG
SEQ ID NO: 174 - DsiRNA 83 guide (antisense) strand
CUGUUGCAGAUGCCCUUCUCUUCCAAG
SEQ ID NO: 175 - DsiRNA 84 passenger (sense) strand
UGAAGUCAUCCUCAGAAGGGAUGGA
SEQ ID NO: 176 - DsiRNA 84 guide (antisense) strand
UCCAUCCCUUCUGAGGAUGACUUCAGG
SEQ ID NO: 177 - DsiRNA 85 passenger (sense) strand
GAAGUCAUCCUCAGAAGGGAUGGAU
SEQ ID NO: 178 - DsiRNA 85 guide (antisense) strand
AUCCAUCCCUUCUGAGGAUGACUUCAG
SEQ ID NO: 179 - DsiRNA 86 passenger (sense) strand
AAGUCAUCCUCAGAAGGGAUGGAUC
SEQ ID NO: 180 - DsiRNA 86 guide (antisense) strand
GAUCCAUCCCUUCUGAGGAUGACUUCA
SEQ ID NO: 181 - DsiRNA 87 passenger (sense) strand
GUCAUCCUCAGAAGGGAUGGAUCCU
SEQ ID NO: 182 - DsiRNA 87 guide (antisense) strand
AGGAUCCAUCCCUUCUGAGGAUGACUU
SEQ ID NO: 183 - DsiRNA 88 passenger (sense) strand
CAUCCUCAGAAGGGAUGGAUCCUGA
SEQ ID NO: 184 - DsiRNA 88 guide (antisense) strand
UCAGGAUCCAUCCCUUCUGAGGAUGAC
SEQ ID NO: 185 - DsiRNA 89 passenger (sense) strand
AUCCUCAGAAGGGAUGGAUCCUGAG
SEQ ID NO: 186 - DsiRNA 89 guide (antisense) strand)
CUCAGGAUCCAUCCCUUCUGAGGAUGA
SEQ ID NO: 187 - DsiRNA 90 passenger (sense) strand
CUAGACCACCUGCGUCUCAGCAUCC
SEQ ID NO: 188 - DsiRNA 90 guide (antisense) strand
GGAUGCUGAGACGCAGGUGGUCUAGCA
SEQ ID NO: 189 - DsiRNA 91 passenger (sense) strand
AGUGAAGAAAUGAAAGACAAAGGUG
SEQ ID NO: 190 - DsiRNA 91 guide (antisense) strand
CACCUUUGUCUUUCAUUUCUUCACUCA
SEQ ID NO: 191 - DsiRNA 92 passenger (sense) strand
GUGAAGAAAUGAAAGACAAAGGUGG
SEQ ID NO: 192 - DsiRNA 92 guide (antisense) strand
CCACCUUUGUCUUUCAUUUCUUCACUC
SEQ ID NO: 193 - DsiRNA 93 passenger (sense) strand
UGAAGAAAUGAAAGACAAAGGUGGA
SEQ ID NO: 194 - DsiRNA 93 guide (antisense) strand
UCCACCUUUGUCUUUCAUUUCUUCACU
SEQ ID NO: 195 - DsiRNA 94 passenger (sense) strand
GAAGAAAUGAAAGACAAAGGUGGAU
SEQ ID NO: 196 - DsiRNA 94 guide (antisense) strand
AUCCACCUUUGUCUUUCAUUUCUUCAC
SEQ ID NO: 197 - DsiRNA 95 passenger (sense) strand
AAGAAAUGAAAGACAAAGGUGGAUA
SEQ ID NO: 198 - DsiRNA 95 guide (antisense) strand
UAUCCACCUUUGUCUUUCAUUUCUUCA
SEQ ID NO: 199 - DsiRNA 96 passenger (sense) strand
AGAAAUGAAAGACAAAGGUGGAUAC
SEQ ID NO: 200 - DsiRNA 96 guide (antisense) strand
GUAUCCACCUUUGUCUUUCAUUUCUUC
SEQ ID NO: 201 - DsiRNA 97 passenger (sense) strand
GAAAUGAAAGACAAAGGUGGAUACA
SEQ ID NO: 202 - DsiRNA 97 guide (antisense) strand
UGUAUCCACCUUUGUCUUUCAUUUCUU
SEQ ID NO: 203 - DsiRNA 98 passenger (sense) strand
AAAUGAAAGACAAAGGUGGAUACAU
SEQ ID NO: 204 - DsiRNA 98 guide (antisense) strand
AUGUAUCCACCUUUGUCUUUCAUUUCU
SEQ ID NO: 205 - DsiRNA 99 passenger (sense) strand
AAUGAAAGACAAAGGUGGAUACAUG
SEQ ID NO: 206 - DsiRNA 99 guide (antisense) strand
CAUGUAUCCACCUUUGUCUUUCAUUUC
SEQ ID NO: 207 - DsiRNA 100 passenger (sense) strand
AUGAAAGACAAAGGUGGAUACAUGA
SEQ ID NO: 208 - DsiRNA 100 guide (antisense) strand
UCAUGUAUCCACCUUUGUCUUUCAUUU
SEQ ID NO: 209 - DsiRNA 101 passenger (sense) strand
UGAAAGACAAAGGUGGAUACAUGAG
SEQ ID NO: 210 - DsiRNA 101 guide (antisense) strand
CUCAUGUAUCCACCUUUGUCUUUCAUU
SEQ ID NO: 211 - DsiRNA 102 passenger (sense) strand
GAAAGACAAAGGUGGAUACAUGAGC
SEQ ID NO: 212 - DsiRNA 102 guide (antisense) strand
GCUCAUGUAUCCACCUUUGUCUUUCAU
SEQ ID NO: 213 - DsiRNA 103 passenger (sense) strand
AAGACAAAGGUGGAUACAUGAGCAA
SEQ ID NO: 214 - DsiRNA 103 guide (antisense) strand
UUGCUCAUGUAUCCACCUUUGUCUUUC
SEQ ID NO: 215 - DsiRNA 104 passenger (sense) strand
AGACAAAGGUGGAUACAUGAGCAAG
SEQ ID NO: 216 - DsiRNA 104 guide (antisense) strand
CUUGCUCAUGUAUCCACCUUUGUCUUU
SEQ ID NO: 217 - DsiRNA 105 passenger (sense) strand
GACAAAGGUGGAUACAUGAGCAAGA
SEQ ID NO: 218 - DsiRNA 105 guide (antisense) strand
UCUUGCUCAUGUAUCCACCUUUGUCUU
SEQ ID NO: 219 - DsiRNA 106 passenger (sense) strand
ACAAAGGUGGAUACAUGAGCAAGAU
SEQ ID NO: 220 - DsiRNA 106 guide (antisense) strand
AUCUUGCUCAUGUAUCCACCUUUGUCU
SEQ ID NO: 221 - DsiRNA 107 passenger (sense) strand
AAGGUGGAUACAUGAGCAAGAUUUG
SEQ ID NO: 222 - DsiRNA 107 guide (antisense) strand
CAAAUCUUGCUCAUGUAUCCACCUUUG
SEQ ID NO: 223 - DsiRNA 108 passenger (sense) strand
AGGUGGAUACAUGAGCAAGAUUUGC
SEQ ID NO: 224 - DsiRNA 108 guide (antisense) strand
GCAAAUCUUGCUCAUGUAUCCACCUUU
SEQ ID NO: 225 - DsiRNA 109 passenger (sense) strand
UGGAUACAUGAGCAAGAUUUGCAAC
SEQ ID NO: 226 - DsiRNA 109 guide (antisense) strand
GUUGCAAAUCUUGCUCAUGUAUCCACC
SEQ ID NO: 227 - DsiRNA 110 passenger (sense) strand
GGAUACAUGAGCAAGAUUUGCAACU
SEQ ID NO: 228 - DsiRNA 110 guide (antisense) strand
AGUUGCAAAUCUUGCUCAUGUAUCCAC
SEQ ID NO: 229 - DsiRNA 111 passenger (sense) strand
GAUACAUGAGCAAGAUUUGCAACUU
SEQ ID NO: 230 - DsiRNA 111 guide (antisense) strand
AAGUUGCAAAUCUUGCUCAUGUAUCCA
SEQ ID NO: 231 - DsiRNA 112 passenger (sense) strand
AUACAUGAGCAAGAUUUGCAACUUG
SEQ ID NO: 232 -DsiRNA 112 guide (antisense) strand
CAAGUUGCAAAUCUUGCUCAUGUAUCC
SEQ ID NO: 233 - DsiRNA 113 passenger (sense) strand
UACAUGAGCAAGAUUUGCAACUUGC
SEQ ID NO: 234 - DsiRNA 113 guide (antisense) strand
GCAAGUUGCAAAUCUUGCUCAUGUAUC
SEQ ID NO: 235 - DsiRNA 114 passenger (sense) strand
ACAUGAGCAAGAUUUGCAACUUGCU
SEQ ID NO: 236 - DsiRNA 114 guide (antisense) strand
AGCAAGUUGCAAAUCUUGCUCAUGUAU
SEQ ID NO: 237 - DsiRNA 115 passenger (sense) strand
CAUGAGCAAGAUUUGCAACUUGCUA
SEQ ID NO: 238 - DsiRNA 115 guide (antisense) strand
UAGCAAGUUGCAAAUCUUGCUCAUGUA
SEQ ID NO: 239: DsiRNA 116 passenger (sense) strand
AUGAGCAAGAUUUGCAACUUGCUAC
SEQ ID NO: 240 - DsiRNA 116 guide (antisense) strand
GUAGCAAGUUGCAAAUCUUGCUCAUGU
SEQ ID NO: 241 - DsiRNA 117 passenger (sense) strand
UGAGCAAGAUUUGCAACUUGCUACC
SEQ ID NO: 242 - DsiRNA 117 guide (antisense) strand
GGUAGCAAGUUGCAAAUCUUGCUCAUG
SEQ ID NO: 243 - DsiRNA 118 passenger (sense) strand
GAGCAAGAUUUGCAACUUGCUACCC
SEQ ID NO: 244 - DsiRNA 118 guide (antisense) strand
GGGUAGCAAGUUGCAAAUCUUGCUCAU
SEQ ID NO: 245 - DsiRNA 119 passenger (sense) strand
AGCAAGAUUUGCAACUUGCUACCCA
SEQ ID NO: 246 - DsiRNA 119 guide (antisense) strand
UGGGUAGCAAGUUGCAAAUCUUGCUCA
SEQ ID NO: 247 - DsiRNA 120 passenger (sense) strand
GCAAGAUUUGCAACUUGCUACCCAU
SEQ ID NO: 248 - DsiRNA 120 guide (antisense) strand
AUGGGUAGCAAGUUGCAAAUCUUGCUC
SEQ ID NO: 249 - DsiRNA 121 passenger (sense) strand
CAAGAUUUGCAACUUGCUACCCAUU
SEQ ID NO: 250 - DsiRNA 121 guide (antisense) strand
AAUGGGUAGCAAGUUGCAAAUCUUGCU
SEQ ID NO: 251 - DsiRNA 122 passenger (sense) strand
AAGAUUUGCAACUUGCUACCCAUUA
SEQ ID NO: 252 - DsiRNA 122 guide (antisense) strand
UAAUGGGUAGCAAGUUGCAAAUCUUGC
SEQ ID NO: 253 - DsiRNA 123 passenger (sense) strand
AGAUUUGCAACUUGCUACCCAUUAG
SEQ ID NO: 254 - DsiRNA 123 guide (antisense) strand
CUAAUGGGUAGCAAGUUGCAAAUCUUG
SEQ ID NO: 255 - DsiRNA 124 passenger (sense) strand
GAUUUGCAACUUGCUACCCAUUAGG
SEQ ID NO: 256 - DsiRNA 124 guide (antisense) strand
CCUAAUGGGUAGCAAGUUGCAAAUCUU
SEQ ID NO: 257 - DsiRNA 125 passenger (sense) strand
AUUUGCAACUUGCUACCCAUUAGGA
SEQ ID NO: 258 - DsiRNA 125 guide (antisense) strand
UCCUAAUGGGUAGCAAGUUGCAAAUCU
SEQ ID NO: 259 - DsiRNA 126 passenger (sense) strand
UUUGCAACUUGCUACCCAUUAGGAU
SEQ ID NO: 260 - DsiRNA 126 guide (antisense) strand
AUCCUAAUGGGUAGCAAGUUGCAAAUC
SEQ ID NO: 261 - DsiRNA 127 passenger (sense) strand
UUGCAACUUGCUACCCAUUAGGAUA
SEQ ID NO: 262 - DsiRNA 127 guide (antisense) strand
UAUCCUAAUGGGUAGCAAGUUGCAAAU
SEQ ID NO: 263 - DsiRNA 128 passenger (sense) strand
UGCAACUUGCUACCCAUUAGGAUAA
SEQ ID NO: 264 - DsiRNA 128 guide (antisense) strand
UUAUCCUAAUGGGUAGCAAGUUGCAAA
SEQ ID NO: 265 - DsiRNA 129 - passenger (sense) strand
GCAACUUGCUACCCAUUAGGAUAAU
SEQ ID NO: 266 - DsiRNA 129 guide (antisense) strand
AUUAUCCUAAUGGGUAGCAAGUUGCAA
SEQ ID NO: 267 - DsiRNA 130 passenger (sense) strand
CAACUUGCUACCCAUUAGGAUAAUG
SEQ ID NO: 268 - DsiRNA 130 guide (antisense) strand
CAUUAUCCUAAUGGGUAGCAAGUUGCA
SEQ ID NO: 269 - DsiRNA 131 passenger (sense) strand
CUUGCUACCCAUUAGGAUAAUGUCU
SEQ ID NO: 270 - DsiRNA 131 guide (antisense) strand
AGACAUUAUCCUAAUGGGUAGCAAGUU
SEQ ID NO: 271 - DsiRNA 132 passenger (sense) strand
UUGCUACCCAUUAGGAUAAUGUCUU
SEQ ID NO: 272 - DsiRNA 132 guide (antisense) strand
AAGACAUUAUCCUAAUGGGUAGCAAGU
SEQ ID NO: 273 - DsiRNA 133 passenger (sense) strand
UGCUACCCAUUAGGAUAAUGUCUUA
SEQ ID NO: 274 - DsiRNA 133 guide (antisense) strand
UAAGACAUUAUCCUAAUGGGUAGCAAG
SEQ ID NO: 275 - DsiRNA 134 passenger (sense) strand
AUUAGGAUAAUGUCUUAUGUAAUGC
SEQ ID NO: 276 - DsiRNA 134 guide (antisense) strand
GCAUUACAUAAGACAUUAUCCUAAUGG
SEQ ID NO: 277 - DsiRNA 135 passenger (sense) strand
UUAGGAUAAUGUCUUAUGUAAUGCU
SEQ ID NO: 278 - DsiRNA 135 guide (antisense) strand
AGCAUUACAUAAGACAUUAUCCUAAUG
SEQ ID NO: 279 - DsiRNA 136 passenger (sense) strand
CUGUGGAAUCUGCCAUUGCGAUUGU
SEQ ID NO: 280 - DsiRNA 136 guide (antisense) strand
ACAAUCGCAAUGGCAGAUUCCACAGGC
SEQ ID NO: 281 - DsiRNA 137 passenger (sense) strand
GGAAUCUGCCAUUGCGAUUGUCCAG
SEQ ID NO: 282 - DsiRNA 137 guide (antisense) strand
CUGGACAAUCGCAAUGGCAGAUUCCAC
SEQ ID NO: 283 - DsiRNA 138 passenger (sense) strand
GAAUCUGCCAUUGCGAUUGUCCAGA
SEQ ID NO: 284 - DsiRNA 138 guide (antisense) strand
UCUGGACAAUCGCAAUGGCAGAUUCCA
SEQ ID NO: 285 - DsiRNA 139 passenger (sense) strand
AAUCUGCCAUUGCGAUUGUCCAGAG
SEQ ID NO: 286 - DsiRNA 139 guide (antisense) strand
CUCUGGACAAUCGCAAUGGCAGAUUCC
SEQ ID NO: 287 - DsiRNA 140 passenger (sense) strand
AUCUGCCAUUGCGAUUGUCCAGAGA
SEQ ID NO: 288 - DsiRNA 140 guide (antisense) strand
UCUCUGGACAAUCGCAAUGGCAGAUUC
SEQ ID NO: 289 - DsiRNA 141 passenger (sense) strand
CCAUUGCGAUUGUCCAGAGACUGGU
SEQ ID NO: 290 - DsiRNA 141 guide (antisense) strand
ACCAGUCUCUGGACAAUCGCAAUGGCA
SEQ ID NO: 291 - DsiRNA 142 passenger (sense) strand
CAUUGCGAUUGUCCAGAGACUGGUG
SEQ ID NO: 292 - DsiRNA 142 guide (antisense) strand
CACCAGUCUCUGGACAAUCGCAAUGGC
SEQ ID NO: 293 - DsiRNA 143 passenger (sense) strand
AUUGCGAUUGUCCAGAGACUGGUGA
SEQ ID NO: 294 - DsiRNA 143 guide (antisense) strand
UCACCAGUCUCUGGACAAUCGCAAUGG
SEQ ID NO: 295 - DsiRNA 144 passenger (sense) strand
UUGCGAUUGUCCAGAGACUGGUGAC
SEQ ID NO: 296 - DsiRNA 144 guide (antisense) strand
GUCACCAGUCUCUGGACAAUCGCAAUG
SEQ ID NO: 297 - DsiRNA 145 passenger (sense) strand
UGCGAUUGUCCAGAGACUGGUGACA
SEQ ID NO: 298 - DsiRNA 145 guide (antisense) strand
UGUCACCAGUCUCUGGACAAUCGCAAU
SEQ ID NO: 299 - DsiRNA 146 passenger (sense) strand
GCGAUUGUCCAGAGACUGGUGACAU
SEQ ID NO: 300 - DsiRNA 146 guide (antisense) strand
AUGUCACCAGUCUCUGGACAAUCGCAA
SEQ ID NO: 301 - DsiRNA 147 passenger (sense) strand
CGAUUGUCCAGAGACUGGUGACAUG
SEQ ID NO: 302 - DsiRNA 147 guide (antisense) strand
CAUGUCACCAGUCUCUGGACAAUCGCA
SEQ ID NO: 303 - DsiRNA 148 passenger (sense)
AUUGUCCAGAGACUGGUGACAUGGC
SEQ ID NO: 304 - DsiRNA 148 guide (antisense) strand
GCCAUGUCACCAGUCUCUGGACAAUCG
SEQ ID NO: 305 - DsiRNA 149 passenger (sense) strand
GUCCAGAGACUGGUGACAUGGCUUC
SEQ ID NO: 306 - DsiRNA 149 guide (antisense) strand
GAAGCCAUGUCACCAGUCUCUGGACAA
SEQ ID NO: 307 - DsiRNA 150 passenger (sense) strand
CCAGAGACUGGUGACAUGGCUUCCA
SEQ ID NO: 308 - DsiRNA 150 guide (antisense) strand
UGGAAGCCAUGUCACCAGUCUCUGGAC
SEQ ID NO: 309 - DsiRNA 151 passenger (sense)
CAGAGACUGGUGACAUGGCUUCCAG
SEQ ID NO: 310 - DsiRNA 151 guide (antisense) strand
CUGGAAGCCAUGUCACCAGUCUCUGGA
SEQ ID NO: 311 - DsiRNA 152 passenger (sense) strand
AGAGACUGGUGACAUGGCUUCCAGA
SEQ ID NO: 312 - DsiRNA 152 guide (antisense) strand
UCUGGAAGCCAUGUCACCAGUCUCUGG
SEQ ID NO: 313 - DsiRNA 153 passenger (sense) strand
GAGACUGGUGACAUGGCUUCCAGAU
SEQ ID NO: 314 - DsiRNA 153 guide (antisense) strand
AUCUGGAAGCCAUGUCACCAGUCUCUG
SEQ ID NO: 315 - DsiRNA 154 passenger (sense) strand
AGACUGGUGACAUGGCUUCCAGAUA
SEQ ID NO: 316 - DsiRNA 154 guide (antisense) strand
UAUCUGGAAGCCAUGUCACCAGUCUCU
SEQ ID NO: 317 - DsiRNA 155 passenger (sense) strand
GACUGGUGACAUGGCUUCCAGAUAU
SEQ ID NO: 318 - DsiRNA 155 guide (antisense) strand
AUAUCUGGAAGCCAUGUCACCAGUCUC
SEQ ID NO: 319 - DsiRNA 156 passenger (sense) strand
ACUGGUGACAUGGCUUCCAGAUAUG
SEQ ID NO: 320 - DsiRNA 156 guide (antisense) strand
CAUAUCUGGAAGCCAUGUCACCAGUCU
SEQ ID NO: 321 - DsiRNA 157 passenger (sense) strand
CUGGUGACAUGGCUUCCAGAUAUGC
SEQ ID NO: 322 - DsiRNA 157 guide (antisense) strand
GCAUAUCUGGAAGCCAUGUCACCAGUC
SEQ ID NO: 323 - DsiRNA 158 passenger (sense) strand
GACAUGGCUUCCAGAUAUGCCCGAC
SEQ ID NO: 324 - DsiRNA 158 guide (antisense) strand
GUCGGGCAUAUCUGGAAGCCAUGUCAC
SEQ ID NO: 325 - DsiRNA 159 passenger (sense) strand
CAUGGCUUCCAGAUAUGCCCGACGA
SEQ ID NO: 326 - DsiRNA 159 guide (antisense) strand
UCGUCGGGCAUAUCUGGAAGCCAUGUC
SEQ ID NO: 327 - DsiRNA 160 passenger (sense) strand
CAGAUAUGCCCGACGAUGUCCUGUG
SEQ ID NO: 328 - DsiRNA 160 guide (antisense) strand
CACAGGACAUCGUCGGGCAUAUCUGGA
SEQ ID NO: 329 - DsiRNA 161 passenger (sense) strand
CUCACAGGUGUUCACUCGAGUGCUG
SEQ ID NO: 330 - DsiRNA 161 guide (antisense) strand
CAGCACUCGAGUGAACACCUGUGAGGU
SEQ ID NO: 331 - DsiRNA 162 passenger (sense) strand
CACUCGAGUGCUGAUGUGUCUGCUC
SEQ ID NO: 332 - DsiRNA 162 guide (antisense) strand
GAGCAGACACAUCAGCACUCGAGUGAA
SEQ ID NO: 333 - DsiRNA 163 passenger (sense) strand
CUCGAGUGCUGAUGUGUCUGCUCCC
SEQ ID NO: 334 - DsiRNA 163 guide (antisense) strand
GGGAGCAGACACAUCAGCACUCGAGUG
SEQ ID NO: 335 - DsiRNA 164 passenger (sense) strand
CCCAAAUGCCAGUGAGCAGCCAACA
SEQ ID NO: 336 - DsiRNA 164 guide (antisense) strand
UGUUGGCUGCUCACUGGCAUUUGGGAC
SEQ ID NO: 337 - DsiRNA 165 passenger (sense) strand
UCAGGUCCAGCCUGAACUUCUUCUU
SEQ ID NO: 338 - DsiRNA 165 guide (antisense) strand
AAGAAGAAGUUCAGGCUGGACCUGAGG
SEQ ID NO: 339 - DsiRNA 166 passenger (sense) strand
CAGGUCCAGCCUGAACUUCUUCUUG
SEQ ID NO: 340 - DsiRNA 166 guide (antisense) strand
CAAGAAGAAGUUCAGGCUGGACCUGAG
SEQ ID NO: 341 - DsiRNA 167 passenger (sense) strand
AGGUCCAGCCUGAACUUCUUCUUGG
SEQ ID NO: 342 - DsiRNA 167 guide (antisense) strand
CCAAGAAGAAGUUCAGGCUGGACCUGA
SEQ ID NO: 343 - DsiRNA 168 passenger (sense) strand
CAAUAAAGUACCUGCUGGUGCUGAG
SEQ ID NO: 344 - DsiRNA 168 guide (antisense) strand
CUCAGCACCAGCAGGUACUUUAUUGCC
SEQ ID NO: 345 - DsiRNA 169 passenger (sense) strand
AAUAAAGUACCUGCUGGUGCUGAGG
SEQ ID NO: 346 - DsiRNA 169 guide (antisense) strand
CCUCAGCACCAGCAGGUACUUUAUUGC
SEQ ID NO: 347 - DsiRNA 170 passenger (sense) strand
AUAAAGUACCUGCUGGUGCUGAGGG
SEQ ID NO: 348 - DsiRNA 170 guide (antisense) strand
CCCUCAGCACCAGCAGGUACUUUAUUG
SEQ ID NO: 349 - DsiRNA 171 passenger (sense) strand
CUCUCCACCUUUCCCAGUUUUUCAC
SEQ ID NO: 350 - DsiRNA 171 guide (antisense) strand
GUGAAAAACUGGGAAAGGUGGAGAGCC
SEQ ID NO: 351 - DsiRNA 172 passenger (sense) strand
CUCCACCUUUCCCAGUUUUUCACUA
SEQ ID NO: 352 - DsiRNA 172 guide (antisense) strand
UAGUGAAAAACUGGGAAAGGUGGAGAG
SEQ ID NO: 353 - DsiRNA 173 passenger (sense) strand
UCCACCUUUCCCAGUUUUUCACUAG
SEQ ID NO: 354 - DsiRNA 173 guide (antisense) strand
CUAGUGAAAAACUGGGAAAGGUGGAGA
SEQ ID NO: 355 - DsiRNA 174 passenger (sense) strand
CCACCUUUCCCAGUUUUUCACUAGA
SEQ ID NO: 356 - DsiRNA 174 guide (antisense) strand
UCUAGUGAAAAACUGGGAAAGGUGGAG
SEQ ID NO: 357 - DsiRNA 175 passenger (sense) strand
CACCUUUCCCAGUUUUUCACUAGAG
SEQ ID NO: 358 - DsiRNA 175 guide (antisense) strand
CUCUAGUGAAAAACUGGGAAAGGUGGA
SEQ ID NO: 359 - DsiRNA 176 passenger (sense) strand
ACCUUUCCCAGUUUUUCACUAGAGA
SEQ ID NO: 360 - DsiRNA 176 guide (antisense) strand
UCUCUAGUGAAAAACUGGGAAAGGUGG
SEQ ID NO: 361 - DsiRNA 177 passenger (sense) strand
CCUUUCCCAGUUUUUCACUAGAGAA
SEQ ID NO: 362 - DsiRNA 177 guide (antisense) strand
UUCUCUAGUGAAAAACUGGGAAAGGUG
SEQ ID NO: 363 - DsiRNA 178 passenger (sense) strand
AGUUUUUCACUAGAGAAGAGUCUGU
SEQ ID NO: 364 - DsiRNA 178 guide (antisense) strand
ACAGACUCUUCUCUAGUGAAAAACUGG
SEQ ID NO: 365 - DsiRNA 179 passenger (sense) strand
UCUAGCAGAUUCUUUCAGAGGUGCU
SEQ ID NO: 366 - DsiRNA 179 guide (antisense) strand
AGCACCUCUGAAAGAAUCUGCUAGACU
SEQ ID NO: 367 - DsiRNA 180 passenger (sense) strand
AGCAGAUUCUUUCAGAGGUGCUAAA
SEQ ID NO: 368 - DsiRNA 180 guide (antisense) strand
UUUAGCACCUCUGAAAGAAUCUGCUAG
SEQ ID NO: 369 - DsiRNA 181 passenger (sense) strand
GCAGAUUCUUUCAGAGGUGCUAAAG
SEQ ID NO: 370 - DsiRNA 181 guide (antisense) strand
CUUUAGCACCUCUGAAAGAAUCUGCUA
SEQ ID NO: 371 - DsiRNA 182 passenger (sense) strand
CAGAUUCUUUCAGAGGUGCUAAAGU
SEQ ID NO: 372 - DsiRNA 182 guide (antisense) strand
ACUUUAGCACCUCUGAAAGAAUCUGCU
SEQ ID NO: 373 - DsiRNA 183 passenger (sense) strand
AGAUUCUUUCAGAGGUGCUAAAGUU
SEQ ID NO: 374 - DsiRNA 183 guide (antisense) strand
AACUUUAGCACCUCUGAAAGAAUCUGC
SEQ ID NO: 375 - DsiRNA 184 passenger (sense) strand
GAUUCUUUCAGAGGUGCUAAAGUUU
SEQ ID NO: 376 - DsiRNA 184 guide (antisense) strand
AAACUUUAGCACCUCUGAAAGAAUCUG
SEQ ID NO: 377 - DsiRNA 185 passenger (sense) strand
AUUCUUUCAGAGGUGCUAAAGUUUC
SEQ ID NO: 378 - DsiRNA 185 guide (antisense) strand
GAAACUUUAGCACCUCUGAAAGAAUCU
SEQ ID NO: 379 - DsiRNA 186 passenger (sense) strand
AGGUGCUAAAGUUUCCCAUCUUUGU
SEQ ID NO: 380 - DsiRNA 186 guide (antisense) strand
ACAAAGAUGGGAAACUUUAGCACCUCU
SEQ ID NO: 381 - DsiRNA 187 passenger (sense) strand
GGUGCUAAAGUUUCCCAUCUUUGUG
SEQ ID NO: 382 - DsiRNA 187 guide (antisense) strand
CACAAAGAUGGGAAACUUUAGCACCUC
SEQ ID NO: 383 - DsiRNA 188 passenger (sense) strand
GUGCUAAAGUUUCCCAUCUUUGUGC
SEQ ID NO: 384 - DsiRNA 188 guide (antisense) strand
GCACAAAGAUGGGAAACUUUAGCACCU
SEQ ID NO: 385 - DsiRNA 189 passenger (sense) strand
GCUAAAGUUUCCCAUCUUUGUGCAG
SEQ ID NO: 386 - DsiRNA 189 guide (antisense) strand
CUGCACAAAGAUGGGAAACUUUAGCAC
SEQ ID NO: 387 - DsiRNA 190 passenger (sense) strand
CUAAAGUUUCCCAUCUUUGUGCAGC
SEQ ID NO: 388 - DsiRNA 190 guide (antisense) strand
GCUGCACAAAGAUGGGAAACUUUAGCA
SEQ ID NO: 389 - DsiRNA 191 passenger (sense) strand
UAAAGUUUCCCAUCUUUGUGCAGCU
SEQ ID NO: 390 - DsiRNA 191 guide (antisense) strand
AGCUGCACAAAGAUGGGAAACUUUAGC
SEQ ID NO: 391 - DsiRNA 192 passenger (sense) strand
AAAGUUUCCCAUCUUUGUGCAGCUA
SEQ ID NO: 392 - DsiRNA 192 guide (antisense) strand
UAGCUGCACAAAGAUGGGAAACUUUAG
SEQ ID NO: 393 - DsiRNA 193 passenger (sense) strand
AAGUUUCCCAUCUUUGUGCAGCUAC
SEQ ID NO: 394 - DsiRNA 193 guide (antisense) strand
GUAGCUGCACAAAGAUGGGAAACUUUA
SEQ ID NO: 395 - DsiRNA 194 passenger (sense) strand
AGUUUCCCAUCUUUGUGCAGCUACC
SEQ ID NO: 396 - DsiRNA 194 guide (antisense) strand
GGUAGCUGCACAAAGAUGGGAAACUUU
SEQ ID NO: 397 - DsiRNA 195 passenger (sense) strand
GUUUCCCAUCUUUGUGCAGCUACCU
SEQ ID NO: 398 - DsiRNA 195 guide (antisense) strand
AGGUAGCUGCACAAAGAUGGGAAACUU
SEQ ID NO: 399 - DsiRNA 196 passenger (sense) strand
AUCUUUGUGCAGCUACCUCCGCAUU
SEQ ID NO: 400 - DsiRNA 196 guide (antisense) strand
AAUGCGGAGGUAGCUGCACAAAGAUGG
SEQ ID NO: 401 - DsiRNA 197 passenger (sense) strand
GUGCAGCUACCUCCGCAUUGCUGUG
SEQ ID NO: 402 - DsiRNA 197 guide (antisense) strand
CACAGCAAUGCGGAGGUAGCUGCACAA
SEQ ID NO: 403 - DsiRNA 198 passenger (sense) strand
CCAGCCUCUGAGCUGAGUUGGUUUU
SEQ ID NO: 404 - DsiRNA 198 guide (antisense) strand
AAAACCAACUCAGCUCAGAGGCUGGGA
SEQ ID NO: 405 - DsiRNA 199 passenger (sense) strand
CAGCCUCUGAGCUGAGUUGGUUUUA
SEQ ID NO: 406 - DsiRNA 199 guide (antisense) strand
UAAAACCAACUCAGCUCAGAGGCUGGG
SEQ ID NO: 407 - DsiRNA 200 passenger (sense) strand
AGCCUCUGAGCUGAGUUGGUUUUAU
SEQ ID NO: 408 - DsiRNA 200 guide (antisense) strand
AUAAAACCAACUCAGCUCAGAGGCUGG
SEQ ID NO: 409 - DsiRNA 201 passenger (sense) strand
GCCUCUGAGCUGAGUUGGUUUUAUG
SEQ ID NO: 410 - DsiRNA 201 guide (antisense) strand
CAUAAAACCAACUCAGCUCAGAGGCUG
SEQ ID NO: 411 - DsiRNA 202 passenger (sense) strand
CCUCUGAGCUGAGUUGGUUUUAUGA
SEQ ID NO: 412 - DsiRNA 202 guide (antisense) strand
UCAUAAAACCAACUCAGCUCAGAGGCU
SEQ ID NO: 413 - DsiRNA 203 passenger (sense) strand
CUCUGAGCUGAGUUGGUUUUAUGAA
SEQ ID NO: 414 - DsiRNA 203 guide (antisense) strand
UUCAUAAAACCAACUCAGCUCAGAGGC
SEQ ID NO: 415 - DsiRNA 204 passenger (sense) strand
UCUGAGCUGAGUUGGUUUUAUGAAA
SEQ ID NO: 416 - DsiRNA 204 guide (antisense) strand
UUUCAUAAAACCAACUCAGCUCAGAGG
SEQ ID NO: 417 - DsiRNA 205 passenger (sense) strand
UGAGCUGAGUUGGUUUUAUGAAAAG
SEQ ID NO: 418 - DsiRNA 205 guide (antisense) strand
CUUUUCAUAAAACCAACUCAGCUCAGA
SEQ ID NO: 419 - DsiRNA 206 passenger (sense) strand
UGAGUUGGUUUUAUGAAAAGCUAGG
SEQ ID NO: 420 - DsiRNA 206 guide (antisense) strand
CCUAGCUUUUCAUAAAACCAACUCAGC
SEQ ID NO: 421 - DsiRNA 207 passenger (sense) strand
GAGUUGGUUUUAUGAAAAGCUAGGA
SEQ ID NO: 422 - DsiRNA 207 guide (antisense) strand
UCCUAGCUUUUCAUAAAACCAACUCAG
SEQ ID NO: 423 - DsiRNA 208 passenger (sense) strand
UUGGUUUUAUGAAAAGCUAGGAAGC
SEQ ID NO: 424 - DsiRNA 208 guide (antisense) strand
GCUUCCUAGCUUUUCAUAAAACCAACU
SEQ ID NO: 425 - DsiRNA 209 passenger (sense) strand
UGGUUUUAUGAAAAGCUAGGAAGCA
SEQ ID NO: 426 - DsiRNA 209 guide (antisense) strand
UGCUUCCUAGCUUUUCAUAAAACCAAC
SEQ ID NO: 427 - DsiRNA 210 passenger (sense) strand
GGUUUUAUGAAAAGCUAGGAAGCAA
SEQ ID NO: 428 - DsiRNA 210 guide (antisense) strand
UUGCUUCCUAGCUUUUCAUAAAACCAA
SEQ ID NO: 429 - DsiRNA 211 passenger (sense) strand
UUUUAUGAAAAGCUAGGAAGCAACC
SEQ ID NO: 430 - DsiRNA 211 guide (antisense) strand
GGUUGCUUCCUAGCUUUUCAUAAAACC
SEQ ID NO: 431 - DsiRNA 212 passenger (sense) strand
UUAUGAAAAGCUAGGAAGCAACCUU
SEQ ID NO: 432 - DsiRNA 212 guide (antisense) strand
AAGGUUGCUUCCUAGCUUUUCAUAAAA
SEQ ID NO: 433 - DsiRNA 213 passenger (sense) strand
UAUGAAAAGCUAGGAAGCAACCUUU
SEQ ID NO: 434 - DsiRNA 213 guide (antisense) strand
AAAGGUUGCUUCCUAGCUUUUCAUAAA
SEQ ID NO: 435 - DsiRNA 214 passenger (sense) strand
AUGAAAAGCUAGGAAGCAACCUUUC
SEQ ID NO: 436 - DsiRNA 214 guide (antisense) strand
GAAAGGUUGCUUCCUAGCUUUUCAUAA
SEQ ID NO: 437 - DsiRNA 215 passenger (sense) strand
CCAGCACUUAACUCUAAUACAUCAG
SEQ ID NO: 438 - DsiRNA 215 guide (antisense) strand
CUGAUGUAUUAGAGUUAAGUGCUGGAC
SEQ ID NO: 439 - DsiRNA 216 passenger (sense) strand
CAGCACUUAACUCUAAUACAUCAGC
SEQ ID NO: 440 - DsiRNA 216 guide (antisense) strand
GCUGAUGUAUUAGAGUUAAGUGCUGGA
SEQ ID NO: 441 - DsiRNA 217 passenger (sense) strand
UAAUACAUCAGCAUGCGUUAAUUCA
SEQ ID NO: 442 - DsiRNA 217 guide (antisense) strand
UGAAUUAACGCAUGCUGAUGUAUUAGA
SEQ ID NO: 443 - DsiRNA 218 passenger (sense) strand
AAUACAUCAGCAUGCGUUAAUUCAG
SEQ ID NO: 444 - DsiRNA 218 guide (antisense) strand
CUGAAUUAACGCAUGCUGAUGUAUUAG
SEQ ID NO: 445 - DsiRNA 219 passenger (sense) strand
AGCAUGCGUUAAUUCAGCUGGUUGG
SEQ ID NO: 446 - DsiRNA 219 guide (antisense) strand
CCAACCAGCUGAAUUAACGCAUGCUGA
SEQ ID NO: 447 - DsiRNA 220 passenger (sense) strand
GCAUGCGUUAAUUCAGCUGGUUGGG
SEQ ID NO: 448 - DsiRNA 220 guide (antisense) strand
CCCAACCAGCUGAAUUAACGCAUGCUG
SEQ ID NO: 449 - DsiRNA 221 passenger (sense) strand
UGCGUUAAUUCAGCUGGUUGGGAAA
SEQ ID NO: 450 - DsiRNA 221 guide (antisense) strand
UUUCCCAACCAGCUGAAUUAACGCAUG
SEQ ID NO: 451 - DsiRNA 222 passenger (sense) strand
GCGUUAAUUCAGCUGGUUGGGAAAU
SEQ ID NO: 452 - DsiRNA 222 guide (antisense) strand
AUUUCCCAACCAGCUGAAUUAACGCAU
SEQ ID NO: 453 - DsiRNA 223 passenger (sense) strand
CGUUAAUUCAGCUGGUUGGGAAAUG
SEQ ID NO: 454 - DsiRNA 223 guide (antisense) strand
CAUUUCCCAACCAGCUGAAUUAACGCA
SEQ ID NO: 455 - DsiRNA 224 passenger (sense) strand
GUUAAUUCAGCUGGUUGGGAAAUGA
SEQ ID NO: 456 - DsiRNA 224 guide (antisense) strand
UCAUUUCCCAACCAGCUGAAUUAACGC
SEQ ID NO: 457 - DsiRNA 225 passenger (sense) strand
UUAAUUCAGCUGGUUGGGAAAUGAC
SEQ ID NO: 458 - DsiRNA 225 guide (antisense) strand
GUCAUUUCCCAACCAGCUGAAUUAACG
SEQ ID NO: 459 - DsiRNA 226 passenger (sense) strand
UAAUUCAGCUGGUUGGGAAAUGACA
SEQ ID NO: 460 - DsiRNA 226 guide (antisense) strand
UGUCAUUUCCCAACCAGCUGAAUUAAC
SEQ ID NO: 461 - DsiRNA 227 passenger (sense) strand
AUUCAGCUGGUUGGGAAAUGACACC
SEQ ID NO: 462 - DsiRNA 227 guide (antisense) strand
GGUGUCAUUUCCCAACCAGCUGAAUUA
SEQ ID NO: 463 - DsiRNA 228 passenger (sense) strand
UUCAGCUGGUUGGGAAAUGACACCA
SEQ ID NO: 464 - DsiRNA 228 guide (antisense) strand
UGGUGUCAUUUCCCAACCAGCUGAAUU
SEQ ID NO: 465 - DsiRNA 229 passenger (sense) strand
UCAGCUGGUUGGGAAAUGACACCAG
SEQ ID NO: 466 - DsiRNA 229 guide (antisense) strand
CUGGUGUCAUUUCCCAACCAGCUGAAU
SEQ ID NO: 467 - DsiRNA 230 passenger (sense) strand
CAGCUGGUUGGGAAAUGACACCAGG
SEQ ID NO: 468 - DsiRNA 230 guide (antisense) strand
CCUGGUGUCAUUUCCCAACCAGCUGAA
SEQ ID NO: 469 - DsiRNA 231 passenger (sense) strand
AGCUGGUUGGGAAAUGACACCAGGA
SEQ ID NO: 470 - DsiRNA 231 guide (antisense) strand
UCCUGGUGUCAUUUCCCAACCAGCUGA
SEQ ID NO: 471 - DsiRNA 232 passenger (sense) strand
GCUGGUUGGGAAAUGACACCAGGAA
SEQ ID NO: 472 - DsiRNA 232 guide (antisense) strand
UUCCUGGUGUCAUUUCCCAACCAGCUG
SEQ ID NO: 473 - DsiRNA 233 passenger (sense) strand
GCAGAGGGUCCCUUACUGACUGUUU
SEQ ID NO: 474 - DsiRNA 233 guide (antisense) strand
AAACAGUCAGUAAGGGACCCUCUGCAC
SEQ ID NO: 475 - DsiRNA 234 passenger (sense) strand
CAGAGGGUCCCUUACUGACUGUUUC
SEQ ID NO: 476 - DsiRNA 234 guide (antisense) strand
GAAACAGUCAGUAAGGGACCCUCUGCA
SEQ ID NO: 477 - DsiRNA 235 passenger (sense) strand
AGAGGGUCCCUUACUGACUGUUUCG
SEQ ID NO: 478 - DsiRNA 235 guide (antisense) strand
CGAAACAGUCAGUAAGGGACCCUCUGC
SEQ ID NO: 479 - DsiRNA 236 passenger (sense) strand
CCUAUUAAUGGUCAGACUGUUCCAG
SEQ ID NO: 480 - DsiRNA 236 guide (antisense) strand
CUGGAACAGUCUGACCAUUAAUAGGGC
SEQ ID NO: 481 - DsiRNA 237 passenger (sense) strand
CUAUUAAUGGUCAGACUGUUCCAGC
SEQ ID NO: 482 - DsiRNA 237 guide (antisense) strand
GCUGGAACAGUCUGACCAUUAAUAGGG
SEQ ID NO: 483 - DsiRNA 238 passenger (sense) strand
UAUUAAUGGUCAGACUGUUCCAGCA
SEQ ID NO: 484 - DsiRNA 238 guide (antisense) strand
UGCUGGAACAGUCUGACCAUUAAUAGG
SEQ ID NO: 485 - DsiRNA 239 passenger (sense) strand
AUUAAUGGUCAGACUGUUCCAGCAU
SEQ ID NO: 486 - DsiRNA 239 guide (antisense) strand
AUGCUGGAACAGUCUGACCAUUAAUAG
SEQ ID NO: 487 - DsiRNA 240 passenger (sense) strand
UUAAUGGUCAGACUGUUCCAGCAUG
SEQ ID NO: 488 - DsiRNA 240 guide (antisense) strand
CAUGCUGGAACAGUCUGACCAUUAAUA
SEQ ID NO: 489 - DsiRNA 241 passenger (sense) strand
UAAUGGUCAGACUGUUCCAGCAUGA
SEQ ID NO: 490 - DsiRNA 241 guide (antisense) strand
UCAUGCUGGAACAGUCUGACCAUUAAU
SEQ ID NO: 491 - DsiRNA 242 passenger (sense) strand
AAUGGUCAGACUGUUCCAGCAUGAG
SEQ ID NO: 492 - DsiRNA 242 guide (antisense) strand
CUCAUGCUGGAACAGUCUGACCAUUAA
SEQ ID NO: 493 - DsiRNA 243 passenger (sense) strand
AGAACGACACUGCCUGUCAGGUGGU
SEQ ID NO: 494 - DsiRNA 243 guide (antisense) strand
ACCACCUGACAGGCAGUGUCGUUCUUG
SEQ ID NO: 495 - DsiRNA 244 passenger (sense) strand
CGACACUGCCUGUCAGGUGGUCUGC
SEQ ID NO: 496 - DsiRNA 244 guide (antisense) strand
GCAGACCACCUGACAGGCAGUGUCGUU
SEQ ID NO: 497 - DsiRNA 245 passenger (sense) strand
AACCUUGACUACUAAAAACGUCUCC
SEQ ID NO: 498 - DsiRNA 245 guide (antisense) strand
GGAGACGUUUUUAGUAGUCAAGGUUAU
SEQ ID NO: 499 - DsiRNA 246 passenger (sense) strand
UUUAGAACACCUUUUUCACCUAACU
SEQ ID NO: 500 - DsiRNA 246 guide (antisense) strand
AGUUAGGUGAAAAAGGUGUUCUAAAAU
SEQ ID NO: 501 - DsiRNA 247 passenger (sense) strand
UUAGAACACCUUUUUCACCUAACUA
SEQ ID NO: 502 - DsiRNA 247 guide (antisense) strand
UAGUUAGGUGAAAAAGGUGUUCUAAAA
SEQ ID NO: 503 - DsiRNA 248 passenger (sense) strand
UAGAACACCUUUUUCACCUAACUAA
SEQ ID NO: 504 - DsiRNA 248 guide (antisense) strand
UUAGUUAGGUGAAAAAGGUGUUCUAAA
SEQ ID NO: 505 - DsiRNA 249 passenger (sense) strand
AGAACACCUUUUUCACCUAACUAAA
SEQ ID NO: 506 - DsiRNA 249 guide (antisense) strand
UUUAGUUAGGUGAAAAAGGUGUUCUAA
SEQ ID NO: 507 - DsiRNA 250 passenger (sense) strand
GAACACCUUUUUCACCUAACUAAAA
SEQ ID NO: 508 - DsiRNA 250 guide (antisense) strand
UUUUAGUUAGGUGAAAAAGGUGUUCUA
SEQ ID NO: 509 - DsiRNA 251 passenger (sense) strand
AACACCUUUUUCACCUAACUAAAAU
SEQ ID NO: 510 - DsiRNA 251 guide (antisense) strand
AUUUUAGUUAGGUGAAAAAGGUGUUCU
SEQ ID NO: 511 - DsiRNA 252 passenger (sense) strand
ACACCUUUUUCACCUAACUAAAAUA
SEQ ID NO: 512 - DsiRNA 252 guide (antisense) strand
UAUUUUAGUUAGGUGAAAAAGGUGUUC
SEQ ID NO: 513 - DsiRNA 253 passenger (sense) strand
CACCUUUUUCACCUAACUAAAAUAA
SEQ ID NO: 514 - DsiRNA 253 guide (antisense) strand
UUAUUUUAGUUAGGUGAAAAAGGUGUU
SEQ ID NO: 515 - DsiRNA 254 passenger (sense) strand
ACCUUUUUCACCUAACUAAAAUAAU
SEQ ID NO: 516 - DsiRNA 254 guide (antisense) strand
AUUAUUUUAGUUAGGUGAAAAAGGUGU
SEQ ID NO: 517 - DsiRNA 255 passenger (sense) strand
CUUUUUCACCUAACUAAAAUAAUGU
SEQ ID NO: 518 - DsiRNA 255 guide (antisense) strand
ACAUUAUUUUAGUUAGGUGAAAAAGGU
SEQ ID NO: 519 - DsiRNA 256 passenger (sense) strand
UUUUCACCUAACUAAAAUAAUGUUU
SEQ ID NO: 520 - DsiRNA 256 guide (antisense) strand
AAACAUUAUUUUAGUUAGGUGAAAAAG
SEQ ID NO: 521 - DsiRNA 257 passenger (sense) strand
UUCACCUAACUAAAAUAAUGUUUAA
SEQ ID NO: 522 - DsiRNA 257 guide (antisense) strand
UUAAACAUUAUUUUAGUUAGGUGAAAA
SEQ ID NO: 523 - DsiRNA 258 passenger (sense) strand
UCACCUAACUAAAAUAAUGUUUAAA
SEQ ID NO: 524 - DsiRNA 258 guide (antisense) strand
UUUAAACAUUAUUUUAGUUAGGUGAAA
SEQ ID NO: 525 - DsiRNA 259 passenger (sense) strand
CACCUAACUAAAAUAAUGUUUAAAG
SEQ ID NO: 526 - DsiRNA 259 guide (antisense) strand
CUUUAAACAUUAUUUUAGUUAGGUGAA
SEQ ID NO: 527 - DsiRNA 260 passenger (sense) strand
ACCUAACUAAAAUAAUGUUUAAAGA
SEQ ID NO: 528 - DsiRNA 260 guide (antisense) strand
UCUUUAAACAUUAUUUUAGUUAGGUGA
SEQ ID NO: 529 - DsiRNA 261 passenger (sense) strand
CCUAACUAAAAUAAUGUUUAAAGAG
SEQ ID NO: 530 - DsiRNA 261 guide (antisense) strand
CUCUUUAAACAUUAUUUUAGUUAGGUG
SEQ ID NO: 531 - DsiRNA 262 passenger (sense) strand
CUAACUAAAAUAAUGUUUAAAGAGU
SEQ ID NO: 532 - DsiRNA 262 guide (antisense) strand
ACUCUUUAAACAUUAUUUUAGUUAGGU
SEQ ID NO: 533 - DsiRNA 263 passenger (sense) strand
UAACUAAAAUAAUGUUUAAAGAGUU
SEQ ID NO: 534 - DsiRNA 263 guide (antisense) strand
AACUCUUUAAACAUUAUUUUAGUUAGG
SEQ ID NO: 535 - DsiRNA 264 passenger (sense) strand
AACUAAAAUAAUGUUUAAAGAGUUU
SEQ ID NO: 536 - DsiRNA 264 guide (antisense) strand
AAACUCUUUAAACAUUAUUUUAGUUAG
SEQ ID NO: 537 - DsiRNA 265 passenger (sense) strand
ACUAAAAUAAUGUUUAAAGAGUUUU
SEQ ID NO: 538 - DsiRNA 265 guide (antisense) strand
AAAACUCUUUAAACAUUAUUUUAGUUA
SEQ ID NO: 539 - DsiRNA 266 passenger (sense) strand
CUAAAAUAAUGUUUAAAGAGUUUUG
SEQ ID NO: 540 - DsiRNA 266 guide (antisense) strand
CAAAACUCUUUAAACAUUAUUUUAGUU
SEQ ID NO: 541 - DsiRNA 267 passenger (sense) strand
UAAAAUAAUGUUUAAAGAGUUUUGU
SEQ ID NO: 542 - DsiRNA 267 guide (antisense) strand
ACAAAACUCUUUAAACAUUAUUUUAGU
SEQ ID NO: 543 - DsiRNA 268 passenger (sense) strand
AAGAGUUUUGUAUAAAAAUGUAAGG
SEQ ID NO: 544 - DsiRNA 268 guide (antisense) strand
CCUUACAUUUUUAUACAAAACUCUUUA
SEQ ID NO: 545 - DsiRNA 269 passenger (sense) strand
GUUUUGUAUAAAAAUGUAAGGAAGC
SEQ ID NO: 546 - DsiRNA 269 guide (antisense) strand
GCUUCCUUACAUUUUUAUACAAAACUC
SEQ ID NO: 547 - DsiRNA 270 passenger (sense) strand
UUUUGUAUAAAAAUGUAAGGAAGCG
SEQ ID NO: 548 - DsiRNA 270 guide (antisense) strand
CGCUUCCUUACAUUUUUAUACAAAACU
SEQ ID NO: 549 - DsiRNA 271 passenger (sense) strand
UUUGUAUAAAAAUGUAAGGAAGCGU
SEQ ID NO: 550 - DsiRNA 271 guide (antisense) strand
ACGCUUCCUUACAUUUUUAUACAAAAC
SEQ ID NO: 551 - DsiRNA 272 passenger (sense) strand
UUGUAUAAAAAUGUAAGGAAGCGUU
SEQ ID NO: 552 - DsiRNA 272 guide (antisense) strand
AACGCUUCCUUACAUUUUUAUACAAAA
SEQ ID NO: 553 - DsiRNA 273 passenger (sense) strand
UGUAUAAAAAUGUAAGGAAGCGUUG
SEQ ID NO: 554 - DsiRNA 273 guide (antisense) strand
CAACGCUUCCUUACAUUUUUAUACAAA
SEQ ID NO: 555 - DsiRNA 274 passenger (sense) strand
GUAUAAAAAUGUAAGGAAGCGUUGU
SEQ ID NO: 556 - DsiRNA 274 guide (antisense) strand
ACAACGCUUCCUUACAUUUUUAUACAA
SEQ ID NO: 557 - DsiRNA 275 passenger (sense) strand
AUGUAAGGAAGCGUUGUUACCUGUU
SEQ ID NO: 558 - DsiRNA 275 guide (antisense) strand
AACAGGUAACAACGCUUCCUUACAUUU
SEQ ID NO: 559 - DsiRNA 276 passenger (sense) strand
UUUUGUAUUAUGUGAAUCAGUGAGA
SEQ ID NO: 560 - DsiRNA 276 guide (antisense) strand
UCUCACUGAUUCACAUAAUACAAAAUU
SEQ ID NO: 561 - DsiRNA 277 passenger (sense) strand
UUUGUAUUAUGUGAAUCAGUGAGAU
SEQ ID NO: 562 - DsiRNA 277 guide (antisense) strand
AUCUCACUGAUUCACAUAAUACAAAAU
SEQ ID NO: 563 - DsiRNA 278 passenger (sense) strand
UUGUAUUAUGUGAAUCAGUGAGAUG
SEQ ID NO: 564 - DsiRNA 278 guide (antisense) strand
CAUCUCACUGAUUCACAUAAUACAAAA
SEQ ID NO: 565 - DsiRNA 279 passenger (sense) strand
UGUAUUAUGUGAAUCAGUGAGAUGU
SEQ ID NO: 566 - DsiRNA 279 guide (antisense) strand
ACAUCUCACUGAUUCACAUAAUACAAA
SEQ ID NO: 567 - DsiRNA 280 passenger (sense) strand
GUAUUAUGUGAAUCAGUGAGAUGUU
SEQ ID NO: 568 - DsiRNA 280 guide (antisense) strand
AACAUCUCACUGAUUCACAUAAUACAA
SEQ ID NO: 569 - DsiRNA 281 passenger (sense) strand
UAUUAUGUGAAUCAGUGAGAUGUUA
SEQ ID NO: 570 - DsiRNA 281 guide (antisense) strand
UAACAUCUCACUGAUUCACAUAAUACA
SEQ ID NO: 571 - DsiRNA 282 passenger (sense) strand
AUUAUGUGAAUCAGUGAGAUGUUAG
SEQ ID NO: 572 - DsiRNA 282 guide (antisense) strand
CUAACAUCUCACUGAUUCACAUAAUAC
SEQ ID NO: 573 - DsiRNA 283 passenger (sense) strand
UUAUGUGAAUCAGUGAGAUGUUAGU
SEQ ID NO: 574 - DsiRNA 283 guide (antisense) strand
ACUAACAUCUCACUGAUUCACAUAAUA
SEQ ID NO: 575 - DsiRNA 284 passenger (sense) strand
UAUGUGAAUCAGUGAGAUGUUAGUA
SEQ ID NO: 576 - DsiRNA 284 guide (antisense) strand
UACUAACAUCUCACUGAUUCACAUAAU
SEQ ID NO: 577 - DsiRNA 285 passenger (sense) strand
AUGUGAAUCAGUGAGAUGUUAGUAG
SEQ ID NO: 578 - DsiRNA 285 guide (antisense) strand
CUACUAACAUCUCACUGAUUCACAUAA
SEQ ID NO: 579 - DsiRNA 286 passenger (sense) strand
UGUGAAUCAGUGAGAUGUUAGUAGA
SEQ ID NO: 580 - DsiRNA 286 guide (antisense) strand
UCUACUAACAUCUCACUGAUUCACAUA
SEQ ID NO: 581 - DsiRNA 287 passenger (sense) strand
GUGAAUCAGUGAGAUGUUAGUAGAA
SEQ ID NO: 582 - DsiRNA 287 guide (antisense) strand
UUCUACUAACAUCUCACUGAUUCACAU
SEQ ID NO: 583 - DsiRNA 288 passenger (sense) strand
GUGAGAUGUUAGUAGAAUAAGCCUU
SEQ ID NO: 584 - DsiRNA 288 guide (antisense) strand
AAGGCUUAUUCUACUAACAUCUCACUG
SEQ ID NO: 585 - DsiRNA 289 passenger (sense) strand
UUUUCUAUUUAUGCAUUUGAGUACA
SEQ ID NO: 586 - DsiRNA 289 guide (antisense) strand
UGUACUCAAAUGCAUAAAUAGAAAAAA
SEQ ID NO: 587 - DsiRNA 290 passenger (sense) strand
UUUCUAUUUAUGCAUUUGAGUACAG
SEQ ID NO: 588 - DsiRNA 290 guide (antisense) strand
CUGUACUCAAAUGCAUAAAUAGAAAAA
SEQ ID NO: 589 - DsiRNA 291 passenger (sense) strand
UUCUAUUUAUGCAUUUGAGUACAGT
SEQ ID NO: 590 - DsiRNA 291 guide (antisense) strand
ACUGUACUCAAAUGCAUAAAUAGAAAA
SEQ ID NO: 591 - DsiRNA 292 passenger (sense) strand
CUAUUUAUGCAUUUGAGUACAGUAC
SEQ ID NO: 592 - DsiRNA 292 guide (antisense) strand
GUACUGUACUCAAAUGCAUAAAUAGAA
SEQ ID NO: 593 - DsiRNA 293 passenger (sense) strand
UGCUCAAACUGUUAAAUGUUGGAAA
SEQ ID NO: 594 - DsiRNA 293 guide (antisense) strand
UUUCCAACAUUUAACAGUUUGAGCACA
SEQ ID NO: - DsiRNA 294 passenger (sense) strand
GCUCAAACUGUUAAAUGUUGGAAAA 595
SEQ ID NO: 596 - DsiRNA 294 guide (antisense) strand
UUUUCCAACAUUUAACAGUUUGAGCAC
SEQ ID NO: 597 - DsiRNA 295 passenger (sense) strand
CUCAAACUGUUAAAUGUUGGAAAAG
SEQ ID NO: 598 - DsiRNA 295 guide (antisense) strand
CUUUUCCAACAUUUAACAGUUUGAGCA
SEQ ID NO: 599 - DsiRNA 296 passenger (sense) strand
UCAAACUGUUAAAUGUUGGAAAAGA
SEQ ID NO: 600 - DsiRNA 296 guide (antisense) strand
UCUUUUCCAACAUUUAACAGUUUGAGC
SEQ ID NO: 601 - DsiRNA 297 passenger (sense) strand
CAAACUGUUAAAUGUUGGAAAAGAA
SEQ ID NO: 602 - DsiRNA 297 guide (antisense) strand
UUCUUUUCCAACAUUUAACAGUUUGAG
SEQ ID NO: 603 - DsiRNA 298 passenger (sense) strand
AAACUGUUAAAUGUUGGAAAAGAAA
SEQ ID NO: 604 - DsiRNA 298 guide (antisense) strand
UUUCUUUUCCAACAUUUAACAGUUUGA
SEQ ID NO: 605 - DsiRNA 299 passenger (sense) strand
AACUGUUAAAUGUUGGAAAAGAAAG
SEQ ID NO: 606 - DsiRNA 299 guide (antisense) strand
CUUUCUUUUCCAACAUUUAACAGUUUG
SEQ ID NO: 607 - DsiRNA 300 passenger (sense) strand
ACUGUUAAAUGUUGGAAAAGAAAGA
SEQ ID NO: 608 - DsiRNA 300 guide (antisense) strand
UCUUUCUUUUCCAACAUUUAACAGUUU
SEQ ID NO: 609 - DsiRNA 301 passenger (sense) strand
CUGUUAAAUGUUGGAAAAGAAAGAT
SEQ ID NO: 610 - DsiRNA 301 guide (antisense) strand
AUCUUUCUUUUCCAACAUUUAACAGUU
SEQ ID NO: 611 - DsiRNA 302 passenger (sense) strand
UGUUAAAUGUUGGAAAAGAAAGATA
SEQ ID NO: 612 - DsiRNA 302 guide (antisense) strand
UAUCUUUCUUUUCCAACAUUUAACAGU
SEQ ID NO: 613 - DsiRNA 303 passenger (sense) strand
GUUAAAUGUUGGAAAAGAAAGAUAC
SEQ ID NO: 614 - DsiRNA 303 guide (antisense) strand
GUAUCUUUCUUUUCCAACAUUUAACAG
SEQ ID NO: 615 - DsiRNA 304 passenger (sense) strand
UUAAAUGUUGGAAAAGAAAGAUACA
SEQ ID NO: 616 - DsiRNA 304 guide (antisense) strand
UGUAUCUUUCUUUUCCAACAUUUAACA
SEQ ID NO: 617 - DsiRNA 305 passenger (sense) strand
UAAAUGUUGGAAAAGAAAGAUACAA
SEQ ID NO: 618 - DsiRNA 305 guide (antisense) strand
UUGUAUCUUUCUUUUCCAACAUUUAAC
SEQ ID NO: 619 - DsiRNA 306 passenger (sense) strand
GCACUUGACUGAGAAGACAGACCCT
SEQ ID NO: 620 - DsiRNA 306 guide (antisense) strand
AGGGUCUGUCUUCUCAGUCAAGUGCUU
SEQ ID NO: 621 - DsiRNA 307 passenger (sense) strand
GAGAAAAGAGGCUACUUGUGAAAAT
SEQ ID NO: 622 - DsiRNA 307 guide (antisense) strand
AUUUUCACAAGUAGCCUCUUUUCUCAA
SEQ ID NO: 623 - DsiRNA 308 passenger (sense) strand
AGAAAAGAGGCUACUUGUGAAAATA
SEQ ID NO: 624 - DsiRNA 308 guide (antisense) strand
UAUUUUCACAAGUAGCCUCUUUUCUCA
SEQ ID NO: 625 - DsiRNA 309 passenger (sense) strand
GAAAAGAGGCUACUUGUGAAAAUAA
SEQ ID NO: 626 - DsiRNA 309 guide (antisense) strand
UUAUUUUCACAAGUAGCCUCUUUUCUC
SEQ ID NO: 627 - DsiRNA 310 passenger (sense) strand
AAAAGAGGCUACUUGUGAAAAUAAT
SEQ ID NO: 628 - DsiRNA 310 guide (antisense) strand
AUUAUUUUCACAAGUAGCCUCUUUUCU
SEQ ID NO: 629 - DsiRNA 311 passenger (sense) strand
AAAGAGGCUACUUGUGAAAAUAATG
SEQ ID NO: 630 - DsiRNA 311 guide (antisense) strand
CAUUAUUUUCACAAGUAGCCUCUUUUC
SEQ ID NO: 631 - DsiRNA 312 passenger (sense) strand
AAGAGGCUACUUGUGAAAAUAAUGA
SEQ ID NO: 632 - DsiRNA 312 guide (antisense) strand
UCAUUAUUUUCACAAGUAGCCUCUUUU
SEQ ID NO: 633 - DsiRNA 313 passenger (sense) strand
AGAGGCUACUUGUGAAAAUAAUGAG
SEQ ID NO: 634 - DsiRNA 313 guide (antisense) strand
CUCAUUAUUUUCACAAGUAGCCUCUUU
SEQ ID NO: 635 - DsiRNA 314 passenger (sense) strand
GAGGCUACUUGUGAAAAUAAUGAGC
SEQ ID NO: 636 - DsiRNA 314 guide (antisense) strand
GCUCAUUAUUUUCACAAGUAGCCUCUU
SEQ ID NO: 637 - DsiRNA 315 passenger (sense) strand
GGCUACUUGUGAAAAUAAUGAGCCC
SEQ ID NO: 638 - DsiRNA 315 guide (antisense) strand
GGGCUCAUUAUUUUCACAAGUAGCCUC
SEQ ID NO: 639 - DsiRNA 316 passenger (sense) strand
CUACUUGUGAAAAUAAUGAGCCCCC
SEQ ID NO: 640 - DsiRNA 316 guide (antisense) strand
GGGGGCUCAUUAUUUUCACAAGUAGCC
SEQ ID NO: 641 - DsiRNA 317 passenger (sense) strand
UGAACCUGCCUUCUUACAUCUUGAG
SEQ ID NO: 642 - DsiRNA 317 guide (antisense) strand
CUCAAGAUGUAAGAAGGCAGGUUCAAA
SEQ ID NO: 643 - DsiRNA 318 passenger (sense) strand
AAAGUUACAAGUUUCUUUUCCCAAG
SEQ ID NO: 644 - DsiRNA 318 guide (antisense) strand
CUUGGGAAAAGAAACUUGUAACUUUCC
SEQ ID NO: 645 - DsiRNA 319 passenger (sense) strand
AAGUUACAAGUUUCUUUUCCCAAGT
SEQ ID NO: 646 - DsiRNA 319 guide (antisense) strand
ACUUGGGAAAAGAAACUUGUAACUUUC
SEQ ID NO: 647 - DsiRNA 320 passenger (sense) strand
AGUUACAAGUUUCUUUUCCCAAGTT
SEQ ID NO: 648 - DsiRNA 320 guide (antisense) strand
AACUUGGGAAAAGAAACUUGUAACUUU
SEQ ID NO: 649 - DsiRNA 321 passenger (sense) strand
AGUUUCUUUUCCCAAGUUUCCCAGT
SEQ ID NO: 650 - DsiRNA 321 guide (antisense) strand
ACUGGGAAACUUGGGAAAAGAAACUUG
SEQ ID NO: 651 - DsiRNA 322 passenger (sense) strand
CAACAGUAUUUUCUAAUAACCAGTA
SEQ ID NO: 652 - DsiRNA 322 guide (antisense) strand
UACUGGUUAUUAGAAAAUACUGUUGGC
SEQ ID NO: 653 - DsiRNA 323 passenger (sense) strand
AACAGUAUUUUCUAAUAACCAGUAT
SEQ ID NO: 654 - DsiRNA 323 guide (antisense) strand
AUACUGGUUAUUAGAAAAUACUGUUGG
SEQ ID NO: 655 - DsiRNA 324 passenger (sense) strand
ACAGUAUUUUCUAAUAACCAGUATA
SEQ ID NO: 656 - DsiRNA 324 guide (antisense) strand
UAUACUGGUUAUUAGAAAAUACUGUUG
SEQ ID NO: 657 - DsiRNA 325 passenger (sense) strand
CAGUAUUUUCUAAUAACCAGUAUAT
SEQ ID NO: 658 - DsiRNA 325 guide (antisense) strand
AUAUACUGGUUAUUAGAAAAUACUGUU
SEQ ID NO: 659 - DsiRNA 326 passenger (sense) strand
UUGUGAUUGUUAUCAGGAAAAAATA
SEQ ID NO: 660 - DsiRNA 326 guide (antisense) strand
UAUUUUUUCCUGAUAACAAUCACAAUA
SEQ ID NO: 661 - DsiRNA 327 passenger (sense) strand
UGUGAUUGUUAUCAGGAAAAAAUAT
SEQ ID NO: 662 - DsiRNA 327 guide (antisense) strand
AUAUUUUUUCCUGAUAACAAUCACAAU
SEQ ID NO: 663 - DsiRNA 328 passenger (sense) strand
GUGAUUGUUAUCAGGAAAAAAUATA
SEQ ID NO: 664 - DsiRNA 328 guide (antisense) strand
UAUAUUUUUUCCUGAUAACAAUCACAA
SEQ ID NO: 665 - DsiRNA 329 passenger (sense) strand
UGAUUGUUAUCAGGAAAAAAUAUAT
SEQ ID NO: 666 - DsiRNA 329 guide (antisense) strand
AUAUAUUUUUUCCUGAUAACAAUCACA
SEQ ID NO: 667 - DsiRNA 330 passenger (sense) strand
GAUUGUUAUCAGGAAAAAAUAUATT
SEQ ID NO: 668 - DsiRNA 330 guide (antisense) strand
AAUAUAUUUUUUCCUGAUAACAAUCAC
SEQ ID NO: 669 - DsiRNA 331 passenger (sense) strand
AUUGUUAUCAGGAAAAAAUAUAUTA
SEQ ID NO: 670 - DsiRNA 331 guide (antisense) strand
UAAUAUAUUUUUUCCUGAUAACAAUCA
SEQ ID NO: 671 - DsiRNA 332 passenger (sense) strand
UUGUUAUCAGGAAAAAAUAUAUUAA
SEQ ID NO: 672 - DsiRNA 332 guide (antisense) strand
UUAAUAUAUUUUUUCCUGAUAACAAUC
SEQ ID NO: 673 - DsiRNA 333 passenger (sense) strand
UGUUAUCAGGAAAAAAUAUAUUAAA
SEQ ID NO: 674 - DsiRNA 333 guide (antisense) strand
UUUAAUAUAUUUUUUCCUGAUAACAAU
SEQ ID NO: 675 - DsiRNA 334 passenger (sense) strand
GUUAUCAGGAAAAAAUAUAUUAAAT
SEQ ID NO: 676 - DsiRNA 334 guide (antisense) strand
AUUUAAUAUAUUUUUUCCUGAUAACAA
SEQ ID NO: 677 - DsiRNA 335 passenger (sense) strand
UUAUCAGGAAAAAAUAUAUUAAATG
SEQ ID NO: 678 - DsiRNA 335 guide (antisense) strand
CAUUUAAUAUAUUUUUUCCUGAUAACA
SEQ ID NO: 679 - DsiRNA 336 passenger (sense) strand
UAUCAGGAAAAAAUAUAUUAAAUGG
SEQ ID NO: 680 - DsiRNA 336 guide (antisense) strand
CCAUUUAAUAUAUUUUUUCCUGAUAAC
SEQ ID NO: 681 - DsiRNA 337 passenger (sense) strand
AUCAGGAAAAAAUAUAUUAAAUGGC
SEQ ID NO: 682 - DsiRNA 337 guide (antisense) strand
GCCAUUUAAUAUAUUUUUUCCUGAUAA
SEQ ID NO: 683 - DsiRNA 338 passenger (sense) strand
AGGAAAAAAUAUAUUAAAUGGCUGA
SEQ ID NO: 684 - DsiRNA 338 guide (antisense) strand
UCAGCCAUUUAAUAUAUUUUUUCCUGA
SEQ ID NO: 685 - DsiRNA 339 passenger (sense) strand
GGAAAAAAUAUAUUAAAUGGCUGAT
SEQ ID NO: 686 - DsiRNA 339 guide (antisense) strand
AUCAGCCAUUUAAUAUAUUUUUUCCUG
SEQ ID NO: 687 - DsiRNA 340 passenger (sense) strand
GAAAAAAUAUAUUAAAUGGCUGATA
SEQ ID NO: 688 - DsiRNA 340 guide (antisense) strand
UAUCAGCCAUUUAAUAUAUUUUUUCCU
SEQ ID NO: 689 - DsiRNA 341 passenger (sense) strand
AAAAAAUAUAUUAAAUGGCUGAUAG
SEQ ID NO: 690 - DsiRNA 341 guide (antisense) strand
CUAUCAGCCAUUUAAUAUAUUUUUUCC
SEQ ID NO: 691 - DsiRNA 342 passenger (sense) strand
UAUUUUCUUUCUGCUUUUAAAAATT
SEQ ID NO: 692 - DsiRNA 342 guide (antisense) strand
AAUUUUUAAAAGCAGAAAGAAAAUACG
SEQ ID NO: 693 - DsiRNA 343 passenger (sense) strand
AUUUUCUUUCUGCUUUUAAAAAUTA
SEQ ID NO: 694 - DsiRNA 343 guide (antisense) strand
UAAUUUUUAAAAGCAGAAAGAAAAUAC
SEQ ID NO: 695 - DsiRNA 344 passenger (sense) strand
UCUUUCUGCUUUUAAAAAUUAUUCA
SEQ ID NO: 696 - DsiRNA 344 guide (antisense) strand
UGAAUAAUUUUUAAAAGCAGAAAGAAA
SEQ ID NO: 697 - DsiRNA 345 passenger (sense) strand
CUUUCUGCUUUUAAAAAUUAUUCAG
SEQ ID NO: 698 - DsiRNA 345 guide (antisense) strand
CUGAAUAAUUUUUAAAAGCAGAAAGAA
SEQ ID NO: 699 - DsiRNA 346 passenger (sense) strand
UUUCUGCUUUUAAAAAUUAUUCAGG
SEQ ID NO: 700 - DsiRNA 346 guide (antisense) strand
CCUGAAUAAUUUUUAAAAGCAGAAAGA
SEQ ID NO: 701 - DsiRNA 347 passenger (sense) strand
CUACUAAAAACACAAAAAUUAGCCA
SEQ ID NO: 702 - DsiRNA 347 guide (antisense) strand
UGGCUAAUUUUUGUGUUUUUAGUAGAG
SEQ ID NO: 703 - DsiRNA 348 passenger (sense) strand
CAAGAUAAGGAAAUCAGGAAGUGTA
SEQ ID NO: 704 - DsiRNA 348 guide (antisense) strand
UACACUUCCUGAUUUCCUUAUCUUGAU
SEQ ID NO: 705 - DsiRNA 349 passenger (sense) strand
AAGAUAAGGAAAUCAGGAAGUGUAA
SEQ ID NO: 706 - DsiRNA 349 guide (antisense) strand
UUACACUUCCUGAUUUCCUUAUCUUGA
SEQ ID NO: 707 - DsiRNA 350 passenger (sense) strand
AGAUAAGGAAAUCAGGAAGUGUAAT
SEQ ID NO: 708 - DsiRNA 350 guide (antisense) strand
AUUACACUUCCUGAUUUCCUUAUCUUG
SEQ ID NO: 709 - DsiRNA 351 passenger (sense) strand
GAUAAGGAAAUCAGGAAGUGUAATA
SEQ ID NO: 710 - DsiRNA 351 guide (antisense) strand
UAUUACACUUCCUGAUUUCCUUAUCUU
SEQ ID NO: 711 - DsiRNA 352 passenger (sense) strand
AUAAGGAAAUCAGGAAGUGUAAUAT
SEQ ID NO: 712 - DsiRNA 352 guide (antisense) strand
AUAUUACACUUCCUGAUUUCCUUAUCU
SEQ ID NO: 713 - DsiRNA 353 passenger (sense) strand
UAAGGAAAUCAGGAAGUGUAAUATT
SEQ ID NO: 714 - DsiRNA 353 guide (antisense) strand
AAUAUUACACUUCCUGAUUUCCUUAUC
SEQ ID NO: 715 - DsiRNA 354 passenger (sense) strand
AAGGAAAUCAGGAAGUGUAAUAUTC
SEQ ID NO: 716 - DsiRNA 354 guide (antisense) strand
GAAUAUUACACUUCCUGAUUUCCUUAU
SEQ ID NO: 717 - DsiRNA 355 passenger (sense) strand
AGGAAAUCAGGAAGUGUAAUAUUCT
SEQ ID NO: 718 - DsiRNA 355 guide (antisense) strand
AGAAUAUUACACUUCCUGAUUUCCUUA
SEQ ID NO: 719 - DsiRNA 356 passenger (sense) strand
GGAAAUCAGGAAGUGUAAUAUUCTT
SEQ ID NO: 720 - DsiRNA 356 guide (antisense) strand
AAGAAUAUUACACUUCCUGAUUUCCUU
SEQ ID NO: 721 - DsiRNA 357 passenger (sense) strand
GAAAUCAGGAAGUGUAAUAUUCUTA
SEQ ID NO: 722 - DsiRNA 357 guide (antisense) strand
UAAGAAUAUUACACUUCCUGAUUUCCU
SEQ ID NO: 723 - DsiRNA 358 passenger (sense) strand
CUAUGAAUGCAUUCUUAUUUCUUCT
SEQ ID NO: 724 - DsiRNA 358 guide (antisense) strand
AGAAGAAAUAAGAAUGCAUUCAUAGGC
SEQ ID NO: 725 - DsiRNA 359 passenger (sense) strand
UAUGAAUGCAUUCUUAUUUCUUCTT
SEQ ID NO: 726 - DsiRNA 359 guide (antisense) strand
AAGAAGAAAUAAGAAUGCAUUCAUAGG
SEQ ID NO: 727 - DsiRNA 360 passenger (sense) strand
CUACCACACCCAGCUAGUUUUUUTT
SEQ ID NO: 728 - DsiRNA 360 guide (antisense) strand
AAAAAAAACUAGCUGGGUGUGGUAGUG
SEQ ID NO: 729 - DsiRNA 361 passenger (sense) strand
CCACACCCAGCUAGUUUUUUUUUGT
SEQ ID NO: 730 - DsiRNA 361 guide (antisense) strand
ACAAAAAAAAACUAGCUGGGUGUGGUA
SEQ ID NO: 731 - DsiRNA 362 passenger (sense) strand
CACACCCAGCUAGUUUUUUUUUGTA
SEQ ID NO: 732 - DsiRNA 362 guide (antisense) strand
UACAAAAAAAAACUAGCUGGGUGUGGU
SEQ ID NO: 733 - DsiRNA 363 passenger (sense) strand
GCUAGGAUUACAGGUGUGAGCUACC
SEQ ID NO: 734 - DsiRNA 363 guide (antisense) strand
GGUAGCUCACACCUGUAAUCCUAGCAC
SEQ ID NO: 735 - DsiRNA 364 passenger (sense) strand
CUAGGAUUACAGGUGUGAGCUACCA
SEQ ID NO: 736 - DsiRNA 364 guide (antisense) strand
UGGUAGCUCACACCUGUAAUCCUAGCA
SEQ ID NO: 737 - DsiRNA 365 passenger (sense) strand
CCAUGCCUGGUCCAACAUUCUUCAT
SEQ ID NO: 738 - DsiRNA 365 guide (antisense) strand
AUGAAGAAUGUUGGACCAGGCAUGGUA
SEQ ID NO: 739 - DsiRNA 366 passenger (sense) strand
UGCAGAGUAUGAGCCUGAUUUUGTT
SEQ ID NO: 740 - DsiRNA 366 guide (antisense) strand
AACAAAAUCAGGCUCAUACUCUGCACU
SEQ ID NO: 741 - DsiRNA 367 passenger (sense) strand
GCAGAGUAUGAGCCUGAUUUUGUTT
SEQ ID NO: 742 - DsiRNA 367 guide (antisense) strand
AAACAAAAUCAGGCUCAUACUCUGCAC
SEQ ID NO: 743 - DsiRNA 368 passenger (sense) strand
CAGAGUAUGAGCCUGAUUUUGUUTA
SEQ ID NO: 744 - DsiRNA 368 guide (antisense) strand
UAAACAAAAUCAGGCUCAUACUCUGCA
SEQ ID NO: 745 - DsiRNA 369 passenger (sense) strand
AGAGUAUGAGCCUGAUUUUGUUUAA
SEQ ID NO: 746 - DsiRNA 369 guide (antisense) strand
UUAAACAAAAUCAGGCUCAUACUCUGC
SEQ ID NO: 747 - DsiRNA 370 passenger (sense) strand
GAGUAUGAGCCUGAUUUUGUUUAAA
SEQ ID NO: 748 - DsiRNA 370 guide (antisense) strand
UUUAAACAAAAUCAGGCUCAUACUCUG
SEQ ID NO: 749 - DsiRNA 371 passenger (sense)
GGGUGAAACCCCAUCUCUACUAAAA
SEQ ID NO: 750 - DsiRNA 371 guide (antisense) strand
UUUUAGUAGAGAUGGGGUUUCACCCAG
SEQ ID NO: 751 - DsiRNA 372 passenger (sense) strand
GUGAAACCCCAUCUCUACUAAAAAA
SEQ ID NO: 752 - DsiRNA 372 guide (antisense) strand
UUUUUUAGUAGAGAUGGGGUUUCACCC
SEQ ID NO: 753 - DsiRNA 373 passenger (sense) strand
UGAAACCCCAUCUCUACUAAAAAAT
SEQ ID NO: 754 - DsiRNA 373 guide (antisense) strand
AUUUUUUAGUAGAGAUGGGGUUUCACC
SEQ ID NO: 755 - DsiRNA 374 passenger (sense) strand
GAAACCCCAUCUCUACUAAAAAATG
SEQ ID NO: 756 - DsiRNA 374 guide (antisense) strand
CAUUUUUUAGUAGAGAUGGGGUUUCAC
SEQ ID NO: 757 - DsiRNA 375 guide (antisense) strand
AAACCCCAUCUCUACUAAAAAAUGC
SEQ ID NO: 758 - DsiRNA 375 guide (antisense) strand
GCAUUUUUUAGUAGAGAUGGGGUUUCA
SEQ ID NO: 759 - DsiRNA 376 passenger (sense) strand
AACCCCAUCUCUACUAAAAAAUGCA
SEQ ID NO: 760 - DsiRNA 376 guide (antisense) strand
UGCAUUUUUUAGUAGAGAUGGGGUUUC
SEQ ID NO: 761 - DsiRNA 377 passenger (sense) strand
CCCAUCUCUACUAAAAAAUGCAAAA
SEQ ID NO: 762 - DsiRNA 377 guide (antisense) strand
UUUUGCAUUUUUUAGUAGAGAUGGGGU
SEQ ID NO: 763 - DsiRNA 378 passenger (sense) strand
AUCAAAACCCUUAUGGCAGACUGTT
SEQ ID NO: 764 - DsiRNA 378 guide (antisense) strand
AACAGUCUGCCAUAAGGGUUUUGAUAU
SEQ ID NO: 765 - DsiRNA 379 passenger (sense) strand
UAUUUUAUUUGUCGUGCUUAUAUGT
SEQ ID NO: 766 - DsiRNA 379 guide (antisense) strand
ACAUAUAAGCACGACAAAUAAAAUACA
SEQ ID NO: 767 - DsiRNA 380 passenger (sense) strand
GUGUUGCCCAAGUUUCUAUGGUGAA
SEQ ID NO: 768 - DsiRNA 380 guide (antisense) strand
UUCACCAUAGAAACUUGGGCAACACAU
SEQ ID NO: 769 - DsiRNA 381 passenger (sense) strand
GCCCAAGUUUCUAUGGUGAACGGTA
SEQ ID NO: 770 - DsiRNA 381 guide (antisense) strand
UACCGUUCACCAUAGAAACUUGGGCAA
SEQ ID NO: 771 - DsiRNA 382 passenger (sense) strand
CCCAAGUUUCUAUGGUGAACGGUAT
SEQ ID NO: 772 - DsiRNA 382 guide (antisense) strand
AUACCGUUCACCAUAGAAACUUGGGCA
SEQ ID NO: 773 - DsiRNA 383 passenger (sense) strand
ACUUUCAGCAUGAGAAAAUAACUCC
SEQ ID NO: 774 - DsiRNA 383 guide (antisense) strand
GGAGUUAUUUUCUCAUGCUGAAAGUGA
SEQ ID NO: 775 - DsiRNA 384 passenger (sense) strand
CUUUCAGCAUGAGAAAAUAACUCCT
SEQ ID NO: 776 - DsiRNA 384 guide (antisense) strand
AGGAGUUAUUUUCUCAUGCUGAAAGUG
SEQ ID NO: 777 -PNPLA3 oligonucleotide 1 passenger (sense) strand
UGAGUGACAACGUACCCUUAGCAGCCGAAAGGCUGC
SEQ ID NO: 778 - PNPLA3 oligonucleotide 1 guide (antisense) strand
UAAGGGUACGUUGUCACUCAGG
SEQ ID NO: 779 - PNPLA3 oligonucleotide 2 passenger (sense) strand
GAGUGACAACGUACCCUUCAGCAGCCGAAAGGCUGC
SEQ ID NO: 780 - PNPLA3 oligonucleotide 2 guide (antisense) strand
UGAAGGGUACGUUGUCACUCGG
SEQ ID NO: 781 - PNPLA3 oligonucleotide 3 passenger (sense) strand
AGUGACAACGUACCCUUCAAGCAGCCGAAAGGCUGC
SEQ ID NO: 782 - PNPLA3 oligonucleotide 3 guide (antisense) strand
UUGAAGGGUACGUUGUCACUGG
SEQ ID NO: 783 - PNPLA3 oligonucleotide 4 passenger (sense) strand
UGACAACGUACCCUUCAUUAGCAGCCGAAAGGCUGC
SEQ ID NO: 784 - PNPLA3 oligonucleotide 4 guide (antisense) strand
UAAUGAAGGGUACGUUGUCAGG
SEQ ID NO: 785 - PNPLA3 oligonucleotide 5 passenger (sense) strand
GACAACGUACCCUUCAUUGAGCAGCCGAAAGGCUGC
SEQ ID NO: 786 - PNPLA3 oligonucleotide 5 guide (antisense) strand
UCAAUGAAGGGUACGUUGUCGG
SEQ ID NO: 787 - PNPLA3 oligonucleotide 6 passenger (sense) strand
CAACGUACCCUUCAUUGAUAGCAGCCGAAAGGCUGC
SEQ ID NO: 788 - PNPLA3 oligonucleotide 6 guide (antisense) strand
UAUCAAUGAAGGGUACGUUGGG
SEQ ID NO: 789 - PNPLA3 oligonucleotide 7 passenger (sense) strand
AACGUACCCUUCAUUGAUGAGCAGCCGAAAGGCUGC
SEQ ID NO: 790 - PNPLA3 oligonucleotide 7 guide (antisense) strand
UCAUCAAUGAAGGGUACGUUGG
SEQ ID NO: 791 - PNPLA3 oligonucleotide 8 passenger (sense) strand
ACGUACCCUUCAUUGAUGCAGCAGCCGAAAGGCUGC
SEQ ID NO: 792 - PNPLA3 oligonucleotide 8 guide (antisense) strand
UGCAUCAAUGAAGGGUACGUGG
SEQ ID NO: 793 - PNPLA3 oligonucleotide 9 passenger (sense) strand
CGUACCCUUCAUUGAUGCCAGCAGCCGAAAGGCUGC
SEQ ID NO: 794 - PNPLA3 oligonucleotide 9 guide (antisense) strand
UGGCAUCAAUGAAGGGUACGGG
SEQ ID NO: 795 - PNPLA3 oligonucleotide 10 passenger (sense) strand
UACCCUUCAUUGAUGCCAAAGCAGCCGAAAGGCUGC
SEQ ID NO: 796 - PNPLA3 oligonucleotide 10 guide (antisense) strand
UUUGGCAUCAAUGAAGGGUAGG
SEQ ID NO: 797 - PNPLA3 oligonucleotide 11 passenger (sense) strand
CACGAACUUUCUUCAUGUGAGCAGCCGAAAGGCUGC
SEQ ID NO: 798 - PNPLA3 oligonucleotide 11 guide (antisense) strand
UCACAUGAAGAAAGUUCGUGGG
SEQ ID NO: 799 - PNPLA3 oligonucleotide 12 passenger (sense) strand
ACGAACUUUCUUCAUGUGGAGCAGCCGAAAGGCUGC
SEQ ID NO: 800 - PNPLA3 oligonucleotide 12 guide (antisense) strand
UCCACAUGAAGAAAGUUCGUGG
SEQ ID NO: 801 - PNPLA3 oligonucleotide 13 passenger (sense) strand
CGAACUUUCUUCAUGUGGAAGCAGCCGAAAGGCUGC
SEQ ID NO: 802 - PNPLA3 oligonucleotide 13 guide (antisense) strand
UUCCACAUGAAGAAAGUUCGGG
SEQ ID NO: 803 - PNPLA3 oligonucleotide 14 passenger (sense) strand
ACUUUCUUCAUGUGGACAUAGCAGCCGAAAGGCUGC
SEQ ID NO: 804 - PNPLA3 oligonucleotide 14 guide (antisense) strand
UAUGUCCACAUGAAGAAAGUGG
SEQ ID NO: 805 - PNPLA3 oligonucleotide 15 passenger (sense) strand
UUUCUUCAUGUGGACAUCAAGCAGCCGAAAGGCUGC
SEQ ID NO: 806 - PNPLA3 oligonucleotide 15 guide (antisense) strand
UUGAUGUCCACAUGAAGAAAGG
SEQ ID NO: 807 - PNPLA3 oligonucleotide 16 passenger (sense) strand
CUUCAUGUGGACAUCACCAAGCAGCCGAAAGGCUGC
SEQ ID NO: 808 - PNPLA3 oligonucleotide 16 guide (antisense) strand
UUGGUGAUGUCCACAUGAAGGG
SEQ ID NO: 809 - PNPLA3 oligonucleotide 17 passenger (sense) strand
AUGUGGACAUCACCAAGCUAGCAGCCGAAAGGCUGC
SEQ ID NO: 810 - PNPLA3 oligonucleotide 17 guide (antisense) strand
UAGCUUGGUGAUGUCCACAUGG
SEQ ID NO: 811 -PNPLA3 oligonucleotide 18 passenger (sense) strand
UGUGGACAUCACCAAGCUCAGCAGCCGAAAGGCUGC
SEQ ID NO: 812 - PNPLA3 oligonucleotide 18 guide (antisense) strand
UGAGCUUGGUGAUGUCCACAGG
SEQ ID NO: 813 - PNPLA3 oligonucleotide 19 passenger (sense) strand
GUGGACAUCACCAAGCUCAAGCAGCCGAAAGGCUGC
SEQ ID NO: 814 - PNPLA3 oligonucleotide 19 guide (antisense) strand
UUGAGCUUGGUGAUGUCCACGG
SEQ ID NO: 815 - PNPLA3 oligonucleotide 20 passenger (sense) strand
UGGACAUCACCAAGCUCAGAGCAGCCGAAAGGCUGC
SEQ ID NO: 816 - PNPLA3 oligonucleotide 20 guide (antisense) strand
UCUGAGCUUGGUGAUGUCCAGG
SEQ ID NO: 817 - PNPLA3 oligonucleotide 21 passenger (sense) strand
AGAUAUGCCUUCGAGGAUAAGCAGCCGAAAGGCUGC
SEQ ID NO: 818 - PNPLA3 oligonucleotide 21 guide (antisense) strand
UUAUCCUCGAAGGCAUAUCUGG
SEQ ID NO: 819 - PNPLA3 oligonucleotide 22 passenger (sense) strand
AUGCCUUCGAGGAUAUUUGAGCAGCCGAAAGGCUGC
SEQ ID NO: 820 - PNPLA3 oligonucleotide 22 guide (antisense) strand
UCAAAUAUCCUCGAAGGCAUGG
SEQ ID NO: 821 - PNPLA3 oligonucleotide 23 passenger (sense) strand
GCCUUCGAGGAUAUUUGGAAGCAGCCGAAAGGCUGC
SEQ ID NO: 822 - PNPLA3 oligonucleotide 23 guide (antisense) strand
UUCCAAAUAUCCUCGAAGGCGG
SEQ ID NO: 823 - PNPLA3 oligonucleotide 24 passenger (sense) strand
UGAAGUCAUCCUCAGAAGGAGCAGCCGAAAGGCUGC
SEQ ID NO: 824 - PNPLA3 oligonucleotide 24 guide (antisense) strand
UCCUUCUGAGGAUGACUUCAGG
SEQ ID NO: 825 - PNPLA3 oligonucleotide 25 passenger (sense) strand
GAAGUCAUCCUCAGAAGGGAGCAGCCGAAAGGCUGC
SEQ ID NO: 826 - PNPLA3 oligonucleotide 25 guide (antisense) strand
UCCCUUCUGAGGAUGACUUCGG
SEQ ID NO: 827 - PNPLA3 oligonucleotide 26 passenger (sense) strand
AUCCUCAGAAGGGAUGGAUAGCAGCCGAAAGGCUGC
SEQ ID NO: 828 - PNPLA3 oligonucleotide 26 guide (antisense) strand
UAUCCAUCCCUUCUGAGGAUGG
SEQ ID NO: 829 - PNPLA3 oligonucleotide 27 passenger (sense) strand
AGUGAAGAAAUGAAAGACAAGCAGCCGAAAGGCUGC
SEQ ID NO: 830 - PNPLA3 oligonucleotide 27 guide (antisense) strand
UUGUCUUUCAUUUCUUCACUGG
SEQ ID NO: 831 - PNPLA3 oligonucleotide 28 passenger (sense) strand
AAGAAAUGAAAGACAAAGGAGCAGCCGAAAGGCUGC
SEQ ID NO: 832 - PNPLA3 oligonucleotide 28 guide (antisense) strand
UCCUUUGUCUUUCAUUUCUUGG
SEQ ID NO: 833 - PNPLA3 oligonucleotide 29 passenger (sense) strand
AGAAAUGAAAGACAAAGGUAGCAGCCGAAAGGCUGC
SEQ ID NO: 834 - PNPLA3 oligonucleotide 29 guide (antisense) strand
UACCUUUGUCUUUCAUUUCUGG
SEQ ID NO: 835 - PNPLA3 oligonucleotide 30 passenger (sense) strand
GAAAUGAAAGACAAAGGUGAGCAGCCGAAAGGCUGC
SEQ ID NO: 836 - PNPLA3 oligonucleotide 30 guide (antisense) strand
UCACCUUUGUCUUUCAUUUCGG
SEQ ID NO: 837 - PNPLA3 oligonucleotide 31 passenger (sense) strand
AAAUGAAAGACAAAGGUGGAGCAGCCGAAAGGCUGC
SEQ ID NO: 838 - PNPLA3 oligonucleotide 31 guide (antisense) strand
UCCACCUUUGUCUUUCAUUUGG
SEQ ID NO: 839 - PNPLA3 oligonucleotide 32 passenger (sense) strand
AAUGAAAGACAAAGGUGGAAGCAGCCGAAAGGCUGC
SEQ ID NO: 840 - PNPLA3 oligonucleotide 32 guide (antisense) strand
UUCCACCUUUGUCUUUCAUUGG
SEQ ID NO: 841 - PNPLA3 oligonucleotide 33 passenger (sense) strand
AUGAAAGACAAAGGUGGAUAGCAGCCGAAAGGCUGC
SEQ ID NO: 842 - PNPLA3 oligonucleotide 33 guide (antisense) strand
UAUCCACCUUUGUCUUUCAUGG
SEQ ID NO: 843 - PNPLA3 oligonucleotide 34 passenger (sense) strand
UGAAAGACAAAGGUGGAUAAGCAGCCGAAAGGCUGC
SEQ ID NO: 844 - PNPLA3 oligonucleotide 34 guide (antisense) strand
UUAUCCACCUUUGUCUUUCAGG
SEQ ID NO: 845 - PNPLA3 oligonucleotide 35 passenger (sense) strand
GAAAGACAAAGGUGGAUACAGCAGCCGAAAGGCUGC
SEQ ID NO: 846 - PNPLA3 oligonucleotide 35 guide (antisense) strand
UGUAUCCACCUUUGUCUUUCGG
SEQ ID NO: 847 - PNPLA3 oligonucleotide 36 passenger (sense) strand
AAGACAAAGGUGGAUACAUAGCAGCCGAAAGGCUGC
SEQ ID NO: 848 - PNPLA3 oligonucleotide 36 guide (antisense) strand
UAUGUAUCCACCUUUGUCUUGG
SEQ ID NO: 849 - PNPLA3 oligonucleotide 37 passenger (sense) strand
AGACAAAGGUGGAUACAUGAGCAGCCGAAAGGCUGC
SEQ ID NO: 850 - PNPLA3 oligonucleotide 37 guide (antisense) strand
UCAUGUAUCCACCUUUGUCUGG
SEQ ID NO: 851 - PNPLA3 oligonucleotide 38 passenger (sense) strand
GACAAAGGUGGAUACAUGAAGCAGCCGAAAGGCUGC
SEQ ID NO: 852 - PNPLA3 oligonucleotide 38 guide (antisense) strand
UUCAUGUAUCCACCUUUGUCGG
SEQ ID NO: 853 - PNPLA3 oligonucleotide 39 passenger guide (sense) strand
ACAAAGGUGGAUACAUGAGAGCAGCCGAAAGGCUGC
SEQ ID NO: 854 - PNPLA3 oligonucleotide 39 guide (antisense) strand
UCUCAUGUAUCCACCUUUGUGG
SEQ ID NO: 855 - PNPLA3 oligonucleotide 40 passenger (sense) strand
AAGGUGGAUACAUGAGCAAAGCAGCCGAAAGGCUGC
SEQ ID NO: 856 - PNPLA3 oligonucleotide 40 guide (antisense) strand
UUUGCUCAUGUAUCCACCUUGG
SEQ ID NO: 857 - PNPLA3 oligonucleotide 41 passenger (sense) strand
AGGUGGAUACAUGAGCAAGAGCAGCCGAAAGGCUGC
SEQ ID NO: 858 - PNPLA3 oligonucleotide 41 guide (antisense) strand
UCUUGCUCAUGUAUCCACCUGG
SEQ ID NO: 859 - PNPLA3 oligonucleotide 42 passenger (sense) strand
UGGAUACAUGAGCAAGAUUAGCAGCCGAAAGGCUGC
SEQ ID NO: 860 - PNPLA3 oligonucleotide 42 guide (antisense) strand
UAAUCUUGCUCAUGUAUCCAGG
SEQ ID NO: 861 - PNPLA3 oligonucleotide 43 passenger (sense) strand
GGAUACAUGAGCAAGAUUUAGCAGCCGAAAGGCUGC
SEQ ID NO: 862 - PNPLA3 oligonucleotide 43 guide (antisense) strand
UAAAUCUUGCUCAUGUAUCCGG
SEQ ID NO: 863 - PNPLA3 oligonucleotide 44 passenger (sense) strand
GAUACAUGAGCAAGAUUUGAGCAGCCGAAAGGCUGC
SEQ ID NO: 864 - PNPLA3 oligonucleotide 44 guide (antisense) strand
UCAAAUCUUGCUCAUGUAUCGG
SEQ ID NO: 865 - PNPLA3 oligonucleotide 45 passenger (sense) strand
AUACAUGAGCAAGAUUUGCAGCAGCCGAAAGGCUGC
SEQ ID NO: 866 - PNPLA3 oligonucleotide 45 guide (antisense) strand
UGCAAAUCUUGCUCAUGUAUGG
SEQ ID NO: 867 - PNPLA3 oligonucleotide 46 passenger (sense) strand
UACAUGAGCAAGAUUUGCAAGCAGCCGAAAGGCUGC
SEQ ID NO: 868 - PNPLA3 oligonucleotide 46 guide (antisense) strand
UUGCAAAUCUUGCUCAUGUAGG
SEQ ID NO: 869 - PNPLA3 oligonucleotide 47 passenger (sense) strand
ACAUGAGCAAGAUUUGCAAAGCAGCCGAAAGGCUGC
SEQ ID NO: 870 - PNPLA3 oligonucleotide 47 guide (antisense) strand
UUUGCAAAUCUUGCUCAUGUGG
SEQ ID NO: 871 - PNPLA3 oligonucleotide 48 passenger (sense) strand
UGAGCAAGAUUUGCAACUUAGCAGCCGAAAGGCUGC
SEQ ID NO: 872 - PNPLA3 oligonucleotide 48 guide (antisense) strand
UAAGUUGCAAAUCUUGCUCAGG
SEQ ID NO: 873 - PNPLA3 oligonucleotide 49 passenger (sense) strand
GAGCAAGAUUUGCAACUUGAGCAGCCGAAAGGCUGC
SEQ ID NO: 874 - PNPLA3 oligonucleotide 49 guide (antisense) strand
UCAAGUUGCAAAUCUUGCUCGG
SEQ ID NO: 875 - PNPLA3 oligonucleotide 50 passenger (sense) strand
AGCAAGAUUUGCAACUUGCAGCAGCCGAAAGGCUGC
SEQ ID NO: 876 - PNPLA3 oligonucleotide 50 guide (antisense) strand
UGCAAGUUGCAAAUCUUGCUGG
SEQ ID NO: 877 - PNPLA3 oligonucleotide 51 passenger (sense) strand
GCAAGAUUUGCAACUUGCUAGCAGCCGAAAGGCUGC
SEQ ID NO: 878 - PNPLA3 oligonucleotide 51 guide (antisense) strand
UAGCAAGUUGCAAAUCUUGCGG
SEQ ID NO: 879 - PNPLA3 oligonucleotide 52 passenger (sense) strand
CAAGAUUUGCAACUUGCUAAGCAGCCGAAAGGCUGC
SEQ ID NO: 880 - PNPLA3 oligonucleotide 52 guide (antisense) strand
UUAGCAAGUUGCAAAUCUUGGG
SEQ ID NO: 881 - PNPLA3 oligonucleotide 53 passenger (sense) strand
AAGAUUUGCAACUUGCUACAGCAGCCGAAAGGCUGC
SEQ ID NO: 882 - PNPLA3 oligonucleotide 53 guide (antisense) strand
UGUAGCAAGUUGCAAAUCUUGG
SEQ ID NO: 883 - PNPLA3 oligonucleotide 54 passenger (sense) strand
AGAUUUGCAACUUGCUACCAGCAGCCGAAAGGCUGC
SEQ ID NO: 884 - PNPLA3 oligonucleotide 54 guide (antisense) strand
UGGUAGCAAGUUGCAAAUCUGG
SEQ ID NO: 885 - PNPLA3 oligonucleotide 55 passenger (sense) strand
AUUUGCAACUUGCUACCCAAGCAGCCGAAAGGCUGC
SEQ ID NO: 886 - PNPLA3 oligonucleotide 55 guide (antisense) strand
UUGGGUAGCAAGUUGCAAAUGG
SEQ ID NO: 887 - PNPLA3 oligonucleotide 56 passenger (sense) strand
UUGCAACUUGCUACCCAUUAGCAGCCGAAAGGCUGC
SEQ ID NO: 888 - PNPLA3 oligonucleotide 56 guide (antisense) strand
UAAUGGGUAGCAAGUUGCAAGG
SEQ ID NO: 889 - PNPLA3 oligonucleotide 57 passenger (sense) strand
UGCAACUUGCUACCCAUUAAGCAGCCGAAAGGCUGC
SEQ ID NO: 890 - PNPLA3 oligonucleotide 57 guide (antisense) strand
UUAAUGGGUAGCAAGUUGCAGG
SEQ ID NO: 891 - PNPLA3 oligonucleotide 58 passenger (sense) strand
GCAACUUGCUACCCAUUAGAGCAGCCGAAAGGCUGC
SEQ ID NO: 892 - PNPLA3 oligonucleotide 58 guide (antisense) strand
UCUAAUGGGUAGCAAGUUGCGG
SEQ ID NO: 893 - PNPLA3 oligonucleotide 59 passenger (sense) strand
CAACUUGCUACCCAUUAGGAGCAGCCGAAAGGCUGC
SEQ ID NO: 894 - PNPLA3 oligonucleotide 59 guide (antisense) strand
UCCUAAUGGGUAGCAAGUUGGG
SEQ ID NO: 895 - PNPLA3 oligonucleotide 60 passenger (sense) strand
CUUGCUACCCAUUAGGAUAAGCAGCCGAAAGGCUGC
SEQ ID NO: 896 - PNPLA3 oligonucleotide 60 guide (antisense) strand
UUAUCCUAAUGGGUAGCAAGGG
SEQ ID NO: 897 - PNPLA3 oligonucleotide 61 passenger (sense) strand
UGCUACCCAUUAGGAUAAUAGCAGCCGAAAGGCUGC
SEQ ID NO: 898 - PNPLA3 oligonucleotide 61 guide (antisense) strand
UAUUAUCCUAAUGGGUAGCAGG
SEQ ID NO: 899 - PNPLA3 oligonucleotide 62 passenger (sense) strand
AUUAGGAUAAUGUCUUAUGAGCAGCCGAAAGGCUGC
SEQ ID NO: 900 - PNPLA3 oligonucleotide 62 guide (antisense) strand
UCAUAAGACAUUAUCCUAAUGG
SEQ ID NO: 901 - PNPLA3 oligonucleotide 63 passenger (sense) strand
UUAGGAUAAUGUCUUAUGUAGCAGCCGAAAGGCUGC
SEQ ID NO: 902 - PNPLA3 oligonucleotide 63 guide (antisense) strand
UACAUAAGACAUUAUCCUAAGG
SEQ ID NO: 903 - PNPLA3 oligonucleotide 64 passenger (sense) strand
UGCGAUUGUCCAGAGACUGAGCAGCCGAAAGGCUGC
SEQ ID NO: 904 - PNPLA3 oligonucleotide 64 guide (antisense) strand
UCAGUCUCUGGACAAUCGCAGG
SEQ ID NO: 905 - PNPLA3 oligonucleotide 65 passenger (sense) strand
CGAUUGUCCAGAGACUGGUAGCAGCCGAAAGGCUGC
SEQ ID NO: 906 - PNPLA3 oligonucleotide 65 guide (antisense) strand
UACCAGUCUCUGGACAAUCGGG
SEQ ID NO: 907 - PNPLA3 oligonucleotide 66 passenger (sense) strand
GUCCAGAGACUGGUGACAUAGCAGCCGAAAGGCUGC
SEQ ID NO: 908 - PNPLA3 oligonucleotide 66 guide (antisense) strand
UAUGUCACCAGUCUCUGGACGG
SEQ ID NO: 909 - PNPLA3 oligonucleotide 67 passenger (sense) strand
AGAGACUGGUGACAUGGCUAGCAGCCGAAAGGCUGC
SEQ ID NO: 910 - PNPLA3 oligonucleotide 67 guide (antisense) strand
UAGCCAUGUCACCAGUCUCUGG
SEQ ID NO: 911 - PNPLA3 oligonucleotide 68 passenger (sense) strand
ACUGGUGACAUGGCUUCCAAGCAGCCGAAAGGCUGC
SEQ ID NO: 912 - PNPLA3 oligonucleotide 68 guide (antisense) strand
UUGGAAGCCAUGUCACCAGUGG
SEQ ID NO: 913 - PNPLA3 oligonucleotide 69 passenger (sense) strand
CUCCACCUUUCCCAGUUUUAGCAGCCGAAAGGCUGC
SEQ ID NO: 914 - PNPLA3 oligonucleotide 69 guide (antisense) strand
UAAAACUGGGAAAGGUGGAGGG
SEQ ID NO: 915 - PNPLA3 oligonucleotide 70 passenger (sense) strand
CACCUUUCCCAGUUUUUCAAGCAGCCGAAAGGCUGC
SEQ ID NO: 916 - PNPLA3 oligonucleotide 70 guide (antisense) strand
UUGAAAAACUGGGAAAGGUGGG
SEQ ID NO: 917 - PNPLA3 oligonucleotide 71 passenger (sense) strand
AGUUUUUCACUAGAGAAGAAGCAGCCGAAAGGCUGC
SEQ ID NO: 918 - PNPLA3 oligonucleotide 71 guide (antisense) strand
UUCUUCUCUAGUGAAAAACUGG
SEQ ID NO: 919 - PNPLA3 oligonucleotide 72 passenger (sense) strand
AGCAGAUUCUUUCAGAGGUAGCAGCCGAAAGGCUGC
SEQ ID NO: 920 - PNPLA3 oligonucleotide 72 guide (antisense) strand
UACCUCUGAAAGAAUCUGCUGG
SEQ ID NO: 921 - PNPLA3 oligonucleotide 73 passenger (sense) strand
GCAGAUUCUUUCAGAGGUGAGCAGCCGAAAGGCUGC
SEQ ID NO: 922 - PNPLA3 oligonucleotide 73 guide (antisense) strand
UCACCUCUGAAAGAAUCUGCGG
SEQ ID NO: 923 - PNPLA3 oligonucleotide 74 passenger (sense) strand
AGAUUCUUUCAGAGGUGCUAGCAGCCGAAAGGCUGC
SEQ ID NO: 924 - PNPLA3 oligonucleotide 74 guide (antisense) strand
UAGCACCUCUGAAAGAAUCUGG
SEQ ID NO: 925 - PNPLA3 oligonucleotide 75 passenger (sense) strand
AGGUGCUAAAGUUUCCCAUAGCAGCCGAAAGGCUGC
SEQ ID NO: 926 - PNPLA3 oligonucleotide 75 guide (antisense) strand
UAUGGGAAACUUUAGCACCUGG
SEQ ID NO: 927 - PNPLA3 oligonucleotide 76 passenger (sense) strand
AAAGUUUCCCAUCUUUGUGAGCAGCCGAAAGGCUGC
SEQ ID NO: 928 - PNPLA3 oligonucleotide 76 guide (antisense) strand
UCACAAAGAUGGGAAACUUUGG
SEQ ID NO: 929 - PNPLA3 oligonucleotide 77 passenger (sense) strand
AGUUUCCCAUCUUUGUGCAAGCAGCCGAAAGGCUGC
SEQ ID NO: 930 - PNPLA3 oligonucleotide 77 guide (antisense) strand
UUGCACAAAGAUGGGAAACUGG
SEQ ID NO: 931 - PNPLA3 oligonucleotide 78 passenger (sense) strand
AGCCUCUGAGCUGAGUUGGAGCAGCCGAAAGGCUGC
SEQ ID NO: 932 - PNPLA3 oligonucleotide 78 guide (antisense) strand
UCCAACUCAGCUCAGAGGCUGG
SEQ ID NO: 933 - PNPLA3 oligonucleotide 79 passenger (sense) strand
GCCUCUGAGCUGAGUUGGUAGCAGCCGAAAGGCUGC
SEQ ID NO: 934 - PNPLA3 oligonucleotide 79 guide (antisense) strand
UACCAACUCAGCUCAGAGGCGG
SEQ ID NO: 935 - PNPLA3 oligonucleotide 80 passenger (sense) strand
CCUCUGAGCUGAGUUGGUUAGCAGCCGAAAGGCUGC
SEQ ID NO: 936 - PNPLA3 oligonucleotide 80 guide (antisense) strand
UAACCAACUCAGCUCAGAGGGG
SEQ ID NO: 937 - PNPLA3 oligonucleotide 81 passenger (sense) strand
CUCUGAGCUGAGUUGGUUUAGCAGCCGAAAGGCUGC
SEQ ID NO: 938 - PNPLA3 oligonucleotide 81 guide (antisense) strand
UAAACCAACUCAGCUCAGAGGG
SEQ ID NO: 939 - PNPLA3 oligonucleotide 82 passenger (sense) strand
UCUGAGCUGAGUUGGUUUUAGCAGCCGAAAGGCUGC
SEQ ID NO: 940 - PNPLA3 oligonucleotide 82 guide (antisense) strand
UAAAACCAACUCAGCUCAGAGG
SEQ ID NO: 941 - PNPLA3 oligonucleotide 83 passenger (sense) strand
UGAGCUGAGUUGGUUUUAUAGCAGCCGAAAGGCUGC
SEQ ID NO: 942 - PNPLA3 oligonucleotide 83 guide (antisense) strand
UAUAAAACCAACUCAGCUCAGG
SEQ ID NO: 943 - PNPLA3 oligonucleotide 84 passenger (sense) strand
UGAGUUGGUUUUAUGAAAAAGCAGCCGAAAGGCUGC
SEQ ID NO: 944 - PNPLA3 oligonucleotide 84 guide (antisense) strand
UUUUUCAUAAAACCAACUCAGG
SEQ ID NO: 945 - PNPLA3 oligonucleotide 85 passenger (sense) strand
GAGUUGGUUUUAUGAAAAGAGCAGCCGAAAGGCUGC
SEQ ID NO: 946 - PNPLA3 oligonucleotide 85 guide (antisense) strand
UCUUUUCAUAAAACCAACUCGG
SEQ ID NO: 947 - PNPLA3 oligonucleotide 86 passenger (sense) strand
UUGGUUUUAUGAAAAGCUAAGCAGCCGAAAGGCUGC
SEQ ID NO: 948 - PNPLA3 oligonucleotide 86 guide (antisense) strand
UUAGCUUUUCAUAAAACCAAGG
SEQ ID NO: 949 - PNPLA3 oligonucleotide 87 passenger (sense) strand
UGGUUUUAUGAAAAGCUAGAGCAGCCGAAAGGCUGC
SEQ ID NO: 950 - PNPLA3 oligonucleotide 87 guide (antisense) strand
UCUAGCUUUUCAUAAAACCAGG
SEQ ID NO: 951 - PNPLA3 oligonucleotide 88 passenger (sense) strand
GGUUUUAUGAAAAGCUAGGAGCAGCCGAAAGGCUGC
SEQ ID NO: 952 - PNPLA3 oligonucleotide 88 guide (antisense) strand
UCCUAGCUUUUCAUAAAACCGG
SEQ ID NO: 953 - PNPLA3 oligonucleotide 89 passenger (sense) strand
UUUUAUGAAAAGCUAGGAAAGCAGCCGAAAGGCUGC
SEQ ID NO: 954 - PNPLA3 oligonucleotide 89 guide (antisense) strand
UUUCCUAGCUUUUCAUAAAAGG
SEQ ID NO: 955 - PNPLA3 oligonucleotide 90 passenger (sense) strand
UAUGAAAAGCUAGGAAGCAAGCAGCCGAAAGGCUGC
SEQ ID NO: 956 - PNPLA3 oligonucleotide 90 guide (antisense) strand
UUGCUUCCUAGCUUUUCAUAGG
SEQ ID NO: 957 - PNPLA3 oligonucleotide 91 passenger (sense) strand
AUGAAAAGCUAGGAAGCAAAGCAGCCGAAAGGCUGC
SEQ ID NO: 958 - PNPLA3 oligonucleotide 91 guide (antisense) strand
UUUGCUUCCUAGCUUUUCAUGG
SEQ ID NO: 959 - PNPLA3 oligonucleotide 92 passenger (sense) strand
CCAGCACUUAACUCUAAUAAGCAGCCGAAAGGCUGC
SEQ ID NO: 960 - PNPLA3 oligonucleotide 92 guide (antisense) strand
UUAUUAGAGUUAAGUGCUGGGG
SEQ ID NO: 961 - PNPLA3 oligonucleotide 93 passenger (sense) strand
CAGCACUUAACUCUAAUACAGCAGCCGAAAGGCUGC
SEQ ID NO: 962 - PNPLA3 oligonucleotide 93 guide (antisense) strand
UGUAUUAGAGUUAAGUGCUGGG
SEQ ID NO: 963 - PNPLA3 oligonucleotide 94 passenger (sense) strand
UAAUACAUCAGCAUGCGUUAGCAGCCGAAAGGCUGC
SEQ ID NO: 964 - PNPLA3 oligonucleotide 94 guide (antisense) strand
UAACGCAUGCUGAUGUAUUAGG
SEQ ID NO: 965 - PNPLA3 oligonucleotide 95 passenger (sense) strand
AAUACAUCAGCAUGCGUUAAGCAGCCGAAAGGCUGC
SEQ ID NO: 966 - PNPLA3 oligonucleotide 95 guide (antisense) strand
UUAACGCAUGCUGAUGUAUUGG
SEQ ID NO: 967 - PNPLA3 oligonucleotide 96 passenger (sense) strand
UGCGUUAAUUCAGCUGGUUAGCAGCCGAAAGGCUGC
SEQ ID NO: 968 - PNPLA3 oligonucleotide 96 guide (antisense) strand
UAACCAGCUGAAUUAACGCAGG
SEQ ID NO: 969 - PNPLA3 oligonucleotide 97 passenger (sense) strand
GCGUUAAUUCAGCUGGUUGAGCAGCCGAAAGGCUGC
SEQ ID NO: 970 - PNPLA3 oligonucleotide 97 guide (antisense) strand
UCAACCAGCUGAAUUAACGCGG
SEQ ID NO: 971 - PNPLA3 oligonucleotide 98 passenger (sense) strand
CGUUAAUUCAGCUGGUUGGAGCAGCCGAAAGGCUGC
SEQ ID NO: 972 - PNPLA3 oligonucleotide 98 guide (antisense) strand
UCCAACCAGCUGAAUUAACGGG
SEQ ID NO: 973 - PNPLA3 oligonucleotide 99 passenger (sense) strand
UUAAUUCAGCUGGUUGGGAAGCAGCCGAAAGGCUGC
SEQ ID NO: 974 - PNPLA3 oligonucleotide 99 guide (antisense) strand
UUCCCAACCAGCUGAAUUAAGG
SEQ ID NO: 975 - PNPLA3 oligonucleotide 100 passenger (sense) strand
UAAUUCAGCUGGUUGGGAAAGCAGCCGAAAGGCUGC
SEQ ID NO: 976 - PNPLA3 oligonucleotide 100 guide (antisense) strand
UUUCCCAACCAGCUGAAUUAGG
SEQ ID NO: 977 - PNPLA3 oligonucleotide 101 passenger (sense) strand
GCAGAGGGUCCCUUACUGAAGCAGCCGAAAGGCUGC
SEQ ID NO: 978 - PNPLA3 oligonucleotide 101 guide (antisense) strand
UUCAGUAAGGGACCCUCUGCGG
SEQ ID NO: 979 - PNPLA3 oligonucleotide 102 passenger (sense) strand
CUAUUAAUGGUCAGACUGUAGCAGCCGAAAGGCUGC
SEQ ID NO: 980 - PNPLA3 oligonucleotide 102 guide (antisense) strand
UACAGUCUGACCAUUAAUAGGG
SEQ ID NO: 981 - PNPLA3 oligonucleotide 103 passenger (sense) strand
UAUUAAUGGUCAGACUGUUAGCAGCCGAAAGGCUGC
SEQ ID NO: 982 - PNPLA3 oligonucleotide 103 guide (antisense) strand
UAACAGUCUGACCAUUAAUAGG
SEQ ID NO: 983 - PNPLA3 oligonucleotide 104 passenger (sense) strand
AUUAAUGGUCAGACUGUUCAGCAGCCGAAAGGCUGC
SEQ ID NO: 984 - PNPLA3 oligonucleotide 104 guide (antisense) strand
UGAACAGUCUGACCAUUAAUGG
SEQ ID NO: 985 - PNPLA3 oligonucleotide 105 passenger (sense) strand
UUAAUGGUCAGACUGUUCCAGCAGCCGAAAGGCUGC
SEQ ID NO: 986 - PNPLA3 oligonucleotide 105 guide (antisense) strand
UGGAACAGUCUGACCAUUAAGG
SEQ ID NO: 987 - PNPLA3 oligonucleotide 106 passenger (sense) strand
UAAUGGUCAGACUGUUCCAAGCAGCCGAAAGGCUGC
SEQ ID NO: 988 - PNPLA3 oligonucleotide 106 guide (antisense) strand
UUGGAACAGUCUGACCAUUAGG
SEQ ID NO: 989 - PNPLA3 oligonucleotide 107 passenger (sense) strand
AAUGGUCAGACUGUUCCAGAGCAGCCGAAAGGCUGC
SEQ ID NO: 990 - PNPLA3 oligonucleotide 107 guide (antisense) strand
UCUGGAACAGUCUGACCAUUGG
SEQ ID NO: 991 - PNPLA3 oligonucleotide 108 passenger (sense) strand
AGAACACCUUUUUCACCUAAGCAGCCGAAAGGCUGC
SEQ ID NO: 992 - PNPLA3 oligonucleotide 108 guide (antisense) strand
UUAGGUGAAAAAGGUGUUCUGG
SEQ ID NO: 993 - PNPLA3 oligonucleotide 109 passenger (sense) strand
GAACACCUUUUUCACCUAAAGCAGCCGAAAGGCUGC
SEQ ID NO: 994 - PNPLA3 oligonucleotide 109 guide (antisense) strand
UUUAGGUGAAAAAGGUGUUCGG
SEQ ID NO: 995 - PNPLA3 oligonucleotide 110 passenger (sense) strand
AACACCUUUUUCACCUAACAGCAGCCGAAAGGCUGC
SEQ ID NO: 996 - PNPLA3 oligonucleotide 110 guide (antisense) strand
UGUUAGGUGAAAAAGGUGUUGG
SEQ ID NO: 997 - PNPLA3 oligonucleotide 111 passenger (sense) strand
ACACCUUUUUCACCUAACUAGCAGCCGAAAGGCUGC
SEQ ID NO: 998 - PNPLA3 oligonucleotide 111 guide (antisense) strand
UAGUUAGGUGAAAAAGGUGUGG
SEQ ID NO: 999 - PNPLA3 oligonucleotide 112 passenger (sense) strand
CACCUUUUUCACCUAACUAAGCAGCCGAAAGGCUGC
SEQ ID NO: 1000 - PNPLA3 oligonucleotide 112 guide (antisense) strand
UUAGUUAGGUGAAAAAGGUGGG
SEQ ID NO: 1001 - PNPLA3 oligonucleotide 113 passenger (sense) strand
ACCUUUUUCACCUAACUAAAGCAGCCGAAAGGCUGC
SEQ ID NO: 1002 - PNPLA3 oligonucleotide 113 guide (antisense) strand
UUUAGUUAGGUGAAAAAGGUGG
SEQ ID NO: 1003 - PNPLA3 oligonucleotide 114 passenger (sense) strand
CUUUUUCACCUAACUAAAAAGCAGCCGAAAGGCUGC
SEQ ID NO: 1004 - PNPLA3 oligonucleotide 114 guide (antisense) strand
UUUUUAGUUAGGUGAAAAAGGG
SEQ ID NO: 1005 - PNPLA3 oligonucleotide 115 passenger (sense) strand
UUUUCACCUAACUAAAAUAAGCAGCCGAAAGGCUGC
SEQ ID NO: 1006 - PNPLA3 oligonucleotide 115 guide (antisense) strand
UUAUUUUAGUUAGGUGAAAAGG
SEQ ID NO: 1007 - PNPLA3 oligonucleotide 116 passenger (sense) strand
UUCACCUAACUAAAAUAAUAGCAGCCGAAAGGCUGC
SEQ ID NO: 1008 - PNPLA3 oligonucleotide 116 guide (antisense) strand
UAUUAUUUUAGUUAGGUGAAGG
SEQ ID NO: 1009 - PNPLA3 oligonucleotide 117 passenger (sense) strand
UCACCUAACUAAAAUAAUGAGCAGCCGAAAGGCUGC
SEQ ID NO: 1010 - PNPLA3 oligonucleotide 117 guide (antisense) strand
UCAUUAUUUUAGUUAGGUGAGG
SEQ ID NO: 1011 -PNPLA3 oligonucleotide 118 passenger (sense) strand
CACCUAACUAAAAUAAUGUAGCAGCCGAAAGGCUGC
SEQ ID NO: 1012 - PNPLA3 oligonucleotide 118 guide (antisense) strand
UACAUUAUUUUAGUUAGGUGGG
SEQ ID NO: 1013 - PNPLA3 oligonucleotide 119 passenger (sense) strand
ACCUAACUAAAAUAAUGUUAGCAGCCGAAAGGCUGC
SEQ ID NO: 1014 - PNPLA3 oligonucleotide 119 guide (antisense) strand
UAACAUUAUUUUAGUUAGGUGG
SEQ ID NO: 1015 - PNPLA3 oligonucleotide 120 passenger (sense) strand
CCUAACUAAAAUAAUGUUUAGCAGCCGAAAGGCUGC
SEQ ID NO: 1016 - PNPLA3 oligonucleotide 120 guide (antisense) strand
UAAACAUUAUUUUAGUUAGGGG
SEQ ID NO: 1017 - PNPLA3 oligonucleotide 121 passenger (sense) strand
CUAACUAAAAUAAUGUUUAAGCAGCCGAAAGGCUGC
SEQ ID NO: 1018 - PNPLA3 oligonucleotide 121 guide (antisense) strand
UUAAACAUUAUUUUAGUUAGGG
SEQ ID NO: 1019 - PNPLA3 oligonucleotide 122 passenger (sense) strand
UAACUAAAAUAAUGUUUAAAGCAGCCGAAAGGCUGC
SEQ ID NO: 1020 - PNPLA3 oligonucleotide 122 guide (antisense) strand
UUUAAACAUUAUUUUAGUUAGG
SEQ ID NO: 1021 - PNPLA3 oligonucleotide 123 passenger (sense) strand
AACUAAAAUAAUGUUUAAAAGCAGCCGAAAGGCUGC
SEQ ID NO: 1022 - PNPLA3 oligonucleotide 123 guide (antisense) strand
UUUUAAACAUUAUUUUAGUUGG
SEQ ID NO: 1023 - PNPLA3 oligonucleotide 124 passenger (sense) strand
ACUAAAAUAAUGUUUAAAGAGCAGCCGAAAGGCUGC
SEQ ID NO: 1024 - PNPLA3 oligonucleotide 124 guide (antisense) strand
UCUUUAAACAUUAUUUUAGUGG
SEQ ID NO: 1025 - PNPLA3 oligonucleotide 125 passenger (sense) strand
CUAAAAUAAUGUUUAAAGAAGCAGCCGAAAGGCUGC
SEQ ID NO: 1026 - PNPLA3 oligonucleotide 125 guide (antisense) strand
UUCUUUAAACAUUAUUUUAGGG
SEQ ID NO: 1027 - PNPLA3 oligonucleotide 126 passenger (sense) strand
UAAAAUAAUGUUUAAAGAGAGCAGCCGAAAGGCUGC
SEQ ID NO: 1028 - PNPLA3 oligonucleotide 126 guide (antisense) strand
UCUCUUUAAACAUUAUUUUAGG
SEQ ID NO: 1029 - PNPLA3 oligonucleotide 127 passenger (sense) strand
AAGAGUUUUGUAUAAAAAUAGCAGCCGAAAGGCUGC
SEQ ID NO: 1030 - PNPLA3 oligonucleotide 127 guide (antisense) strand
UAUUUUUAUACAAAACUCUUGG
SEQ ID NO: 1031 - PNPLA3 oligonucleotide 128 passenger (sense) strand
GUUUUGUAUAAAAAUGUAAAGCAGCCGAAAGGCUGC
SEQ ID NO: 1032 - PNPLA3 oligonucleotide 128 guide (antisense) strand
UUUACAUUUUUAUACAAAACGG
SEQ ID NO: 1033 - PNPLA3 oligonucleotide 129 passenger (sense) strand
UUGUAUAAAAAUGUAAGGAAGCAGCCGAAAGGCUGC
SEQ ID NO: 1034 - PNPLA3 oligonucleotide 129 guide (antisense) strand
UUCCUUACAUUUUUAUACAAGG
SEQ ID NO: 1035 - PNPLA3 oligonucleotide 130 passenger (sense) strand
UGUAUAAAAAUGUAAGGAAAGCAGCCGAAAGGCUGC
SEQ ID NO: 1036 - PNPLA3 oligonucleotide 130 guide (antisense) strand
UUUCCUUACAUUUUUAUACAGG
SEQ ID NO: 1037 - PNPLA3 oligonucleotide 131 passenger (sense) strand
GUAUAAAAAUGUAAGGAAGAGCAGCCGAAAGGCUGC
SEQ ID NO: 1038 - PNPLA3 oligonucleotide 131 guide (antisense) strand
UCUUCCUUACAUUUUUAUACGG
SEQ ID NO: 1039 - PNPLA3 oligonucleotide 132 passenger (sense) strand
AUGUAAGGAAGCGUUGUUAAGCAGCCGAAAGGCUGC
SEQ ID NO: 1040 - PNPLA3 oligonucleotide 132 guide (antisense) strand
UUAACAACGCUUCCUUACAUGG
SEQ ID NO: 1041 - PNPLA3 oligonucleotide 133 passenger (sense) strand
UUUUGUAUUAUGUGAAUCAAGCAGCCGAAAGGCUGC
SEQ ID NO: 1042 - PNPLA3 oligonucleotide 133 guide (antisense) strand
UUGAUUCACAUAAUACAAAAGG
SEQ ID NO: 1043 - PNPLA3 oligonucleotide 134 passenger (sense) strand
UUUGUAUUAUGUGAAUCAGAGCAGCCGAAAGGCUGC
SEQ ID NO: 1044 - PNPLA3 oligonucleotide 134 guide (antisense) strand
UCUGAUUCACAUAAUACAAAGG
SEQ ID NO: 1045 - PNPLA3 oligonucleotide 135 passenger (sense) strand
UUGUAUUAUGUGAAUCAGUAGCAGCCGAAAGGCUGC
SEQ ID NO: 1046 - PNPLA3 oligonucleotide 135 guide (antisense) strand
UACUGAUUCACAUAAUACAAGG
SEQ ID NO: 1047 - PNPLA3 oligonucleotide 136 passenger (sense) strand
GUAUUAUGUGAAUCAGUGAAGCAGCCGAAAGGCUGC
SEQ ID NO: 1048 - PNPLA3 oligonucleotide 136 guide (antisense) strand
UUCACUGAUUCACAUAAUACGG
SEQ ID NO: 1049 - PNPLA3 oligonucleotide 137 passenger (sense) strand
UAUUAUGUGAAUCAGUGAGAGCAGCCGAAAGGCUGC
SEQ ID NO: 1050 - PNPLA3 oligonucleotide 137 guide (antisense) strand
UCUCACUGAUUCACAUAAUAGG
SEQ ID NO: 1051 - PNPLA3 oligonucleotide 138 passenger (sense) strand
AUUAUGUGAAUCAGUGAGAAGCAGCCGAAAGGCUGC
SEQ ID NO: 1052 - PNPLA3 oligonucleotide 138 guide (antisense) strand
UUCUCACUGAUUCACAUAAUGG
SEQ ID NO: 1053 - PNPLA3 oligonucleotide 139 passenger (sense) strand
UUAUGUGAAUCAGUGAGAUAGCAGCCGAAAGGCUGC
SEQ ID NO: 1054 - PNPLA3 oligonucleotide 139 guide (antisense) strand
UAUCUCACUGAUUCACAUAAGG
SEQ ID NO: 1055 - PNPLA3 oligonucleotide 140 passenger (sense) strand
UAUGUGAAUCAGUGAGAUGAGCAGCCGAAAGGCUGC
SEQ ID NO: 1056 - PNPLA3 oligonucleotide 140 guide (antisense) strand
UCAUCUCACUGAUUCACAUAGG
SEQ ID NO: 1057 - PNPLA3 oligonucleotide 141 passenger (sense) strand
AUGUGAAUCAGUGAGAUGUAGCAGCCGAAAGGCUGC
SEQ ID NO: 1058 - PNPLA3 oligonucleotide 141 guide (antisense) strand
UACAUCUCACUGAUUCACAUGG
SEQ ID NO: 1059 - PNPLA3 oligonucleotide 142 passenger (sense) strand
UGUGAAUCAGUGAGAUGUUAGCAGCCGAAAGGCUGC
SEQ ID NO: 1060 - PNPLA3 oligonucleotide 142 guide (antisense) strand
UAACAUCUCACUGAUUCACAGG
SEQ ID NO: 1061 - PNPLA3 oligonucleotide 143 passenger (sense) strand
GUGAAUCAGUGAGAUGUUAAGCAGCCGAAAGGCUGC
SEQ ID NO: 1062 - PNPLA3 oligonucleotide 143 guide (antisense) strand
UUAACAUCUCACUGAUUCACGG
SEQ ID NO: 1063 - PNPLA3 oligonucleotide 144 passenger (sense) strand
GUGAGAUGUUAGUAGAAUAAGCAGCCGAAAGGCUGC
SEQ ID NO: 1064 - PNPLA3 oligonucleotide 144 guide (antisense) strand
UUAUUCUACUAACAUCUCACGG
SEQ ID NO: 1065 - PNPLA3 oligonucleotide 145 passenger (sense) strand
UUUCUAUUUAUGCAUUUGAAGCAGCCGAAAGGCUGC
SEQ ID NO: 1066 - PNPLA3 oligonucleotide 145 guide (antisense) strand
UUCAAAUGCAUAAAUAGAAAGG
SEQ ID NO: 1067 - PNPLA3 oligonucleotide 146 passenger (sense) strand
CUAUUUAUGCAUUUGAGUAAGCAGCCGAAAGGCUGC
SEQ ID NO: 1068 - PNPLA3 oligonucleotide 146 guide (antisense) strand
UUACUCAAAUGCAUAAAUAGGG
SEQ ID NO: 1069 - PNPLA3 oligonucleotide 147 passenger (sense) strand
UGCUCAAACUGUUAAAUGUAGCAGCCGAAAGGCUGC
SEQ ID NO: 1070 - PNPLA3 oligonucleotide 147 guide (antisense) strand
UACAUUUAACAGUUUGAGCAGG
SEQ ID NO: 1071 - PNPLA3 oligonucleotide 148 passenger (sense) strand
GCUCAAACUGUUAAAUGUUAGCAGCCGAAAGGCUGC
SEQ ID NO: 1072 - PNPLA3 oligonucleotide 148 guide (antisense) strand
UAACAUUUAACAGUUUGAGCGG
SEQ ID NO: 1073 - PNPLA3 oligonucleotide 149 passenger (sense) strand
CUCAAACUGUUAAAUGUUGAGCAGCCGAAAGGCUGC
SEQ ID NO: 1074 - PNPLA3 oligonucleotide 149 guide (antisense) strand
UCAACAUUUAACAGUUUGAGGG
SEQ ID NO: 1075 - PNPLA3 oligonucleotide 150 passenger (sense) strand
UCAAACUGUUAAAUGUUGGAGCAGCCGAAAGGCUGC
SEQ ID NO: 1076 - PNPLA3 oligonucleotide 150 guide (antisense) strand
UCCAACAUUUAACAGUUUGAGG
SEQ ID NO: 1077 - PNPLA3 oligonucleotide 151 passenger (sense) strand
CAAACUGUUAAAUGUUGGAAGCAGCCGAAAGGCUGC
SEQ ID NO: 1078 - PNPLA3 oligonucleotide 151 guide (antisense) strand
UUCCAACAUUUAACAGUUUGGG
SEQ ID NO: 1079 - PNPLA3 oligonucleotide 152 passenger (sense) strand
AAACUGUUAAAUGUUGGAAAGCAGCCGAAAGGCUGC
SEQ ID NO: 1080 - PNPLA3 oligonucleotide 152 guide (antisense) strand
UUUCCAACAUUUAACAGUUUGG
SEQ ID NO: 1081 - PNPLA3 oligonucleotide 153 passenger (sense) strand
AACUGUUAAAUGUUGGAAAAGCAGCCGAAAGGCUGC
SEQ ID NO: 1082 - PNPLA3 oligonucleotide 153 guide (antisense) strand
UUUUCCAACAUUUAACAGUUGG
SEQ ID NO: 1083 - PNPLA3 oligonucleotide 154 passenger (sense) strand
UGUUAAAUGUUGGAAAAGAAGCAGCCGAAAGGCUGC
SEQ ID NO: 1084 - PNPLA3 oligonucleotide 154 guide (antisense) strand
UUCUUUUCCAACAUUUAACAGG
SEQ ID NO: 1085 - PNPLA3 oligonucleotide 155 passenger (sense) strand
GUUAAAUGUUGGAAAAGAAAGCAGCCGAAAGGCUGC
SEQ ID NO: 1086 - PNPLA3 oligonucleotide 155 guide (antisense) strand
UUUCUUUUCCAACAUUUAACGG
SEQ ID NO: 1087 - PNPLA3 oligonucleotide 156 passenger (sense) strand
UUAAAUGUUGGAAAAGAAAAGCAGCCGAAAGGCUGC
SEQ ID NO: 1088 - PNPLA3 oligonucleotide 156 guide (antisense) strand
UUUUCUUUUCCAACAUUUAAGG
SEQ ID NO: 1089 - PNPLA3 oligonucleotide 157 passenger (sense) strand
UAAAUGUUGGAAAAGAAAGAGCAGCCGAAAGGCUGC
SEQ ID NO: 1090 - PNPLA3 oligonucleotide 157 guide (antisense) strand
UCUUUCUUUUCCAACAUUUAGG
SEQ ID NO: 1091 - PNPLA3 oligonucleotide 158 passenger (sense) strand
AGAAAAGAGGCUACUUGUGAGCAGCCGAAAGGCUGC
SEQ ID NO: 1092 - PNPLA3 oligonucleotide 158 guide (antisense) strand
UCACAAGUAGCCUCUUUUCUGG
SEQ ID NO: 1093 - PNPLA3 oligonucleotide 159 passenger (sense) strand
GAAAAGAGGCUACUUGUGAAGCAGCCGAAAGGCUGC
SEQ ID NO: 1094 - PNPLA3 oligonucleotide 159 guide (antisense) strand
UUCACAAGUAGCCUCUUUUCGG
SEQ ID NO: 1095 - PNPLA3 oligonucleotide 160 passenger (sense) strand
AAAAGAGGCUACUUGUGAAAGCAGCCGAAAGGCUGC
SEQ ID NO: 1096 - PNPLA3 oligonucleotide 160 guide (antisense) strand
UUUCACAAGUAGCCUCUUUUGG
SEQ ID NO: 1097 - PNPLA3 oligonucleotide 161 passenger (sense) strand
AGAGGCUACUUGUGAAAAUAGCAGCCGAAAGGCUGC
SEQ ID NO: 1098 - PNPLA3 oligonucleotide 161 guide (antisense) strand
UAUUUUCACAAGUAGCCUCUGG
SEQ ID NO: 1099 - PNPLA3 oligonucleotide 162 passenger (sense) strand
GAGGCUACUUGUGAAAAUAAGCAGCCGAAAGGCUGC
SEQ ID NO: 1100 - PNPLA3 oligonucleotide 162 guide (antisense) strand
UUAUUUUCACAAGUAGCCUCGG
SEQ ID NO: 1101 - PNPLA3 oligonucleotide 163 passenger (sense) strand
AAAGUUACAAGUUUCUUUUAGCAGCCGAAAGGCUGC
SEQ ID NO: 1102 - PNPLA3 oligonucleotide 163 guide (antisense) strand
UAAAAGAAACUUGUAACUUUGG
SEQ ID NO: 1103 - PNPLA3 oligonucleotide 164 passenger (sense) strand
CAACAGUAUUUUCUAAUAAAGCAGCCGAAAGGCUGC
SEQ ID NO: 1104 - PNPLA3 oligonucleotide 164 guide (antisense) strand
UUUAUUAGAAAAUACUGUUGGG
SEQ ID NO: 1105 - PNPLA3 oligonucleotide 165 passenger (sense) strand
AACAGUAUUUUCUAAUAACAGCAGCCGAAAGGCUGC
SEQ ID NO: 1106 - PNPLA3 oligonucleotide 165 guide (antisense) strand
UGUUAUUAGAAAAUACUGUUGG
SEQ ID NO: 1107 - PNPLA3 oligonucleotide 166 passenger (sense) strand
ACAGUAUUUUCUAAUAACCAGCAGCCGAAAGGCUGC
SEQ ID NO: 1108 - PNPLA3 oligonucleotide 166 guide (antisense) strand
UGGUUAUUAGAAAAUACUGUGG
SEQ ID NO: 1109 - PNPLA3 oligonucleotide 167 passenger (sense) strand
CAGUAUUUUCUAAUAACCAAGCAGCCGAAAGGCUGC
SEQ ID NO: 1110 - PNPLA3 oligonucleotide 167 guide (antisense) strand
UUGGUUAUUAGAAAAUACUGGG
SEQ ID NO: 1111 - PNPLA3 oligonucleotide 168 passenger (sense) strand
UUGUGAUUGUUAUCAGGAAAGCAGCCGAAAGGCUGC
SEQ ID NO: 1112 - PNPLA3 oligonucleotide 168 guide (antisense) strand
UUUCCUGAUAACAAUCACAAGG
SEQ ID NO: 1113 - PNPLA3 oligonucleotide 169 passenger (sense) strand
UGUGAUUGUUAUCAGGAAAAGCAGCCGAAAGGCUGC
SEQ ID NO: 1114 - PNPLA3 oligonucleotide 169 guide (antisense) strand
UUUUCCUGAUAACAAUCACAGG
SEQ ID NO: 1115 - PNPLA3 oligonucleotide 170 passenger (sense) strand
GUGAUUGUUAUCAGGAAAAAGCAGCCGAAAGGCUGC
SEQ ID NO: 1116 - PNPLA3 oligonucleotide 170 guide (antisense) strand
UUUUUCCUGAUAACAAUCACGG
SEQ ID NO: 1117 - PNPLA3 oligonucleotide 171 passenger (sense) strand
UGAUUGUUAUCAGGAAAAAAGCAGCCGAAAGGCUGC
SEQ ID NO: 1118 - PNPLA3 oligonucleotide 171 guide (antisense) strand
UUUUUUCCUGAUAACAAUCAGG
SEQ ID NO: 1119 - PNPLA3 oligonucleotide 172 passenger (sense) strand
GAUUGUUAUCAGGAAAAAAAGCAGCCGAAAGGCUGC
SEQ ID NO: 1120 - PNPLA3 oligonucleotide 172 guide (antisense) strand
UUUUUUUCCUGAUAACAAUCGG
SEQ ID NO: 1121 - PNPLA3 oligonucleotide 173 passenger (sense) strand
AUUGUUAUCAGGAAAAAAUAGCAGCCGAAAGGCUGC
SEQ ID NO: 1122 - PNPLA3 oligonucleotide 173 guide (antisense) strand
UAUUUUUUCCUGAUAACAAUGG
SEQ ID NO: 1123 - PNPLA3 oligonucleotide 174 passenger (sense) strand
UUGUUAUCAGGAAAAAAUAAGCAGCCGAAAGGCUGC
SEQ ID NO: 1124 - PNPLA3 oligonucleotide 174 guide (antisense) strand
UUAUUUUUUCCUGAUAACAAGG
SEQ ID NO: 1125 - PNPLA3 oligonucleotide 175 passenger (sense) strand
UGUUAUCAGGAAAAAAUAUAGCAGCCGAAAGGCUGC
SEQ ID NO: 1126 - PNPLA3 oligonucleotide 175 guide (antisense) strand
UAUAUUUUUUCCUGAUAACAGG
SEQ ID NO: 1127 - PNPLA3 oligonucleotide 176 passenger (sense) strand
GUUAUCAGGAAAAAAUAUAAGCAGCCGAAAGGCUGC
SEQ ID NO: 1128 - PNPLA3 oligonucleotide 176 guide (antisense) strand
UUAUAUUUUUUCCUGAUAACGG
SEQ ID NO: 1129 - PNPLA3 oligonucleotide 177 passenger (sense) strand
UUAUCAGGAAAAAAUAUAUAGCAGCCGAAAGGCUGC
SEQ ID NO: 1130 - PNPLA3 oligonucleotide 177 guide (antisense) strand
UAUAUAUUUUUUCCUGAUAAGG
SEQ ID NO: 1131 - PNPLA3 oligonucleotide 178 passenger (sense) strand
AGGAAAAAAUAUAUUAAAUAGCAGCCGAAAGGCUGC
SEQ ID NO: 1132 - PNPLA3 oligonucleotide 178 guide (antisense) strand
UAUUUAAUAUAUUUUUUCCUGG
SEQ ID NO: 1133 - PNPLA3 oligonucleotide 179 passenger (sense) strand
GGAAAAAAUAUAUUAAAUGAGCAGCCGAAAGGCUGC
SEQ ID NO: 1134 - PNPLA3 oligonucleotide 179 guide (antisense) strand
UCAUUUAAUAUAUUUUUUCCGG
SEQ ID NO: 1135 - PNPLA3 oligonucleotide 180 passenger (sense) strand
GAAAAAAUAUAUUAAAUGGAGCAGCCGAAAGGCUGC
SEQ ID NO: 1136 - PNPLA3 oligonucleotide 180 guide (antisense) strand
UCCAUUUAAUAUAUUUUUUCGG
SEQ ID NO: 1137 - PNPLA3 oligonucleotide 181 passenger (sense) strand
AUUUUCUUUCUGCUUUUAAAGCAGCCGAAAGGCUGC
SEQ ID NO: 1138 - PNPLA3 oligonucleotide 181 guide (antisense) strand
UUUAAAAGCAGAAAGAAAAUGG
SEQ ID NO: 1139 - PNPLA3 oligonucleotide 182 passenger (sense) strand
UCUUUCUGCUUUUAAAAAUAGCAGCCGAAAGGCUGC
SEQ ID NO: 1140 - PNPLA3 oligonucleotide 182 guide (antisense) strand
UAUUUUUAAAAGCAGAAAGAGG
SEQ ID NO: 1141 - PNPLA3 oligonucleotide 183 passenger (sense) strand
UAAGGAAAUCAGGAAGUGUAGCAGCCGAAAGGCUGC
SEQ ID NO: 1142 - PNPLA3 oligonucleotide 183 guide (antisense) strand
UACACUUCCUGAUUUCCUUAGG
SEQ ID NO: 1143 - PNPLA3 oligonucleotide 184 passenger (sense) strand
AAGGAAAUCAGGAAGUGUAAGCAGCCGAAAGGCUGC
SEQ ID NO: 1144 - PNPLA3 oligonucleotide 184 guide (antisense) strand
UUACACUUCCUGAUUUCCUUGG
SEQ ID NO: 1145 - PNPLA3 oligonucleotide 185 passenger (sense) strand
GGAAAUCAGGAAGUGUAAUAGCAGCCGAAAGGCUGC
SEQ ID NO: 1146 - PNPLA3 oligonucleotide 185 guide (antisense) strand
UAUUACACUUCCUGAUUUCCGG
SEQ ID NO: 1147 - PNPLA3 oligonucleotide 186 passenger (sense) strand
CCACACCCAGCUAGUUUUUAGCAGCCGAAAGGCUGC
SEQ ID NO: 1148 - PNPLA3 oligonucleotide 186 guide (antisense) strand
UAAAAACUAGCUGGGUGUGGGG
SEQ ID NO: 1149 - PNPLA3 oligonucleotide 187 passenger (sense) strand
CACACCCAGCUAGUUUUUUAGCAGCCGAAAGGCUGC
SEQ ID NO: 1150 - PNPLA3 oligonucleotide 187 guide (antisense) strand
UAAAAAACUAGCUGGGUGUGGG
SEQ ID NO: 1151 - PNPLA3 oligonucleotide 188 passenger (sense) strand
CUAGGAUUACAGGUGUGAGAGCAGCCGAAAGGCUGC
SEQ ID NO: 1152 - PNPLA3 oligonucleotide 188 guide (antisense) strand
UCUCACACCUGUAAUCCUAGGG
SEQ ID NO: 1153 - PNPLA3 oligonucleotide 189 passenger (sense) strand
AGAGUAUGAGCCUGAUUUUAGCAGCCGAAAGGCUGC
SEQ ID NO: 1154 - PNPLA3 oligonucleotide 189 guide (antisense) strand
UAAAAUCAGGCUCAUACUCUGG
SEQ ID NO: 1155 - PNPLA3 oligonucleotide 190 passenger (sense) strand
GAGUAUGAGCCUGAUUUUGAGCAGCCGAAAGGCUGC
SEQ ID NO: 1156 - PNPLA3 oligonucleotide 190 guide (antisense) strand
UCAAAAUCAGGCUCAUACUCGG
SEQ ID NO: 1157 - PNPLA3 oligonucleotide 191 passenger (sense) strand
CCCAUCUCUACUAAAAAAUAGCAGCCGAAAGGCUGC
SEQ ID NO: 1158 - PNPLA3 oligonucleotide 191 guide (antisense) strand
UAUUUUUUAGUAGAGAUGGGGG
SEQ ID NO: 1159 - PNPLA3 oligonucleotide 192 passenger (sense) strand
ACUUUCAGCAUGAGAAAAUAGCAGCCGAAAGGCUGC
SEQ ID NO: 1160 - PNPLA3 oligonucleotide 192 guide (antisense) strand
UAUUUUCUCAUGCUGAAAGUGG
SEQ ID NO: 1161 - PNPLA3 oligonucleotide 193 passenger (sense) strand
CUGAGCUGAGUUGGUUUUAGCAGCCGAAAGGCUGC
SEQ ID NO: 1162 - PNPLA3 oligonucleotide 193 guide (antisense) strand
UUAAAACCAACUCAGCUCAGGG
SEQ ID NO: 1163 - PNPLA3 oligonucleotide 194 passenger (sense) strand
GCUGAGUUGGUUUUAUGAAAGCAGCCGAAAGGCUGC
SEQ ID NO: 1164 - PNPLA3 oligonucleotide 194 guide (sense) strand
UUUCAUAAAACCAACUCAGCGG
SEQ ID NO: 1165 - PNPLA3 control passenger (sense) strand
CUGUUGAAUUUUGUAUUAUA
SEQ ID NO: 1166 - PNPLA3 control guide (antisense) strand
UAUAAUACAAAAUUCAACAGGG
SEQ ID NO: 1167 - Target Sequence 1
CAACGTACCCTTCATTGAT
SEQ ID NO: 1168 - Target Sequence 2
TGAAAGACAAAGGTGGATA
SEQ ID NO: 1169 - Target Sequence 3
TACATGAGCAAGATTTGCA
SEQ ID NO: 1170 - Target Sequence 4
TGAGCAAGATTTGCAACTT
SEQ ID NO: 1171 - Target Sequence 5
CTCTGAGCTGAGTTGGTTT
SEQ ID NO: 1172 - Target Sequence 6
CTTTTTCACCTAACTAAAA
SEQ ID NO: 1173 - Target Sequence 7
TTCACCTAACTAAAATAAT
SEQ ID NO: 1174 - Target Sequence 8
CTAACTAAAATAATGTTTA
SEQ ID NO: 1175 - Target Sequence 9
CTGAGCTGAGTTGGTTTTA
SEQ ID NO: 1176 - Target Sequence 10
GCTGAGTTGGTTTTATGAA
SEQ ID NO: 1177 - Artificial Sequence
GCAGCCGAAAGGCUGC

TABLE A

| | SEQ ID | | |
|---|---|---|---|
| | NO. and | | |
| | Mod. | DP | |
| No. | Pattern | Number | Modified Oligonucleotide |
| 1 | 1170 | DP17736P: | [mCs][mC][fC][mA][mU][mU][mA][fG][fG][fA][mU][fA][fA][mU] |
| | SM1224/ | DP17735G | [mG][mU][fC][mU][mU][mA][mG][mC][mA][mG][mC][mC][mG] |
| | ASM1508 | | [ademA-GalNAc][ademA-GalNAc][ademA- |
| | | | GalNAc][mG][mG][mC][mU][mG][mC] (SEQ ID NO: 1178) |
| | | | [MePhosphonate-4O- |
| | | | mUs][fAs][fAs][mG][fA][mC][fA][fU][mU][fA][mU][fC][mC][fU] |
| | | | [mA][fA][mU][mG][fG][mGs][mGs][mG] (SEQ ID NO: 1179) |
| 2 | 0643 | DP17919P: | [mAs][mC][mA][mA][mC][mG][mU][fA][fC][fC][fC][mU][mU][mC] |
| | SM1405/ | DP17918G | [mA][mU][mU][mG][mA][mA][mG][mC][mA][mG][mC][mC][mG] |
| | ASM2028 | | [ademA-GalNAc][ademA-GalNAc][ademA- |
| | | | GalNAc][mG][mG][mC][mU][mG][mC] (SEQ ID NO: 1180) |
| | | | [MePhosphonate-4O- |
| | | | mUs][fUs][fCs][fA][fA][mU][fG][mA][mA][fG][mG][mG][mU][fA] |
| | | | [mC][mG][mU][mU][mG][mUs][mGs][mG] (SEQ ID NO: 1181) |
| 3 | 1110 | DP17921P: | [mGs][mC][mA][mC][mU][mG][mA][fG][fU][fG][fA][mA][mG] |
| | SM1405/ | DP17920G | [mA][mA][mA][mU][mG][mA][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1182) |
| | | | [MePhosphonate-4O- |
| | | | mUs][fUs][fCs][fA][fU][mU][fU][mC][mU][fU][mC][mA][mC][fU] |
| | | | [mC][mA][mG][mU][mG][mCs][mGs][mG] (SEQ ID NO: 1183) |
| 4 | 1162 | DP17923P: | [mAs][mC][mU][mU][mG][mC][mU][fA][fC][fC][fC][mA][mU][mU] |
| | SM1405/ | DP17922G | [mA][mG][mG][mA][mU][mA][mG][mC][mA][mG][mC][mC][mG] |
| | ASM2028 | | [ademA-GalNAc][ademA-GalNAc][ademA- |
| | | | GalNAc][mG][mG][mC][mU][mG][mC] (SEQ ID NO: 1184) |
| | | | [MePhosphonate-4O- |
| | | | mUs][fAs][fUs][fC][fC][mU][fA][mA][mU][fG][mG][mG][mU][fA] |
| | | | [mG][mC][mA][mA][mG][mUs][mGs][mG] (SEQ ID NO: 1185) |
| 5 | 1170 | DP17925P: | [mCs][mC][mC][mA][mU][mU][mA][fG][fG][fA][fU][mA][mA] |
| | SM1405/ | DP17924G | [mU][mG][mU][mC][mU][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1186) |
| | | | [MePhosphonate-4O- |
| | | | mUs][fAs][fAs][fG][fA][mC][fA][mU][mU][fA][mU][mC][mC][fU] |
| | | | [mA][mA][mU][mG][mG][mGs][mGs][mG] (SEQ ID NO: 1187) |
| 6 | 1699 | DP17927P: | [mCs][mU][mG][mA][mG][mC][mU][fG][fA][fG][fU][mU][mG] |
| | SM1405/ | DP17926G | [mG][mU][mU][mU][mU][mA][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1188) |
| | | | [MePhosphonate-4O- |
| | | | mUs][fUs][fAs][fA][fA][mA][fC][mC][mA][fA][mC][mU][mC][fA] |
| | | | [mG][mC][mU][mC][mA][mGs][mGs][mG] (SEQ ID NO: 1189) |
| 7 | 1703 | DP17929P: | [mGs][mC][mU][mG][mA][mG][mU][fU][fG][fG][fU][mU][mU] |
| | SM1405/ | DP17928G | [mU][mA][mU][mG][mA][mA][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1190) |
| | | | [MePhosphonate-4O- |
| | | | mUs][fUs][fUs][fC][fA][mU][fA][mA][mA][fA][mC][mC][mA][fA] |
| | | | [mC][mU][mC][mA][mG][mCs][mGs][mG] (SEQ ID NO: 1191) |
| 8 | 1708 | DP17931P: | [mGs][mU][mU][mG][mG][mU][mU][fU][fU][fA][fU][mG][mA] |
| | SM1405/ | DP17930G | [mA][mA][mA][mG][mC][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1192) |
| | | | [MePhosphonate-4O- |
| | | | mUs][fAs][fGs][fC][fU][mU][fU][mU][mC][fA][mU][mA][mA][fA] |
| | | | [mA][mC][mC][mA][mA][mCs][mGs][mG] (SEQ ID NO: 1193) |
| 9 | 2104 | DP17933P: | [mGs][mG][mU][mA][mA][mC][mA][fA][fG][fA][fU][mG][mA] |
| | SM1405/ | DP17932G | [mU][mA][mA][mU][mC][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1194) |
| | | | [MePhosphonate-4O- |
| | | | mUs][fAs][fGs][fA][fU][mU][fA][mU][mC][fA][mU][mC][mU][fU] |
| | | | [mG][mU][mU][mA][mC][mCs][mGs][mG] (SEQ ID NO: 1195) |
| 10 | 2143 | DP17935P: | [mUs][mU][mU][mC][mA][mC][mC][fU][fA][fA][fC][mU][mA][mA] |
| | SM1405/ | DP17934G | [mA][mA][mU][mA][mA][mA][mG][mC][mA][mG][mC][mC][mG] |
| | ASM2028 | | [ademA-GalNAc][ademA-GalNAc][ademA- |
| | | | GalNAc][mG][mG][mC][mU][mG][mC] (SEQ ID NO: 1196) |
| | | | [MePhosphonate-4O- |
| | | | mUs][fUs][fUs][fA][fU][mU][fU][mU][mA][fG][mU][mU][mA][fG] |
| | | | [mG][mU][mG][mA][mA][mAs][mGs][mG] (SEQ ID NO: 1197) |
| 11 | 2155 | DP17937P: | [mAs][mA][mA][mA][mU][mA][mA][fU][fG][fU][fU][mU][mA] |
| | SM1405/ | DP17936G | [mA][mA][mG][mA][mG][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1198) |
| | | | [MePhosphonate-4O- |
| | | | mUs][fAs][fCs][fU][fC][mU][fU][mU][mA][fA][mA][mC][mA][fU] |
| | | | [mU][mA][mU][mU][mU][mUs][mGs][mG] (SEQ ID NO: 1199) |
| 12 | 2156 | DP17939P: | [mAs][mA][mA][mU][mA][mA][mU][fG][fU][fU][fU][mA][mA] |
| | SM1405/ | DP17938G | [mA][mG][mA][mG][mU][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1200) |
| | | | [MePhosphonate-4O- |
| | | | mUs][fAs][fAs][fC][fU][mC][fU][mU][mU][fA][mA][mA][mC][fA] |
| | | | [mU][mU][mA][mU][mU][mUs][mGs][mG] (SEQ ID NO: 1201) |
| 13 | 2157 | DP17941P: | [mAs][mA][mU][mA][mA][mU][mG][fU][fU][fU][fA][mA][mA] |
| | SM1405/ | DP17940G | [mG][mA][mG][mU][mU][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1202) |
| | | | [MePhosphonate-4O- |
| | | | mUs][fAs][fAs][fA][fC][mU][fC][mU][mU][fU][mA][mA][mA][fC] |
| | | | [mA][mU][mU][mA][mU][mUs][mGs][mG] (SEQ ID NO: 1203) |
| 14 | 2163 | DP17943P: | [mGs][mU][mU][mU][mA][mA][mA][fG][fA][fG][fU][mU][mU] |
| | SM1405/ | DP17942G | [mU][mG][mU][mA][mU][mA][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1204) |
| | | | [MePhosphonate-4O- |
| | | | mUs][fUs][fAs][fU][fA][mC][fA][mA][mA][fA][mC][mU][mC][fU] |
| | | | [mU][mU][mA][mA][mA][mCs][mGs][mG] (SEQ ID NO: 1205) |
| 15 | 2165 | DP17945P: | [mUs][mU][mA][mA][mA][mG][mA][fG][fU][fU][fU][mU][mG] |
| | SM1405/ | DP17944G | [mU][mA][mU][mA][mA][mA][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1206) |
| | | | [MePhosphonate-4O- |
| | | | mUs][fUs][fUs][fU][fA][mU][fA][mC][mA][fA][mA][mA][mC][fU] |
| | | | [mC][mU][mU][mU][mA][mAs][mGs][mG] (SEQ ID NO: 1207) |
| 16 | 2170 | DP17947P: | [mGs][mA][mG][mU][mU][mU][mU][fG][fU][fA][fU][mA][mA] |
| | SM1405/ | DP17946G | [mA][mA][mA][mU][mG][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1208) |
| | | | [MePhosphonate-4O- |
| | | | mUs][fAs][fCs][fA][fU][mU][fU][mU][mU][fA][mU][mA][mC][fA] |
| | | | [mA][mA][mA][mC][mU][mCs][mGs][mG] (SEQ ID NO: 1209) |
| 17 | 2195 | DP17949P: | [mGs][mC][mG][mU][mU][mG][mU][fU][fA][fC][fC][mU][mG] |
| | SM1405/ | DP17948G | [mU][mU][mG][mA][mA][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1210) |
| | | | [MePhosphonate-4O- |
| | | | mUs][fAs][fUs][fU][fC][mA][fA][mC][mA][fG][mG][mU][mA][fA] |
| | | | [mC][mA][mA][mC][mG][mCs][mGs][mG] (SEQ ID NO: 1211) |
| 18 | 2200 | DP17951P: | [mGs][mU][mU][mA][mC][mC][mU][fG][fU][fU][fG][mA][mA] |
| | SM1405/ | DP17950G | U[m][mU][mU][mU][mG][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1212) |
| | | | [MePhosphonate-4O- |
| | | | mUs][fAs][fCs][fA][fA][mA][fA][mU][mU][fC][mA][mA][mC][fA] |
| | | | [mG][mG][mU][mA][mA][mCs][mGs][mG] (SEQ ID NO: 1213) |
| 19 | 2204 | DP17953P: | [mCs][mC][mU][mG][mU][mU][mG][fA][fA][fU][fU][mU][mU] |
| | SM1405/ | DP17952G | [mG][mU][mA][mU][mU][mA][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1214) |
| | | | [MePhosphonate-4O- |
| | | | mUs][fUs][fAs][fA][fU][mA][fC][mA][mA][fA][mA][mU][mU][fC] |
| | | | [mA][mA][mC][mA][mG][mGs][mGs][mG] (SEQ ID NO: 1215) |
| 20 | 2205 | DP17955P: | [mCs][mU][mG][mU][mU][mG][mA][fA][fU][fU][fU][mU][mG] |
| | SM1405/ | DP17954G | [mU][mA][mU][mU][mA][mU][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1216) |
| | | | [MePhosphonate-4O- |
| | | | mUs][fAs][fUs][fA][fA][mU][fA][mC][mA][fA][mA][mA][mU][fU] |
| | | | [mC][mA][mA][mC][mA][mGs][mGs][mG] (SEQ ID NO: 1217) |
| 21 | 2210 | DP17957P: | [mGs][mA][mA][mU][mU][mU][mU][fG][fU][fA][fU][mU][mA] |
| | SM1405/ | DP17956G | [mU][mG][mU][mG][mA][mA][mA][mG][mC][mA][mG][mC][mC] |
| | ASM2028 | | [mG][ademA-GalNAc][ademA-GalNAc][ademA- |
| | | | GalNAc][mG][mG][mC][mU][mG][mC] (SEQ ID NO: 1218) |
| | | | [MePhosphonate-4O- |
| | | | mUs][fUs][fUs][fC][fA][mC][fA][mU][mA][fA][mU][mA][mC][fA] |
| | | | [mA][mA][mA][mU][mU][mCs][mGs][mG] (SEQ ID NO: 1219) |

TABLE B

| SEQ ID | | |
|---|---|---|
| NO. and | | |
| Mod. | | |
| Pattern | DP Number | Modified Oligonucleotide |
| 0644 | DP18056P: | [mCs][mA][mA][mC][mG][mU][mA][fC][fC][fC][fU][mU][mC][mA] |
| SM1405/ | DP18055G | [mU][mU][mG][mA][mU][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1220) |
| | | [MePhosphonate-4O- |
| | | mUs][fAs][fUs][fC][fA][mA][fU][mG][mA][fA][mG][mG][mG][fU] |
| | | [mA][mC][mG][mU][mU][mGs][mGs][mG] (SEQ ID NO: 1221) |
| 0745 | DP18084P: | [mUs][mG][mG][mA][mC][mA][mU][fC][fA][fC][fC][mA][mA][mG] |
| SM1405/ | DP18083G | [mC][mU][mC][mA][mG][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1222) |
| | | [MePhosphonate-4O- |
| | | mUs][fCs][fUs][fG][fA][mG][fC][mU][mU][fG][mG][mU][mG][fA] |
| | | [mU][mG][mU][mC][mC][mAs][mGs][mG] (SEQ ID NO: 1223) |
| 1126 | DP18112P: | [mUs][mG][mA][mA][mA][mG][mA][fC][fA][fA][fA][mG][mG][mU] |
| SM1405/ | 8DP1111G | [mG][mG][mA][mU][mA][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1224) |
| | | [MePhosphonate-4O- |
| | | mUs][fUs][fAs][fU][fC][mC][fA][mC][mC][fU][mU][mU][mG][fU] |
| | | [mC][mU][mU][mU][mC][mAs][mGs][mG] (SEQ ID NO: 1225) |
| 1129 | DP18116P: | [mAs][mA][mG][mA][mC][mA][mA][fA][fG][fG][fU][mG][mG][mA] |
| SM1405/ | DP18115G | [mU][mA][mC][mA][mU][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1226) |
| | | [MePhosphonate-4O- |
| | | mUs][fAs][fUs][fG][fU][mA][fU][mC][mC][fA][mC][mC][mU][fU] |
| | | [mU][mG][mU][mC][mU][mUs][mGs][mG] (SEQ ID NO: 1227) |
| 1130 | DP18118P: | [mAs][mG][mA][mC][mA][mA][mA][fG][fG][fU][fG][mG][mA][mU] |
| SM1405/ | DP18117G | [mA][mC][mA][mU][mG][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1228) |
| | | [MePhosphonate-4O- |
| | | mUs][fCs][fAs][fU][fG][mU][fA][mU][mC][fC][mA][mC][mC][fU] |
| | | [mU][mU][mG][mU][mC][mUs][mGs][mG] (SEQ ID NO: 1229) |
| 1143 | DP18136P: | [mUs][mA][mC][mA][mU][mG][mA][fG][fC][fA][fA][mG][mA][mU] |
| SM1405/ | DP18135G | [mU][mU][mG][mC][mA][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1230) |
| | | [MePhosphonate-4O- |
| | | mUs][fUs][fGs][fC][fA][mA][fA][mU][mC][fU][mU][mG][mC][fU] |
| | | [mC][mA][mU][mG][mU][mAs][mGs][mG] (SEQ ID NO: 1231) |
| 1147 | DP18140P: | [mUs][mG][mA][mG][mC][mA][mA][fG][fA][fU][fU][mU][mG][mC] |
| SM1405/ | DP18139G | [mA][mA][mC][mU][mU][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1232) |
| | | [MePhosphonate-4O- |
| | | mUs][fAs][fAs][fG][fU][mU][fG][mC][mA][fA][mA][mU][mC][fU] |
| | | [mU][mG][mC][mU][mC][mAs][mGs][mG] (SEQ ID NO: 1233) |
| 1152 | DP18150P: | [mAs][mA][mG][mA][mU][mU][mU][fG][fC][fA][fA][mC][mU][mU] |
| SM1405/ | DP18149G | [mG][mC][mU][mA][mC][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1234) |
| | | [MePhosphonate-4O- |
| | | mUs][fGs][fUs][fA][fG][mC][fA][mA][mG][fU][mU][mG][mC][fA] |
| | | [mA][mA][mU][mC][mU][mUs][mGs][mG] (SEQ ID NO: 1235) |
| 1229 | DP18172P: | [mUs][mG][mC][mG][mA][mU][mU][fG][fU][fC][fC][mA][mG][mA] |
| SM1405/ | DP18171G | [mG][mA][mC][mU][mG][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1236) |
| | | [MePhosphonate-4O- |
| | | mUs][fCs][fAs][fG][fU][mC][fU][mC][mU][fG][mG][mA][mC][fA] |
| | | [mA][mU][mC][mG][mC][mAs][mGs][mG] (SEQ ID NO: 1237) |
| 2142 | DP18274P: | [mUs][mU][mU][mU][mC][mA][mC][fC][fU][fA][fA][mC][mU][mA] |
| SM1405/ | DP18273G | [mA][mA][mA][mU][mA][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1238) |
| | | [MePhosphonate-4O- |
| | | mUs][fUs][fAs][fU][fU][mU][fU][mA][mG][fU][mU][mA][mG][fG] |
| | | [mU][mG][mA][mA][mA][mAs][mGs][mG] (SEQ ID NO: 1239) |
| 1698 | DP18208P: | [mUs][mC][mU][mG][mA][mG][mC][fU][fG][fA][fG][mU][mU][mG] |
| SM1405/ | DP18207G | [mG][mU][mU][mU][mU][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1240) |
| | | [MePhosphonate-4O- |
| | | mUs][fAs][fAs][fA][fA][mC][fC][mA][mA][fC][mU][mC][mA][fG] |
| | | [mC][mU][mC][mA][mG][mAs][mGs][mG] (SEQ ID NO: 1241) |
| 2138 | DP18270P: | [mAs][mC][mC][mU][mU][mU][mU][fU][fC][fA][fC][mC][mU][mA] |
| SM1405/ | 8DP1269G | [mA][mC][mU][mA][mA][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1242) |
| | | [MePhosphonate-4O- |
| | | mUs][fUs][fUs][fA][fG][mU][fU][mA][mG][fG][mU][mG][mA][fA] |
| | | [mA][mA][mA][mG][mG][mUs][mGs][mG] (SEQ ID NO: 1243) |
| 2140 | DP18272P: | [mCs][mU][mU][mU][mU][mU][mC][fA][fC][fC][fU][mA][mA][mC] |
| SM1405/ | DP18271G | [mU][mA][mA][mA][mA][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1244) |
| | | [MePhosphonate-4O- |
| | | mUs][fUs][fUs][fU][fU][mA][fG][mU][mU][fA][mG][mG][mU][fG] |
| | | [mA][mA][mA][mA][mA][mGs][mGs][mG] (SEQ ID NO: 1245) |
| 2144 | DP18276P: | [mUs][mU][mC][mA][mC][mC][mU][fA][fA][fC][fU][mA][mA][mA] |
| SM1405/ | DP18275G | [mA][mU][mA][mA][mU][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1246) |
| | | [MePhosphonate-4O- |
| | | mUs][fAs][fUs][fU][fA][mU][fU][mU][mU][fA][mG][mU][mU][fA] |
| | | [mG][mG][mU][mG][mA][mAs][mGs][mG] (SEQ ID NO: 1247) |
| 2146 | DP18280P: | [mCs][mA][mC][mC][mU][mA][mA][fC][fU][fA][fA][mA][mA][mU] |
| SM1405/ | DP18279G | [mA][mA][mU][mG][mU][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1248) |
| | | [MePhosphonate-4O- |
| | | mUs][fAs][fCs][fA][fU][mU][fA][mU][mU][fU][mU][mA][mG][fU] |
| | | [mU][mA][mG][mG][mU][mGs][mGs][mG] (SEQ ID NO: 1249) |
| 2149 | DP18286P: | [mCs][mU][mA][mA][mC][mU][mA][fA][fA][fA][fU][mA][mA][mU] |
| SM1405/ | DP18285G | [mG][mU][mU][mU][mA][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1250) |
| | | [MePhosphonate-4O- |
| | | mUs][fUs][fAs][fA][fA][mC][fA][mU][mU][fA][mU][mU][mU][fU] |
| | | [mA][mG][mU][mU][mA][mGs][mGs][mG] (SEQ ID NO: 1251) |
| 2137 | DP18268P: | [mCs][mA][mC][mC][mU][mU][mU][fU][fU][fC][fA][mC][mC][mU] |
| SM1405/ | 8DP1267G | [mA][mA][mC][mU][mA][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1252) |
| | | [MePhosphonate-4O- |
| | | mUs][fUs][fAs][fG][fU][mU][fA][mG][mG][fU][mG][mA][mA][fA] |
| | | [mA][mA][mG][mG][mU][mGs][mGs][mG] (SEQ ID NO: 1253) |
| 2205 | DP17955P: | [mCs][mU][mG][mU][mU][mG][mA][fA][fU][fU][fU][mU][mG][mU] |
| SM1405/ | DP17954G | [mA][mU][mU][mA][mU][mA][mG][mC][mA][mG][mC][mC][mG] |
| ASM2028 | | [ademA-GalNAc][ademA-GalNAc][ademA- |
| | | GalNAc][mG][mG][mC][mU][mG][mC] (SEQ ID NO: 1216) |
| | | [MePhosphonate-4O- |
| | | mUs][fAs][fUs][fA][fA][mU][fA][mC][mA][fA][mA][mA][mU][fU] |
| | | [mC][mA][mA][mC][mA][mGs][mGs][mG] (SEQ ID NO: 1217) |

TABLE C

| SEQ ID NO. | | |
|---|---|---|
| and Mod. | DP | |
| Pattern | Number | Modified Oligonucleotide |
| 1697 | DP18206P: | [mCs][mU][mC][mU][mG][mA][mG][fC][fU][fG][fA][mG] |
| SM1405/ | DP18205G | [mU][mU][mG][mG][mU][mU][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1254) |
| | | [MePhosphonate-4O- |
| | | mUs][fAs][fAs][fA][fC][mC][fA][mA][mC][fU][mC][mA][mG] |
| | | [fC][mU][mC][mA][mG][mA][mGs][mGs][mG] (SEQ ID NO: |
| | | 1255) |
| 1614 | DP18194P: | [mAs][mG][mG][mU][mG][mC][mU][fA][fA][fA][fG][mU] |
| SM1405/ | DP18193G | [mU][mU][mC][mC][mC][mA][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1256) |
| | | [MePhosphonate-4O- |
| | | mUs][fAs][fUs][fG][fG][mG][fA][mA][mA][fC][mU][mU][mU] |
| | | [fA][mG][mC][mA][mC][mC][mUs][mGs][mG] (SEQ ID NO: |
| | | 1257) |
| 2176 | DP18304P: | [mUs][mG][mU][mA][mU][mA][mA][fA][fA][fA][fU][mG] |
| SM1405/ | DP18303G | [mU][mA][mA][mG][mG][mA][mA][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1258) |
| | | [MePhosphonate-4O- |
| | | mUs][fUs][fUs][fC][fC][mU][fU][mA][mC][fA][mU][mU][mU] |
| | | [fU][mU][mA][mU][mA][mC][mAs][mGs][mG] (SEQ ID NO: |
| | | 1259) |
| 2222 | DP18326P: | [mAs][mU][mG][mU][mG][mA][mA][fU][fC][fA][fG][mU] |
| SM1405/ | DP18325G | [mG][mA][mG][mA][mU][mG][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1260) |
| | | [MePhosphonate-4O- |
| | | mUs][fAs][fCs][fA][fU][mC][fU][mC][mA][fC][mU][mG][mA] |
| | | [fU][mU][mC][mA][mC][mA][mUs][mGs][mG] (SEQ ID NO: |
| | | 1261) |
| 1173 | DP18168P: | [mAs][mU][mU][mA][mG][mG][mA][fU][fA][fA][fU][mG] |
| SM1405/ | DP18167G | [mU][mC][mU][mU][mA][mU][mG][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1262) |
| | | [MePhosphonate-4O- |
| | | mUs][fCs][fAs][fU][fA][mA][fG][mA][mC][fA][mU][mU][mA] |
| | | [fU][mC][mC][mU][mA][mA][mUs][mGs][mG] (SEQ ID NO: |
| | | 1263) |
| 2136 | DP18266P: | [mAs][mC][mA][mC][mC][mU][mU][fU][fU][fU][fC][mA] |
| SM1405/ | DP18265G | [mC][mC][mU][mA][mA][mC][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1264) |
| | | [MePhosphonate-4O- |
| | | mUs][fAs][fGs][fU][fU][mA][fG][mG][mU][fG][mA][mA][mA] |
| | | [fA][mA][mG][mG][mU][mG][mUs][mGs][mG] (SEQ ID NO: |
| | | 1265) |
| 0637 | DP18046P: | [mUs][mG][mA][mG][mU][mG][mA][fC][fA][fA][fC][mG] |
| SM1405/ | DP18045G | [mU][mA][mC][mC][mC][mU][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1266) |
| | | [MePhosphonate-4O- |
| | | mUs][fAs][fAs][fG][fG][mG][fU][mA][mC][fG][mU][mU][mG] |
| | | [fU][mC][mA][mC][mU][mC][mAs][mGs][mG] (SEQ ID NO: |
| | | 1267) |
| 1116 | DP18098P: | [mAs][mG][mU][mG][mA][mA][mG][fA][fA][fA][fU][mG] |
| SM1405/ | DP18097G | [mA][mA][mA][mG][mA][mC][mA][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1268) |
| | | [MePhosphonate-4O- |
| | | mUs][fUs][fGs][fU][fC][mU][fU][mU][mC][fA][mU][mU][mU] |
| | | [fC][mU][mU][mC][mA][mC][mUs][mGs][mG] (SEQ ID NO: |
| | | 1269) |
| 0923 | DP18096P: | [mAs][mU][mC][mC][mU][mC][mA][fG][fA][fA][fG][mG] |
| SM1405/ | DP18095G | [mG][mA][mU][mG][mG][mA][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1270) |
| | | [MePhosphonate-4O- |
| | | mUs][fAs][fUs][fC][fC][mA][fU][mC][mC][fC][mU][mU][mC] |
| | | [fU][mG][mA][mG][mG][mA][mUs][mGs][mG] (SEQ ID NO: |
| | | 1271) |
| 0641 | DP18052P: | [mUs][mG][mA][mC][mA][mA][mC][fG][fU][fA][fC][mC] |
| SM1405/ | DP18051G | [mC][mU][mU][mC][mA][mU][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1272) |
| | | [MePhosphonate-4O- |
| | | mUs][fAs][fAs][fU][fG][mA][fA][mG][mG][fG][mU][mA][mC] |
| | | [fG][mU][mU][mG][mU][mC][mAs][mGs][mG] (SEQ ID NO: |
| | | 1273) |
| 1136 | DP18126P: | [mAs][mG][mG][mU][mG][mG][mA][fU][fA][fC][fA][mU] |
| SM1405/ | DP18125G | [mG][mA][mG][mC][mA][mA][mG][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1274) |
| | | [MePhosphonate-4O- |
| | | mUs][fCs][fUs][fU][fG][mC][fU][mC][mA][fU][mG][mU][mA] |
| | | [fU][mC][mC][mA][mC][mC][mUs][mGs][mG] (SEQ ID NO: |
| | | 1275) |
| 1158 | DP18158P: | [mUs][mG][mC][mA][mA][mC][mU][fU][fG][fC][fU][mA] |
| SM1405/ | DP18157G | [mC][mC][mC][mA][mU][mU][mA][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1276) |
| | | [MePhosphonate-4O- |
| | | mUs][fUs][fAs][fA][fU][mG][fG][mG][mU][fA][mG][mC][mA] |
| | | [fA][mG][mU][mU][mG][mC][mAs][mGs][mG] (SEQ ID NO: |
| | | 1277) |
| 1151 | DP18148P: | [mCs][mA][mA][mG][mA][mU][mU][fU][fG][fC][fA][mA] |
| SM1405/ | DP18147G | [mC][mU][mU][mG][mC][mU][mA][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1278) |
| | | [MePhosphonate-4O- |
| | | mUs][fUs][fAs][fG][fC][mA][fA][mG][mU][fU][mG][mC][mA] |
| | | [fA][mA][mU][mC][mU][mU][mGs][mGs][mG] (SEQ ID NO: |
| | | 1279) |
| 1541 | DP18184P: | [mCs][mA][mC][mC][mU][mU][mU][fC][fC][fC][fA][mG][mU] |
| SM1405/ | DP18183G | [mU][mU][mU][mU][mC][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1280) |
| | | [MePhosphonate-4O- |
| | | mUs][fUs][fGs][fA][fA][mA][fA][mA][mC][fU][mG][mG][mG] |
| | | [fA][mA][mA][mG][mG][mU][mGs][mGs][mG] (SEQ ID NO: |
| | | 1281) |
| 1157 | DP18156P: | [mUs][mU][mG][mC][mA][mA][mC][fU][fU][fG][fC][mU] |
| SM1405/ | DP18155G | [mA][mC][mC][mC][mA][mU][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1282) |
| | | [MePhosphonate-4O- |
| | | mUs][fAs][fAs][fU][fG][mG][fG][mU][mA][fG][mC][mA][mA] |
| | | [fG][mU][mU][mG][mC][mA][mAs][mGs][mG] (SEQ ID NO: |
| | | 1283) |
| 1163 | DP18164P: | [mCs][mU][mU][mG][mC][mU][mA][fC][fC][fC][fA][mU] |
| SM1405/ | DP18163G | [mU][mA][mG][mG][mA][mU][mA][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1284) |
| | | [MePhosphonate-4O- |
| | | mUs][fUs][fAs][fU][fC][mC][fU][mA][mA][fU][mG][mG][mG] |
| | | [fU][mA][mG][mC][mA][mA][mGs][mGs][mG] (SEQ ID NO: |
| | | 1285) |
| 1125 | DP18110P: | [mAs][mU][mG][mA][mA][mA][mG][fA][fC][fA][fA][mA] |
| SM1405/ | DP18109G | [mG][mG][mU][mG][mG][mA][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1286) |
| | | [MePhosphonate-4O- |
| | | mUs][fAs][fUs][fC][fC][mA][fC][mC][mU][fU][mU][mG][mU] |
| | | [fC][mU][mU][mU][mC][mA][mUs][mGs][mG] (SEQ ID NO: |
| | | 1287) |
| 2205 | DP17955P: | [mCs][mU][mG][mU][mU][mG][mA][fA][fU][fU][fU][mU] |
| SM1405/ | DP17954G | [mG][mU][mA][mU][mU][mA][mU][mA][mG][mC][mA][mG] |
| ASM2028 | | [mC][mC][mG][ademA-GalNAc][ademA-GalNAc][ademA- |
| | | GalNAc][mG][mG][mC][mU][mG][mC] (SEQ ID NO: 1216) |
| | | [MePhosphonate-4O- |
| | | mUs][fAs][fUs][fA][fA][mU][fA][mC][mA][fA][mA][mA][mU] |
| | | [fU][mC][mA][mA][mC][mA][mGs][mGs][mG] (SEQ ID NO: |
| | | 1217) |

TABLE D

| SEQ ID | | |
|---|---|---|
| NO. and | | |
| Mod. | DP | |
| Pattern | Number | Modified Oligonucleotide |
| 0639 | DP18050P: | [mAs][mG][mU][mG][mA][mC][mA][fA][fC][fG][fU][mA][mC] |
| SM1405/ | DP18049G | [mC][mC][mU][mU][mC][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1288) |
| | | [MePhosphonate-4O- |
| | | mUs][fUs][fGs][fA][fA][mG][fG][mG][mU][fA][mC][mG][mU] |
| | | [fU][mG][mU][mC][mA][mC][mUs][mGs][mG] (SEQ ID NO: |
| | | 1289) |
| 0644 | DP18056P: | [mCs][mA][mA][mC][mG][mU][mA][fC][fC][fC][fU][mU][mC] |
| SM1405/ | DP18055G | [mA][mU][mU][mG][mA][mU][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1220) |
| | | [MePhosphonate-4O- |
| | | mUs][fAs][fUs][fC][fA][mA][fU][mG][mA][fA][mG][mG][mG] |
| | | [fU][mA][mC][mG][mU][mU][mGs][mGs][mG] (SEQ ID NO: |
| | | 1221) |
| 0729 | DP18068P: | [mAs][mC][mG][mA][mA][mC][mU][fU][fU][fC][fU][mU][mC] |
| SM1405/ | DP18067G | [mA][mU][mG][mU][mG][mG][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1290) |
| | | [MePhosphonate-4O- |
| | | mUs][fCs][fCs][fA][fC][mA][fU][mG][mA][fA][mG][mA][mA] |
| | | [fA][mG][mU][mU][mC][mG][mUs][mGs][mG] (SEQ ID NO: |
| | | 1291) |
| 0735 | DP18074P: | [mUs][mU][mU][mC][mU][mU][mC][fA][fU][fG][fU][mG][mG] |
| SM1405/ | DP18073G | [mA][mC][mA][mU][mC][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1292) |
| | | [MePhosphonate-4O- |
| | | mUs][fUs][fGs][fA][fU][mG][fU][mC][mC][fA][mC][mA][mU] |
| | | [fG][mA][mA][mG][mA][mA][mAs][mGs][mG] (SEQ ID NO: |
| | | 1293) |
| 0743 | DP18080P: | [mUs][mG][mU][mG][mG][mA][mC][fA][fU][fC][fA][mC][mC] |
| SM1405/ | DP18079G | [mA][mA][mG][mC][mU][mC][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1294) |
| | | [MePhosphonate-4O- |
| | | mUs][fGs][fAs][fG][fC][mU][fU][mG][mG][fU][mG][mA][mU] |
| | | [fG][mU][mC][mC][mA][mC][mAs][mGs][mG] (SEQ ID NO: |
| | | 1295) |
| 0744 | DP18082P: | [mGs][mU][mG][mG][mA][mC][mA][fU][fC][fA][fC][mC][mA] |
| SM1405/ | DP18081G | [mA][mG][mC][mU][mC][mA][mA][mG][mC][mA][mG][mC] |
| SM2028A | | [mC][mG][ademA-GalNAc][ademA-GalNAc][ademA- |
| | | GalNAc][mG][mG][mC][mU][mG][mC] (SEQ ID NO: 1296) |
| | | [MePhosphonate-4O- |
| | | mUs][fUs][fGs][fA][fG][mC][fU][mU][mG][fG][mU][mG][mA] |
| | | [fU][mG][mU][mC][mC][mA][mCs][mGs][mG] (SEQ ID NO: |
| | | 1297) |
| 0838 | DP18086P: | [mAs][mG][mA][mU][mA][mU][mG][fC][fC][fU][fU][mC][mG] |
| SM1405/ | DP18085G | [mA][mG][mG][mA][mU][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1298) |
| | | [MePhosphonate-4O- |
| | | mUs][fUs][fAs][fU][fC][mC][fU][mC][mG][fA][mA][mG][mG] |
| | | [fC][mA][mU][mA][mU][mC][mUs][mGs][mG] (SEQ ID NO: |
| | | 1299) |
| 1126 | DP18112P: | [mUs][mG][mA][mA][mA][mG][mA][fC][fA][fA][fA][mG][mG] |
| SM1405/ | DP18111G | [mU][mG][mG][mA][mU][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1224) |
| | | [MePhosphonate-4O- |
| | | mUs][fUs][fAs][fU][fC][mC][fA][mC][mC][fU][mU][mU][mG] |
| | | [fU][mC][mU][mU][mU][mC][mAs][mGs][mG] (SEQ ID NO: |
| | | 1225) |
| 1143 | DP18136P: | [mUs][mA][mC][mA][mU][mG][mA][fG][fC][fA][fA][mG][mA] |
| SM1405/ | DP18135G | [mU][mU][mU][mG][mC][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1230) |
| | | [MePhosphonate-4O- |
| | | mUs][fUs][fGs][fC][fA][mA][fA][mU][mC][fU][mU][mG][mC] |
| | | [fU][mC][mA][mU][mG][mU][mAs][mGs][mG] (SEQ ID NO: |
| | | 1231) |
| 1147 | DP18140P: | [mUs][mG][mA][mG][mC][mA][mA][fG][fA][fU][fU][mU][mG] |
| SM1405/ | DP18139G | [mC][mA][mA][mC][mU][mU][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1232) |
| | | [MePhosphonate-4O- |
| | | mUs][fAs][fAs][fG][fU][mU][fG][mC][mA][fA][mA][mU][mC] |
| | | [fU][mU][mG][mC][mU][mC][mAs][mGs][mG] (SEQ ID NO: |
| | | 1233) |
| 1152 | DP18150P: | [mAs][mA][mG][mA][mU][mU][mU][fG][fC][fA][fA][mC][mU] |
| SM1405/ | DP18149G | [mU][mG][mC][mU][mA][mC][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1234) |
| | | [MePhosphonate-4O- |
| | | mUs][fGs][fUs][fA][fG][mC][fA][mA][mG][fU][mU][mG][mC] |
| | | [fA][mA][mA][mU][mC][mU][mUs][mGs][mG] (SEQ ID NO: |
| | | 1235) |
| 1173 | DP18168P: | [mAs][mU][mU][mA][mG][mG][mA][fU][fA][fA][fU][mG][mU] |
| SM1405/ | DP18167G | [mC][mU][mU][mA][mU][mG][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1262) |
| | | [MePhosphonate-4O- |
| | | mUs][fCs][fAs][fU][fA][mA][fG][mA][mC][fA][mU][mU][mA] |
| | | [fU][mC][mC][mU][mA][mA][mUs][mGs][mG] (SEQ ID NO: |
| | | 1263) |
| 1697 | DP18206P: | [mCs][mU][mC][mU][mG][mA][mG][fC][fU][fG][fA][mG][mU] |
| SM1405/ | DP18205G | [mU][mG][mG][mU][mU][mU][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1254) |
| | | [MePhosphonate-4O- |
| | | mUs][fAs][fAs][fA][fC][mC][fA][mA][mC][fU][mC][mA][mG] |
| | | [fC][mU][mC][mA][mG][mA][mGs][mGs][mG] (SEQ ID NO: |
| | | 1255) |
| 1699 | DP17927P: | [mCs][mU][mG][mA][mG][mC][mU][fG][fA][fG][fU][mU][mG] |
| SM1405/ | DP17926G | [mG][mU][mU][mU][mU][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1188) |
| | | [MePhosphonate-4O- |
| | | mUs][fUs][fAs][fA][fA][mA][fC][mC][mA][fA][mC][mU][mC] |
| | | [fA][mG][mC][mU][mC][mA][mGs][mGs][mG] (SEQ ID NO: |
| | | 1189) |
| 1703 | DP17929P: | [mGs][mC][mU][mG][mA][mG][mU][fU][fG][fG][fU][mU][mU] |
| SM1405/ | DP17928G | [mU][mA][mU][mG][mA][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1190) |
| | | [MePhosphonate-4O- |
| | | mUs][fUs][fUs][fC][fA][mU][fA][mA][mA][fA][mC][mC][mA] |
| | | [fA][mC][mU][mC][mA][mG][mCs][mGs][mG] (SEQ ID NO: |
| | | 1191) |
| 2140 | DP18272P: | [mCs][mU][mU][mU][mU][mU][mC][fA][fC][fC][fU][mA][mA] |
| SM1405/ | DP18271G | [mC][mU][mA][mA][mA][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1244) |
| | | [MePhosphonate-4O- |
| | | mUs][fUs][fUs][fU][fU][mA][fG][mU][mU][fA][mG][mG][mU] |
| | | [fG][mA][mA][mA][mA][mA][mGs][mGs][mG] (SEQ ID NO: |
| | | 1245) |
| 2144 | DP18276P: | [mUs][mU][mC][mA][mC][mC][mU][fA][fA][fC][fU][mA][mA] |
| SM1405/ | DP18275G | [mA][mA][mU][mA][mA][mU][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1246) |
| | | [MePhosphonate-4O- |
| | | mUs][fAs][fUs][fU][fA][mU][fU][mU][mU][fA][mG][mU][mU] |
| | | [fA][mG][mG][mU][mG][mA][mAs][mGs][mG] (SEQ ID NO: |
| | | 1247) |
| 2149 | DP18286P: | [mCs][mU][mA][mA][mC][mU][mA][fA][fA][fA][fU][mA][mA] |
| SM1405/ | DP18285G | [mU][mG][mU][mU][mU][mA][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1250) |
| | | [MePhosphonate-4O- |
| | | mUs][fUs][fAs][fA][fA][mC][fA][mU][mU][fA][mU][mU][mU] |
| | | [fU][mA][mG][mU][mU][mA][mGs][mGs][mG] (SEQ ID NO: |
| | | 1251) |
| 2156 | DP17939P: | [mAs][mA][mA][mU][mA][mA][mU][fG][fU][fU][fU][mA][mA] |
| SM1405/ | DP17938G | [mA][mG][mA][mG][mU][mU][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1200) |
| | | [MePhosphonate-4O- |
| | | mUs][fAs][fAs][fC][fU][mC][fU][mU][mU][fA][mA][mA][mC] |
| | | [fA][mU][mU][mA][mU][mU][mUs][mGs][mG] (SEQ ID NO: |
| | | 1201) |
| 2195 | DP17949P: | [mGs][mC][mG][mU][mU][mG][mU][fU][fA][fC][fC][mU][mG] |
| SM1405/ | DP17948G | [mU][mU][mG][mA][mA][mU][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1300) |
| | | [MePhosphonate-4O- |
| | | mUs][fAs][fUs][fU][fC][mA][fA][mC][mA][fG][mG][mU][mA] |
| | | [fA][mC][mA][mA][mC][mG][mCs][mGs][mG] (SEQ ID NO: |
| | | 1301) |
| 2205 | DP17955P: | [mCs][mU][mG][mU][mU][mG][mA][fA][fU][fU][fU][mU][mG] |
| SM1405/ | DP17954G | [mU][mA][mU][mU][mA][mU][mA][mG][mC][mA][mG][mC] |
| ASM2028 | | [mC][mG][ademA-GalNAc][ademA-GalNAc][ademA- |
| | | GalNAc][mG][mG][mC][mU][mG][mC] (SEQ ID NO: 1216) |
| | | [MePhosphonate-4O- |
| | | mUs][fAs][fUs][fA][fA][mU][fA][mC][mA][fA][mA][mA][mU] |
| | | [fU][mC][mA][mA][mC][mA][mGs][mGs][mG] (SEQ ID NO: |
| | | 1217) |

The invention claimed is:

1. An RNAi oligonucleotide for reducing PNPLA3 expression, the oligonucleotide comprising a sense strand and an antisense strand, wherein the sense strand and the antisense strand form a duplex region, wherein the antisense strand comprises a region of complementarity to a PNPLA3 mRNA target sequence of any one of SEQ ID NOS:1167 to 1176, and wherein the region of complementarity is at least 15 contiguous nucleotides in length.

2. The RNAi oligonucleotide of claim 1, wherein the sense strand is 15 to 50 nucleotides in length.

3. The RNAi oligonucleotide of claims 1 or 2, wherein the sense strand is 18 to 36 nucleotides in length.

4. The RNAi oligonucleotide of any one of claims 1 to 3, wherein the antisense strand is 15 to 30 nucleotides in length.

5. The RNAi oligonucleotide of any one of claims 1 to 4, wherein the antisense strand is 22 nucleotides in length and where in the antisense strand and the sense strand form a duplex region of at least 19 nucleotides in length, optionally at least 20 nucleotides in length.

6. The RNAi oligonucleotide of any one of claims 1 to 5, wherein the region of complementarity is at least 19 contiguous nucleotide in length, optionally at least 20 nucleotides in length.

7. The RNAi oligonucleotide of any one of claims 1 to 6, wherein the 3′ end of the sense strand comprises a stem-loop set forth as S1-L-S2, wherein S1 is complementarity to S2, and wherein L forms a loop between S1 and S2 of 3 to 5 nucleotides in length.

8. An RNAi oligonucleotide for reducing PNPLA3 expression, the oligonucleotide comprising a sense strand of 15 to 50 nucleotides in length and an antisense strand, wherein the sense strand and the antisense strand form a duplex region, wherein the wherein the antisense strand comprises a region of complementarity to a PNPLA3 mRNA target sequence of any one of SEQ ID NOS:1167 to 1176, and wherein the region of complementarity is at least 15 contiguous nucleotides in length.

9. An RNAi oligonucleotide for reducing PNPLA3 expression, the oligonucleotide comprising a sense strand of 15 to 50 nucleotides in length and an antisense strand of 15 to 30 nucleotides in length, wherein the sense strand and the antisense strand form a duplex region, wherein the antisense strand comprises a region of complementarity to a PNPLA3 mRNA target sequence of any one of SEQ ID NOS:1167 to 1176, and wherein the region of complementarity is at least 15 contiguous nucleotides in length.

10. An RNAi oligonucleotide for reducing PNPLA3 expression, the oligonucleotide comprising a sense strand of 15 to 50 nucleotides in length and an antisense strand, wherein the sense strand and the antisense strand form a duplex region, wherein the antisense strand comprises a region of complementarity to a PNPLA3 mRNA target sequence of any one of SEQ ID NOS:1167 to 1176, and wherein the region of complementarity is 19 contiguous nucleotides in length, optionally 20 nucleotides in length.

11. An RNAi oligonucleotide for reducing PNPLA3 expression, the oligonucleotide comprising a sense strand of 18 to 36 nucleotides in length and an antisense strand, wherein the sense strand and the antisense strand form a duplex region, wherein the antisense strand comprises a region of complementarity to a PNPLA3 mRNA target sequence of any one of SEQ ID NOS:1167 to 1176, and wherein the region of complementarity is 19 contiguous nucleotides in length, optionally 20 nucleotides in length.

12. An RNAi oligonucleotide for reducing PNPLA3 expression, the oligonucleotide comprising a sense strand of 18 to 36 nucleotides in length and an antisense strand of 22 nucleotides in length, wherein the sense strand and the antisense strand form a duplex region, wherein the antisense strand comprises a region of complementarity to a PNPLA3 mRNA target sequence of any one of SEQ ID NOS:1167 to 1176, and wherein the region of complementarity is 19 contiguous nucleotides in length, optionally 20 nucleotides in length.

13. An RNAi oligonucleotide for reducing PNPLA3 expression, the oligonucleotide comprising a sense strand of 18 to 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3 to 5 nucleotides in length, wherein the antisense strand comprises a region of complementarity to a PNPLA3 mRNA target sequence of any one of SEQ ID NOS:1167 to 1176, and wherein the region of complementarity is 19 contiguous nucleotides in length, optionally 20 nucleotides in length.

14. An RNAi oligonucleotide for reducing PNPLA3 expression, the oligonucleotide comprising a sense strand of 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3 to 5 nucleotides in length, wherein the antisense strand comprises a region of complementarity to a PNPLA3 mRNA target sequence of any one of SEQ ID NOS:1167 to 1176, and wherein the region of complementarity is 19 contiguous nucleotides in length, optionally 20 nucleotides in length.

15. An RNAi oligonucleotide for reducing PNPLA3 expression, the oligonucleotide comprising a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3 to 5 nucleotides in length, wherein the antisense strand comprises a region of complementarity to a PNPLA3 mRNA target sequence of any one of SEQ ID NOS:1167 to 1176, and wherein the region of complementarity is 19 contiguous nucleotides in length, optionally 20 nucleotides in length.

16. The RNAi oligonucleotide of any one of claims 7 and 13 to 15, wherein L is a triloop or a tetraloop.

17. The RNAi oligonucleotide of claim 16, wherein L is a tetraloop.

18. The RNAi oligonucleotide of claim 17, wherein the tetraloop comprises the sequence 5′-GAAA-3′.

19. The RNAi oligonucleotide of any one of claims 16 to 18, wherein S1 and S2 are 1-10 nucleotides in length and have the same length.

20. The RNAi oligonucleotide of claim 19, wherein S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length.

21. The RNAi oligonucleotide of claim 20, wherein S1 and S2 are 6 nucleotides in length.

22. The RNAi oligonucleotide of any one of claims 16 to 21, wherein the stem-loop comprises the sequence 5′-GCAGCCGAAAGGCUGC-3′ (SEQ ID NO:1177).

23. The RNAi oligonucleotide of any one of claims 1 to 22, wherein the antisense strand comprises a 3′ overhang sequence of one or more nucleotides in length.

24. The RNAi oligonucleotide of claim 23, wherein the 3′ overhang sequence is 2 nucleotides in length, optionally wherein the 3′ overhang sequence is GG.

25. The RNAi oligonucleotide of any one of the preceding claims, wherein the oligonucleotide comprises at least one modified nucleotide.

26. The RNAi oligonucleotide of claim 25, wherein the modified nucleotide comprises a 2′-modification.

27. The RNAi oligonucleotide of claim 26, wherein the 2′-modification is a modification selected from the group consisting of 2′-aminoethyl, 2′-fluoro, 2′-O-methyl, 2′-O-methoxyethyl, and 2′-deoxy-2′-fluoro-β-d-arabinonucleic acid.

28. The RNAi oligonucleotide of any one of claims 25 to 27, wherein all nucleotides comprising the oligonucleotide are modified, optionally wherein the modification is a 2′-modification selected from the group consisting of 2′-fluoro and 2′-O-methyl.

29. The RNAi oligonucleotide of any one of the preceding claims, wherein the oligonucleotide comprises at least one modified internucleotide linkage.

30. The RNAi oligonucleotide of claim 29, wherein the at least one modified internucleotide linkage is a phosphorothioate linkage.

31. The RNAi oligonucleotide of any one of the preceding claims, wherein the 4′-carbon of the sugar of the 5′-nucleotide of the antisense strand comprises a phosphate analog.

32. The RNAi oligonucleotide of claim 31, wherein the phosphate analog is oxymethylphosphonate, vinylphosphonate or malonylphosphonate, optionally wherein the phosphate analog is a 4′-phosphate analog comprising 5′-methoxyphosphonate-4′-oxy.

33. The RNAi oligonucleotide of any one of the preceding claims, wherein at least one nucleotide of the oligonucleotide is conjugated to one or more targeting ligands.

34. The RNAi oligonucleotide of claim 33, wherein each targeting ligand comprises a carbohydrate, amino sugar, cholesterol, polypeptide, or lipid.

35. The RNAi oligonucleotide of claim 33, wherein each targeting ligand comprises a N-acetylgalactosamine (GalNAc) moiety.

36. The RNAi oligonucleotide of claim 35, wherein the GalNAc moiety is a monovalent GalNAc moiety, a bivalent GalNAc moiety, a trivalent GalNAc moiety, or a tetravalent GalNAc moiety.

37. The RNAi oligonucleotide of any one of claims 16 to 32, wherein up to 4 nucleotides of L of the stem-loop are each conjugated to a monovalent GalNAc moiety.

38. The RNAi oligonucleotide of any one of claims 1 to 37, wherein the sense strand comprises a nucleotide sequence of any one of SEQ ID NOs: 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, and 775, or
wherein the sense strand comprises a nucleotide sequence of any one of SEQ ID NOs:777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, and 1163 or wherein the sense strand comprises a nucleotide sequence of any one of SEQ ID NOs: 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298 or 1300.

39. The RNAi oligonucleotide of any one of claims 1 to 38, wherein the antisense strand comprises a nucleotide sequence of any one of SEQ ID NOs:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, and 776, or
wherein the antisense strand comprises a nucleotide sequence of any one of SEQ ID NOs:778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, and 1164;
or wherein the antisense strand comprises a nucleotide sequence of any one of SEQ ID Nos: 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299 or 1301.

40. The RNAi oligonucleotide of any one of claims 1 to 39, wherein the sense strand and antisense strands comprise nucleotide sequences selected from the group consisting of:
(a) SEQ ID NOS:787 and 788,
(b) SEQ ID NOS:843 and 844,
(c) SEQ ID NOS:867 and 868,
(d) SEQ ID NOS:871 and 872,
(e) SEQ ID NOS:937 and 938,
(f) SEQ ID NOS:1003 and 1004,
(g) SEQ ID NOS:1007 and 1008,
(h) SEQ ID NOS:1017 and 1018,
(i) SEQ ID NOS:1161 and 1162,
(j) SEQ ID NOS: 1163 and 1164,
(k) SEQ ID NOs: 1220 and 1221,
(l) SEQ ID NOs: 1224 and 1225,
(m) SEQ ID NOs: 1230 and 1231,
(n) SEQ ID NOs: 1232 and 1233,
(o) SEQ ID NOs: 1188 and 1189,
(p) SEQ ID NOs: 1190 and 1191,
(q) SEQ ID NOs: 1244 and 1245,
(r) SEQ ID NOs: 1250 and 1251-,
(s) SEQ ID NOs: 1254 and 1255, and
(t) SEQ ID NOs: 1246 and 1247.

41. The RNAi oligonucleotide of any one of claims 1 to 39, wherein the sense strand comprises a nucleotide sequence as set forth in SEQ ID NO: 1220 and wherein the antisense strand comprises a nucleotide sequence as set forth in SEQ ID NO:1221.

42. The RNAi oligonucleotide of any one of claims 1 to 39, wherein the sense strand comprises a nucleotide sequence as set forth in SEQ ID NO:1224 and wherein the antisense strand comprises a nucleotide sequence as set forth in SEQ ID NO:1225.

43. The RNAi oligonucleotide of any one of claims 1 to 39, wherein the sense strand comprises a nucleotide sequence as set forth in SEQ ID NO: 1230, and wherein the antisense strand comprises a nucleotide sequence as set forth in SEQ ID NO:1231.

44. The RNAi oligonucleotide of any one of claims 1 to 39, wherein the sense strand comprises a nucleotide sequence as set forth in SEQ ID NO: 1232, and wherein the antisense strand comprises a nucleotide sequence as set forth in SEQ ID NO: 1233.

45. The RNAi oligonucleotide of any one of claims 1 to 39, wherein the sense strand comprises a nucleotide sequence as set forth in SEQ ID NO:1188, and wherein the antisense strand comprises a nucleotide sequence as set forth in SEQ ID NO: 1189.

46. The RNAi oligonucleotide of any one of claims 1 to 39, wherein the sense strand comprises a nucleotide sequence as set forth in SEQ ID NO: 1190, and wherein the antisense strand comprises a nucleotide sequence as set forth in SEQ ID NO:1191.

47. The RNAi oligonucleotide of any one of claims 1 to 39, wherein the sense strand comprises a nucleotide sequence as set forth in SEQ ID NO: 1244, and wherein the antisense strand comprises a nucleotide sequence as set forth in SEQ ID NO: 1245.

48. The RNAi oligonucleotide of any one of claims 1 to 39, wherein the sense strand comprises a nucleotide sequence as set forth in SEQ ID NO: 1250, and wherein the antisense strand comprises a nucleotide sequence as set forth in SEQ ID NO:1251.

49. The RNAi oligonucleotide of any one of claims 1 to 39, wherein the sense strand comprises a nucleotide sequence as set forth in SEQ ID NO: 1254, and wherein the antisense strand comprises a nucleotide sequence as set forth in SEQ ID NO: 1255.

50. The RNAi oligonucleotide of any one of claims 1 to 39, wherein the sense strand comprises a nucleotide sequence as set forth in SEQ ID NO: 1246, and wherein the antisense strand comprises a nucleotide sequence as set forth in SEQ ID NO: 1247.

51. An RNAi oligonucleotide for reducing PNPLA3 expression, the oligonucleotide comprising a sense strand and an antisense strand, wherein the sense strand and the antisense strand form a duplex region, wherein all nucleotides comprising the sense strand and antisense strand are modified, wherein the antisense strand comprises a region of complementarity to a PNPLA3 mRNA target sequence of any one of SEQ ID NOS:1167 to 1176, and wherein the region of complementarity is at least 15 contiguous nucleotides in length.

52. An RNAi oligonucleotide for reducing PNPLA3 expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a PNPLA3 mRNA target sequence of any one of SEQ ID NOS:1167 to 1176, and wherein the region of complementarity is at least 15 contiguous nucleotides in length.

53. An RNAi oligonucleotide for reducing PNPLA3 expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a PNPLA3 mRNA target sequence of any one of SEQ ID NOS:1167 to 1176, and wherein the region of complementarity is at least 15 contiguous nucleotides in length.

54. An RNAi oligonucleotide for reducing PNPLA3 expression, the oligonucleotide comprising a sense strand and an antisense strand, wherein the sense strand and the antisense strand form a duplex region, wherein all nucleotides comprising the sense strand and the antisense strand are modified, wherein the antisense strand and the sense strand comprise one or more 2′-fluoro and 2′-O-methyl modified nucleotides and at least one phosphorothioate linkage, wherein the 4′-carbon of the sugar of the 5′-nucleotide of the antisense strand comprises a phosphate analog, wherein the antisense strand comprises a region of complementarity to a PNPLA3 mRNA target sequence of any one of SEQ ID NOs:1167 to 1176, and wherein the region of complementarity is at least 15 contiguous nucleotides in length.

55. The RNAi oligonucleotide of any one of claims 51 to 54, wherein the sense strand comprises of any one of SEQ ID NOs:787, 843, 867, 871, 937, 1003, 1007, 1017, 1161, or 1163, or wherein the sense strand comprises of any one of SEQ ID Nos: 1188, 1190, 1220, 1224, 1230, 1232, 1244, 1246, 1250 or 1254.

56. The RNAi oligonucleotide of any one of claims 51 to 55, wherein the antisense strand comprises of any one of SEQ ID NOs: 788, 844, 868, 872, 938, 1004, 1008, 1018, 1162, or 1164, or wherein the antisense strand comprises of any one of SEQ ID NOs: 1189, 1191, 1221, 1225, 1231, 1233, 1245, 1247, 1251 or 1255.

57. The RNAi oligonucleotide of any one of claims 51 to 56, wherein the sense strand and antisense strands are selected from the group consisting of:
(a) SEQ ID NOS:787 and 788,
(b) SEQ ID NOS:843 and 844,
(c) SEQ ID NOS:867 and 868,
(d) SEQ ID NOS:871 and 872,
(e) SEQ ID NOS:937 and 938,
(f) SEQ ID NOS:1003 and 1004,
(g) SEQ ID NOS:1007 and 1008,
(h) SEQ ID NOS:1017 and 1018,
(i) SEQ ID NOS:1161 and 1162, and
(j) SEQ ID NOS: 1163 and 1164,
(k) SEQ ID NOs: 1220 and 1221,
(l) SEQ ID NOs: 1224 and 1225,
(m) SEQ ID NOs: 1230 and 1231,
(n) SEQ ID NOs: 1232 and 1233,
(o) SEQ ID NOs: 1188 and 1189,
(p) SEQ ID NOs: 1190 and 1191,
(q) SEQ ID NOs: 1244 and 1245,
(r) SEQ ID NOs: 1250 and 1251-,
(s) SEQ ID NOs: 1254 and 1255, and
(t) SEQ ID NOs: 1246 and 1247.

58. The RNAi oligonucleotide of any one of claims 51 to 56, wherein the sense strand comprises SEQ ID NO: 1220 and wherein the antisense strand comprises SEQ ID NO:1221.

59. The RNAi oligonucleotide of any one of claims 51 to 56, wherein the sense strand comprises SEQ ID NO:1224, and wherein the antisense strand comprises SEQ ID NO:1225.

60. The RNAi oligonucleotide of any one of claims 51 to 56, wherein the sense strand comprises SEQ ID NO:1230, and wherein the antisense strand comprises SEQ ID NO:1231.

61. The RNAi oligonucleotide of any one of claims 51 to 56, wherein the sense strand comprises SEQ ID NO:1232, and wherein the antisense strand comprises SEQ ID NO:1233.

62. The RNAi oligonucleotide of any one of claims 51 to 56, wherein the sense strand comprises SEQ ID NO:1188, and wherein the antisense strand comprises SEQ ID NO:1189.

63. The RNAi oligonucleotide of any one of claims 51 to 56, wherein the sense strand comprises SEQ ID NO:1190, and wherein the antisense strand comprises SEQ ID NO:1191.

64. The RNAi oligonucleotide of any one of claims 51 to 56, wherein the sense strand comprises SEQ ID NO:1244, and wherein the antisense strand comprises SEQ ID NO:1245.

65. The RNAi oligonucleotide of any one of claims 51 to 56, wherein the sense strand comprises SEQ ID NO:1250, and wherein the antisense strand comprises SEQ ID NO:1251.

66. The RNAi oligonucleotide of any one of claims 51 to 56, wherein the sense strand comprises SEQ ID NO:1254, and wherein the antisense strand comprises SEQ ID NO:1255.

67. The RNAi oligonucleotide of any one of claims 51 to 56, wherein the sense strand comprises SEQ ID NO:1246, and wherein the antisense strand comprises SEQ ID NO:1247.

68. A method for treating a subject having a disease, disorder or condition associated with PNPLA3 expression, the method comprising administering to the subject a therapeutically effective amount of the RNAi oligonucleotide of any one of the preceding claims, or pharmaceutical composition thereof, thereby treating the subject.

69. A pharmaceutical composition comprising the RNAi oligonucleotide of any one of claims 1 to 67, and a pharmaceutically acceptable carrier, delivery agent or excipient.

70. A method of delivering an oligonucleotide to a subject, the method comprising administering pharmaceutical composition of claim 69 to the subject.

71. A method for reducing PNPLA3 expression in a cell, a population of cells or a subject, the method comprising the step of:
i. contacting the cell or the population of cells with the RNAi oligonucleotide of any one of claims 1 to 67, or the pharmaceutical composition of claim 69; or
ii. administering to the subject the RNAi oligonucleotide of any one of claims 1 to 67, or the pharmaceutical composition of claim 69.

72. The method of claim 71, wherein reducing PNPLA3 expression comprises reducing an amount or level of PNPLA3 mRNA, an amount or level of PNPLA3 protein, or both.

73. The method of claim 71 or 72, wherein the subject has a disease, disorder or condition associated with PNPLA3 expression.

74. The method of claim 73, wherein the disease, disorder or condition associated with PNPLA3 expression is cardiometabolic disease, alcoholic hepatitis (AH), alcoholic liver disease (ALD), cirrhosis, hepatocellular carcinoma (HCC), non-alcoholic fatty liver disease (NAFLD), or non-alcoholic steatohepatitis (NASH).

75. The method of any one of claims 68 and 70 to 74, wherein the RNAi oligonucleotide, or pharmaceutical composition, is administered in combination with a second composition or therapeutic agent.

76. A method for treating a subject having a disease, disorder or condition associated with PNPLA3 expression, the method comprising administering to the subject a therapeutically effective amount of an RNAi oligonucleotide comprising a sense strand and an antisense strand, wherein the sense strand and the antisense strand form a duplex region, wherein the antisense strand comprises a region of complementarity to a PNPLA3 mRNA target sequence of any one of SEQ ID NOS:1167 to 1176, and wherein the region of complementarity is at least 15 contiguous nucleotides in length.

77. A method for treating a subject having a disease, disorder or condition associated with PNPLA3 expression, the method comprising administering to the subject a therapeutically effective amount of an RNAi oligonucleotide comprising a sense strand and an antisense strand selected from a row set forth in Table 1, Table 3, Table A, Table B, Table C or Table D or a pharmaceutical composition thereof, thereby treating the subject.

78. A method for treating a subject having a disease, disorder or condition associated with PNPLA3 expression, the method comprising administering to the subject a therapeutically effective amount of an RNAi oligonucleotide comprising a sense strand and an antisense strand, wherein the sense strand and antisense strands comprise nucleotide sequences selected from the group consisting of:
(a) SEQ ID NOS:787 and 788,
(b) SEQ ID NOS:843 and 844,
(c) SEQ ID NOS:867 and 868,
(d) SEQ ID NOS:871 and 872,
(e) SEQ ID NOS:937 and 938,
(f) SEQ ID NOS:1003 and 1004,
(g) SEQ ID NOS:1007 and 1008,
(h) SEQ ID NOS:1017 and 1018,
(i) SEQ ID NOS:1161 and 1162, and
(j) SEQ ID NOS: 1163 and 1164,
(k) SEQ ID NOs: 1220 and 1221,
(l) SEQ ID NOs: 1224 and 1225,
(m) SEQ ID NOs: 1230 and 1231,
(n) SEQ ID NOs: 1232 and 1233,
(o) SEQ ID NOs: 1188 and 1189,
(p) SEQ ID NOs: 1190 and 1191,
(q) SEQ ID NOs: 1244 and 1245,
(r) SEQ ID NOs: 1250 and 1251-,
(s) SEQ ID NOs: 1254 and 1255, and
(t) SEQ ID NOs: 1246 and 1247.

79. A method for treating a subject having a disease, disorder or condition associated with PNPLA3 expression, the method comprising administering to the subject a therapeutically effective amount of an RNAi oligonucleotide comprising a sense strand and an antisense strand, wherein the sense strand and antisense strands are selected from the group consisting of:
(a) SEQ ID NOS:787 and 788,
(b) SEQ ID NOS:843 and 844,
(c) SEQ ID NOS:867 and 868,
(d) SEQ ID NOS:871 and 872,
(e) SEQ ID NOS:937 and 938,
(f) SEQ ID NOS:1003 and 1004,
(g) SEQ ID NOS:1007 and 1008,
(h) SEQ ID NOS:1017 and 1018,
(i) SEQ ID NOS:1161 and 1162, and
(j) SEQ ID NOS: 1163 and 1164,
(k) SEQ ID NOs: 1220 and 1221,
(l) SEQ ID NOs: 1224 and 1225,
(m) SEQ ID NOs: 1230 and 1231,
(n) SEQ ID NOs: 1232 and 1233,
(o) SEQ ID NOs: 1188 and 1189,
(p) SEQ ID NOs: 1190 and 1191,
(q) SEQ ID NOs: 1244 and 1245,
(r) SEQ ID NOs: 1250 and 1251-,
(s) SEQ ID NOs: 1254 and 1255, and
(t) SEQ ID NOs: 1246 and 1247.

80. The method of claim 79, wherein the sense strand comprises SEQ ID NO:1224, and wherein the antisense strand comprises SEQ ID NO: 1225; or wherein the sense strand comprises a nucleotide sequence as set forth in SEQ ID NO: 1220 and wherein the antisense strand comprises a nucleotide sequence as set forth in SEQ ID NO: 1221.

81. The method of claim 79, wherein the sense strand comprises SEQ ID NO: 1230, and wherein the antisense strand comprises SEQ ID NO: 1231.

82. The method of claim 79, wherein the sense strand comprises SEQ ID NO: 1232, and wherein the antisense strand comprises SEQ ID NO: 1233.

83. The method of claim 79, wherein the sense strand comprises SEQ ID NO: 1188, and wherein the antisense strand comprises SEQ ID NO: 1189.

84. The method of claim 79, wherein the sense strand comprises SEQ ID NO: 1190, and wherein the antisense strand comprises SEQ ID NO: 1191.

85. The method of claim 79, wherein the sense strand comprises SEQ ID NO: 1244, and wherein the antisense strand comprises SEQ ID NO: 1245.

86. The method of claim 79, wherein the sense strand comprises SEQ ID NO:1250, and wherein the antisense strand comprises SEQ ID NO:1251.

87. The method of claim 79, wherein the sense strand comprises SEQ ID NO:1254, and wherein the antisense strand comprises SEQ ID NO:1255.

88. The method of claim 79, wherein the sense strand comprises SEQ ID NO:1246, and wherein the antisense strand comprises SEQ ID NO:1247.

89. The method of any one of claims 76 to 88, wherein the disease, disorder or condition associated with PNPLA3 expression is selected from the group consisting of cardiometabolic disease, alcoholic hepatitis (AH), alcoholic liver disease (ALD), cirrhosis, hepatocellular carcinoma (HCC), non-alcoholic fatty liver disease (NAFLD), and non-alcoholic steatohepatitis (NASH).

90. Use of the RNAi oligonucleotide of any one of claims 1 to 67, or the pharmaceutical composition of claim 69, in the manufacture of a medicament for the treatment of a disease, disorder or condition associated with PNPLA3 expression, optionally for the treatment of cardiometabolic disease, alcoholic hepatitis (AH), alcoholic liver disease (ALD), cirrhosis, hepatocellular carcinoma (HCC), non-alcoholic fatty liver disease (NAFLD), or non-alcoholic steatohepatitis (NASH).

91. The RNAi oligonucleotide of any one of claims 1 to 67, or the pharmaceutical composition of claim 69, for use, or adaptable for use, in the treatment of a disease, disorder or condition associated with PNPLA3 expression, optionally for the treatment of cardiometabolic disease, alcoholic hepatitis (AH), CCA, PSC, alcoholic liver disease (ALD), cirrhosis, hepatocellular carcinoma (HCC), non-alcoholic fatty liver disease (NAFLD), or non-alcoholic steatohepatitis (NASH).

92. A kit comprising the RNAi oligonucleotide of any one of claims 1 to 67, an optional pharmaceutically acceptable carrier, and a package insert comprising instructions for administration to a subject having a disease, disorder or condition associated with PNPLA3 expression.

93. The use of claim 90, the RNAi oligonucleotide or pharmaceutical composition for use, or adaptable for use, of claim 91, or the kit of claim 92, wherein the disease, disorder or condition associated with PNPLA3 expression is cardiometabolic disease, alcoholic hepatitis (AH), alcoholic liver disease (ALD), cirrhosis, hepatocellular carcinoma (HCC), non-alcoholic fatty liver disease (NAFLD), or non-alcoholic steatohepatitis (NASH).
